SEQUENCE LISTING (132 sequences)

SEQ ID NO: 1 - DNA, 966 bp, Escherichia coli
atgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaataa

SEQ ID NO: 2 - protein, 321 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 3 - protein, 13 aa, Artificial sequence
  Synthetic BirA biotin ligase substrate domain
LXXIXXXXKXXXX

SEQ ID NO: 4 - protein, 15 aa, Artificial sequence
  Synthetic BirA biotin ligase substrate domain (Avi-tag)
GLNDIFEAQKIEWHE

SEQ ID NO: 5 - protein, 325 aa, Bacillus subtilis
MRSTLRKDLIELFSQAGNEFISGQKISDALGCSRTAVWKHIEELRKEGYEVEAVRRKGYRLIKKPGKLSESEIRFGLKTEVMGQHLIYHDVLSSTQKTAHELANNNAPEGTLVVADKQTAGRGRMSRVWHSQEGNGVWMSLILRPDIPLQKTPQLTLLAAVAVVQGIEEAAGIQTDIKWPNDILINGKKTVGILTEMQAEEDRVRSVIIGIGINVNQQPNDFPDELKDIATSLSQAAGEKIDRAGVIQHILLCFEKRYRDYMTHGFTPIKLLWESYALGIGTNMRARTLNGTFYGKALGIDDEGVLLLETNEGIKKIYSADIELG

SEQ ID NO: 6 - protein, 15 aa, Bacillus subtilis
TVVCIVEAMKLFIEI

SEQ ID NO: 7 - protein, 237 aa, Methanococcus jannaschii
MEIIHLSEIDSTNDYAKELAKEGKRNFIVLADKQNNGKGRWGRVWYSDEGGLYFSMVLDSKLYNPKVINLLVPICIIEVLKNYVDKELGLKFPNDIMVKVNDNYKKLGGILTELTDDYMIIGIGINVNNQIRNEIREIAISLKEITGKELDKVEILSNFLKTFESYLEKLKNKEIDDYEILKKYKKYSITIGKQVKILLSNNEIITGKVYDIDFDGIVLGTEKGIERIPSGICIHVR

SEQ ID NO: 8 - protein, 15 aa, Methanococcus jannaschii
DVIVVLEAMKMEHPI

SEQ ID NO: 9 - protein, 690 aa, Saccharomyces cerevisiae
MNVLVYNGPGTTPGSVKHAVESLRDFLEPYYAVSTVNVKVLQTEPWMSKTSAVVFPGGADLPYVQACQPIISRLKHFVSKQGGVFIGFCAGGYFGTSRVEFAQGDPTMEVSGSRDLRFFPGTSRGPAYNGFQYNSEAGARAVKLNLPDGSQFSTYFNGGAVFVDADKFDNVEILATYAEHPDVPSSDSGKGQSENPAAVVLCTVGRGKVLLTGPHPEFNVRFMRKSTDKHFLETVVENLKAQEIMRLKFMRTVLTKTGLNCNNDFNYVRAPNLTPLFMASAPNKRNYLQEMENNLAHHGMHANNVELCSELNAETDSFQFYRGYRASYDAASSSLLHKEPDEVPKTVIFPGVDEDIPPFQYTPNFDMKEYFKYLNVQNTIGSLLLYGEVVTSTSTILNNNKSLLSSIPESTLLHVGTIQVSGRGRGGNTWINPKGVCASTAVVTMPLQSPVTNRNISVVFVQYLSMLAYCKAILSYAPGFSDIPVRIKWPNDLYALSPTYYKRKNLKLVNTGFEHTKLPLGDIEPAYLKISGLLVNTHFINNKYCLLLGCGINLTSDGPTTSLQTWIDILNEERQQLHLDLLPAIKAEKLQALYMNNLEVILKQFINYGAAEILPSYYELWLHSNQIVTLPDHGNTQAMITGITEDYGLLIAKELVSGSSTQFTGNVYNLQPDGNTFDIFKSLIAKKVQS

SEQ ID NO: 10 - protein, 15 aa, Saccharomyces cerevisiae
QPVAVLSAMKMEMII

SEQ ID NO: 11 - DNA, 45 bp, Artificial sequence
  Synthetic S. cerevisiae specific biotin ligase substrate domain encoding oligonucleotide
acgactaattgggttgctcaggctttcaagatgacgtttgatccg

SEQ ID NO: 12 - protein, 15 aa, Artificial sequence
  Synthetic S. cerevisiae specific biotin ligase substrate domain
TTNWVAQAFKMTFDP

SEQ ID NO: 13 - protein, 15 aa, Saccharomyces cerevisiae
DTLCIVEAMKMMNQI

SEQ ID NO: 14 - protein, 665 aa, Candida albicans
MNVLVYSGPGTTTEGVKHCLETLRLHLGSYYAVLPVNETVLLNEPWMRKTSLLVIPGGADLPYCNVLDGNGTRKISKYVKQGGKFLGLCAGGYFGSARCEFEVGNPTMEVTGPRELGFFPGTAKGCAFKGFKYESRTGARAVKLSVNTAALPGCASHIYNYYDGGAVFANAEKYKDVEILARYDDKTDIVDLEKAAVVYRKVGKGGVILSGTHPEFAPHLLHPRDEDGAGYFIVVDTLRAYDHNKKVFMRDCLKKLGLRVAESVDTTIPRVTPMYVVSPFKDKVRDVYSILTSKLGKSFEDSNDAFYFADETQETSEYVGSEEDPVKYINFLTSAGIPDLKMVPYFDIQKYFDNLRMLSGGDIKFGSILGYSEVITSTNTIMDKNPQWLEHLPNGFTITATTQIAGRGRGGNVWVNPRGVLATSVLFKIPPSPSSSSTVVTLQYLCGLALIESILGYGSNVSGQGVGYEDMPLRLKWPNDIFIMKPEYFKSLDDKSDISATVDGDDEKFVKVSGALINSQFINKTFYLVWGGGVNVSNPAPTTSLNLVLEKLNEIRRGKGLSPLPPYEPEILLAKLMFTIDQFYSVFEKSGLQPFLPLYYKRWFHTNQKVDVDNGSGKQRTCIIKGITPDYGLLIAEDVETKKVLHLQPDGNSFDIFKGLVYKKN

SEQ ID NO: 15 - protein, 329 aa, Arabidopsis thaliana
MDIDASCSLVLYGKSSVETDTATRLKNNNVLKLPDNSKVSIFLQSEIKNLVRDDDSSFNLSLFMNSISTHRFGRFLIWSPYLSSTHDVVSHNFSEIPVGSVCVSDIQLKGRGRTKNVWESPKGCLMYSFTLEMEDGRVVPLIQYVVSLAVTEAVKDVCDKKGLSYNDVKIKWPNDLYLNGLKIGGILCTSTYRSRKFLVSVGVGLNVDNEQPTTCLNAVLKDVCPPSNLLKREEILGAFFKKFENFFDLFMEQGFKSLEELYYRTWLHSGQRVIAEEKNEDQVVQNVVTIQGLTSSGYLLAIGDDNVMYELHPDGNSFDFFKGLVRRKL

SEQ ID NO: 16 - protein, 367 aa, Arabidopsis thaliana
MEAVRSTTTLSNFHLLNILVLRSLKPLHRLSFSFSASAMESDASCSLVLCGKSSVETEVAKGLKNKNSLKLPDNTKVSLILESEAKNLVKDDDNSFNLSLFMNSIITHRFGRFLIWSPRLSSTHDVVSHNFSELPVGSVCVTDIQFKGRGRTKNVWESPKGCLMYSFTLEMEDGRVVPLIQYVVSLAVTEAVKDVCDKKGLPYIDVKIKWPNDLYVNGLKVGGILCTSTYRSKKFNVSVGVGLNVDNGQPTTCLNAVLKGMAPESNLLKREEILGAFFHKFEKFFDLFMDQGFKSLEELYYRTWLHSEQRVIVEDKVEDQVVQNVVTIQGLTSSGYLLAVGDDNQMYELHPDGNSFDFFKGLVRRKI

SEQ ID NO: 17 - protein, 722 aa, Mus musculus
MEDRLQMDNGLIAQKIVSVHLKDPALKELGKASDKQVQGPPPGPEASPEAQPAQGVMEHAGQGDCKAAGEGPSPRRRGCAPESEPAADGDPGLSSPELCQLHLSICHECLELENSTIDSVRSASAENIPDLPCDHSGVEGAAGELCPERKGKRVNISGKAPNILLYVGSGSEEALGRLQQVRSVLTDCVDTDSYTLYHLLEDSALRDPWSDNCLLLVIASRDPIPKDIQHKFMAYLSQGGKVLGLSSPFTLGGFRVTRRDVLRNTVQNLVFSKADGTEVRLSVLSSGYVYEEGPSLGRLQGHLENEDKDKMIVHVPFGTLGGEAVLCQVHLELPPGASLVQTADDFNVLKSSNVRRHEVLKEILTALGLSCDAPQVPALTPLYLLLAAEETQDPFMQWLGRHTDPEGIIKSSKLSLQFVSSYTSEAEITPSSMPVVTDPEAFSSEHFSLETYRQNLQTTRLGKVILFAEVTSTTMSLLDGLMFEMPQEMGLIAIAVRQTQGKGRGPNAWLSPVGCALSTLLVFIPLRSQLGQRIPFVQHLMSLAVVEAVRSIPGYEDINLRVKWPNDIYYSDLMKIGGVLVNSTLMGETFYILIGCGFNVTNSNPTICINDLIEEHNKQHGAGLKPLRADCLIARAVTVLEKLIDRFQDQGPDGVLPLYYKYWVHGGQQVRLGSTEGPQASIVGLDDSGFLQVHQEDGGVVTVHPDGNSFDMLRNLIVPKRQ

SEQ ID NO: 18 - protein, 726 aa, Homo sapiens
MEDRLHMDNGLVPQKIVSVHLQDSTLKEVKDQVSNKQAQILEPKPEPSLEIKPEQDGMEHVGRDDPKALGEEPKQRRGSASGSEPAGDSDRGGGPVEHYHLHLSSCHECLELENSTIESVKFASAENIPDLPYDYSSSLESVADETSPEREGRRVNLTGKAPNILLYVGSDSQEALGRFHEVRSVLADCVDIDSYILYHLLEDSALRDPWTDNCLLLVIATRESIPEDLYQKFMAYLSQGGKVLGLSSSFTFGGFQVTSKGALHKTVQNLVFSKADQSEVKLSVLSSGCRYQEGPVRLSPGRLQGHLENEDKDRMIVHVPFGTRGGEAVLCQVHLELPPSSNIVQTPEDFNLLKSSNFRRYEVLREILTTLGLSCDMKQVPALTPLYLLSAAEEIRDPLMQWLGKHVDSEGEIKSGQLSLRFVSSYVSEVEITPSCIPVVTNMEAFSSEHFNLEIYRQNLQTKQLGKVILFAEVTPTTMRLLDGLMFQTPQEMGLIVIAARQTEGKGRGGNVWLSPVGCALSTLLISIPLRSQLGQRIPFVQHLMSVAVVEAVRSIPEYQDINLRVKWPNDIYYSDLMKIGGVLVNSTLMGETFYILIGCGFNVTNSNPTICINDLITEYNKQHKAELKPLRADYLIARVVTVLEKLIKEFQDKGPNSVLPLYYRYWVHSGQQVHLGSAEGPKVSIVGLDDSGFLQVHQEGGEVVTVHPDGNSFDMLRNLILPKRR

SEQ ID NO: 19 - DNA, 57 bp, Escherichia coli
atgaaaaagatttggctggcgctggctggtttagttttagcgtttagcgcatcggcg

SEQ ID NO: 20 - protein, 19 aa, Escherichia coli
MKKIWLALAGLVLAFSASA

SEQ ID NO: 21 - protein, 18 aa, Escherichia coli
MRVLLFLLLSLFMLPAFS

SEQ ID NO: 22 - protein, 21 aa, Escherichia coli
MKQALRVAFGFLILWASVLHA

SEQ ID NO: 23 - protein, 23 aa, Escherichia coli
MMTKIKLLMLIIFYLIISASAHA

SEQ ID NO: 24 - protein, 25 aa, Escherichia coli
MMITLRKLPLAVAVAAGVMSAQAMA

SEQ ID NO: 25 - protein, 26 aa, Escherichia coli
MKIKTGARILALSALTTMMFSASALA

SEQ ID NO: 26 - protein, 23 aa, Escherichia coli
MNKKVLTLSAVMASMLFGAAAHA

SEQ ID NO: 27 - protein, 21 aa, Escherichia coli
MKKTAIAIAVALAGFATVAQA

SEQ ID NO: 28 - DNA, 112 bp, Escherichia coli
atgaacaataacgatctctttcaggcatcacgtcggcgttttctggcacaactcggcggcttaaccgtcgccgggatgctggggccgtcattgttaacgccgcgacgtgcga

SEQ ID NO: 29 - protein, 37 aa, Escherichia coli
MNNNDLFQASRRRFLAQLGGLTVAGMLGPSLLTPRRA

SEQ ID NO: 30 - DNA, 66 bp, Escherichia coli
atgaaatacctattgcctacggcagccgctggattgttattactcgctgcccaaccagccatggcc

SEQ ID NO: 31 - protein, 22 aa, Erwinia carotovora
MKYLLPTAAAGLLLLAAQPAMA

SEQ ID NO: 32 - DNA, 18 bp, Artificial sequence
  Synthetic hexahistidine oligonucleotide
caccatcaccatcaccat

SEQ ID NO: 33 - protein, 6 aa, Artificial sequence
  Synthetic hexahistidine tag
HHHHHH

SEQ ID NO: 34 - DNA, 30 bp, Artificial sequence
  Synthetic dodecahistidine oligonucleotide
caccaccatcatcaccaccatcaccatcac

SEQ ID NO: 35 - protein, 10 aa, Artificial sequence
  Synthetic dodecahistidine tag
HHHHHHHHHH

SEQ ID NO: 36 - DNA, 6 bp, Artificial sequence
  Synthetic GA oligonucleotide
ggcgca

SEQ ID NO: 37 - protein, 2 aa, Artificial sequence
  Synthetic GA peptide
GA

SEQ ID NO: 38 - DNA, 27 bp, Artificial sequence
  Synthetic hemagglutinin oligonucleotide
tacccatacgatgttccagattacgct

SEQ ID NO: 39 - protein, 9 aa, Artificial sequence
  Synthetic hemagglutinin peptide
YPYDVPDYA

SEQ ID NO: 40 - DNA, 630 bp, Artificial sequence
  Synthetic minor coat protein pIII
ccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 41 - protein, 209 aa, Artificial sequence
  Synthetic minor coat protein pIII
PFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 42 - DNA, 153 bp, Artificial sequence
  Synthetic major  coat protein pVIII
gctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 43 - protein, 50 aa, Artificial sequence
  Synthetic major coat protein pVIII
AEGDDPAKAAFNSLQASATEYIGYAWAMVVVIVGATIGIKLFKKFTSKAS

SEQ ID NO: 44 - DNA, 3797 bp, Artificial sequence
  DsbA-Avitag-pIII vector
ggtggcggccgcaaattctatttcaaggagacagtcataatgaaaaagatttggctggcgctggctggtttagttttagcgtttagcgcatcggcggagctcgaattcggtcgacctccaccatcaccatcaccattccggtggtggttacccatacgatgttccagattacgctggcgcaggcctgaacgacatcttcgaggctcagaaaatcgaatggcacgaaagtggtggcggtggctctccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaaagtggtggtggccttaattaattgactcgagtcaattaattaaggccttaataattgactcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 45 - DNA, 837 bp, Artificial sequence
  Synthetic DsbA-Avitag-pIII  encoding oligonucleotide
atgaaaaagatttggctggcgctggctggtttagttttagcgtttagcgcatcggcggagctcgnnaattcggtcgacctccaccatcaccatcaccattccggtggtggttacccatacgatgttccagattacgctggcgcaggcctgaacgacatcttcgaggctcagaaaatcgaatggcacgaaagtggtggcggtggctctccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 46 - protein, 278 aa, Artificial sequence
  Synthetic DsbA-Avitag-pIII fusion peptide
MKKIWLALAGLVLAFSASAELXNSVDLHHHHHHSGGGYPYDVPDYAGAGLNDIFEAQKIEWHESGGGGSPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 47 - DNA, 3701 bp, Artificial sequence
  TorA-Avitag-pIII vector
ggtggcggccgcaaattctatttcaaggagacagctagcatgaacaataacgatctctttcaggcatcacgtcggcgttttctggcacaactcggcggcttaaccgtcgccgggatgctggggccgtcattgttaacgccgcgacgtgcgactgcggagctcgaattcggtcgacctccaccatcaccatcaccatggcgcatacccatacgatgttccagattacgctggcgcaggcctgaacgacatcttcgaggctcagaaaatcgaatggcacgaaagtggtggcggtggatccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaactgcagagtggtggtggccttaattaattgactcgagtcaattaattaaggccttaataattgactcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 48 - DNA, 735 bp, Artificial sequence
  Synthetic TorA-Avitag-pIII encoding oligonucleotide
atgaacaataacgatctctttcaggcatcacgtcggcgttttctggcacaactcggcggcttaaccgtcgccgggatgctggggccgtcattgttaacgccgcgacgtgcgactgcggagctcgnnaattcggtcgacctccaccatcaccatcaccatggcgcatacccatacgatgttccagattacgctggcgcaggcctgaacgacatcttcgaggctcagaaaatcgaatggcacgaaagtggtggcggtggatccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 49 - protein, 244 aa, Artificial sequence
  Synthetic TorA-Avitag-pIII fusion peptide
MNNNDLFQASRRRFLAQLGGLTVAGMLGPSLLTPRRATAELXNSVDLHHHHHHGAYPYDVPDYAGAGLNDIFEAQKIEWHESGGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 50 - DNA, 3800 bp, Artificial sequence
  PelB-Avitag-pIII vector
ggtggcggccgcaaattctatttcaaggagacagtcataatgaaatacctattgcctacggcagccgctggattgttattactcgctgcccaaccagccatggccgagctcgaattcggtcgacctccaccatcaccatcaccatggcgcatacccatacgatgttccagattacgctggcgcaggcctgaacgacatcttcgaggctcagaaaatcgaatggcacgaaagtggtggcggtggctctccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaaagtggtggtggccttaattaattgactcgagtcaattaattaaggccttaataattgactcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 51 - DNA, 840 bp, Artificial sequence
  Synthetic PelB-Avitag-pIII encoding oligonucleotide
atgaaatacctattgcctacggcagccgctggattgttattactcgctgcccaaccagccatggccgagctcgnnaattcggtcgacctccaccatcaccatcaccatggcgcatacccatacgatgttccagattacgctggcgcaggcctgaacgacatcttcgaggctcagaaaatcgaatggcacgaaagtggtggcggtggctctccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaa

SEQ ID NO: 52 - protein, 279 aa, Artificial sequence
  Synthetic PelBsbA-Avitag-pIII fusion peptide
MKYLLPTAAAGLLLLAAQPAMAELXNSVDLHHHHHHGAYPYDVPDYAGAGLNDIFEAQKIEWHESGGGGSPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 53 - DNA, 3299 bp, Artificial sequence
  DsbA-Avitag-pVIII vector
ggtggcggccgcaaattctatttcaaggagacagtcataatgaaaaagatttggctggcgctggctggtttagttttagcgtttagcgcatcggcggagctcgaattcggtcgacctccaccaccatcatcaccaccatcaccatcactccggtggtggttacccatacgatgttccagattacgctggcgcaggcctgaacgacatcttcgaggctcagaaaatcgaatggcacgaaggatccggtggcggtggctctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaagctagtcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 54 - DNA, 375 bp, Artificial sequence
  Synthetic DsbA-Avitag-pVIII encoding oligonucleotide
atgaaaaagatttggctggcgctggctggtttagttttagcgtttagcgcatcggcggagctcgnnaattcggtcgacctccaccaccatcatcaccaccatcaccatcactccggtggtggttacccatacgatgttccagattacgctggcgcaggcctgaacgacatcttcgaggctcagaaaatcgaatggcacgaaggatccggtggcggtggctctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 55 - protein, 124 aa, Artificial sequence
  Synthetic DsbA-Avitag-pVIII encoding peptide
MKKIWLALAGLVLAFSASAELXNSVDLHHHHHHHHHHSGGGYPYDVPDYAGAGLNDIFEAQKIEWHEGSGGGGSAEGDDPAKAAFNSLQASATEYIGYAWAMVVVIVGATIGIKLFKKFTSKAS

SEQ ID NO: 56 - DNA, 3302 bp, Artificial sequence
  PelB-Avitag-pVIII vector
ggtggcggccgcaaattctatttcaaggagacagtcataatgaaatacctattgcctacggcagccgctggattgttattactcgctgcccaaccagccatggccgagctcgaattcggtcgacctccaccaccatcatcaccaccatcaccatcacggcgcatacccatacgatgttccagattacgctggcgcaggcctgaacgacatcttcgaggctcagaaaatcgaatggcacgaaggatccggtggcggtggctctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaagctagtcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 57 - DNA, 378 bp, Artificial sequence
  Synthetic PelB-Avitag-pVIII  encoding oligonucleotide
atgaaatacctattgcctacggcagccgctggattgttattactcgctgcccaaccagccatggccgagctcgnnaattcggtcgacctccaccaccatcatcaccaccatcaccatcacggcgcatacccatacgatgttccagattacgctggcgcaggcctgaacgacatcttcgaggctcagaaaatcgaatggcacgaaggatccggtggcggtggctctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctga

SEQ ID NO: 58 - protein, 125 aa, Artificial sequence
  Synthetic PelB-Avitag-pVIII fusion peptide
MKYLLPTAAAGLLLLAAQPAMAELXNSVDLHHHHHHHHHHGAYPYDVPDYAGAGLNDIFEAQKIEWHEGSGGGGSAEGDDPAKAAFNSLQASATEYIGYAWAMVVVIVGATIGIKLFKKFTSKAS

SEQ ID NO: 59 - DNA, 4435 bp, Artificial sequence
  pJuFo-pIII vector
ggtggcggccgcaaattctatttcaaggagacagtcataatgaaatacctattgcctacggcagccgctggattgttattactcgctgcccaaccagccatggcccaggtgaaactgctcgacggtatcgataagctttgcggtggtcggatcgcccggcttgaggaaaaagtgaaaaccttgaaagcgcaaaactccgagctggcgtccacggccaacatgctcagggaacaggtggcacagcttaaacagaaagtcatgaaccacggtggttgcggatccactagtggtggcggtggctctccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattatgctagctagtaacacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgaattaattctaaactagctagtcgccaaggagacagtcataatgaaatacctattgcctacggcagccgctggattgttattactcgctgcccaaccagccatggccgagctctgcggtggtttgaccgacaccctgcaggcggaaaccgaccagctggaagacgaaaaatccgcgctgcaaaccgaaatcgcgaacctgctgaaagaaaaagaaaagctggagttcatcctggcggcacacggtggttgcagatctcaccatcaccatcaccatgaattgggcggttccggtctgaatgatatcttcgaagcccagaagattgaatggcacgaaggcgcttacccgtatgatgtcccggattatgctgaattcgttaattaattgaaatcgagggggggccttaattaattgactcgagtcaattaattaaggccttaataattgactcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 60 - protein, 297 aa, Artificial sequence
  Synthetic PelB-c-Jun-pIII fusion peptide
MKYLLPTAAAGLLLLAAQPAMAQVKLLDGIDKLCGGRIARLEEKVKTLKAQNSELASTANMLREQVAQLKQKVMNHGGCGSTSGGGGSPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 61 - protein, 113 aa, Artificial sequence
  Synthetic PelB-cFos-Avitag fusion peptide
MKYLLPTAAAGLLLLAAQPAMAELCGGLTDTLQAETDQLEDEKSALQTEIANLLKEKEKLEFILAAHGGCRSHHHHHHELGGSGLNDIFEAQKIEWHEGAYPYDVPDYAEFVN

SEQ ID NO: 62 - DNA, 3943 bp, Artificial sequence
  pJuFo-pVIII vector
ggtggcggccgcaaattctatttcaaggagacagtcataatgaaatacctattgcctacggcagccgctggattgttattactcgctgcccaaccagccatggcccaggtgaaactgctcgacggtatcgataagctttgcggtggtcggatcgcccggcttgaggaaaaagtgaaaaccttgaaagcgcaaaactccgagctggcgtccacggccaacatgctcagggaacaggtggcacagcttaaacagaaagtcatgaaccacggtggttgcggatccggtggcggtggctctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaagctagctagtaacacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacgaattaattctaaactagctagtcgccaaggagacagtcataatgaaatacctattgcctacggcagccgctggattgttattactcgctgcccaaccagccatggccgagctctgcggtggtttgaccgacaccctgcaggcggaaaccgaccagctggaagacgaaaaatccgcgctgcaaaccgaaatcgcgaacctgctgaaagaaaaagaaaagctggagttcatcctggcggcacacggtggttgcagatctcaccatcaccatcaccatgaattgggcggttccggtctgaatgatatcttcgaagcccagaagattgaatggcacgaaggcgcttacccgtatgatgtcccggattatgctgaattcgttaattaattgacatatgaatcgagggggggccttaattaattgactcgagtcaattaattaaggccttaataattgactcgagcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggccaccgc

SEQ ID NO: 63 - protein, 136 aa, Artificial sequence
  Synthetic PelB-c-Jun-pIIIV fusion peptide
MKYLLPTAAAGLLLLAAQPAMAQVKLLDGIDKLCGGRIARLEEKVKTLKAQNSELASTANMLREQVAQLKQKVMNHGGCGSGGGGSAEGDDPAKAAFNSLQASATEYIGYAWAMVVVIVGATIGIKLFKKFTSKAS

SEQ ID NO: 64 - protein, 113 aa, Artificial sequence; identical to SEQ ID NO: 61
  Synthetic PelB-cFos-Avitag fusion peptide

SEQ ID NO: 65 - DNA, 36330 bp, Artificial sequence
  T7Select-Avitag-N vector
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataatgatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctaactagatctgtgctcaaagaggaatctatcatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattggggcgcatacccatacgatgttccagattacgctggtggatccggtctgaacgacatctttgaagcacaaaaaatcgaatggcacgaaggatccgaattcgttaattaattgaagcttgcggccgcactcgagtaactagttaaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 66 - DNA, 36298 bp, Artificial sequence
  T7Select-Avitag-C vector
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataatgatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctaactagatctgtgctcaaagaggaatctatcatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattcgggcggttccggtctgaatgatatttttgaagctcagaagatcgaatggcacgaaggcgcacatcatcatcaccaccactaagcttgcggccgcactcgagtaactagttaaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 67 - DNA, 36286 bp, Artificial sequence
  T7Select*-Avitag-N vector
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataatgatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctaactagatctcattatcatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattcgggcggttccggtctgaatgatatttttgaagctcagaagatcgaatggcacgaaggcgcacatcatcatcaccaccactaagcttgcggccgcactcgagtaactagttaaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 68 - DNA, 7391 bp, Artificial sequence
  SUMO-(Avitag)3 vector
aattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgcagcatttatcagggttattgtctcatgagcggatacctatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctgaatcgatggttaagtctagaattaacactcattcctgttgaagctcttgacaatgggtgaagttgatgtcttgtgagtggcctcacaggtatagctgttatgtcgttcatactcgtccttggtcaacgtgagggtgctgctcatgctgtaggtgctgtctttgctgtcctgatcagtccaactgttcaggacgccattttgtcgttcactgccatcaatcttccacttgacattgatgtctttggggtagaagttgttcaagaagcacacgactgaggcacctccagatgttaactgctcactggatggagggaagatggatacagntggtgcagcatcannnccgtttgatttggagtttggtgcctccaccggacgtccgaggataactagcatattgtagacagtaatagtctgcaaaatcttcagactcaaggctgctggtggtgagagaataatctgacccagacctactgccactgaacctttttgggacaccagaatgtaaagtggatgcggcgtagatcaggcgtttaatagttccatctggtttctgctgaagccagcctaagtaaccattaatttcctgacttgcccgacaagtgagactgactctttctcccagagaggcagataaggaggatggagactgggtgagcacgagctcttattcatgccactcaatcttttgcgcttcgaagatatcattaagcccggagccaccttcgtgccattcgatcttctgagcttcaaaaatatcattcagaccggaaccgccttcgtgccattcgattttctgagcctcgaagatgtcgttcaggccgctcgagccaccaatctgttctctgtgagcctcaataatatcgttatcctccatgtccaaatcttcaggggtctgatcagcttgaatcctaataccgtcgtacaagaatcttaaggagtccatttccttaccctgtcttttagcgaacgcttccatcagccttcttaaaggagtggtctttttgatcttgaagaagatttctgaagatccatcggacacctttaaattgatgtgagtctcaggcttgacttctggcttgacctctggcttagcttcttgattgacttctgagtccgacatatgtgtatcctccattagttagctagtttagaattcatgccgtcagcttaattctgtttcctgtgtgaaattgttatccgctcacaattccacacattatacgagccgatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgataagcccggatcagcttgcaattcgcgcgcgaaggcgaagcggcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacga

SEQ ID NO: 69 - DNA, 456 bp, Artificial sequence
  Synthetic SUMO-(Avitag)3 encoding oligonucleotide
atgtcggactcagaagtcaatcaagaagctaagccagaggtcaagccagaagtcaagcctgagactcacatcaatttaaaggtgtccgatggatcttcagaaatcttcttcaagatcaaaaagaccactcctttaagaaggctgatggaagcgttcgctaaaagacagggtaaggaaatggactccttaagattcttgtacgacggtattaggattcaagctgatcagacccctgaagatttggacatggaggataacgatattattgaggctcacagagaacagattggtggctcgagcggcctgaacgacatcttcgaggctcagaaaatcgaatggcacgaaggcggttccggtctgaatgatatttttgaagctcagaagatcgaatggcacgaaggtggctccgggcttaatgatatcttcgaagcgcaaaagattgagtggcatgaataa

SEQ ID NO: 70 - protein, 151 aa, Artificial sequence
  Synthetic SUMO-(Avitag)3 fusion peptide
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSSGLNDIFEAQKIEWHEGGSGLNDIFEAQKIEWHEGGSGLNDIFEAQKIEWHE

SEQ ID NO: 71 - DNA, 7901 bp, Artificial sequence
  pBirA vector
aattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgcagcatttatcagggttattgtctcatgagcggatacctatttgaatgtatttagaaaaataaacaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctgaatcgatggttaagtctagaattaacactcattcctgttgaagctcttgacaatgggtgaagttgatgtcttgtgagtggcctcacaggtatagctgttatgtcgttcatactcgtccttggtcaacgtgagggtgctgctcatgctgtaggtgctgtctttgctgtcctgatcagtccaactgttcaggacgccattttgtcgttcactgccatcaatcttccacttgacattgatgtctttggggtagaagttgttcaagaagcacacgactgaggcacctccagatgttaactgctcactggatggagggaagatggatacagntggtgcagcatcannnccgtttgatttggagtttggtgcctccaccggacgtccgaggataactagcatattgtagacagtaatagtctgcaaaatcttcagactcaaggctgctggtggtgagagaataatctgacccagacctactgccactgaacctttttgggacaccagaatgtaaagtggatgcggcgtagatcaggcgtttaatagttccatctggtttctgctgaagccagcctaagtaaccattaatttcctgacttgcccgacaagtgagactgactctttctcccagagaggcagataaggaggatggagactgggtgagcacgagctcttatttttctgcactacgcagggatatttcaccgcccatccagggttttattattccatcctgctcaagtaataaagccccctgtttgtctattccgcgtgaaatgccaaatatttctttatcaccaatgataagtttcactgggcgattaataaaattatccagcttttcccagcgcgacagataaggtgccaatccttcttgttcgaagagttccaacgcagcacgtaattcacgtattagcatggccgccaacgtattacgatcgagattgatccccgcttcctgcagcgtgatccacccctgattaacgacactctcttcaacacggcgcattgccatgttgatcccggctccaatgactatttgcgccgcatcgccagttttgccagtcagctccaccagaatgcctgccagcttgcgatcctgcagatagaggtcattaggccatttaacacgaactttatctgcacccagcttgcgtaatacttccgccatcacgataccgataaccagacttaaaccaatcgccgccgccgggccttgttccagacgccagaacatcgacaaatataagtttgcgccaaaaggcgaaaaccatttccgaccccggcgaccacggccagcctgctggtattctgcaatgcaagcatcgcccgatttaagctctccgatacgatcaagaaggtactgattcgtggagtcaatcactggcagcacggctacactaccgccatccagctgacccaatatctgtttagcattaagtaactggataggctcaggcaggctgtatcctttacccggaacggtaaagacatcaacgccccagtcacgcagtgtctgaatgtgtttattaatagccgcccggctcattcccagcgtttcacccaactgctcgccagagtgaaattcaccgttcgctaacagggcaatcaatttcagtggcacggtgttatccttcatttatgtatcctccattagttagctagtttagaattcatgccgtcagcttaattctgtttcctgtgtgaaattgttatccgctcacaattccacacattatacgagccgatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcaccgtgcagtcgataagcccggatcagcttgcaattcgcgcgcgaaggcgaagcggcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgg

SEQ ID NO: 72 - protein, 255 aa, Artificial sequence
  Synthetic GFP peptide
GSSHHHHHHSSGLVPRGSHMGGTSSKGEELFTGVVPILVELDGDVNGHKFSVRGEGEGDATIGKLTLKFICTTGKLPVPWPTLVTTLSYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGTRDHMVLHEYVNAAGIT

SEQ ID NO: 73 - protein, 239 aa, Artificial sequence
  Synthetic GFP 1-10 peptide
GSSHHHHHHSSGLVPRGSHMGGTSSKGEELFTGVVPILVELDGDVNGHKFSVRGEGEGDATIGKLTLKFICTTGKLPVPWPTLVTTLSYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGT

SEQ ID NO: 74 - protein, 16 aa, Artificial sequence
  Synthetic GFP 11 peptide
RDHMVLHEYVNAAGIT

SEQ ID NO: 75 - DNA, 5422 bp, Artificial sequence
  pET-GFP 11 vector
gttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatgttttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggaggcctgaacgatatttttgaagcgcagaaaattgaatggcatgaacaccatcaccatcaccatgaaaacctgtacttccaatccaatattggtagtgggagcaacggcagcagcggatcccgcgatcacatggtcctgcacgagtacgtgaacgccgccgggatcacttagtaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaactagtaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagactt

SEQ ID NO: 76 - DNA, 2614 bp, Artificial sequence
  SITS-Avitag vector
gacgtctaatacgactcactatagggacatcttaagtttattttattttattttattttattttattttattttattttattttattttattttatttaaccatgacagtaatgtataaagtctgtaaagacattaaacacgtaagtgaaaccatggcacaccatcaccaccatcacagcagcggtctggaagttctgtttcagggtacctccggcctgaacgacatcttcgaggctcagaaaatcgaatggcacgaaggcgcgcaattgtaagctttctagctgcaggaaggaagctgagttggctgctgccaccgctgagcaataactagtaattactagcataaccccttggggcctctaaacgggtcttgagggggttttttgctgaaaggaggacagctgatgattgtcatgcttgccatctgttttcttgcaaggtcagaggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaggatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctagttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 77 - DNA, 6388 bp, Artificial sequence
  pBirA*  vector
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccatgaaggacaacaccgtgcccctgaagctgatcgccctgctggccaacggcgagttccacagcggcgagcagctgggcgagaccctgggcatgagccgcgccgccatcaacaagcacatccagaccctgcgcgactggggcgtggacgtgttcaccgtgcccggcaagggctacagcctgcccgagcccatccagctgctgaacgccaagcagatcctgggccagctggacggcggcagcgtggccgtgctgcccgtgatcgacagcaccaaccagtacctgctggaccgcatcggcgagctgaagagcggcgacgcctgcatcgccgagtaccagcaggccggccgcggccgccgcggccgcaagtggttcagccccttcggcgccaacctgtacctgagcatgttctggcgcctggagcagggccccgccgccgccatcggcctgagcctggtgatcggcatcgtgatggccgaggtgctgcgcaagctgggcgccgacaaggtgcgcgtgaagtggcccaacgacctgtacctgcaggaccgcaagctggccggcatcctggtggagctgaccggcaagaccggcgacgccgcccagatcgtgatcggcgccggcatcaacatggccatgcgccgcgtggaggagagcgtggtgaaccagggctggatcaccctgcaggaggccggcatcaacctggaccgcaacaccctggccgccatgctgatcagcgagctgcgcgccgccctggagctgttcgagcaggagggcctggccccctacctgagccgctgggagaagctggacaacttcatcaaccgccccgtgaagctgatcatcggcctggaggagaaggacaaggagatcttcggcatcagccgcggcatcgacaagcagggcgccctgctgcaggacggcatcatcaagccctggatgggcggcgagatcagcctgcgcagcgcctaaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 78 - DNA, 966 bp, Artificial sequence
  Synthetic BirA* encoding oligonucleotide
atgaaggacaacaccgtgcccctgaagctgatcgccctgctggccaacggcgagttccacagcggcgagcagctgggcgagaccctgggcatgagccgcgccgccatcaacaagcacatccagaccctgcgcgactggggcgtggacgtgttcaccgtgcccggcaagggctacagcctgcccgagcccatccagctgctgaacgccaagcagatcctgggccagctggacggcggcagcgtggccgtgctgcccgtgatcgacagcaccaaccagtacctgctggaccgcatcggcgagctgaagagcggcgacgcctgcatcgccgagtaccagcaggccggccgcggccgccgcggccgcaagtggttcagccccttcggcgccaacctgtacctgagcatgttctggcgcctggagcagggccccgccgccgccatcggcctgagcctggtgatcggcatcgtgatggccgaggtgctgcgcaagctgggcgccgacaaggtgcgcgtgaagtggcccaacgacctgtacctgcaggaccgcaagctggccggcatcctggtggagctgaccggcaagaccggcgacgccgcccagatcgtgatcggcgccggcatcaacatggccatgcgccgcgtggaggagagcgtggtgaaccagggctggatcaccctgcaggaggccggcatcaacctggaccgcaacaccctggccgccatgctgatcagcgagctgcgcgccgccctggagctgttcgagcaggagggcctggccccctacctgagccgctgggagaagctggacaacttcatcaaccgccccgtgaagctgatcatcggcctggaggagaaggacaaggagatcttcggcatcagccgcggcatcgacaagcagggcgccctgctgcaggacggcatcatcaagccctggatgggcggcgagatcagcctgcgcagcgcctaa

SEQ ID NO: 79 - protein, 321 aa, Artificial sequence
  Synthetic BirA* peptide
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLISELRAALELFEQEGLAPYLSRWEKLDNFINRPVKLIIGLEEKDKEIFGISRGIDKQGALLQDGIIKPWMGGEISLRSA

SEQ ID NO: 80 - DNA, 4245 bp, Artificial sequence
  pACYC-184 vector
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 81 - DNA, 36249 bp, Artificial sequence
  T7Select 10-3b
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataatgatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctaactagatctgtgctcaaagaggaatctatcatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattctcctgcagggatatcccgggagctcgtcgacaagcttgcggccgcactcgagtaactagttaaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 82 - DNA, 36219 bp, Artificial sequence
  T7Select 1-1b
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctggtcttcgcgcccatcatgaggtggcgccgaaggcttgcccttcgttcgaccttaagcgttggtgggagaagaacgaactggtcacttctgaccgtggataatgatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctaactagatctcattatcatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgcaggagctgtcgtattccagtcaggtgtgatgctcggggatccgaattcgagctccgtcgacaagcttgcggccgcactcgagtaactagttaaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagtaacaccagagatggtagagaagtacgctatggataaggcttatggtatctcccactcagaccagttcaccaacagttccataatagaagagaacattgagggcttagtaggtatcgagaataactcattccttgaggcacgtaacttgtttgattcggacctatccatcactatgccagacggacagcaattctcagtgaatgacctaagggacttcgatatgttccgcatcatgccagcgtatgaccgccgtgtcaatggtgacatcgccatcatggggtctactggtaaaaccactaaggaacttaaggatgagattttggctctcaaagcgaaagctgagggagacggtaagaagactggcgaggtacatgctttaatggataccgttaagattcttactggtcgtgctagacgcaatcaggacactgtgtgggaaacctcactgcgtgccatcaatgacctagggttcttcgctaagaacgcctacatgggtgctcagaacattacggagattgctgggatgattgtcactggtaacgttcgtgctctagggcatggtatcccaattctgcgtgatacactctacaagtctaaaccagtttcagctaaggaactcaaggaactccatgcgtctctgttcgggaaggaggtggaccagttgattcggcctaaacgtgctgacattgtgcagcgcctaagggaagcaactgataccggacctgccgtggcgaacatcgtagggaccttgaagtattcaacacaggaactggctgctcgctctccgtggactaagctactgaacggaaccactaactaccttctggatgctgcgcgtcaaggtatgcttggggatgttattagtgccaccctaacaggtaagactacccgctgggagaaagaaggcttccttcgtggtgcctccgtaactcctgagcagatggctggcatcaagtctctcatcaaggaacatatggtacgcggtgaggacgggaagtttaccgttaaggacaagcaagcgttctctatggacccacgggctatggacttatggagactggctgacaaggtagctgatgaggcaatgctgcgtccacataaggtgtccttacaggattcccatgcgttcggagcactaggtaagatggttatgcagtttaagtctttcactatcaagtcccttaactctaagttcctgcgaaccttctatgatggatacaagaacaaccgagcgattgacgctgcgctgagcatcatcacctctatgggtctcgctggtggtttctatgctatggctgcacacgtcaaagcatacgctctgcctaaggagaaacgtaaggagtacttggagcgtgcactggacccaaccatgattgcccacgctgcgttatctcgtagttctcaattgggtgctcctttggctatggttgacctagttggtggtgttttagggttcgagtcctccaagatggctcgctctacgattctacctaaggacaccgtgaaggaacgtgacccaaacaaaccgtacacctctagagaggtaatgggcgctatgggttcaaaccttctggaacagatgccttcggctggctttgtggctaacgtaggggctaccttaatgaatgctgctggcgtggtcaactcacctaataaagcaaccgagcaggacttcatgactggtcttatgaactccacaaaagagttagtaccgaacgacccattgactcaacagcttgtgttgaagatttatgaggcgaacggtgttaacttgagggagcgtaggaaataatacgactcactatagggagaggcgaaataatcttctccctgtagtctcttagatttactttaaggaggtcaaatggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaattggtaaatcacaaggaaagacgtgtagtccacggatggactctcaaggaggtacaaggtgctatcattagactttaacaacgaattgattaaggctgctccaattgttgggacgggtgtagcagatgttagtgctcgactgttctttgggttaagccttaacgaatggttctacgttgctgctatcgcctacacagtggttcagattggtgccaaggtagtcgataagatgattgactggaagaaagccaataaggagtgatatgtatggaaaaggataagagccttattacattcttagagatgttggacactgcgatggctcagcgtatgcttgcggacctttcggaccatgagcgtcgctctccgcaactctataatgctattaacaaactgttagaccgccacaagttccagattggtaagttgcagccggatgttcacatcttaggtggccttgctggtgctcttgaagagtacaaagagaaagtcggtgataacggtcttacggatgatgatatttacacattacagtgatatactcaaggccactacagatagtggtctttatggatgtcattgtctatacgagatgctcctacgtgaaatctgaaagttaacgggaggcattatgctagaatttttacgtaagctaatcccttgggttctcgctgggatgctattcgggttaggatggcatctagggtcagactcaatggacgctaaatggaaacaggaggtacacaatgagtacgttaagagagttgaggctgcgaagagcactcaaagagcaatcgatgcggtatctgctaagtatcaagaagaccttgccgcgctggaagggagcactgataggattatttctgatttgcgtagcgacaataagcggttgcgcgtcagagtcaaaactaccggaacctccgatggtcagtgtggattcgagcctgatggtcgagccgaacttgacgaccgagatgctaaacgtattctcgcagtgacccagaagggtgacgcatggattcgtgcgttacaggatactattcgtgaactgcaacgtaagtaggaaatcaagtaaggaggcaatgtgtctactcaatccaatcgtaatgcgctcgtagtggcgcaactgaaaggagacttcgtggcgttcctattcgtcttatggaaggcgctaaacctaccggtgcccactaagtgtcagattgacatggctaaggtgctggcgaatggagacaacaagaagttcatcttacaggctttccgtggtatcggtaagtcgttcatcacatgtgcgttcgttgtgtggtccttatggagagaccctcagttgaagatacttatcgtatcagcctctaaggagcgtgcagacgctaactccatctttattaagaacatcattgacctgctgccattcctatctgagttaaagccaagacccggacagcgtgactcggtaatcagctttgatgtaggcccagccaatcctgaccactctcctagtgtgaaatcagtaggtatcactggtcagttaactggtagccgtgctgacattatcattgcggatgacgttgagattccgtctaacagcgcaactatgggtgcccgtgagaagctatggactctggttcaggagttcgctgcgttacttaaaccgctgccttcctctcgcgttatctaccttggtacacctcagacagagatgactctctataaggaacttgaggataaccgtgggtacacaaccattatctggcctgctctgtacccaaggacacgtgaagagaacctctattactcacagcgtcttgctcctatgttacgcgctgagtacgatgagaaccctgaggcacttgctgggactccaacagacccagtgcgctttgaccgtgatgacctgcgcgagcgtgagttggaatacggtaaggctggctttacgctacagttcatgcttaaccctaaccttagtgatgccgagaagtacccgctgaggcttcgtgacgctatcgtagcggccttagacttagagaaggccccaatgcattaccagtggcttccgaaccgtcagaacatcattgaggaccttcctaacgttggccttaagggtgatgacctgcatacgtaccacgattgttccaacaactcaggtcagtaccaacagaagattctggtcattgaccctagtggtcgcggtaaggacgaaacaggttacgctgtgctgtacacactgaacggttacatctaccttatggaagctggaggtttccgtgatggctactccgataagacccttgagttactcgctaagaaggcaaagcaatggggagtccagacggttgtctacgagagtaacttcggtgacggtatgttcggtaaggtattcagtcctatccttcttaaacaccacaactgtgcgatggaagagattcgtgcccgtggtatgaaagagatgcgtatttgcgatacccttgagccagtcatgcagactcaccgccttgtaattcgtgatgaggtcattagggccgactaccagtccgctcgtgacgtagacggtaagcatgacgttaagtactcgttgttctaccagatgacccgtatcactcgtgagaaaggcgctctggctcatgatgaccgattggatgcccttgcgttaggcattgagtatctccgtgagtccatgcagttggattccgttaaggtcgagggtgaagtacttgctgacttccttgaggaacacatgatgcgtcctacggttgctgctacgcatatcattgagatgtctgtgggaggagttgatgtgtactctgaggacgatgagggttacggtacgtctttcattgagtggtgatttatgcattaggactgcatagggatgcactatagaccacggatggtcagttctttaagttactgaaaagacacgataaattaatacgactcactatagggagaggagggacgaaaggttactatatagatactgaatgaatacttatagagtgcataaagtatgcataatggtgtacctagagtgacctctaagaatggtgattatattgtattagtatcaccttaacttaaggaccaacataaagggaggagactcatgttccgcttattgttgaacctactgcggcatagagtcacctaccgatttcttgtggtactttgtgctgcccttgggtacgcatctcttactggagacctcagttcactggagtctgtcgtttgctctatactcacttgtagcgattagggtcttcctgaccgactgatggctcaccgagggattcagcggtatgattgcatcacaccacttcatccctatagagtcaagtcctaaggtatacccataaagagcctctaatggtctatcctaaggtctatacctaaagataggccatcctatcagtgtcacctaaagagggtcttagagagggcctatggagttcctatagggtcctttaaaatataccataaaaatctgagtgactatctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccct

SEQ ID NO: 83 - protein, 33 aa, Artificial sequence
  Artificial synthetic peptide
RASPSEQRRKRRRCHRGETQRPDFEAEIEKQQR

SEQ ID NO: 84 - protein, 33 aa, Artificial sequence
  Artificial synthetic peptide
RKQKSLQTKLAENPPVPRKKRQSRPRWKQWLQK

SEQ ID NO: 85 - protein, 32 aa, Artificial sequence
  Artificial synthetic peptide
PSSTPATNVARPRLNPIRGHKFALAVPNSRTR

SEQ ID NO: 86 - protein, 43 aa, Artificial sequence
  Artificial synthetic peptide
PLTQRTLQRGKKPKQRQNWKKARTSSAKTAPKTVVSRTTSQRK

SEQ ID NO: 87 - protein, 31 aa, Artificial sequence
  Artificial synthetic peptide
LFVDKATPQIYYTPCESVTVKSKGKNRRKKS

SEQ ID NO: 88 - protein, 59 aa, Artificial sequence
  Artificial synthetic peptide
PKQPPKPKKPKTQEKKKKQPAKPKPGKRQRMALKLEADRLFDVKNEDGDVIGHALDMKA

SEQ ID NO: 89 - protein, 47 aa, Artificial sequence
  Artificial synthetic peptide
PPHPRPLPAPAQSRKKQKGRAGRGHEKTGASVLRGPQKPHPLPAQLR

SEQ ID NO: 90 - protein, 38 aa, Artificial sequence
  Artificial synthetic peptide
PLKPKKPKTQEKKKKQPPKPKKPKTQEKKKKQPPKPKR

SEQ ID NO: 91 - protein, 44 aa, Artificial sequence
  Artificial synthetic peptide
PWAKRSLSSLQTSSRPVGRPSRQPRRGSSSKRRPRFRPTQAVSS

SEQ ID NO: 92 - protein, 24 aa, Artificial sequence
  Artificial synthetic peptide
PGRVGISLKVESVRNKDRKKPYKG

SEQ ID NO: 93 - protein, 20 aa, Artificial sequence
  Artificial synthetic peptide
LGGSLHLRRPLKKEKVSISI

SEQ ID NO: 94 - protein, 27 aa, Artificial sequence
  Artificial synthetic peptide
LAQPFAHSRRGDPIGAGRFRHTNLMGD

SEQ ID NO: 95 - protein, 35 aa, Artificial sequence
  Artificial synthetic peptide
RIPGRIQPIDSSHLAVLHEYPSSHRHHHHRHAAPR

SEQ ID NO: 96 - protein, 38 aa, Artificial sequence
  Artificial synthetic peptide
PTSKQNTAHSPGPSKSYATSNEPSKKTAKSSTSSSRGK

SEQ ID NO: 97 - protein, 32 aa, Artificial sequence
  Artificial synthetic peptide
LALTKKGRQYVEDELDLEAKHRGRGGVVHRYW

SEQ ID NO: 98 - protein, 23 aa, Artificial sequence
  Artificial synthetic peptide
LRDADEEHSPRTHHTQYLTKHRR

SEQ ID NO: 99 - protein, 8 aa, Artificial sequence
  Artificial synthetic peptide
LDDPRQRN

SEQ ID NO: 100 - protein, 37 aa, Artificial sequence
  Artificial synthetic peptide
PKSRPPKASEKETTPAETNTENSSHKPRNNWRNAASK

SEQ ID NO: 101 - protein, 31 aa, Artificial sequence
  Artificial synthetic peptide
QAGRGESPLSDNKTSLVRRPVHPICTAPSHS

SEQ ID NO: 102 - protein, 9 aa, Artificial sequence
  Artificial synthetic peptide
LSVSSTMSP

SEQ ID NO: 103 - protein, 15 aa, Artificial sequence
  Artificial synthetic peptide
MRNEVPPHKAINKTR

SEQ ID NO: 104 - protein, 57 aa, Artificial sequence
  Artificial synthetic peptide
LWCRSSTSGPGKNTWPPAPTSGCRRKSTCRATSPSGRTPTGSPRTNAVSSSATWASS

SEQ ID NO: 105 - protein, 12 aa, Artificial sequence
  Artificial synthetic peptide
SGNRVTANGYRR

SEQ ID NO: 106 - protein, 60 aa, Artificial sequence
  Artificial synthetic peptide
RPALDNTTNPTAYHKEPLTRLALPYTAPHRVLATVYNGSSKYGDTSTNNVRGDLQVLAKK

SEQ ID NO: 107 - protein, 42 aa, Artificial sequence
  Artificial synthetic peptide
PGRIQPIDSSQLHDRVVHRGFRRQMKNSSSAQRGTPMPGGRS

SEQ ID NO: 108 - protein, 12 aa, Artificial sequence
  Artificial synthetic peptide
SGNRVTANGYRR

SEQ ID NO: 109 - protein, 87 aa, Artificial sequence
  Artificial synthetic peptide
RSVGGIDWALEGLDRIRDVIPQIRPDLAEVGGVGVGPLERNGGGGGLSNCGRHGVGPRRSEPALDRPRSRGRGRDLGWSGQERVERM

SEQ ID NO: 110 - protein, 52 aa, Artificial sequence
  Artificial synthetic peptide
QMEGMIYNKRGLGYFVSPNAREEILASRRKKFVEEVVPALLNSIWAPEDIEQ

SEQ ID NO: 111 - protein, 34 aa, Artificial sequence
  Artificial synthetic peptide
RGRGGSREETILGRDSQRSSSWSMQGHARSAETV

SEQ ID NO: 112 - protein, 45 aa, Artificial sequence
  Artificial synthetic peptide
PGTGSVPAFEVAERGRRERGIRLANERHLDWGRESTGRVRPRRQA

SEQ ID NO: 113 - protein, 45 aa, Artificial sequence
  Artificial synthetic peptide
LCERSEDAPHENSVLYHLRTKFDLETLEQVGNMLPQKDVLDVLPQ

SEQ ID NO: 114 - protein, 29 aa, Artificial sequence
  Artificial synthetic peptide
PWTSGASTSQETWNRQDLLVTFKTAHAKK

SEQ ID NO: 115 - protein, 48 aa, Artificial sequence
  Artificial synthetic peptide
PFSNMSLSLLDLYLSRGYNVSSIVTMTSQGMYGGTYLVGKPNLSSKRK

SEQ ID NO: 116 - protein, 45 aa, Artificial sequence
  Artificial synthetic peptide
LSDTRGDVTTCRNTCRVGEVSFIHDDHVVVRDANRRQQTHRKGGR

SEQ ID NO: 117 - protein, 47 aa, Artificial sequence
  Artificial synthetic peptide
HPEIQYTSNYNKSVNVDFTVDTNGVYSEPRPIGTRYLTRNLGSRARR

SEQ ID NO: 118 - protein, 58 aa, Artificial sequence
  Artificial synthetic peptide
PGKRQRMALKLEADRLFDVKNEDGDVIGHALAMEGKVMKPLHVKGTIDHPVLSKLKKK

SEQ ID NO: 119 - protein, 37 aa, Artificial sequence
  Artificial synthetic peptide
PSIKSGNDIANCLRKNGRRVVQLSHKTFDTEYQKTKK

SEQ ID NO: 120 - protein, 280 aa, Artificial sequence
  His-Bouganin expression cassette
MHHHHHHGGSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGSGATAGSAATGGATGGSTS

SEQ ID NO: 121 - protein, 276 aa, Artificial sequence
  His-Bouganin-LPETGG expression cassette
MGSSHHHHHHGGTSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSGGTLPETGG

SEQ ID NO: 122 - protein, 291 aa, Artificial sequence
  His-Bouganin-RBD-LPETGG expression cassette
MGSSHHHHHHGGTSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSGGTRBDGSSGGAGGAGGSLPETGG

SEQ ID NO: 123 - protein, 282 aa, Artificial sequence
  His-Bouganin-RBD-Gen1 expression cassette
MGHHHHHHGGSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSGGTGGSRBDGTSGGTGGS

SEQ ID NO: 124 - protein, 305 aa, Artificial sequence
  His-Bouganin-RBD-Gen2 expression cassette
MHHHHHHGGSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGSGTGSATSGSLAGSGATAGTGSGGSRBDGTGTASGGAGTGSGTS

SEQ ID NO: 125 - protein, 311 aa, Artificial sequence
  His-RBD-bouganin-Gen1 expression cassette
MHHHHHHGGSGSRBDGTGSGTGSATSGSLAGSGATAGTGSGYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGSGATAGSAATGGATGGSTS

SEQ ID NO: 126 - protein, 311 aa, Artificial sequence
  His-RBD-Bouganin-Gen2 expression cassette
MHHHHHHGGSGSRBDGTGSGTGSATSGRLKRSGATAGTGSGYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGSGATAGSAATGGATGGSTS

SEQ ID NO: 127 - protein, 274 aa, Artificial sequence
  Bouganin-His expression cassette
MGGTSASGGAGTGSGYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSHHHHHH

SEQ ID NO: 128 - protein, 275 aa, Artificial sequence
  RBD-Bouganin-His-Gen1 expression cassette
MGGGRBDGSSGGSSGGTYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKLEHHHHHH

SEQ ID NO: 129 - protein, 282 aa, Artificial sequence
  RBD-Bouganin-His-Gen2 expression cassette
MGGTSGGTGGSRBDGGSGGTGGSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSHHHHHH

SEQ ID NO: 130 - protein, 288 aa, Artificial sequence
  RBD-Bouganin-His-Gen3 expression cassette
RBDGTGSGTGSATSGSLAGSGATAGTGSGYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSHHHHHH

SEQ ID NO: 131 - protein, 305 aa, Artificial sequence
  RBD-Bouganin-His-Gen4 expression cassette
MGGTSASGGAGTGSGGSRBDGTGSGTGSATSGSLAGSGATAGTGSGYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGGSHHHHHH

SEQ ID NO: 132 - protein, 313 aa, Artificial sequence
  Bouganin-RBD-His expression cassette
MGGTSGSGATAGSAATGGATGGSYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKVDRKDLELGVYKLEFSIEAIHGKTINGQEIAKFFLIVIQMVSEAARFKYIETEVVDRGLYGSFKPNFKVLNLENNWGDISDAIHKSSPQCTTINPALQLISPSNDPWVVNKVSQISPDMGILKFKSSKGSGTGSATSGSLAGSGATAGTGSGGSRBDGTGGSHHHHHH

(19) United States
(12) Patent Application Publication
(10) Pub. No.: US 2016/0146786 A1
(43) Pub. Date: May 26, 2016

(54) Method of monitoring cellular trafficking of peptides

(71) Applicant: PHYLOGICA LIMITED, Mount Claremont, Western Australia (AU)

(72) Inventors: Richard Hopkins, North Perth (AU); Katrin Hoffmann, Aubin Grove (AU); Tatjana Heinrich, Mount Pleasant (AU); Paula Cunningham, Atwell (AU); Paul Watt, Mount Claremont (AU); Nadia Milech, Mount Claremont (AU)

(21) Appl. No.: 14/898,064

(22) PCT Filed: Jun. 26, 2014
(86) PCT No.: PCT/AU2014/050094

§ 371 Date: Dec. 11, 2015

(30) Foreign Application Priority Data
Jun. 26, 2013 (AU) 2013902347
Aug. 13, 2013 (AU) 2013903038
May 8, 2014 (AU) 2014901714

(51) Int. Cl. G01N 33/50

(52) U.S. Cl. CPC G01N 33/5035; G01N 2440/32; G01N 2333/9015; G01N 2500/10

(57) ABSTRACT

This disclosure provides a method of isolating peptides having cell-penetrating function, wherein the peptides are detected as biotinylated molecules only following their translocation through the cell membrane. The disclosure also provides methods for validating the cell-penetrating function of the peptides, or that may be employed in their own right to isolate such peptides, wherein the peptides are detectable by virtue of their ability to transport a detectable cargo into the cytoplasm, such as a cargo toxin or a fragment of a green fluorescent protein (GFP) that is required for complementation of a functional GFP. The disclosure also provides non-canonical peptides having cell-penetrating function that differ structurally from known CPPs such as TAT, VP22, transportan and penetratin, and that are capable of translocating cell membranes and escaping the endosome. The disclosed peptides have utility in transporting cargo therapeutics and diagnostics into cells.

### RELATED APPLICATIONS

This application claims Convention priority to Australian Patent Application No 2013902347 filed on 26 Jun. 2013 and Australian Patent Application No 2013903038 filed on 13 Aug. 2013 and Australian Patent Application No 2014901714 filed 9 May 2014, the contents of which are each incorporated herein in their entirety.

### FIELD OF THE INVENTION

The present invention relates generally to the field of pharmaceutical sciences and, in particular, to the targeting of molecules such as therapeutic compounds and peptides, to organs and/or tissues and/or cells and/or sub-cellular localizations.

### BACKGROUND TO THE INVENTION

Many biologically active compounds require intracellular delivery in order to exert their therapeutic action, either inside the cytoplasm, within the nucleus or other organelles. Selective delivery to particular organs, tissues, cells, or sub-cellular localizations, is highly-desirable to avoid or minimize undesirable side-effects in non-target organs, tissues, cells, or sub-cellular localizations. Thus, the ability to deliver molecules of therapeutic benefit efficiently and selectively is important to drug development.

More than two decades ago it was discovered that certain short sequences, composed mostly of basic, positively-charged amino acids, e.g., Arg, Lys or His, have the ability to transport an attached cargo molecule across the plasma membrane of a cell. These basic sequences are commonly referred to as cell-penetrating peptides (CPPs) or protein transduction domains (PTDs). Prior art CPPs are generally short cationic and/or amphipathic peptide sequences, often between 20 and 50 residues in length, characterized by an ability to translocate across the membrane systems of mammalian cells, localize in one or more intracellular compartments, and mediate intracellular delivery of a cargo molecule e.g., a drug or other therapeutic agent, or a diagnostic agent such as an imaging agent.

Arguably, the most widely-studied and utilized CPP is a peptide derived from the human immunodeficiency virus (HIV-1) transactivator of transcription (TAT) protein. A positively-charged fragment of HIV-1 Tat protein comprising residues 47-57 of the full-length protein penetrates cultured mammalian cells. Since the discovery of Tat, other polycationic CPPs such as e.g., penetratin (a fragment of Antennapedia homeodomain) and vp22 (derived from herpes virus structural protein VP22) have been identified and characterized for their ability to translocate and deliver distinct cargos into the cell cytoplasm and nucleus in vitro and in vivo. Exemplary known CPPs are set forth in Table 1.

TABLE 1
Characterized CPPs

| Cell-penetrating peptide | | |
|---|---|---|
| (CPP) | Sequence | Origin |
| Amphipathic peptides | | |
| Penetratin (43-58) | RQIKIWFQNRRMKWKK | Drosophila melanogaster |
| Amphipathic model peptide | KLALKLALKALKAALKLA | Synthetic |
| Transportan | GWTLNSAGYLLK1NLKALAALAKKIL | Chimeric galanin-mastoparan |
| SBP | MGLGLHLLVLAAALQGAWSQPKKKRKV | Caiman crocodylus Ig(v) light |
| | | chain-SV40 large T antigen |
| FBP | GALFLGWLGAAGSTMGAWSQPKKKRKV | Chimeric HIV-1 gp41-SV40 large |
| | | T antigen |
| Cationic peptides | | |
| HIV Tat peptide (48-60) | GRKKRRQRRRPPQ | Viral transcriptional regulator |
| Syn-B1 | RGGRLSYSRRRFSTSTGR | Protegrin 1 |
| Syn-B3 | RRLSYSRRRF | Protegrin 1 |
| homoarginine peptide | RRRRRRR(RR) | Synthetic |
| (Arg)7 and (Arg)9) | | |

The precise mechanism(s) by which CPPs achieve their cellular internalization has been somewhat controversial. However, there is consensus that most CPPs are internalized via an endocytic mechanism. Several endocytic pathways exist, and clathrin-dependent endocytosis, caveolae/lipid raft-mediated endocytosis or macropinocytosis may be involved. The first step in cellular entry of a polycationic CPP is thought to be an electrostatic interaction between the polycation and negatively-charged heparin sulphate proteoglycan (HSPG) of the plasma membrane. Proceeding on this basis, a charge distribution and amphipathicity of the CPP are believed to be critical factors for cell internalization, possibly affecting an electrostatic interaction between the CPP and proteoglycans on the plasma membrane. Endocytosis of the CPP following contact with the cell surface is believed to be driven by a variety of parameters including the secondary structure of the CPP, the nature of the cargo to which the CPP is linked (if any), cell type, and membrane composition. As such, cell internalization is a complex and multi-faceted process.

Notwithstanding that certain CPPs may share some common characteristics that facilitate their cell binding and uptake e.g., polycationic and amphipathic sequences, not all CPPs possess sufficient similarity in their primary structure e.g., amino acid sequence, to readily predict their ability to bind to the cell surface and/or enter the cell based on sequence alone. It is not understood how secondary and/or tertiary structure considerations could effect cellular uptake.

Following endocytosis, the internalized CPP needs to escape the endosome to avoid degradation, and to deliver its cargo to an intended intracellular destination. Escape from the endosome may provide a bottleneck to efficient intracellular delivery of macromolecular cargos. For example, the efficiency of endosome escape appears to be low for Tat, penetratin, Rev, VP22 and transferrin e.g., Sugita et al. Br. J. Pharmacol. 153, 1143-1152 (2008). Delivery of CPP-cargo conjugates in liposomes may assist their escape from the endocytic vesicle e.g., El-Sayed et al. AAPS J. 11, 13-22 (2009). Moreover, the inclusion of fusigenic peptides, such as the HA2 sequence of influenza (Wadia et al. Nat Med. 10, 310-315, 2004) can also enhance endosomal escape somewhat, although much of the cell penetrating peptides remain in the endosome. There remains a need for CPPs having an ability to escape the endocytic vesicle efficiently following their uptake.

One limitation to the in vivo utility of known CPPs for delivery of drug cargos is their non-selectivity. A generalized uptake of many existing CPPs in vivo may limit their clinical application, particularly where targeted drug action is advantageous or necessary, or where non-specific targeting of an organ or tissue type can lead to unwanted side effects. Notwithstanding that selection of a CPP for the presence of polycationic centres may provide peptides that are able to facilitate initiation of the internalization process, peptides selected for a primary structure that is positively charged may not be cell-selective in view of ubiquity of HSPG and phospholipid in the outer leaflet of cell membranes.

There is presently insufficient diversity of cell-type selective CPPs to provide coverage for many clinical applications involving drug delivery to different cells, tissues, organs and across organ systems. Tight junctions (TJs), basolateral membranes, and apical membranes may function to restrict the passage of CPPs into all cell types, especially when administered intravenously. The blood-brain barrier (BBB) is located at the endothelial tight junctions lining the blood vessels surrounding the brain, and the primary physical and/or pharmacological and/or physiological component(s) of the blood-testis barrier (BTB) and blood-epididymis barrier (BEB) consists of tight junctions between adjacent epithelial cells lining the seminiferous tubules (Sertoli cells) and epididymal duct, respectively. Such physical barriers and/or pharmacological barriers and/or physiological barriers may also be provided by the presence of active transporters and channels at the basolateral and/or apical membranes. HIV-1 Tat-derived peptides, penetratin and VP22 appear to have limited cellular uptake across these barriers and in certain cell types, both in vitro and in vivo. See e.g., Trehin and Merkle, Eur. J. Pharm. Biopharm. 58, 209-223 (2004). Thus, the existing bank of CPPs may not be sufficient to deliver therapeutic cargos to all cell types, suggesting a need for further functional diversity of CPPs.

Safety is a particular concern for the clinical application of any therapeutic agent, and no less so for CPPs that are utilized to deliver a cargo to one or more cells, tissues, organs or across organ systems of the human or animal body. For example, amphipathic peptides may be cytotoxic by virtue of perturbing the cell membrane, e.g., Sugita et al., Brit J Pharmacol 153, 1143-1152 (2008), and it may not be a simple matter to reduce the cytotoxicity of such peptides if their amphipathicity is critical to their interaction with the lipid membrane and subsequent internalization. Similarly, intrastriatal injection of penetratin at 10 μg dosage has been demonstrated to cause neurotoxic cell death, and in vitro delivery at concentrations of 40-100 μM has been demonstrated to induce cell lysis and other cytotoxic effects e.g., Trehin and Merkle, Eur. J. Pharm. Biopharm. 58, 209-223 (2004). Poly-L-arginine peptides have also been reported to induce cell membrane damage, increased permeability of cell barriers and reduce cell-cell contacts between epithelial cells in vitro, to the induce an inflammatory response when injected into the pleural cavity of rat lungs e.g., Trehin and Merkle, Eur. J. Pharm. Biopharm. 58, 209-223 (2004). Accordingly, there remains a need for CPPs having low or reduced cytotoxic side-effects relative to known CPPs.

Many of the limitations of known CPPS are a consequence of the processes used for their identification, and their subsequent adoption in the art before adequate testing has taken place to determine their uptake and/or release from the endosome and/or cell-type selectivity and/or tissue-type selectivity and/or organ selectivity and/or ability to cross physical barriers and/or pharmacological barriers and/or physiological barriers, and/or their safety limits.

Phage-display approaches have been successfully applied for the identification of cell-penetrating peptides and are efficient as they can be performed in a high throughput manner with many peptides being interrogated simultaneously e.g., Kamada et al., Biol Pharm Bull 30, 218-223 (2007). Notwithstanding the widespread and successful use of phage display screening techniques for discovery of new CPPs, existing screening methods do not necessarily select peptides for more than the attribute of cellular uptake, and fail to provide validation of cellular internalization or delivery. There remains a need for improved methods for identifying and isolating CPPs.

### SUMMARY OF THE INVENTION

In work leading up to the present invention, the inventors sought to develop improved methods of determining, identifying and/or isolating peptides, or analogues and/or derivatives thereof, having cell-penetrating activity and preferably that provide an advantage over previously-known methods of isolating CPPs.

As used herein, the term “cell-penetrating peptide” or “CPP” or similar term shall be taken to mean peptidyl compound capable of translocating across a membrane system and internalizing within a cell.

By “peptidyl compound” is meant a composition comprising a peptide, or a composition the structure of which is based on a peptide such as an analogue of a peptide.

As used herein, the term “peptide” shall be taken to mean a compound other than a full-length protein that comprises at least 5 or 6 or 7 or 8 or 9 or 10 contiguous amino acids, or amino acid-like residues, and preferably comprises at least 80% or 85% or 90% or 95% or 99% amino acids by weight. Peptides will generally have an upper length of at least 200 residues or 190 residues or 180 residues or residues or 160 residues or 150 residues or 140 residues or 130 residues or 120 residues or 110 residues or 100 residues, however a peptide may have a length in the range of 10-20 residues or 10-30 residues or 10-40 residues or 10-50 residues or 10-60 residues or 10-70 residues or 10-80 residues or 10-90 residues or 10-100 residues, including any length within said range(s). A peptide as defined may be expressed by translation of an open-reading frame in nucleic acid that has been derived from fragments of naturally-occurring nucleic acid e.g., by amplification of genomic DNA fragments or reverse transcription of mRNA. In one example, the open-reading frame encoding a peptide is the same as an open-reading frame employed by a source organism in nature. In another example, the open-reading frame encoding a peptide is an open-reading frame that is not employed in nature. Thus, a peptide may be the expression product of nucleic acid derived directly or indirectly from an organism having a prokaryotic or compact eukaryote genome. Alternatively, a peptide may the expression product of synthetic nucleic acid.

In contrast, a “peptide conjugate” is a molecule that comprises a peptide and a non-peptidyl moiety without limitation as to a percentage weight of amino acids.

As exemplified herein, the inventors employ a whole-cell biopanning of phage display libraries expressing isolated protein domains that are the expression products derived from genome fragments of prokaryotic genomes and/or compact eukaryotic genomes which are not known or predicted as having cell-penetrating activity in their native environments. These expressed protein domains may be expression products derived from fragments of naturally-occurring open-reading frames, or be encoded by nucleic acid that is not translated in its native context, or from synthetic nucleic acid. The inventors adopted the use of such nucleic acid sources to reduce the contribution of uncharacterized nucleic acid e.g., non-sequenced nucleic acid or non-annotated sequence, and to enhance the diversity of expressed protein domains being screened. Without being bound by theory, this approach is believed to enrich for nucleotide sequences which have evolved to encode protein domains exhibiting improved structural stability and/or protease resistance and/or biological compatibility and/or reduced toxicity.

In one example, the present invention provides a method of monitoring cellular trafficking of a peptide e.g., translocation of a peptide across a cell membrane and/or into a subcellular compartment and/or from a sub-cellular compartment, by providing a substantially non-biotinylated fusion protein comprising a cell penetrating peptide and a biotin ligase substrate domain to a cell expressing a biotin ligase capable of biotinylating the non-biotinylated member, incubating the host cells for a time and conditions sufficient for the non-biotinylated member to enter the host cells and then determining sub-cellular localization of a biotinylated form of the fusion protein or biotin ligase substrate domain thereof.

As used herein the term “cellular trafficking” in its broadest context includes movement of the protein within and between cells.

As used herein, the term “biotin ligase” shall be taken to mean protein or fragment thereof that enzymatically attaches a biotin to a specific lysine residue of a distinct domain of an acceptor protein or fragment thereof e.g. a biotin ligase substrate domain.

As used herein, the term “biotin ligase substrate domain” shall be taken to mean a protein domain capable of being biotinylated, or to which a biotin group can be attached. The term “substantially non-biotinylated fusion protein” shall be taken to mean a covalent attachment of a biotin group to one or more molecules. The term “biotinylated form” shall be taken to mean a member that has at least one biotin group attached.

In another example, the present invention provides a method of determining or identifying a peptide capable of translocating a membrane of a cell, the method comprising the steps:

(a) contacting host cells expressing a biotin ligase with a plurality of substantially non-biotinylated members, wherein the members comprise scaffolds displaying fusion proteins, each of the fusion proteins comprising a candidate peptide moiety and a biotin ligase substrate domain, and wherein said contacting is for a time and under conditions sufficient for at least the displayed fusion proteins of members to enter the host cells;
(b) incubating the host cells for a time and under conditions such that the biotin ligase substrate domain of the at least fusion proteins that have translocated a membrane of the host cell are enzymatically biotinylated by the expressed biotin ligase; and
(c) determining or identifying a candidate peptide moiety that has translocated a membrane of the host cell by performing a process comprising:
(i) detecting the presence of a biotinylated fusion protein in a host cell or cell lysate or extract thereof, wherein the presence of a biotinylated fusion protein indicates that the candidate peptide moiety has translocated the cell membrane; and/or
(ii) isolating at least a biotinylated fusion protein from a host cell or cell lysate or extract thereof; and/or
(iii) recovering at least a biotinylated fusion protein from a host cell or cell lysate or extract thereof.

As used herein, the term “plurality of substantially non-biotinylated members” shall be construed broadly to mean more than one member e.g., a mixture of members or a library of members presented as a mixture notwithstanding that each member may be displayed separately from any other members in the mixture or library.

Preferably, the members may comprise a covalent link between the scaffold and the fusion protein, wherein the covalent link is cleavable by exposure to an environment within a cell or an intracellular compartment thereof. For example, the covalent link may be a disulfide bond, or an acid-cleavable link, or a pH-cleavage link such as a hydrazone bond. In one example, the intracellular environment may comprise a reducing environment of the cytoplasm of a cell, wherein the covalent link is a disulphide bond (e.g. Austin et al. Pro. Nat. Acad. Sci U.S.A 102 17987-17992, 2005). Alternatively, the members may comprise an amino acid sequence between the scaffold and the fusion protein, wherein the sequence comprises an enzyme substrate site, and wherein said members are reacted with an enzyme that acts on said enzyme substrate site to cleave the scaffold from the fusion protein, and wherein the cleaved fusion protein enters the endosome of the host cells.

In this example, the incubating at step (ii) may be for a time and under conditions such that the cleaved fusion proteins that have translocated the endosome of the host cells are enzymatically biotinylated by the expressed biotin ligase and wherein determining or identifying at step (iii) comprises determining or identifying a candidate peptide moiety at step (iii) that has translocated the endosome of the host.

In yet another preferred example, the members further comprise a domain to stabilize the expressed fusion protein or allow it to adopt a particular conformation e.g., by extending half-life of the fusion protein and/or assisting in correct presentation of the fusion protein to the host cells or to perform some other function with the host cells. For example, a domain to stabilize the expressed fusion protein may include a protein A-based domain (e.g. Nord, et al. Nat Biotechnol 15 772-777, 1997) or a lipocalin-based domain (e.g. Skerra et al. FEBS J. 275 2677-2683, 2008) or a fibronectin-based domain (e.g. Dineen et al. BMC Cancer 8 352, 2008) or an avimer domain (e.g. Silverman et al. Nat Biotechnol 23 1556-1561, 2005) or an ankyrin-based domain (e.g. Zahnd et al. J Biol. Chem. 281 35167-35175, 2006) or a centyrin domain based on a protein fold having significant structural homology to an Ig domain with loops that are analogous to CDRs.

It is within the scope of the present invention for the members to be labelled e.g., with one or more detectable reporter molecules to facilitate detection of binding, entry and localization e.g., a fluorophore, haloalkane, radioactive label, coloured particle, latex bead, nanoparticle, quantum dot, or stable enzyme such as beta lactamase.

Alternatively, the members may comprise a labile linkage between the scaffold and the fusion protein, such as an ester bond or a specific protease site, so that once the member is released to the cytosol it can be cleaved by esterases or proteases, to fluoresce. One example of such an esterase-cleavable dye is Oregon Green 488 carboxylic acid diacetate (carboxy-DFFDA)-6-isomer.

In one example, the members do not enter endosomes of the host cells. Alternatively, the members translocate the endosome of the hosts intact.

Contacting at step (i) may be for a time and under conditions sufficient for at least the displayed fusion proteins of members to enter the endosome of host cells. In this example, the incubating at step (ii) may be for a time and under conditions such that the biotin ligase substrate domain of the at least fusion proteins that have translocated out of the endosome of the host cells are enzymatically biotinylated by the expressed biotin ligase and wherein determining or identifying at step (iii) comprises determining or identifying a candidate peptide moiety at step (iii) that has translocated the endosome of the host.

In yet another example, the method additionally comprises detecting and/or isolating and/or recovering a biotinylated member. Alternatively, or in addition, the method comprises detecting and/or isolating and/or recovering a biotinylated fusion protein.

Thus, the invention provides for screening of highly diverse pools of nucleic acid encoding peptides to identify and/or isolate peptides having an ability to penetrate one or more cell membranes. In its broadest context, the invention provides peptides having cell translocation ability without reference to a particular cell type. However, the invention may also provide peptides having cell-type specificity/selectivity e.g., by performing one or more rounds of selection for or against binding and/or uptake into one or more different cell types, and/or having low toxicity e.g., by performing one or more rounds of selection for cell survival. Such additional screening for cell-type selectivity and/or low toxicity may be performed, for example, as described in WO 2012/159164.

The present invention provides enhancement of peptides having CPP-like properties relative to art-known methods. For example, relative to a process that does not require biotinylation of a non-biotinylated fusion protein comprising a candidate peptide moiety and a biotin ligase substrate domain, the process of the present invention may provide a pool of peptides wherein at least about 20% of the peptides or at least about 21% of the peptides or at least about 22% or at least about 23% of the peptides or at least about 24% of the peptides or at least about 25% of the peptides or at least about 26% of the peptides or at least about 27% of the peptides or at least about 28% of the peptides or at least about 29% of the peptides or at least about 30% of the peptides identified or isolated prior to validation have one or more CPP-like properties. CPP-like properties are determined e.g., by comparison of their primary sequence on a known database of CPPs.

Particularly-preferred peptides monitored or isolated or identified by performing the process of the invention form secondary or tertiary structures or peptide folds or assemblies of folds e.g., autonomously or by virtue of being induced to do so such as by their cyclization, wherein the structure(s) enhance(s) functionality of the peptide in translocating the membrane of the cell. For example, a peptide having CPP-like secondary structure characteristics such as one or more folds comprising alpha-helix and/or coil properties, is within the context of the invention. For example, the process of the present invention may enrich for peptides having a reduced representation of folds comprising beta-sheets e.g., to assist in penetration or translocation across the cell membrane. For example, relative to a process that does not require biotinylation of a non-biotinylated fusion protein comprising a candidate peptide moiety and a biotin ligase substrate domain, the process of the present invention may provide a pool of peptides having less than about 85% reduced beta sheet composition or less than about 80% reduced beta sheet composition or less than about 75% reduced beta sheet composition or less than about 70% reduced beta sheet composition or less than about 65% or 60% or 55% or 50% reduced beta sheet composition.

Alternatively, or in addition, the process of the present invention may provide peptide pools having reduced hydrophobicity relative to a process that does not require biotinylation of a non-biotinylated fusion protein comprising a candidate peptide moiety and a biotin ligase substrate domain. For example, relative to a process that does not require biotinylation of a non-biotinylated fusion protein comprising a candidate peptide moiety and a biotin ligase substrate domain, the process of the present invention may provide a pool of peptides having less than about 75% lower content of hydrophobic peptides or less than about 70% lower content of hydrophobic peptides or less than about 65% lower content of hydrophobic peptides or less than about 60% lower content of hydrophobic peptides or less than about 55% lower content of hydrophobic peptides or less than about 50% lower content of hydrophobic peptides or less than about 45% lower content of hydrophobic peptides or less than about 40% lower content of hydrophobic peptides or 35% lower content of hydrophobic peptides or about less than about 35-70% lower content of hydrophobic peptides.

Alternatively, or in addition, the process of the present invention may provide peptide pools having a higher isoelectric point (pI) relative to a process that does not require biotinylation of a non-biotinylated fusion protein comprising a candidate peptide moiety and a biotin ligase substrate domain. For example, relative to a process that does not require biotinylation of a non-biotinylated fusion protein comprising a candidate peptide moiety and a biotin ligase substrate domain, the process of the present invention may provide a pool of peptides having an average pI of at least about 8.5 or 8.6 or 8.7 or 8.8 or 8.9 or 9.0 or 9.5 or 10.0 or 10.5.

Alternatively, or in addition, the process of the present invention may provide peptide pools having a higher average charge relative to a process that does not require biotinylation of a non-biotinylated fusion protein comprising a candidate peptide moiety and a biotin ligase substrate domain. For example, relative to a process that does not require biotinylation of a non-biotinylated fusion protein comprising a candidate peptide moiety and a biotin ligase substrate domain, the process of the present invention may provide a pool of peptides having an average charge of at least about 2.0 or 2.1. or 2.2 or 2.3 or 2.4 or 2.5 or 2.6 or 2.7 or 2.8 or 2.9 or 3.0 or 3.1 or 3.2 or 3.3 or 3.4 or 3.5 or 3.6 or 3.7 or 3.8 or 3.9 or 4.0 or 4.1 or 4.2 or 4.3 or 4.4 or 4.5 or 4.6 or 4.7 or 4.8 or 4.9 or 5.0.

As will be known to the skilled artisan, the foregoing effects may be reflected in the amino acid composition of the pool of peptides isolated or identified by performing the process of the invention e.g., as described by way of Table 4 or Table 7 hereof.

The non-biotinylated members may be non-biotinylated by virtue of being produced in cells having no endogenous biotin ligase activity. In another example, the method additionally comprises producing the non-biotinylated members in cells having no endogenous biotin ligase activity. The term “endogenous biotin ligase activity” as used herein, shall be taken to mean that an organism, tissue, or cell expresses endogenous biotin ligase.

Alternatively, the non-biotinylated members may be non-biotinylated by virtue of being produced in cells having a biotin ligase that has a low affinity for the biotin ligase substrate domain. As used herein, the term “low affinity” shall be taken to mean an activity of less than 25% or less than 20% or less than 15% or less than 10% or less than 5% or less than 4% or less than 3% or less than 2% or less that 1% of the native biotin ligase substrate.

Alternatively, the non-biotinylated members may be non-biotinylated by virtue of being produced in cells having a biotin ligase which is active on the biotin ligase substrate domain but not able to access the biotin ligase substrates domain as the members are expressed and secreted (e.g. via the sec secretion pathway), thereby effectively avoiding biotinylation.

In yet another example, the method additionally comprises producing the non-biotinylated members in cells having a biotin ligase that has a low affinity on the biotin ligase substrate domain.

The method may additionally comprise incubating the host cells after step (ii) and prior to step (iii) with one or more agents to inhibit the activity of the biotin ligase. The agent may comprise a pyrophosphate salt and/or adenosine 5′ monophosphate (AMP) salt. The pyrophosphate salt may be a colloidal metal pyrophosphate salt or a disodium pyrophosphate salt or a tetrasodium pyrophosphate salt or a potassium pyrophosphate salt or a calcium pyrophosphate salt or a inositol pyrophosphate salt. For example, the pyrophosphate salt may have a concentration of 0.4 mM or 0.5 mM or 0.6 mM or 0.7 mM or 0.8 mM or 0.9 mM or 1 mM or 2 mM or 5 mM or 10 mM or 20 mM or a concentration in the range of 0.4 mM-20 mM or 0.5 mM-20 mM or 0.6 mM-20 mM or 0.7 mM-20 mM or 0.8 mM-20 mM or 0.9 mM-20 mM or 1 mM-20 mM or 2 mM-20 mM or 5 mM-20 mM or 10 mM-20 mM. The AMP salt may be a disodium salt, or a calcium salt or a magnesium salt. In one example, the agent may comprise the AMP salt at a concentration of no less than 100 mM or no less than 150 mM or no less than 200 mM or no less than 250 mM or no less than 300 mM. Alternatively or in addition, the agent may comprise a chaotropic salt. Alternatively or in addition, the agent may comprise a biotin analogue capable of competing with the biotin ligase substrate domain for binding of the expressed biotin ligase. Examples of biotin analogues are known in the art and are described, for example, in Blanchard et al. Biochem. Biophys. Res. Commun. 266 466-471 (1999); Levert et al. J. Biol. Chem 277 16347-16350 (2002); Eisenberg J. Bacteriol. 123 248-254 (1975). In another example, the agent may comprise ethylenediaminetetraacetic acid (EDTA). Alternatively or in addition, the agent may comprise acetonitrile.

In yet another example, the method additionally comprises treating the host cells at step (i) to remove members that are associated with the membrane of the host cells without disrupting the cell membranes. By “associated with the membrane” is meant that the peptide is in physical relation with the cell other than by means of a mechanism that is capable of transporting the peptide through the membrane of that particular cell or internalizing the peptide in that particular cell. For example, treating the host cells may comprise incubating the host cells with a protease for a time and under conditions sufficient to remove and/or inactivate extrinsic members to the host cells without disrupting the cell membrane.

The protease may be trypsin, or chymotrypsin, or thermolysis, or heparinase, or subtilisin or proteinase K. In another example, treating the cell may comprise washing the host cells for a time and under conditions sufficient to remove members that are associated with the membrane of the host cells. In this example, the cell may be washed n times using a buffer or medium compatible with cell viability or survival or that does not adversely affect the ability of another cell downstream in the subject process to internalize the peptide, wherein n is an integer having a value equal to or greater than 1 e.g., 1 or 2 or 3 or 4 or 5 or 6 or 7 or 8 or 9 or 10.

In yet another example, the method additionally comprises fractionating the plurality of non-biotinylated members prior to step (i) to thereby obtain one or more pools of members each having a net positive or net negative or net neutral charge and then performing step (i) using the one or more pools of members, for example, a pool of members may have an isoelectric point (pI) of 2 or 3 or 4 or 5 or 6 or 7 or 8 or 9 or 10 or 11 or 12, or a pI in the range of 2-10 or 2-9 or 2-8 or 2-7 or 2-6 or 2-5 or 2-4 or 2-3 or 3-10 or 4-10 or 5-10 or 6-10 or 7-10 or 8-10 or 9-10 or 3-9 or 4-9 or 5-9 or 6-9 or 7-9 or 8-9 or 3-8 or 3-7 or 3-6 or 3-5 or 3-4 or 4-8 or 5-8 or 6-8 or 7-8 or 4-7 or 4-6 or 4-5 or 5-7 or 6-7 or 5-6. For example, fractionating the plurality of non-biotinylated members comprises performing ion exchange chromatography and recovering the one or more pools of members. Preferably, the ion exchange chromatography comprises use of an anion exchanger. Alternatively, or in addition, the ion exchange chromatography comprises use of a cation exchanger. Such anion or cation exchangers are well known in the art and are commercially available. In one example, the ion exchange chromatography is a batch process. In another example, the ion exchange chromatography is a moving bed process.

In one example, the biotin ligase expressed at step (i) may be an endogenous biotin ligase of the host cells. Alternatively, the host cells employed to biotinylate the non-biotinylated members may express an endogenous biotin ligase that has a low affinity for the biotin ligase substrate domain and wherein the biotin ligase expressed at step (i) is a recombinant biotin ligase that has a high affinity for the biotin ligase substrate domain. As used herein, the term “high affinity” shall be taken to mean an activity of more than 75% or more than 80% or more than 85% or more than 90% or more than 95% or more than 96% or more than 97% or more than 98% or more that 99% of the native biotin ligase substrate.

Preferably, the recombinant biotin ligase is encoded by a gene construct comprising a promoter operably connected to nucleic acid encoding the biotin ligase, and wherein the promoter confers constitutive expression of the biotin ligase on the host cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid (e.g., a transgene), e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid (e.g., a transgene and/or a selectable marker gene and/or a detectable marker gene) to which it is operably linked Preferred promoters can contain additional copies of one or more specific regulatory elements to further enhance expression and/or alter the spatial expression and/or temporal expression of said nucleic acid. The term “constitutive expression” as used herein shall be taken to include expression under all physiological conditions. For example, a promoter that confers constitutive expression may be a CaMV 35S promoter or an opine promoter or a plant ubiquitin promoter or a rice actin-1 promoter or a maize alcohol dehydrogenase promoter or a simian virus 40 early promoter (SV40) or a cytomegalovirus immediate-early promoter (CMV) or a human Ubiquitin C promoter (UBC) or a human elongation factor 1α promoter (EF1A) or a mouse phosphoglycerate kinase 1 promoter (PGK) or a chicken β-Actin promoter coupled with CMV early enhancer (CAGG) or a copia transposon promoter (COPIA) or an actin 5C promoter (ACT5C).

Alternatively, the recombinant biotin ligase may be encoded by a gene construct comprising a promoter operably connected to nucleic acid encoding the biotin ligase, and wherein the promoter confers inducible expression of the biotin ligase on the host cells, and wherein said method further comprising growing the host cells at (i) under conditions sufficient to induce expression of the biotin ligase in the host cells. The term “inducible expression” as used herein shall is taken in its broadest context to mean activation of gene expression by the presence or absence of a biotic factor or by the presence of absence of an abiotic factor or at certain stages of development or in a particular subcellular localisation or by the presence or absence of a chemical factor or by the presence of absence of a physical factor. Promoters that confer inducible expression are known in the art and are described, for example in Weber et al. Methods Mol. Bio. 267 451-466 (2004); Dohn et al. Methods Mol. Bio. 223, 221-235 (2003); Ting et al. Methods Mol. Med 105 23-46 (2004); Borghi Methods Mol. Bio. 665 65-75 (2010). As used herein, the term “subcellular location” shall be taken to include cytosol, endosome, nucleus, endoplasmic reticulum, golgi, vacuole, mitochondrion, plastid such as chloroplast or amyloplast or chromoplast or leukoplast, nucleus, cytoskeleton, centriole, microtubule-organizing center (MTOC), acrosome, glyoxysome, melanosome, myofibril, nucleolus, peroxisome, nucleosome or microtubule. Alternatively or in addition, the recombinant biotin ligase may be encoded by a gene construct in a transgenic animal or transgenic plant, wherein the gene construct comprises a promoter operably connected to nucleic acid encoding the biotin ligase, and wherein the promoter confers either ubiquitous or tissue specific expression of the biotin ligase. As used herein, the term “tissue specific expression” shall be taken to mean any tissue or cell type within the transgenic animal or plant. For example, the recombinant may be expressed in the cytoplasm or the nucleus of a particular tissue.

In another example, the method additionally comprises producing host cells that are stably or transiently transformed with a gene construct encoding the recombinant biotin ligase. As used herein, the term “stably transformed” shall be taken to mean integration of part of or all of the exogenous nucleic acid to nuclear genomic DNA, mitochondrial or plastid DNA. The term “transiently transformed” used herein refers to introduction of part of or all of the exogenous nucleic acid to a cell that has not yet integrated into genomic, mitochondrial DNA or plastid DNA. Alternatively, the method may additionally comprise producing the transgenic animal or plant expressing a gene construct encoding the recombinant biotin ligase.

Alternatively, the host cells of the invention may lack endogenous biotin ligase activity, and wherein the biotin ligase expressed at step (i) is a recombinant biotin ligase. Preferably, the recombinant biotin ligase may be encoded by a gene construct comprising a promoter operably connected to nucleic acid encoding the biotin ligase, and wherein the promoter confers constitutive expression of the biotin ligase on the host cells. Alternatively, the recombinant biotin ligase may be encoded by a gene construct comprising a promoter operably connected to nucleic acid encoding the biotin ligase, and wherein the promoter confers inducible expression of the biotin ligase on the host cells, and wherein said method further comprising growing the host cells at (i) under conditions sufficient to induce expression of the biotin ligase in the host cells. In another example, the method additionally comprises producing host cells that are stably or transiently transformed with a gene construct encoding the biotin ligase. Alternatively or in addition, the recombinant biotin ligase may be encoded by a gene construct in a transgenic animal or transgenic plant wherein the gene construct comprises a promoter operably connected to nucleic acid encoding the biotin ligase, and wherein the promoter confers tissue specific expression of the biotin ligase. As used herein, the term “tissue specific expression” shall be taken to mean any tissue or cell type within the transgenic animal or plant. For example, the recombinant biotin ligase may be expressed in cytoplasm or mitochondria or a nucleus of a particular tissue.

Alternatively the recombinant biotin ligase may be encoded by a gene construct comprising a promoter operably connected to nucleic acid encoding the biotin ligase wherein the promoter confers expression of the biotin ligase in a particular subcellular location within the host cells. Such promoters are well known in the art and are commercially available.

The biotin ligase expressed at step (i) may comprise an amino acid sequence set forth in any one of SEQ ID NOs: 2 or 5 or 7 or 9 or 14-18 or a variant thereof having an amino acid sequence that is at least 70% identical to a biotin ligase exemplified by any one of the Sequence Listing herein, and wherein said variant has biotin ligase activity. For example, the biotin ligase expressed at step (i) may be encoded by an amino acid sequence that is at least 80% or 90% or 95% or 99% identical to any one of SEQ ID NOs: 2 or 5 or 7 or 9 or 14-18.

In another example, the biotin ligase may be fused to a polypeptide localisation signal capable of directing the biotin ligase to a particular subcellular location of the host cells. For example, the polypeptide localisation signal may be a nuclear localisation signal. Several nuclear localisation signals are known in the art and are described for example by Kalderon et al. Cell 39 499-509 (1984); Blank et al. EMBO 10 4159-4167 (1991); Emmott et al. EMBO Rep. 10 231-238 (2009); Robbins et al. Cell 64 615-623 (1991); Schmidt-Zachmann et al. J. Cell Sci. 105, 799-806 (1993). Alternatively, the polypeptide localisation signal may be a golgi localisation sequence. Several golgi localisation sequences are known in the art and are described for example by Liu et al. Mol. Biol. Cell. 18 1073-1082 (2007), Kjer-Nielsen et al. J. Cell Sci. 112 1645-1654 (1999). Alternatively, the polypeptide localisation signal may be a mitochondria localisation sequence. Several mitochondria localisation sequences are known in the art and are described for example by Neupert Annu. Rev. Biochem. 66 863-917 (1997); Plath et al. Cell 18 795-807 (1998); Rapaport EMBO Rep. 4 948-952 (2003); Beinert, Chem. Rev. 96 2335-2374 (1996); Regev-Rudzki et al. J. Cell Sci. 121 2423-2431 (2008); Horton et al. Chem. Biol. 14 375-382 (2008); Yousif et al. Chembiochem 17 1939-1950 (2009) and Yousif et al. Chembiochem 172081-2088 (2009).

The biotin ligase substrate domain may comprise an amino acid sequence defined by: LX₁X₂IX₃X₄X₅X₆KX₇X₈X₉X₁₀ (SEQ ID NO: 3), where X₁ is any amino acid; X₂ is any amino acid other than L, V, I, W, F, Y; X₃ is F or L; X₄ is E or D; X₅ is A, G, S, or T; X₆ is Q or M; X₇ is I, M, or V; X₈ is E, L, V, Y, or I; X₉ is W, Y, V, F, L, or I; and X₁₀ is preferably R, H, or any amino acid other than D or E. Preferably, the biotin ligase substrate domain may comprise an amino acid sequence defined by: LX₁X₂IX₃X₄X₅X₆KX₇X₈X₉X₁₀ (SEQ ID NO: 3), where X₁ is N; X₂ is D; X₃ is F; X₄ is E; X₅ is A; X₆ is Q; X₇ is I; X₈ is E; X₉ is W; X₁₀ is H. More preferably, the biotin ligase substrate domain may comprise an amino acid sequence set forth in SEQ ID NO: 4.

Alternatively, the biotin ligase substrate domain may comprise the amino acid sequence set forth in SEQ ID NO: 4, 6, 8, 10, 12 or 13.

In one example, the host cells are bacterial cells. In another example, the host cells are eukaryotic cells of a multicellular organism, preferably animal cells or plant cells, including protoplasts of plant cells in which the cell wall has been removed. In preferred examples, the cells are mammalian cells, including human cells. Exemplary mammalian cells are murine cells, rodent cells, hamster cells, human cells, primate cells, chicken cells, etc. Particularly preferred host cells are HEK 293 cells, CHO-K1, NIH-3T3, HeLa or COS-7 cells.

In one particularly preferred example, the scaffold is a bacteriophage.

The bacteriophage may be produced in bacterial cells that do not express a biotin ligase. Alternatively, the bacteriophage is produced in bacterial cells expressing a biotin ligase that biotinylates the biotin ligase substrate domain inefficiently and wherein said method further comprises isolating the non-biotinylated members from biotinylated members prior to step (i) to thereby provide the non-biotinylated members.

Alternatively, the bacteriophage is produced in bacterial cells expressing a biotin ligase, wherein said cells further comprise a polypeptide comprising a biotin ligase substrate domain, and wherein the cellular biotin ligase biotinylates the polypeptide in preference to the members to thereby provide the non-biotinylated members. For example, the polypeptide may comprise a plurality of biotin ligase substrate domains to thereby provide preferential biotinylation of the polypeptide relative to the biotin ligase substrate domain of the fusion protein. For example, the polypeptide may comprise 2 or 3 or 5 or 6 or 7 or 8 or 9 or 10 biotin ligase substrate domains. In one particularly preferred example, the polypeptide comprises three biotin ligase substrate domains. In accordance with this example, the fusion protein may have one biotin ligase substrate domain. In yet another example, the polypeptide further comprises a scaffold moiety. As used herein, the term “scaffold moiety” shall is taken in its broadest context to mean a protein or polypeptide that adopts a stable tertiary structure or a stable quaternary structure. For example, the scaffold moiety may be a small ubiquitin-related modifier peptide.

Preferably, the bacteriophage is a filamentous phage. For example, the filamentous phage may be a M13 phage or a f1 phage or a fd phage or a IKe phage or a If1 or a If2 phage. In one particularity preferred example, the filamentous phage is M13.

In one example, a filamentous phage comprises nucleic acid encoding the fusion protein operably linked to a nucleic acid sequence encoding a signal peptide that promotes translocation of the fusion protein across an inner membrane of a cell.

For example, the signal peptide may direct the fusion protein to the signal recognition particle (SRP) pathway. For example, the signal peptide may be a DsbA signal peptide, a TorT signal peptide, a TolB signal peptide or a Sfm signal peptide (e.g. Steiner et al. Nat. Biotech 24, 823-831, 2006). Preferably, the signal peptide is a DsbA signal peptide comprising the amino acid sequence set forth in SEQ ID NO: 20, or a TorT signal peptide comprising the amino acid sequence set forth in SEQ ID NO: 21, or a TolB signal peptide comprising the amino acid sequence set forth in SEQ ID NO: 22, or a Sfm signal peptide comprising the amino acid sequence set forth in SEQ ID NO: 23. Alternatively, the signal peptide may direct the fusion protein to a general secretory (SEC) pathway. For example, the signal peptide may a Lam signal peptide, a MalE signal peptide, a MglB signal peptide, a OmpA signal peptide, or a Pel signal peptide (e.g. Steiner et al. Nat. Biotech 24, 823-831, 2006). Preferably, the signal peptide may be a Lam signal peptide comprising the amino acid sequence set forth in SEQ ID NO: 24, or a MalE signal peptide comprising the amino acid sequence set forth in SEQ ID NO: 25 or a MglB signal peptide comprising the amino acid sequence set forth in SEQ ID NO: 26, or an OmpA signal peptide comprising the amino acid sequence set forth in SEQ ID NO: 27, or. a PelB signal peptide comprising the amino acid sequence set forth in SEQ ID NO: 31. Alternatively, the signal peptide may direct the fusion protein to the twin-arginine translocation (TAT) pathway. For example, the signal peptide may be a AmiA signal peptide, a AmiC signal peptide, a CueO signal peptide, a DmsA signal peptide, a FdnG signal peptide, a FhuD signal peptide, a HyaA signal peptide, a HybO signal peptide, a MdoD signal peptide, a NapA signal peptide, a NrfC signal peptide, a SufI signal peptide, a TorA signal peptide, a TorZ signal peptide, or a YcdB signal peptide (e.g. Tullman-Ercek et al. J. Biol. Chem. 282 8309-8316, 2007). Preferably, the signal peptide may be a TorA signal peptide comprising the amino acid sequence set forth in SEQ ID NO: 29.

In a particularly preferred example, the signal peptide is selected from the group consisting of pelB, gIII, ompA, phoA, malE, torA and sufI. For example, the present inventors have tested the effect of 11 different signal peptides on a level of expression of recombinant codon-optimized BirA protein in the E. coli periplasm using a low copy plasmid pD881 carrying the p15a ori, the inducible rhamnose promoter and a strong ribosome binding site (RBS), and demonstrated that pelB, gIII, ompA, phoA, malE, torA or sufI provide measurable biotinylation of a biotin ligase substrate (Avi V5) in DELFIA whereas only low biotinylation of the substrate occurs using ompT, dsbA or torT.

In a further preferred example, the signal peptide is a SEC pathway leader selected from the group consisting of pelB, gIII, ompA, phoA, and malE, including a pelB leader or a gIII leader or a ompA leader or a phoA leader or a malE leader. Such a leader provides for enhanced expression and enhanced periplasmic localization of functional BirA protein in bacterial cells, such as E. coli.

In another example, the biotin ligase is co-expressed in the periplasm of a bacterial cell, e.g., E. coli, with a periplasmic chaperone and/or a peptidyl-prolyl isomerase to improve or enhance of facilitate correct folding of the biotin ligase in the periplasm. In a particularly preferred example, FpkA and/or SurA e.g., as described by Schlapschy et al. PEDS, 19(8), pp. 385-390 (2006) is co-expressed with BirA to improve folding in the periplasm of a bacterial cell.

In these examples, the encoded fusion protein is generally linked to a coat protein of the filamentous phage. For example, the coat protein may be a pIII coat protein or a pVI coat protein or a pVII coat protein or a pVIII coat protein or a pIX coat protein. Preferably, the coat protein is a pIII coat protein comprising the amino acid sequence set forth in SEQ ID NO: 41. Alternatively, the coat protein is a pVIII coat protein comprising the amino acid sequence set forth in SEQ ID NO: 41.

In another example, the bacteriophage may be a T phage. For example, the T phage may a T3 phage or a T4 phage or a T7 phage. In a particularity preferred example, the T phage is a T7 phage.

In another example, the bacteriophage may be a lysogenic bacteriophage.

In another example, the bacteriophage may be a lambda phage.

In yet another example, non-biotinylated members may be produced for in vitro display method of the fusion proteins on the scaffolds. For example, the in vitro display may be a ribosome display, a covalent display or a mRNA display. In this example, the scaffold may be a ribosome or a RepA protein or a DNA puromycin linker or an RNA puromycin linker or a nucleic acid.

In one example, the fusion protein additionally comprises a moiety that may interact with a surface bound protein of the host cells, wherein the interaction between the moiety and the surface bound protein induces binding of at least the fusion protein to the host cell and/or induces cellular uptake of at least the fusion protein.

Alternatively, the fusion protein additionally comprises a moiety that may interact with a receptor displayed on a surface of the host cells, wherein the interaction between the moiety and the receptor may induce binding of at least the fusion protein to the host cell and/or induce cellular uptake of at least the fusion protein. The interaction between the moiety and the receptor may initiate internalization for example as described by Doherty et al. Annu. Rev. Biochem. 78 857-902 (2009).

Alternatively, the fusion protein further comprises a moiety that interacts with a polysaccharide displayed on a surface of the host cells, wherein the interaction between the moiety and the polysaccharide induces binding of at least the fusion protein to the host cell and/or induces cellular uptake of at least the fusion protein.

As used herein, the term “polysaccharide” shall be taken to mean a monosaccharide polymer may contain two or more linked monosaccharides. The term “polysaccharide” also includes polysaccharide derivatives, such as amino-functionalized and carboxyl-functionalized polysaccharide derivatives, among many others.

In another example, the fusion protein may additionally comprise one or more moieties that direct targeting of the member to a specific cell type and/or induce a phenotype upon entry into the host cell. For example, the moiety may be employed to induce a lethal phenotype when the member enters the host cell. For example, the moiety may be shepherdin (e.g. Plescia et al. Cancer Cell 7 457-468, 2005) or a peptide such as PRKYLRSVG derived from YB1 (e.g. Law et al. PLoS ONE 5 e12661, 2010).

Determining or identifying a candidate peptide moiety at step (iii) may comprise contacting the host cell or cell lysate or extract thereof with a biotin-binding molecule attached to a solid support for a time and under conditions sufficient for binding of the biotinylated fusion protein to the biotin binding molecule and recovering the biotinylated fusion protein. For example, the biotin-binding molecule comprises avidin or neutravidin or streptavidin or a variant thereof.

As used herein, the term “solid support” shall be taken to include any solid (flexible or rigid) substrate onto which one or more binding agents may be applied. For example, the solid support may be in the form of a bead, column, membrane, microwell or centrifuge tube. Preferably, the solid support may be a bead and wherein the bead is a glass bead, or microbead, magnetic bead, or paramagnetic bead.

As used herein “candidate peptide moiety” shall be taken to include a peptide produced using any nucleic acid isolated, identified and/or characterised nucleic acid. For example, nucleic acid encoding candidate peptide moieties may be comprise genomic DNA and/or cDNA fragments of pathogenic organisms e.g., pathogenic bacteria and viruses. In a particularly preferred example, nucleic acid encoding candidate peptide moieties may be produced from coding and/or non-coding regions of bacterial and/or archeal and/or viral genomes and/or those of eukaryotes having compact genomes.

The peptides monitored or identified by the screening method of the invention are functional in delivering a cargo molecule e.g., a fluorescent molecule, or a toxin or catalytic subunit/fragment thereof or a maltose-binding protein, or a virus particle to a cell. A peptide identified and/or isolated or purified by performing a process of the present invention is readily formulated into a conjugate comprising the peptide, or an analog and/or derivative thereof, and at least one cargo for delivery to a cell or sub-cellular location. A conjugate may be produced by linking at least one peptide or an analog and/or derivative thereof to a cargo molecule of diagnostic or therapeutic utility. Pharmaceutical compositions e.g., formulated for parenteral administration, are also produced comprising at least one such conjugate and a pharmaceutically-acceptable carrier or excipient. It will also be apparent that a cargo molecule is readily transported across a cell membrane and/or internalized within a cell or a sub-cellular location, by contacting the cell with at least one such conjugate or pharmaceutical composition for a time and under conditions sufficient for the conjugate to cross the cell membrane.

Accordingly, the present invention also provides a method of identifying a cell penetrating peptide capable of transporting a cargo moiety to a subcellular location, the method comprising the performing a functional assay to determine the ability of the peptide to translocate a cargo moiety to a subcellular location of a cell.

As used herein, the term “subcellular location” shall be taken to include cytosol, endosome, nucleus, endoplasmic reticulum, golgi, vacuole, mitochondrion, plastid such as chloroplast or amyloplast or chromoplast or leukoplast, nucleus, cytoskeleton, centriole, microtubule-organizing center (MTOC), acrosome, glyoxysome, melanosome, myofibril, nucleolus, peroxisome, nucleosome or microtubule or the cytoplasmic surface such the cytoplasmic membrane or the nuclear membrane.

As used herein, the term “cargo moiety” in its broadest sense includes any small molecule, carbohydrate, lipid, nucleic acid (e.g., DNA, RNA, siRNA duplex or simplex molecule, or miRNA), peptide, polypeptide, protein, bacteriophage or virus particle, synthetic polymer, resin, latex particle, dye or other detectable molecule that are covalently linked to the peptide directly or indirectly via a linker or spacer molecule e.g., a carbon spacer or linker consisting of amino acids of low immunogenicity. In one example, the cargo moiety may comprise a molecule having therapeutic utility or diagnostic utility. Alternatively, the cargo moiety may a toxin or a toxin subunit of fragment thereof.

The present invention also provides a method of identifying a cell penetrating peptide capable of transporting a cargo moiety to a subcellular location, the method comprising

(a) performing the method of the invention to determine or identify a candidate peptide moiety that has translocated through the cell membrane;
(b) recovering at least a biotinylated fusion protein comprising a peptide capable of translocating a cell membrane;
(c) obtaining a nucleic acid sequence encoding at least the peptide of the recovered biotinylated fusion protein;
(d) producing the peptide; and
(e) performing a functional assay to determine the ability of the peptide to translocate a cargo moiety to a subcellular location of a cell.

In one example, the functional assay may comprise:

(f) contacting test cells with a toxin conjugate, wherein the toxin conjugate may comprise a peptide linked to a cargo comprising a toxin or catalytic subunit/fragment thereof, and wherein contacting may be for a time and under conditions sufficient for toxin conjugates to enter the test cells;
(g) incubating the test cells for a time and under conditions sufficient for toxin conjugates to reduce viability of the test cells; and
(h) detecting reduced viability of the test cells, wherein reduced viability of the test cells indicates that the peptide has translocated the toxin or catalytic subunit/fragment to a subcellular location of the cell.

As described herein, the term “toxin conjugate” shall be taken to include a comprise a peptide linked to a cargo comprising a toxin or catalytic subunit/fragment thereof For example, the toxin conjugate may be lethal to the test cells (e.g. Dosio et al. Toxins 3 848-883, 2011).

Any art-recognized method may be employed to determine the viability of the test cells. For example, determining viability of the cell comprises determining the doubling rate of the cell e.g., the period of time required for the cell to divide e.g., nucleic acid content or cell counting such as by FACS.

As used herein, the term “reduced viability” refers to the viability of a cell in the presence of an internalized toxin conjugate as indicated by an inability of the cell to divide or an ability of the cell to divide in less than 10-fold or less than 9-fold or less than 8-fold or less than 7-fold or less than 6-fold or less than 5-fold or less than 4-fold or less than 3-fold or less than 2-fold or less than 1.5-fold the time taken for the cell to divide in the absence of the toxin conjugate.

In another example, viability of the cell is determined by measuring a level of one or more metabolic substrates or enzymes that are indicative of cell viability, wherein a reduce level of the one or more metabolic substrates or enzymes in the cell is indicative of reduced viability of the cell. In one example, a level of adenosine triphosphate (ATP) may be determined e.g., by measuring an increase in luminescence of luciferin in the presence of cell lysates, by virtue of cellular ATP production providing a substrate for luciferase enzyme. In another example, a level of reductase enzyme activity may be determined e.g., by colorimetric assay involving the reduction of a tetrazolium salt dye e.g., 3-(4,5-dimethylthiazol-2-yl)-2<5-diphenyltetrazolium bromide (MMT) or 2,3-6w-(2-methoxy-4-nitro-5-sulfophenyl)-2H-tetrazolium-5-carboxanilide (XTT) to a corresponding formazan in the presence of cellular reductase enzyme. In another example, viability of the cell in the presence of the bound and/or internalized toxin conjugate is indicated by a level of ATP and/or a level of reductase that is more than 50% or more than 60% or more than 70% or more than 80% or more than 85% or more than 90% or more than 95% the level in the cell in the absence of the peptide. More preferably, viability of the cell in the presence of the bound and/or internalized toxin conjugate is indicated by the same level of ATP and/or a reductase in the presence and absence of the peptide.

The toxin may comprise a Diphtheria toxin fragment. Alternatively, the toxin may comprise a Cholera toxin subunit A1. Alternatively, the toxin may comprise a Pseudomonas exotoxin. Alternatively, the toxin may comprise a ribosome inactivating protein. For example, the ribosome inactivating protein may be a type I ribosome inactivating protein. Preferably, the type I ribosome inactivating protein may be bouganin or gelonin or saporin. Alternatively, the ribosome inactivating protein may be a type II ribosome inactivating protein. Preferably, the type II ribosome inactivating protein may be a fragment A1 of the Shiga toxin or ricin or abrin, or nigrin. Alternatively, the ribosome inactivating protein may be a type III ribosome inactivating protein.

Preferably, the toxin is a bouganin polypeptide. Preferably, the bouganin is expressed in a fusion protein construct set forth in any one of SEQ ID Nos: 120-132, further comprising a candidate CPP peptide or CPP fragment or a known CPP for which CPP activity is to be confirmed is presented in a portion thereof. Preferably, the candidate CPP peptide or CPP fragment or a known CPP for which CPP activity is to be confirmed is presented in an N-terminal portion of the bouganin fusion protein e.g., after residue 2 thereof, or in a C-terminal portion of the bouganin fusion protein e.g., within 2 or 3 or 4 or 5 residues of or at the C-terminus thereof.

Detecting expression of a toxin conjugate may comprise performing fluorescence-activated cell sorting (FACS) or live confocal microscopy. The method may additionally comprise producing the toxin conjugate.

In another example, the functional assay may comprise:

(f) expressing a first moiety in a test cell, the first moiety comprising a first fragment of a detectable molecule;
(g) contacting the test cell with a second moiety comprising the peptide linked to a cargo moiety comprising a second fragment of the detectable molecule for a time and under conditions sufficient for binding of the second moiety to the test cell and uptake of the second moiety by the test cell;
(h) incubating the test cells for a time and under conditions sufficient for the first moiety and second moiety to constitute the detectable molecule or produce an activity of the detectable moiety; and
(i) detecting the detectable molecule in the test cell, wherein said detection indicates that the peptide has translocated the second fragment to a subcellular location of the test cell.

In one example, the first fragment and the second fragment of the detectable molecule are not the same. Thus, two different fragments that are essential for functionality of the detectable molecule may be reconstituted to produce a functional detectable molecule in accordance with this example. It is entirely within the scope of this example for the first and second fragment to comprise two different polypeptide monomers of a dimeric detectable molecule to be reconstituted to produce a functional detectable molecule.

In another example, the first fragment and the second fragment of the detectable molecule are the same. It is entirely within the scope of this example for the first and second fragment to comprise two identical polypeptide monomers of a dimeric detectable molecule to be reconstituted to produce a functional detectable molecule.

The constituted detectable molecule may be a fluorescent molecule that is detectable using methods well known in the art. Exemplary fluorescent proteins can include, but are not limited to, green fluorescent protein (GFP) or enhanced green fluorescent protein (EGFP) or AcGFP or TurboGFP or Emerald or Azami Green or ZsGreen, EBFP, or Sapphire or T-Sapphire or ECFP or mCFP or Cerulean or CyPet or AmCyanl or Midori-Ishi Cyan or mTFP1 (Teal) or enhanced yellow fluorescent protein (EYFP) or Topaz or Venus or mCitrine or YPet or PhiYFP or ZsYellow1 or mBanana or Kusabira Orange or mOrange or dTomato or dTomato-Tandem or AsRed2 or mRFP1 or JRed or mCherry or HcRedl or mRaspberry or HcRedl or HcRed-Tandem or mPlum or AQ 143.

A fragment of the detectable molecule may comprises an amino acid sequence comprising a GFP 11 tag and a fragment of the detectable molecule may comprise an amino acid sequence comprising a GFP 1-10 detector (e.g. Cabantous et al. Nat. Biotechnol. 23 102-107, 2005). Preferably, the GFP 11 tag may comprise an amino acid sequence set forth in SEQ ID NO: 81 and the GFP 1-10 detector may comprise an amino acid sequence set forth in SEQ ID NO: 86. The term “split-GFP complementation” is used in the working examples hereof to reference any and all forms of a functional assay employing a GFP 11 tag and GFP 1-10 detector.

In one example, the nucleic acid encoding the GFP 11 tag is linked to a nucleic acid encoding a scaffold molecule, such that a fusion polypeptide comprising the scaffold and the GFP 11 is produced. For example, the scaffold molecule may include a small ubiquitin-related modifier peptide or a tubulin peptide or a β-actin peptide or a protein A-based domain (e.g. Nord, et al. Nat Biotechnol 15 772-777, 1997) or a lipocalin-based domain (Skerra et al. FEBS J. 275 2677-2683, 2008) or a fibronectin-based domain (e.g. Dineen et al. BMC Cancer 8 352, 2008) or an avimer domain or Sumo (e.g. Silverman et al. Nat Biotechnol 23 1556-1561, 2005) or an ankyrin-based domain (e.g. Zahnd et al. J Biol. Chem. 281 35167-35175, 2006) or a centyrin domain based on a protein fold having significant structural homology to an Ig domain with loops that are analogous to CDRs or MyD88 or the T-cell differentiation protein Mal or a viral oncogene such as the protein RelA encoded by the v-rel avian reticuloendotheliosis viral oncogene homolog A.

The GFP 11 tag may comprise a CPP or peptide being screened for CPP activity, or alternatively, the GFP 1-10 detector may comprise a CPP or peptide being screened for CPP activity.

Detecting the detectable molecule may comprise performing a fluorescence-based assay e.g., fluorescence-activated cell sorting (FACS) or fluorescence microscopy or live confocal microscopy or a combination thereof to detect the fluorophore(s). For example, in performing microscopy for determining reconstitution of GFP activity in cells, the cells may be transfected with constructs comprising the GFP1-10 and GFP 11 fragments as described herein, then seeded into chamber slides such as those having a charged surface to facilitate adherence of the cells. For example, CHO-K1 cells may be seeded at 5×10⁴ cells/well and HCC-827 cells may be seeded at 7.5×10⁴ cells/well, in 250 uL of media lacking antibiotic, and left to settle and adhere for up to 8-16 hours e.g., overnight. Following adherence, recombinant protein may be added by removing media e.g., 60 μL media, from the wells and adding an approximately equivalent volume of protein e.g., 60 μL of 40 μM working stock solution of protein, to thereby produce a final concentration of 10 μM protein per well. Following a further incubation period of up to 48 hours, preferably 8-24 hours or 8-16 hours, media are removed from the cells gently such as using a pipette, and the cells are fixed or permeabilized such as by using a commercially-available kit e.g., Image-iT Fix-Perm kit from Molecular Probes, Life Tech, according to the manufacturer's instructions. Slides having the fixed cells adhered thereto are washed and blocked e.g., using BSA in DPBS, and fluorescence is visualized by incubating the cells in the presence of a fluorophore e.g., ActinRed 555 Ready Probes Reagent, then washed, stained e.g., using DAPI/PBS, and washed, flicked dry, and visualised by fluorescence microscopy.

As exemplified herein, the inventors faced several challenges in achieving reconstitution of functional GFP when a fragment such as the GFP 11 tag is covalently-linked to a CPP or peptide being screened for CPP-like activity, including adverse effects on cellular viability. In particular, the data presented in FIGS. 13-22 hereof show that a functional assay that comprises determining reconstitution of split GFP activity in cells expressing GFP 11+GFP 1-10 fragments is useful for (i) detecting CPP-cargo-GFP 11 fusion polypeptide uptake into cells by determining fluorescence of the reconstituted GFP; and/or (ii) determining the ability of the CPP to modulate escape of a linked cargo protein from the endosome of the cell.

Difficulty in achieving adequate fluorescence signal and cellular viability is notwithstanding efficient reconstitution of isolated GFP 11 tag and GFP 1-10 detector fragments in the absence of such covalently-linked additional peptidyl moieties. The inventors found that the signal, reflective of the level of reconstitution of the fragments, was enhanced by employing a GFP 11 fusion, preferably a fusion comprising GFP 11 and a further polypeptide fragment, such as a MyD88 peptide fragment, a Sumo peptide fragment, or a β-actin peptide fragment, however the viability of cells expressing these additional polypeptides was variable. For example, data presented in FIG. 14 hereof demonstrate that co-transfection of cells with a fragment comprising a MyD88-GFP 11 fragment and a GFP1-10 fragment produces dense pockets of reconstituted intracellular GFP mainly in rounded cells; co-transfection of cells with a fragment comprising a β-actin-GFP 11 fragment and a GFP1-10 fragment produces diffuse localization of split GFP labelling throughout the cytoplasm, concentrated at dendritic features; co-transfection of cells with a fragment comprising a RelA-GFP 11 fragment and a GFP1-10 fragment produces diffuse localization of split GFP throughout cytoplasm and sometimes excluded from nucleus; and co-transfection of cells with a fragment comprising a Mal-GFP 11 fragment and a GFP1-10 fragment produces split GFP expression that is diffuse throughout cytoplasm and concentrated in multiple small foci. Cellular viability was higher for cells expressing Mal-GFP 11 fusions or β-actin-GFP 11 fusions, whereas expression of MyD88-GFP 11 fusions or RelA-GFP 11 fusions reduced cellular viability.

Alternatively, or in addition, the nucleic acid encoding one or both fragments of the detectable molecule may be optimized for human codon usage to enhance the level of reconstitution of the detectable molecule ex vivo. As exemplified herein by way of FIG. 15, such human codon optimization improves split GFP signal in human cells, at least for reconstituted GFP 11 and GFP 1-10 fragments. Preferably, the GFP1-10-encoding nucleic acid has been modified further by substituting a mutant nucleotide A of the commercially-available GFP 1-10 for G at the appropriate position, to produce a human-optimized and corrected amino acid sequence (herein “hGFP1-10(g)”). Preferably, a human-codon optimized and corrected GFP 1-10 sequence is expressed from a pcDNA4/TO vector in human cells (herein “hGFP1-10(g)/TO”). Preferably, such codon-optimized GFP 1-10 is employed with a Mal-GFP 11 or MyD88-GFP 11 fusion construct to achieve elevated reconstitution. More preferably, such codon-optimized GFP 1-10 is employed with a Mal-GFP 11 fusion construct to achieve elevated reconstitution of functional GFP with high or enhanced or tolerable cell viability.

In a further example, a linker may be placed between a scaffold and GFP 11. For example, the linker may comprise up to 25 amino acid residues in length or up to 20 amino acid residues in length, such as 20 amino acid residues or 19 amino acid residues or 18 amino acid residues or 17 amino acid residues or 16 amino acid residues or 15 amino acid residues or 14 amino acid residues or 13 amino acid residues or 12 amino acid residues or 11 amino acid residues or 10 amino acid residues or 9 amino acid residues or 8 amino acid residues or 7 amino acid residues or 6 amino acid residues or 5 20 amino acid residues or 4 amino acid residues.

In a further example, the method further comprises performing a process comprising in vitro complementation of tag and detector fusion(s) to thereby determine a combination of fusion polypeptides that provide optimum reconstitution of the detectable molecule for the CPP being tested. This is to minimize adverse effects of the CPP on reconstitution of the detectable molecule. For example, a particular test CPP may be expressed as a fusion with different scaffolds and GFP 11 in human cells e.g., HCC-827 (high receptor expression) and in non-human cells e.g., CHO-K1 (negative receptor expression) cells that are transfected with human codon-optimized hGFP1-10(g)/TO construct, and split GFP complementation detected by measuring GFP fluorescence such as by flow cytometry, gating on the live cell population. The signal is preferably dose-responsive. Preferably, the signal is expressed as percent GFP-positive cells in the total live cell population, and normalized for the level of transfection efficiency as determined for an independent transfection of each cell line with a different construct such as pcDNA3-eGFP. An exemplary workflow of this preferred testing is provided by way of FIG. 19 hereof.

Any cell line may be employed for performing the functional assays described herein. Preferred cells lines are human HCC cells e.g., HCC-827 cells, or non-human cells such as CHO cells or HEK cells. Preferred CHO cells are CHO-K1 cells, Preferred HEK cells are HEK-293 cells.

In yet another example, the functional assay may comprise:

(f) contacting test cells comprising fibroblasts with a fusion protein comprising a peptide and a transcription factor that is functional in a subcellular localisation of the cell and mediates differentiation of the fibroblasts to a different cell type;
(g) incubating the test cells for a time and under conditions sufficient for their differentiation to occur; and
(h) detecting the differentiated cells, wherein the differentiated cells indicate that the peptide has translocated the transcription factor to a subcellular location of the test cells.

In one example, the fibroblasts may be primary fibroblasts of human origin such as human dermal fibroblast or carcinoma associated fibroblasts.

Preferably, the transcription factor is OCT-4 and wherein the differentiation cells are lymphocytes (e.g. Szabo et al. Nature 25, 521-526, 2010). More preferably, the transcription factor is MYOD1 and wherein the differentiation cells are myoblasts (e.g. Fijii et al., Brain Dev. 28, 420-425, 2006).

Detecting the differentiated cells may comprise performing microscopy or fluorescence-activated cell sorting (FACS).

It is to be understood that the present invention also extends to a method for determining activity of a CPP comprising performing a functional assay as described according to any example hereof as a stand-alone process or in isolation from performing any screening to isolate or identify a putative CPP from other peptides. For example, the present invention clearly provides a method for determining activity of a CPP comprising performing a functional assay as described according to any example hereof that comprises determining reconstitution of split GFP activity in cells expressing GFP 11+GFP 1-10 fragments for the detection of CPP-cargo-GFP 11 fusion polypeptide uptake into cells by determining fluorescence of the reconstituted GFP.

In a further example, the present invention provides a recombinant or synthetic CPP comprising an amino acid sequence set forth in any one or more of SEQ ID Nos: 83-119 including SEQ ID NO: 83 and/or SEQ ID NO: 84 and/or SEQ ID NO: 85 and/or SEQ ID NO: 86 and/or SEQ ID NO: 87 and/or SEQ ID NO: 88 and/or SEQ ID NO: 89 and/or SEQ ID NO: 90 and/or SEQ ID NO: 91 and/or SEQ ID NO: 92 and/or SEQ ID NO: 93 and/or SEQ ID NO: 94 and/or SEQ ID NO: 95 and/or SEQ ID NO: 96 and/or SEQ ID NO: 97 and/or SEQ ID NO: 98 and/or SEQ ID NO: 99 and/or SEQ ID NO: 100 and/or SEQ ID NO: 101 and/or SEQ ID NO: 102 and/or SEQ ID NO: 103 and/or SEQ ID NO: 104 and/or SEQ ID NO: 105 and/or SEQ ID NO: 106 and/or SEQ ID NO: 107 and/or SEQ ID NO: 108 and/or SEQ ID NO: 109 and/or SEQ ID NO: 110 and/or SEQ ID NO: 111 and/or SEQ ID NO: 112 and/or SEQ ID NO: 113 and/or SEQ ID NO: 114 and/or SEQ ID NO: 115 and/or SEQ ID NO: 116 and/or SEQ ID NO: 117 and/or SEQ ID NO: 118 and/or SEQ ID NO: 119.

In a further example, the present invention provides a recombinant or synthetic CPP comprising at least about 5 or 6 or 7 or 8 contiguous amino acids of an amino acid sequence set forth in any one of SEQ ID Nos: 83-119, including at least about 15 or 20 or 25 or 30 or 35 contiguous amino acids of an amino acid sequence set forth in any one of SEQ ID Nos: 83-119. It is to be understood in this context that such fragments of a full-length CPP disclosed herein are functional CPPs in the sense that they possess the same functionality, albeit not necessarily the same magnitude of functionality, as the base CPPs form which they are derived, when tested in one or more of the exemplified screens herein for CPP activity.

Particularly preferred CPPs and CPP fragments of the present invention are longer than about 23 amino acid residues in length, preferably at least about 25 or 26 or 27 or 28 or 29 or 30 or 31 or 32 or 33 or 34 or 35 or 36 or 37 or 38 or 39 or 40 residues in length.

In a further example, the present invention provides a conjugate molecule comprising: (i) a recombinant or synthetic CPP or CPP fragment of the present invention according to any example hereof, such as a CPP defined by one or more of SEQ ID NOs: 83-119 or a functional CPP fragment thereof, and (ii) a cargo molecule covalently bound to the CPP or CPP fragment. The cargo may be a small molecule, carbohydrate, lipid, nucleic acid, peptide, polypeptide, protein, cell, bacteriophage particle, virus particle, synthetic polymer, resin, latex particle, or a dye. Alternatively, or in addition, the cargo may comprise or consist of a diagnostic reagent, such as a detectably-labelled molecule e.g., a fluorophore, radioactive label, luminescent molecule, nanoparticle, contrast agent, or quantum dot. Alternatively, or in addition, the cargo may comprise or consist of an enzyme that converts a cell-permeable substrate thereof into a detectable molecule that may be a fluorescent or coloured molecule. For example, the cargo may exhibit β-lactamase activity in the presence of a substrate comprising CCF4-AM. Alternatively, or in addition, the cargo may comprise or consist of a therapeutic or diagnostic reagent having utility in of a disease or condition of the central nervous system, or a cancer.

In a further example, the present invention provides a method of transporting a cargo molecule across a cell membrane or internalizing a cargo molecule within a cell or a sub-cellular location, said method comprising contacting the cell with at least one conjugate according to any example hereof for a time and under conditions sufficient for the conjugate to cross the cell membrane. The method may further comprise producing the conjugate by a process comprising associating or linking covalently a cargo molecule to a CPP or CPP fragment of the invention as described according to any example hereof, such as a CPP defined by one or more of SEQ ID NOs: 83-119 or a functional CPP fragment thereof.

Throughout this specification, unless specifically stated otherwise or the context requires otherwise, reference to a single step, composition of matter, group of steps or group of compositions of matter shall be taken to encompass one and a plurality (e.g. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and/or all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts:
1. Sambrook, Fritsch & Maniatis, Molecular Cloning: A Laboratory Manual, Cold Spring Harbor Laboratories, New York, Third Edition (2001), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp1-22; Atkinson et al., pp35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. Land Fenichel, R. L. (1976). Biochem. Biophys. Res. Commun. 73, 336-342
11. Merrifield, R. B. (1963). J. Am. Chem. Soc. 85, 2149-2154.
12. Barmy, G. and Merrifield, R. B. (1979) in The Peptides (Gross, E. and Meienhofer, J. eds.), vol. 2, pp. 1-284, Academic Press, New York.
13. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
17. Handbook of Experimental Immunology, Vols. I-IV (D. M. Weir and C. C. Blackwell, eds., 1986, Blackwell Scientific Publications).
18. McPherson et al., In: PCR A Practical Approach., IRL Press, Oxford University Press, Oxford, United Kingdom, 1991.
19. Methods in Yeast Genetics: A Cold Spring Harbor Laboratory Course Manual (D. Burke et al., eds) Cold Spring Harbor Press, New York, 2000 (see whole of text).
20. Guide to Yeast Genetics and Molecular Biology. In: Methods in Enzymology Series, Vol. 194 (C. Guthrie and G. R. Fink eds) Academic Press, London, 1991 2000 (see whole of text).

The present invention is described further in the following non-limiting examples, and/or as shown in the figures.

### BRIEF DESCRIPTION OF THE DRAWINGS

FIG. 1a is a schematic representation of the encoded pIII fusion protein of the pNp3 derivative vector PelB-Avitag-pIII. Expression vector PelB-Avitag-pIII comprises nucleic acid encoding a PelB leader signal peptide (PelB), to direct export of any expressed polypeptide to the periplasm of an E. coli cell; nucleic acid encoding a hexahistidine tag (6 His), for detection and/or purification of the fusion protein; nucleic acid encoding a hemagglutinin (HA) tag, for detection and/or purification of the fusion protein; nucleic acid encoding a biotin ligase substrate domain set forth in SEQ ID NO: 4 (Avitag) and nucleic acid encoding a phage coat protein pIII (pIII). Also shown is an EcoRI restriction enzyme site to allow for sub-cloning of a candidate peptide moiety.

FIG. 1b is a schematic representation of the encoded pIII fusion protein of the pNp3 derivative vector DsbA-Avitag-pIII. Expression vector DsbA-Avitag-pIII comprises nucleic acid encoding a DsbA leader signal peptide (DsbA), to direct export of any expressed polypeptide to the periplasm of an E. coli cell; nucleic acid encoding a hexahistidine tag (6 His), for detection and/or purification of the fusion protein; nucleic acid encoding a hemagglutinin (HA) tag, for detection and/or purification of the fusion protein; nucleic acid encoding a biotin ligase substrate domain set forth in SEQ ID NO: 4 (Avitag) and nucleic acid encoding a phage coat protein pIII (pIII). Also shown is an EcoRI restriction enzyme site to allow for sub-cloning of a candidate peptide moiety.

FIG. 1c is a schematic representation of the encoded pIII fusion protein of the pNp3 derivative vector TorA-Avitag-pIII. Expression vector TorA-Avitag-pIII comprises nucleic acid encoding a TorA leader signal peptide (TorA), to direct export of any expressed polypeptide to the periplasm of an E. coli cell; nucleic acid encoding a hexahistidine tag (6 His), for detection and/or purification of the fusion protein; nucleic acid encoding a hemagglutinin (HA) tag, for detection and/or purification of the fusion protein; nucleic acid encoding a biotin ligase substrate domain set forth in SEQ ID NO: 4 (Avitag) and nucleic acid encoding a phage coat protein pIII (pIII). Also shown is an EcoRI restriction enzyme site to allow for sub-cloning of a candidate peptide moiety.

FIG. 2 is a schematic representation of a fusion polypeptide comprising three tandem copies of a biotin ligase substrate domain (Avitag) fused to a Small Ubiquitin-like Modifier (SUMO) protein designed to function as a competitive decoy substrate.

FIG. 3 is a photographic representation of the detection of biotinylated member by western blot analysis. Members comprising scaffolds in the form of filamentous bacteriophage displaying fusion proteins were produced in E. coli cells expressing an endogenous biotin ligase. Molecular weight marker proteins (lane 1), filamentous bacteriophage displaying PelB-Avitag-pIII fusion proteins (lanes 2 and 3), filamentous bacteriophage displaying DsbA-Avitag-pIII fusion proteins (lane 4, 5), filamentous bacteriophage displaying fusion protein lacking a biotin ligase substrate domain (Avitag). Fusion proteins comprising the DsbA signal peptide are not biotinylated in E. coli cells expressing an endogenous biotin ligase.

FIG. 4a is a schematic representation of the encoded pIVII fusion protein of the pNp8 derivative vector PelB-Avitag-pVIII. Expression vector PelB-Avitag-pVIII comprises nucleic acid encoding a PelB leader signal peptide (PelB), to direct export of any expressed polypeptide to the periplasm of an E. coli cell; nucleic acid encoding a hexahistidine tag (10 His), for detection and/or purification of the fusion protein; nucleic acid encoding a hemagglutinin (HA) tag, for detection and/or purification of the fusion protein; nucleic acid encoding a biotin ligase substrate domain set forth in SEQ ID NO: 4 (Avitag) and nucleic acid encoding a phage coat protein pVIII (pVIII). Also shown is an EcoRI restriction enzyme site to allow for sub-cloning of a candidate peptide moiety.

FIG. 4a is a schematic representation of the encoded pIVII fusion protein of the pNp8 derivative vector DsbA-Avitag-pVIII. Expression vector DsbA-Avitag-pVIII comprises nucleic acid encoding a DsbA leader signal peptide (DsbA), to direct export of any expressed polypeptide to the periplasm of an E. coli cell; nucleic acid encoding a hexahistidine tag (10 His), for detection and/or purification of the fusion protein; nucleic acid encoding a hemagglutinin (HA) tag, for detection and/or purification of the fusion protein; nucleic acid encoding a biotin ligase substrate domain set forth in SEQ ID NO: 4 (Avitag) and nucleic acid encoding a phage coat protein pVIII (pVIII). Also shown is an EcoRI restriction enzyme site to allow for sub-cloning of a candidate peptide moiety.

FIG. 5 is a photographic representation of the detection of biotinylation by western blot analysis. Members comprising scaffolds in the form of filamentous bacteriophage displaying fusion proteins were produced in E. coli cells expressing an endogenous biotin ligase. Molecular weight marker proteins (lane 1), filamentous bacteriophage displaying DsbA-Avitag-pIII fusion proteins (lane 4, 5), filamentous bacteriophage displaying fusion protein lacking a biotin ligase substrate domain (Avitag) (negative control, lane 9), biotinylated CD40L fusion protein (positive control, lane 10). Fusion proteins comprising the DsbA signal peptide are not biotinylated in E. coli cells expressing an endogenous biotin ligase.

FIG. 6a is a schematic representation of the PelB-c-Jun-pIII fusion protein encoded by the expression vectors designated pJuFo-pIII. The PelB-c-Jun-pIII fusion protein comprises nucleic acid encoding a PelB leader signal peptide (PelB) for targeting the expressed fusion protein to the bacterial periplasm and cell surface for the purpose of phage display; nucleic acid encoding a c-terminal leucine zipper of Jun (c-Jun) for heterodimer formation with a c-terminal leucine zipper of Fos, and a pIII capsid protein.

FIG. 6b is a schematic representation of the PelB-c-Fos-Avitag fusion protein encoded by the expression vectors designated pJuFo-pIII. The PelB-c-Fos-Avitag fusion protein comprises nucleic acid encoding a PelB leader signal peptide (PelB) for targeting the expressed fusion protein to the bacterial periplasm and cell surface for the purpose of phage display; nucleic acid encoding a c-terminus of a Fos peptide (c-Fos) for formation of a heterodimer with the c-terminal leucine zipper of Jun (c-Jun); nucleic acid encoding a hexahistidine tag (6 His), for detection and/or purification of the fusion protein; nucleic acid encoding a biotin ligase substrate domain set forth in SEQ ID NO: 4 (Avitag) and nucleic acid encoding a hemagglutinin (HA) tag, for detection and/or purification of the fusion protein. Also shown is an EcoRI restriction enzyme site to allow for sub-cloning of a candidate peptide moiety.

FIG. 7a is a schematic representation of the PelB-c-Jun-pVIII fusion protein encoded by the expression vectors designated pJuFo-pVIII. The PelB-c-Jun-pVIII fusion protein comprises nucleic acid encoding a PelB leader signal peptide (PelB) for targeting the expressed fusion protein to the bacterial periplasm and cell surface for the purpose of phage display; nucleic acid encoding a c-terminal leucine zipper of Jun (c-Jun) for heterodimer formation with a c-terminal leucine zipper of Fos, and a pVIII capsid protein.

FIG. 7b is a schematic representation of the PelB-c-Fos-Avitag fusion protein encoded by the expression vectors designated pJuFo-pVIII. The PelB-c-Fos-Avitag fusion protein comprises nucleic acid encoding a PelB leader signal peptide (PelB) for targeting the expressed fusion protein to the bacterial periplasm and cell surface for the purpose of phage display; nucleic acid encoding a c-terminus of a Fos peptide (c-Fos) for formation of a heterodimer with the c-terminal leucine zipper of Jun (c-Jun); nucleic acid encoding a hexahistidine tag (6 His), for detection and/or purification of the fusion protein; nucleic acid encoding a biotin ligase substrate domain (Avi-tag), and nucleic acid encoding a hemagglutinin (HA) tag, for detection and/or purification of the fusion protein. Also shown is an EcoRI restriction enzyme site to allow for sub-cloning of a candidate peptide moiety.

FIG. 8a is a schematic representation the encoded CP 10-Avitag-N fusion protein encoded by the expression vectors designated T7Select-Avitag-N. Expression vector T7Select-Avitag-N comprises nucleic acid encoding a 10B capsid protein for the purpose of phage display, nucleic acid encoding a Hexahistidine tag (6 His) for detection and/or purification of the fusion protein; nucleic acid encoding a hemagglutinin (HA) tag, for detection and/or purification of the fusion protein; and nucleic acid encoding a biotin ligase substrate domain set forth in SEQ ID NO: 4 (Avitag). Also shown is an EcoRI restriction enzyme site to allow for sub-cloning of a candidate peptide moiety.

FIG. 8b is a schematic representation the CP 10-Avitag-N fusion protein encoded by the expression vectors designated T7Select-Avitag-C. Expression vector T7Select-Avitag-C comprises nucleic acid encoding a 10B capsid protein for the purpose of phage display, a nucleic acid encoding a Hexahistidine tag (6 His) for detection and/or purification of the fusion protein; nucleic acid encoding a hemagglutinin (HA) tag, for detection and/or purification of the fusion protein; and nucleic acid encoding a biotin ligase substrate domain (Avitag). Also shown is an EcoRI restriction enzyme site to allow for sub-cloning of a candidate peptide moiety.

FIG. 9 photographic representation of the detection of biotinylation by western blot analysis. Members comprising scaffolds in the form of T phage displaying CP 10-Avitag fusion proteins were produced in E. coli cells expressing a SUMO-(Avitag)₃ fusion protein. Molecular weight marker proteins (lane 1), T phage displaying CP 10B Avitag fusion proteins produced in cells expressing a SUMO-(Avitag)₃ fusion protein (lanes 2, 3, 4, 5), T phage displaying CP 10B Avitag fusion proteins produced in cells expressing a SUMO-(Avitag)₃ fusion protein (lane 6). CP 10B Avitag fusion proteins are not biotinylated in E. coli cells in the presence of a SUMO-(Avitag)₃ fusion polypeptide, whereas CP 10B Avitag fusion proteins are biotinylated in E. coli cells lacking expression of the SUMO-(Avitag)3 fusion polypeptide.

FIG. 10 is a schematic representation of the SITS-Avitag vector for use in a combined transcription-translation system. The SITS-Avitag vector comprises nucleic acid encoding a species independent translation sequence (SITS); nucleic acid encoding a hexahistidine tag (6 His) and nucleic acid encoding a biotin ligase substrate domain (Avitag).

FIG. 11 is a photographic representation of the detection of biotinylation by western blot analysis. Members were produced a eukaryotic cell-free protein expression system supplemented with and without a recombinant biotin ligase. Molecular weight marker proteins (lane 1), SITS-Avitag fusion proteins produced in a eukaryotic cell-free protein expression system in the absence of a recombinant biotin ligase (lanes 2, 4, 6 and 8), SITS-Avitag fusion proteins produced in a eukaryotic cell-free protein expression system in the presence of a recombinant biotin ligase (lanes 3, 5, 7 and 9). Fusion proteins comprising the species independent translation domain and a biotin ligase substrate domain are not biotinylated in an in vitro translation system.

FIG. 12 is a photographic representation of the detection of biotinylation by western blot analysis. Non-biotinylated members were incubated in HEK 293 cells and transfected HEK 293 cells expressing a recombinant biotin ligase (BirA*) cells supplemented with and without exogenous biotin. Molecular weight marker proteins (lane 1), members incubated in mammalian cells (lanes 5, 7 and 9), members incubated in transfected HEK 293 cells expressing a recombinant biotin ligase (lane 6, 8 and 10). Culture media was supplemented with biotin (lane 7 and 8). M-PER cell lysates (lanes 9, 10) were supplemented with exogenous biotin (lane 9 and 10). Transfected HEK 293 cells expressing BirA* biotinylate the non-biotinylated members with or without exogenous biotin, being added to intact HEK 293 cells in culture or to M-PER cell lysates, albeit at a lower level in the absence of exogenous biotin.

FIG. 13a is a graphical representation showing the effect of CPP and cargo on reconstitution of GFP activity in a functional assay of the invention employing GFP 1-10 and GFP 11 fragments. S11 controls (solid bars) as indicated on the figure were unmodified GFP 11 fragment at the concentrations shown on the abscissa. GFP 11 fusion proteins comprised GFP 11 fragment and the published CPP TAT (TAT_S11), HA2TAT (HA2TAT_S11), or PEP1 (PEP1_S11), or a cargo protein designated PYC35 (PYC35_S11), PYR01 (PYR01_S11), PYR02 (PYR02_S11), PYR03 (PYR03_S11), or PYR04 (PYR04_S11), at the concentrations shown on the abscissa. Fluorescence is indicated on the y-axis. Data indicate the adverse effect of additional peptide features on reconstitution of functional GFP activity in vitro.

FIG. 13b is a graphical representation showing the effect of a scaffold moiety on reconstitution of GFP activity in a functional assay of the invention employing GFP 1-10 and GFP 11 fragments. The GFP 1-10 fragment was optimized for human codon usage in a pcDNA4 vector backbone. S11 controls as indicated on the figure were unmodified GFP 11 fragment. GFP 11 fusion proteins comprised GFP 11 fragment and the scaffold moiety MyD88 (MyD88_S11), β-actin β-actin_S11), Sumo (Sumo_S11), or a cargo-scaffold fusion moiety designated PYC35_Sumo (PYC35_Sumo_S11), TAT_Sumo (TAT_Sumo_S11), or PYR01_Sumo (PYR01_Sumo_S11). Relative fluorescence, normalized for activity in the presence of the MyD88_S11 and mGFP1-10 constructs, is indicated on the y-axis. Data indicate that transient transfection of HEK293 cells with constructs expressing mGFP1-10 and GFP 11 does not produce detectable levels of GFP fluorescence, however the addition of a scaffold improves reconstitution of functional GFP.

FIG. 14 is a copy of a photographic representation showing localization of reconstituted GFP (split GFP) in HEK-293 cells transfected with mGFP 1-10 and scaffold-GFP 11 fusion protein. Panel A shows that MyD88_S11+mGFP1-10 co-transfection produces dense pockets of concentrated intracellular GFP mainly in rounded cells. Cells had the brightest fluorescence relative to other GFP 11 fusions indicated. Panel B shows that β-actin_S11+mGFP1-10 co-transfection produces strong fluorescence, diffuse localization of split GFP labelling throughout the cytoplasm and concentrated at dendritic features, and that cell morphology is more dendritic than for other GFP 11 fusions shown. Panel C shows that a RelA-GFP 11 fusion (RelA_S11)+mGFP1-10 co-transfection produces a medium-low fluorescence, diffuse localization of split GFP throughout cytoplasm and sometimes excluded from nucleus. Panel D shows that a Mal-GFP 11 fusion (Mal_S11)+mGFP1-10 co-transfection produces low fluorescence but split GFP expression that is diffuse throughout the cytoplasm, and concentrated in multiple small foci.

FIG. 15 is a graphical representation showing the effect of GFP 1-10 codon usage on reconstituted GFP (split GFP) activity in cells 24 hours (above) and 48 hours (below) after transfection with mGFP 1-10 and scaffold-GFP 11 fusion proteins MyD88_S11, β-actin_S11 and Mal-S11. Constructs are shown on the abscissae. Relative fluorescence for each construct, normalized for activity in the presence of the MyD88_S11 and mGFP1-10 constructs, is indicated on the y-axes. The GFP 1-10 constructs comprised commercially-available mGFP1-10 (“A” variant) expressed from pcDNA4 (mGFP 1-10), a humanized variant of the commercially-available mGFP1-10 (“A” variant) expressed from pcDNA4/TO vector [TO hGFP1-10(a)] or pcDNA4/HM vector [HM hGFP1-10(a)], or a corrected and humanized variant of the commercially-available mGFP1-10 (“G” variant) expressed from pcDNA4/TO vector [TO hGFP1-10(g)] or pcDNA4/HM vector [HM hGFP1-10(a)]. Data indicate that correction of the mutation in commercially-available GFP 1-10 and/or human codon usage enhance(s) reconstitution of split GFP activity especially for cells co-transfected with Mal_S11+mGFP1-10, and that this activity is sustained in transfected cells for up to at least 48 hours. Data suggest that expression of human codon-optimized and corrected GFP 1-10 sequence from pcDNA4/TO vector (hGFP1-10(g)/TO) produces enhanced reconstitution of split GFP activity in the functional assay.

FIG. 16 is a graphical representation showing the effect of different linkers positioned between the scaffold/cargo and GFP 11 fragment on reconstitution of split GFP activity in isolated HEK-293 cells expressing GFP 11+GFP 1-10 fragments. GFP 11 fusions shown on the abscissa are: MyD88-GFP 11 fusion (MyD88); Mal-GFP 11 fusion (Mal), β-actin-GFP 11 fusion β-actin), Sumo-GFP 11 fusion (Sumo), and receptor binding domain (RBD)-GFP 11 fusion (RBD). Average fluorescence for each construct is indicated on the y-axis. Negative controls lacked the GFP 11 fragment (open bars; no S11) or the linker (filled bars; S11v3). Linkers employed were as follows: a 16-mer amino acid sequence consisting of GSSGGSSGGSSGGSSG (S11v4); an 18-mer amino acid sequence consisting of GGTGGSGGAGGTGGSGGA (S11v5); a 14-mer amino acid sequence consisting of GTTGGTTGGGTGGS (S11v6); and a 10-mer amino acid sequence consisting of APAPAPAPAP (S11v7.

FIG. 17 is a graphical representation showing the effect of cargo proteins on reconstitution of split GFP activity in isolated HEK-293 cells expressing GFP 11+GFP 1-10 fragments. HEK-293 cells transfected with GFP 1-10 vectors pcDNA4/TO vector [TO hGFP1-10(a)] or pcDNA4/HM vector [HM hGFP1-10(a)] are shown on the abscissa. Relative fluorescence for each GFP 11 construct added to the cells, normalized for activity in the presence of the MyD88_S11 and mGFP1-10 constructs, is indicated on the y-axis. The GFP 11 constructs lacking cargo peptides were: MyD88-GFP 11 fusion (MyD88_S11); Mal-GFP 11 fusion (Mal_S11), β-actin-GFP 11 fusion β-actin_S11), and Sumo-GFP 11 fusion (Sumo_S11). The GFP 11 constructs comprising cargo peptides were variants of the Sumo-GFP 11 fusion (Sumo_S11) fusion construct, as follows: PYC35-Sumo-GFP 11 fusion (PYC35_Sumo_S11), PYR01-Sumo-GFP 11 fusion (PYR01_Sumo_S11), and TAT-Sumo-GFP 11 fusion (TAT_Sumo_S11). Data indicate that a cargo peptide can modulate reconstitution of split GFP activity in isolated HEK-293 cells expressing GFP 11+GFP 1-10 fragments, independent of cell-penetrating activity of the peptide. PYC35, which is not a CPP, showed no-effect on Sumo_S11 fluorescence, whilst TAT and PYR01, which both exhibit CPP activity, decreased fluorescence of Semo_S11 by more than 50%. This effect was independent of CPP uptake activity, because all moieties were expressed from transiently transfected constructs in HEK293 cells. The same effect was observed for the two different hGFP1-10 expression constructs shown. These data suggest the advantage of performing in vitro complementation to test the effect of specific cargo fusion peptides on reconstitution of split GFP activity in vitro.

FIG. 18 provides a graphical representation showing that reconstitution of split GFP activity in cells expressing GFP 11+GFP 1-10 fragments detects uptake of CPP-cargo-GFP 11 fusion polypeptides into different cell lines by determining fluorescence of reconstituted GFP. Constructs shown on the abscissa comprised the CPPs TAT, PYR01, PYJ04 or PYJ05 linked to the RBD-GFP 11 fusion polypeptide (RBD_S11). Negative controls were HisMBP or RBD-GFP 11 fusion polypeptide without CPP. Percentage of GFP-positive cells in total live cell population, normalized for transfection efficiency as determined in independent transfections of each cell line with pcDNA3-eGFP, is indicated on the y-axis. Cells were either human HCC-827 cells or CHO-K1 cells. Fluorescence was determined on 2.5 μM protein, 5 μM protein, 10 μM protein, 20 μM protein, 40 μM protein or 80 μM protein, as shown. The different CPPs were each expressed as fusions with the receptor binding domain (RBD) cargo protein and GFP 11 (S11v4) in both HCC-827 (high receptor expression) and CHO-K1 (negative receptor expression) cells that had been transiently-transfected with hGFP1-10(g)/TO. Split GFP complementation was detected by measuring GFP fluorescence using flow cytometry, gating on the live cell population. Data indicate that the fluorescence signal was dose-responsive for each construct tested, and obtainable for fresh and frozen protein samples.

FIG. 19 is a schematic representation showing a workflow of a functional assay of the invention comprising determining reconstitution of split GFP activity in cells expressing GFP 11+GFP 1-10 fragments for the detection of CPP-cargo-GFP 11 fusion polypeptide uptake into cells by determining fluorescence of the reconstituted GFP.

FIG. 20 provides graphical representations showing that a functional assay of the invention comprising determining reconstitution of split GFP activity works in different cell lines. Panel A employed CHO-K1 cells transiently transfected with hGFP1-10(g)/TO vector. Panel B employed HCC-827 cells transiently transfected with hGFP1-10(g)/TO vector. Panel C employed HEK-293 cells transiently transfected with hGFP1-10(g)/TO vector. Panel D employed HEK-293 cells stably transformed with hGFP1-10(g)/TO vector. Panel E employed K562 cells transiently transfected with hGFP1-10(g)/TO vector. Constructs shown on the abscissae comprised the CPPs TAT or PYJ01 linked to the RBD-GFP 11 cargo fusion polypeptide (RBD_S11) or thioredoxin-GFP 11 cargo fusion polypeptide. Negative controls were HisMBP or the cargo fusion polypeptides lacking a CPP or comprising the second cargo protein PYC35 in lieu of a CPP. Fluorescence was determined on 5 μM protein, 10 μM protein, 20 μM protein, and 40 μM protein, as shown. Percentage of GFP-positive cells in total live cell population, normalized for transfection efficiency as determined in independent transfections of each cell line with pcDNA3-eGFP, is indicated on the y-axis, except for stable cell line HEK293/GFP1-10 where the % GFP positive cells of total live cell population was not adjusted. Data indicate baseline fluorescence for assays that lacked CPP, with only validated CPPs TAT and PYJ01 providing reconstitution of GFP activity in the functional assay, in a dose-dependent manner and for different cell lines tested: CHO-K1 (adherent, rodent, negative for receptor expression); HCC-827 (adherent, human, strongly positive for receptor expression); HEK293 (adherent, human, moderate/low positive for receptor expression); HEK293/GFP1-10 (adherent, human, moderate/low positive for receptor expression, monoclonal stable transformed with hGFP1-10(g)/TO); and K562 (non-adherent, human, moderate/low positive for receptor expression).

FIG. 21 provides photographic representations showing uptake of highly-purified CPP-cargo-GFP 11 in cell lines that have been transiently transfected with hGFP1-10(g)/TO. Negative controls employed a cargo-GFP 11 fusion polypeptide i.e., without the CPP. The cargo was the receptor binding peptide RBD, and CPP was PYJ01. The cargo-GFP 11 (RBD_S11) and CPP-cargo-GFP 11 fusion (PYJ01_RBD_S11) were each added to CHO-K1 cells or HCC-827 cells at 10 μM concentration. Data indicate that neither cell line had reconstituted split GFP activity when transfected with the RBD_S11 and hGFP1-10(g)/TO constructs, however high nuclear split GFP activity was detected for cells transfected with both PYJ01_RBD_S11 and hGFP1-10(g)/TO constructs. This demonstrates utility of the functional assay for determining CPP activity, especially for demonstrating escape of the fusion polypeptide from the endosome of the cell.

FIG. 22 provides graphical representations showing the ability of a functional assay of the invention that comprises determining reconstitution of split GFP activity in cells expressing GFP 11+GFP 1-10 fragments for the detection of CPP-cargo-GFP 11 fusion polypeptide uptake into cells using canonical CPP peptides. Constructs comprised the canonical CPPs shown at the right of the figure linked to the cargo-GFP 11 fusion polypeptides shown on the abscissae, each at 30 μM concentration. Positive controls were 30 μM AKTA purified TAT-RBD-GFP 11 (TAT_RBD_S11v4) or PYJ01-RBD-GFP 11 (PYJ01_RBD_S11v4) fusion proteins. Negative controls lacked CPP, and the horizontal broken line indicates a maximum threshold fluorescence for negative controls. Cell lines tested were HCC-827 cells transiently transfected with hGFP1-10(g)/TO vector, or CHO-K1 cells transiently transfected with hGFP1-10(g)/TO vector, or HEK-293 cells stably transformed with hGFP1-10(g)/TO vector. Relative fluorescence for each construct, normalized for activity in the presence of the AKTA purified PYJ01-RBD-GFP 11 and hGFP1-10(g)/TO constructs, is indicated on the y-axes. Data verify activities of the canonical CPPs TAT, PYJ01, VP22, SAP, and PTD4, however all other canonical CPPs show marginal split GFP complementation as measured by detection of GFP fluorescence. VP22, SAP and PTD4 showed reduced activity relative to TAT and PYJ01.

FIG. 23 is a graphical representation showing average amino acid compositions of peptides that have been demonstrated herein as having an ability to transport GFP11 into the cytoplasm of cells as determined by reconstitution of functional GFP in the split GFP complementation assay of the present invention (“Split-GFP Positive”), compared to the average amino acid compositions of peptides that have been demonstrated herein not to have this functionality (“Split-GFP negative”). Data indicate that, in general the assay does not discriminate in terms of amino acid composition, however may select against peptides that have a higher composition of cysteine (C), glutamate (E) or lysine (K). However, the inventors do not rule out the possibility that higher compositions of cysteine (C) and/or glutamate (E) and/or lysine (K) may adversely affect CPP activity of certain peptides.

FIG. 24 is a graphical representation showing average charge, hydrophobicity, length and PSI-structure prediction properties of peptides that have been demonstrated herein as having an ability to transport GFP11 into the cytoplasm of cells as determined by reconstitution of functional GFP in the split GFP complementation assay of the present invention (“Split-GFP Positive”), compared to the average charge, hydrophobicity and PSI-structure prediction properties of peptides that have been demonstrated herein not to have this functionality (“Split-GFP negative”). Data indicate that there are significant differences in terms of net charge, hydrophobicity at pH 6.8, and that the assay does not discriminate in terms of predicted structures for peptides, or peptide length. The inventors do not rule out the possibility that peptides that are Split-GFP negative are inherently less likely to exhibit CPP activity.

FIG. 25 is a graphical representation showing average amino acid compositions of isolated CPPs of the present invention that have been demonstrated herein to have an ability to transport GFP11 into the cytoplasm of cells as determined by reconstitution of functional GFP in the split GFP complementation assay of the present invention (“Split-GFP Positive Phylomers”), compared to the average amino acid compositions of known CPPs (“canonical CPP”). Data indicate that canonical CPPs have high levels of alanine (A) and arginine (R), whereas the CPPs of the present invention that are positive in both the endosomal biotinylation trap and split GFP complementation assay of the invention have high levels of lysine (K), arginine (R), and proline (P). Differences in levels of phenylalanine (F), isoleucine (I) and threonine (T) between the CPPs of the present invention and canonical CPPs are also highly-significant.

FIG. 26 is a graphical representation showing average charge, hydrophobicity, and length of isolated CPPs of the present invention that have been demonstrated herein to have an ability to transport GFP11 into the cytoplasm of cells as determined by reconstitution of functional GFP in the split GFP complementation assay of the present invention (“Split-GFP Positive Phylomers”), compared to the average charge, hydrophobicity, length and PSI-structure prediction properties of known CPPs (“canonical CPP”). Data indicate significant differences in each of net charge, hydrophobicity and peptide length between canonical CPPs and CPPs of the present invention, suggesting that the peptides of the present invention may represent a new structural class of non-canonical CPPs.

### DETAILED DESCRIPTION OF THE PREFERRED EMBODIMENTS

### Cellular Trafficking

The present invention encompasses monitoring of cellular trafficking without limitation unless specifically stated otherwise or the context requires a more narrow construction of cellular trafficking.

The skilled artisan is aware that molecules may be trafficked into, out from and within a cell by any one or more of various mechanisms. Membrane trafficking involves transportation of molecules across a biological membrane such as, a plasma membrane or intracellular membrane. Examples of intracellular membranes include, for example, the endoplasmic reticulum membrane, the nuclear membrane, the Golgi apparatus membrane, the mitochondria membrane, the chloroplast membrane, the lysosome membrane, the early endosome membrane, the late endosome membrane and the recycling endosome membrane.

In one example of the invention, endocytosis is monitored. Endocytosis is a mechanism by which cells internalize extracellular material (Conner and Schmid, Nature 422, 37-44, 2003). In eukaryotic cells, internalization may occur via clathrin-dependent endocytosis, or clathrin-independent endocytosis. It is also understood that different mechanisms of endocytosis may occur simultaneously.

In one example, the endocytosis is clathrin-dependent endocytosis. Clathrin-dependent endocytosis is the best characterized mechanism for the entry of molecules and plasma membrane constituents into cells. Clathrin-dependent mechanisms that have been identified include, for example, receptor mediated endocytosis, and cell adhesion molecule assisted endocytosis. In these processes, intracellular vesicles typically form invaginations in the membrane that are coated by clathrin.

In one example, the endocytosis is clathrin-independent endocytosis. Clathrin-independent pathways include, for example, macropinocytosis, caveolae/raft-mediated endocytosis, clathrin- and caveolae-independent endocytosis.

Preferably, the Clathrin-independent pathway comprises macropinocytosis. Macropinocytosis may involve actin-dependent formation of lamellipodia or extensive membrane ruffling followed by the formation of discrete vacuoles i.e. macropinosomes within the cell (Swanson and Watts, Trends Cell Biol. 5, 424-428, 1995).

Alternatively, the Clathrin-independent pathway comprises caveolae-independent endocytosis. Examples of clathrin-independent and caveolae-independent pathways include, for example, Arf6-dependent endocytosis, flotillin-dependent endocytosis, Cdc42-dependent endocytosis, GPI-enriched endocytic compartments (GEEC)-dependent endocytosis, IL-2-dependent endocytosis, RhoA-dependent endocytosis and circular dorsal ruffling. See e.g. Mayor and Pagano, Nat. Rev. Mol. Cell. Biol. 8, 603-612 (2007); Hoon et al. Mol. Cell Biol. 32, 4246-4257 (2012); Kirkham et al. J. Cell Biol 0.168, 465-476 (2005).

In yet another example of the invention, phagocytosis and/or pinocytosis and/or a retrograde transport is monitored. Phagocytosis, pinocytosis and retrograde transport pathways are described, for example, by Johannes and Popoff, Cell 135, 1175-1187 (2008) and Lieu and Gleeson, Histol. Histopathol. 26, 395-408. (2011).

In yet another example of the invention, transcytosis is monitored to determine transportation of molecules across an intracellular membrane or from one cell surface to another cell surface. In one example, a molecule that is to be transcytosed may bind to a receptor. The receptor-ligand complex then enters a cell by endocytosis to form a vesicle. Transcytotic vesicles are subsequently formed which are delivered to the opposite cell surface where they fuse with the plasma membrane and release their contents. Transcytosis may occur in either direction, from the apical to basolateral surface or from the basolateral to apical cell surface.

In yet another example of the invention, exocytosis is monitored to determine transportation of molecules out from a cell and into an extracellular environment.

Methods for monitoring cellular trafficking of a peptide as broadly defined or according to any specific example hereof may comprise monitoring the movement of a candidate peptide moiety across a biological membrane or monitoring the movement of a candidate peptide moiety from one subcellular location to another subcellular location. As will be apparent from the preceding description, movement of the candidate peptide moiety across a plasma membrane may be mediated by clathrin-dependent endocytosis and/or clathrin-independent endocytosis and/or clathrin- and caveolae-independent endocytosis and/or phagocytosis and/or pinocytosis.

In one example, trafficking of biotinylated members or fusion proteins produced in accordance with the present invention is analysed in host cells using standard flow cytometry and/or fluorescence activated cell sorting (FACS) and/or fluorescence microscopy and/or live confocal microscopy. Such visualisation methods detect biotin covalently attached to the biotin ligase substrate domain of a fusion protein to determine the localisation of the biotinylated member or fusion protein within the host cells.

In one example, monitoring cellular trafficking of a peptide comprises determining the localization of a biotinylated member in a sub-cellular location other than the endosome or endosome-lysosome e.g., cytosol, nucleus, endoplasmic reticulum, golgi, vacuole, mitochondrion, plastid such as chloroplast or amyloplast or chromoplast or leukoplast, nucleus, ribosome, cytoskeleton, centriole, microtubule-organizing center (MTOC), acrosome, glyoxysome, melanosome, myofibril, nucleolus, peroxisome, nucleosome or microtubule.

In another example, monitoring cellular trafficking of a peptide comprises determining the localization of a biotinylated member in a sub-cellular location other than in a vesicle of the endomembrane system of the cell e.g., cytosol, nucleus, endoplasmic reticulum, golgi, mitochondrion, plastid, nucleus, ribosome, cytoskeleton, centriole, microtubule-organizing center (MTOC), acrosome, glyoxysome, melanosome, myofibril, nucleolus, peroxisome, nucleosome or microtubule.

Alternatively, monitoring cellular trafficking of a peptide comprises labelling a displayed fusion protein e.g., a fusion protein displayed on a scaffold with a suitable reporter molecule e.g., a fluorophore, radioactive label, luminescent molecule, dye, etc., and determining the localization of the reporter molecule within the cell, wherein localization of the reporter molecule bound to the fusion protein in a sub-cellular location other than the endosome or endosome-lysosome or other vesicle of the endomembrane system indicates release of the peptide from the endosome or endosome-lysosome.

Methods for labelling fusion proteins are known in the art and are described, for example, by Chen and Ting, Curr. Opin. Biotechnol. 16, 35-40 (2005) or Sambrook et al. (In: Molecular Cloning: Molecular Cloning: A Laboratory Manual, Cold Spring Harbor Laboratories, New York, Third Edition 2001).

In a further example, monitoring cellular trafficking of a peptide comprises distinguishing between biotinylated members trapped in the endosome and biotinylated members that have escaped from the endosome. In one example, biotinylated members that have escaped from the endosome are substantially in a sub-cellular location other than in a vesicle of the endomembrane system of the cell e.g., cytosol, nucleus, endoplasmic reticulum, golgi, mitochondrion, plastid, nucleus, ribosome, cytoskeleton, centriole, microtubule-organizing center (MTOC), acrosome, glyoxysome, melanosome, myofibril, nucleolus, peroxisome, nucleosome or microtubule. Exemplary methods for distinguishing between biotinylated members trapped in the endosome and biotinylated members that have escaped from the endosome comprise detecting the presence of biotin covalently attached to the biotin ligase substrate domain of a fusion protein as described in the method of the present invention.

It will be apparent that non-biotinylated members may be readily transported across a cell membrane and/or internalized within a host cell by contacting the cell with a non-biotinylated member for a time and under conditions sufficient for at least the fusion protein to translocate a membrane of the host cell.

### Structure of Non-Biotinylated Members

### Candidate Peptide Moiety

A candidate peptide moiety employed in the method of the present invention may be a synthetic molecule or recombinant molecule by virtue of being encoded by nucleic acid e.g., genome fragments or amplified nucleic acid derived therefrom or mRNA or cDNA.

Preferred candidate peptide moieties do not comprise an entire protein that occurs in nature. In one example, the candidate peptide moiety comprises at least about 15 amino acids in length. Preferred peptides consist of fewer than about 300 amino acids or fewer than about 200 amino acids or fewer than about 150 amino acids or fewer than about 125 amino acids or fewer than about 100 amino acids or fewer than about 90 amino acids, or fewer than about 80 amino acids.

In another example, a preferred candidate peptide has secondary structure characteristics e.g., it forms/produces a fold or protein domain when expressed. Preferably, the peptide produces a fold or protein domain autonomously when expressed in a host cell. Unstructured candidate peptides may also be employed, and optionally induced to form a fold or secondary structure e.g., by introducing cysteine residues to the peptide and/or by promoting intramolecular disulphide linkages between cysteine residues located in the peptide. Preferably, induced secondary structure formation comprises positioning cysteine residues either side of amino acid residues that are sought to contribute to the fold or protein domain so as not to interfere with functionality of the fold or protein domain. In one example, cysteine residues are added to the N-terminus and/or C-terminus of the candidate peptides and the peptides are subjected to appropriate redox conditions to promote their cyclization thereby inducing secondary structure formation.

In one example, the candidate peptide is a synthetic peptide molecule produced according to any method known in the art and described herein. For example, peptides may be synthesized by coupling the carboxyl group or C-terminus of one amino acid to the amino group or N-terminus of another, generally employing one or more protecting groups and starting at a C-terminal end of the peptide and ending at an N-terminal end of the peptide. A liquid-phase synthesis or solid phase synthesis may be employed, and solid phase synthesis is preferred.

Methods for solid phase synthesis of peptides are well-known in the art. See e.g., references [11] to [16] hereof which are incorporated by reference. See also e.g.: Stewart et al., In: Solid phase peptide synthesis (2nd ed.). Rockford: Pierce Chemical Company. p. 91 (1984); Atherton et al., In: Solid Phase peptide synthesis: a practical approach. Oxford, England: IRL Press. (1989); Hermkens et al., Tetrahedron 53 (16), 5643-5678 (1997); and Albericio, In: Solid-Phase Synthesis: A Practical Guide (1 ed.). Boca Raton: CRC Press. p. 848 (2000).

Synthetic candidate peptides will generally comprise a protein domain, preferably a protein domain is not known to be associated with CPP activity or PTD activity. The protein domain may comprise an amino acid sequence that is contained within the amino acid sequence of a full-length protein, such as a sequence of a protein domain not normally associated with CPP or PTD activity. Alternatively, the protein domain may comprise an unknown amino acid sequence not described previously in any known protein. Again, such candidate peptides for use in the method of the invention will preferably comprise a protein domain not known to be associated with CPP activity or PTD activity.

In another example, the candidate peptide is a recombinant peptide molecule produced by translation of mRNA or by transcription of DNA and subsequent translation of an RNA transcript thereof. Nucleic acid fragments for use in the production of such recombinant peptides will generally comprise an open reading frame capable of being translated in vivo or ex vivo or in vitro to produce a polypeptide. Preferably, the candidate peptide does not have an amino acid sequence and/or secondary structure of a known cell-penetrating peptide (CPP) or protein transduction domain (PTD).

In one example, the open reading frame encoding a candidate peptide is a natural open reading frame i.e., an open reading frame employed in protein synthesis in nature. In the case of such natural open reading frames, nucleic acid fragments encoding candidate peptides for use in the method of the invention will preferably comprise a protein domain of the full-length protein encoded by the complete open reading frame in nature. More preferably, the protein domain is not known to be associated with CPP activity or PTD activity.

Alternatively, the open reading frame is non-natural or synthetic or artificial i.e., it is not a natural open reading frame such as because it comprises a reading frame of a gene fragment that is not normally employed in translation of the mRNA transcript of the full-length gene in nature. The skilled artisan is aware that DNA comprises six possible open reading frames, however these are not all employed in nature. In the case of non-natural open reading frames, nucleic acid fragments encoding candidate peptides for use in the method of the invention encode different peptides to that encoded by the open reading frame employed in nature. In one example, the encoded peptide is hitherto unknown. Preferably, such candidate peptides for use in the method of the invention will comprise a protein domain not known to be associated with CPP activity or PTD activity.

It will be apparent from the foregoing description that all that is required to produce a recombinant candidate peptide for use in the method of the invention is an open reading frame of sufficient length to encode a peptide or protein domain.

Nucleic acid fragments may be generated by one or more of a variety of methods known to those skilled in the art.

In one example, nucleic acid fragments are derived from genomic DNA. Methods of isolating genomic DNA from a variety of organism are known in the art. Genomic DNA may also be isolated using commercially available kits, such as, for example, the PureLink Genomic DNA Mini Kit (Invitrogen), the Wizard Genomic DNA purification kit (Promega), the QIAamp kit (Qiagen), the Genomic DNA Purification kit (Thermo Scientific), or PrepEase Genomic DNA Isolation kit (Affymetrix).

In another example, nucleic acid fragments are derived from complementary DNA (cDNA). Those skilled in the art will be aware that cDNA is generated by reverse transcription of RNA using, for example, avian reverse transcriptase (AMV) reverse transcriptase or Moloney Murine Leukemia Virus (MMLV) reverse transcriptase. Such reverse transcriptase enzymes and the methods for their use are known in the art, and are obtainable in commercially available kits, such as, for example, the Powerscript kit (Clontech), the Superscript II kit (Invitrogen), the Thermoscript kit (Invitrogen), the Titanium kit (Clontech), or Omniscript (Qiagen). Methods of generating cDNA from isolated RNA are also commonly known in the art and are described in for example, Ausubel et al., In: Current Protocols in Molecular Biology. Wiley Interscience, ISBN 047 150338, 1987). In addition kits for isolating mRNA and synthesizing cDNA are commercially available e.g. RNeasy Protect Mini kit, RNeasy Protect Cell Mini kit from Qiagen.

Fragments are generated from DNA including genomic DNA or cDNA by any one of a number of methods, for example, mechanical shearing (e.g., by sonication or passing the nucleic acid through a fine gauge needle) and/or digestion with a nuclease (e.g., Dnase 1) and/or digestion with one or more restriction enzymes e.g., frequent cutting enzymes that recognize 4-base restriction enzyme sites and/or by treatment of DNA with radiation e.g., gamma radiation or ultra-violet radiation and/or amplification. Suitable methods are described, for example, in Ausubel et al. (In: Current Protocols in Molecular Biology. Wiley Interscience, ISBN 047 150338, 1987) or Sambrook et al. (In: Molecular Cloning: Molecular Cloning: A Laboratory Manual, Cold Spring Harbor Laboratories, New York, Third Edition 2001).

Amplification of DNA fragments is preferred, because it facilitates the introduction of restriction enzyme cleavage sites for use in subsequent steps in the method of the invention. In one example, copies of nucleic acid fragments isolated from one or more organism(s) are generated by polymerase chain reaction (PCR) or an isothermal amplification method using, for example, random or degenerate oligonucleotides. Such random or degenerate oligonucleotides preferably include restriction enzyme recognition sequences to allow for cloning of the amplified nucleic acid into an appropriate nucleic acid vector. Methods of generating oligonucleotides are known in the art and are described, for example, in Oligonucleotide Synthesis: A Practical Approach (M. J. Gait, ed., 1984) IRL Press, Oxford, whole of text, and particularly the papers therein by Gait, pp 1-22; Atkinson et al., pp35-81; Sproat et al., pp 83-115; and Wu et al., pp 135-151. Methods of performing PCR are also described in detail by McPherson et al., In: PCR A Practical Approach, IRL Press, Oxford University Press, Oxford, United Kingdom, 1991.

Nucleic acid fragments for use in performing the invention are preferably derived from one or two or more prokaryotic organisms such as, for example, Aeropyrum pernix, Agrobacterium tumeficians, Aquifex aeolicus, Archeglobus fulgidis, Bacillus halodurans, Bacillus subtilis, Borrelia burgdorferi, Brucella melitensis, Brucella suis, Bruchnera sp., Caulobacter crescentus, Campylobacter jejuni, Chlamydia pneumoniae, Chlamydia pneumoniae, Chlamydia trachomatis, Chlamydia muridarum, Chlorobium tepidum, Clostridium acetobutylicum, Deinococcus radiodurans, Escherichia coli, Haemophilus influenzae Rd, Halobacterium sp., Helicobacter pylori, Methanobacterium thermoautotrophicum, Lactococcus lactis, Listeria innocua, Listeria monocytogenes, Methanococcus jannaschii, Mesorhizobium loti, Mycobacterium leprae, Mycobacterium tuberculosis, Mycoplasma genitalium, Mycoplasma penetrans, Mycoplasma pneumoniae, Mycoplasma pulmonis, Neisseria meningitidis, Oceanobacillus iheyensis, Pasteurella multocida, Pseudomonas aeruginosa, Pseudomonas putida, Pyrococcus horikoshii, Rickettsia conorii, Rickettsia prowazekii, Salmonella typhi, Salmonella typhimurium, Shewanella oneidensis MR-1, Shigella flexneri 2a, Sinorhizobium meliloti, Staphylococcus aureus, Streptococcus agalactiae, Streptococcus agalactiae, Streptococcus mutans, Streptococcus pneumoniae, Streptococcus pyogenes, Streptomyces avermitilis, Streptomyces coelicolor, Sulfolobus solfataricus, Sulfolobus tokodaii, Synechocystis sp., Thermoanaerobacter tengcongensis, Thermoplasma acidophilum, Thermoplasma volcanium, Thermotoga maritima, Treponema pallidum, Ureaplasma urealyticum, Vibrio cholerae, Xanthomonas axonopodis pv., Citri, Xanthomonas campestris pv., Campestris, Xylella fastidiosa, and Yersinia pestis.

Alternatively, or in addition, the nucleic acid fragments are derived from one or two or more eukaryotic organisms such as, for example, Anopheles gambiae, Arabidopsis thaliana, Babesia microti, Bos taurus, Caenorhabditis elegans, Callithrix jacchus, Canis lupus, Danio rerio, Debaryomyces hansenii, Ectocarpus siliculosus, Eimeria tenella, Fusarium graminearum, Gallus gallus, Glycine max, Hemiselmis andersenii, Hemiselmis andersenii, Kluyveromyces lactis, Komagataella pastoris, Lachancea kluyveri, Lachancea thermotolerans, Macaca fascicularis, Medicago truncatula, Naumovozyma castellii, Neospora caninum, Neospora caninum, Oryctolagus cuniculus, Ostreococcus lucimarinus, Ostreococcus lucimarinus, Paramecium tetraurelia, Rattus norvegicus, Saccharomyces cerevisiae, Sorghum bicolor, Taeniopygia guttata, Thalassiosira pseudonana, Vitis Vinifera, Yarrowia lipolytica and Zea mays.

Preferred nucleic acid fragments from eukaryotes are derived from one or two or more eukaryotes having compact genomes. As used herein the term “compact genome” shall be taken to mean a haploid genome size of less than about 1700 mega base pairs (Mbp), and preferably, less than 100 Mbp. Preference for a compact genome arises from the lower abundance of non-transcribed or intron sequence relative to larger eukaryotic genomes, which enhances representation of natural open reading frames in the nucleic acid pool employed to produce candidate peptides. Exemplary eukaryotes having compact genomes suitable for this purpose include Arabidopsis thaliana, Anopheles gambiae, Brugia malayi, Caenorhabditis elegans, Danio rerio, Drosophila melanogaster, Eimeria tenella, Eimeria acervulina, Entamoeba histolytica, Oryzias latipes, Oryza sativa, Plasmodium falciparum, Plasmodium vivax, Plasmodium yoelii, Sarcocystis cruzi, Saccharomyces cerevesiae, Schizosaccharomyces pombe, Schistosoma mansoni, Takifugu rubripes, Theileria parva, Tetraodon fluviatilis, Toxoplasma gondii, Tryponosoma brucei, and Trypanosoma cruzi.

Alternatively, or in addition, the nucleic acid fragments are derived from one or two or more viruses such as, for example, a virus selected from the group consisting of T7 phage, HIV, equine arteritis virus, lactate dehydrogenase-elevating virus, lelystad virus, porcine reproductive and respiratory syndrome virus, simian hemorrhagic fever virus, avian nephritis virus 1, turkey astro virus 1, human antero virus type 1, 2 or 8, mink astro virus 1, ovine astro virus 1, avian infectious bronchitis virus, bovine coronavirus, human coronavirus, murine hepatitis virus, porcine epidemic diarrhea virus, SARS coronavirus, transmissible gastroenteritis virus, acute bee paralysis virus, aphid lethal paralysis virus, black queen cell virus, cricket paralysis virus, Drosophila C virus, himetobi P virus, kashmir been virus, plautia stali intestine virus, rhopalosiphum padi virus, taura syndrome virus, triatoma virus, alkhurma virus, apoi virus, cell fusing agent virus, deer tick virus, dengue virus type 1, 2, 3 or 4, Japanese encephalitis virus, Kamiti River virus, kunjin virus, langat virus, louping ill virus, modoc virus, Montana myotic leukoencephalitis virus, Murray Valley encephalitis virus, omsk hemorrhagic fever virus, powassan virus, Rio Bravo virus, Tamana bat virus, tick-borne encephalitis virus, West Nile virus, yellow fever virus, yokose virus, Hepatitis C virus, border disease virus, bovine viral diarrhea virus 1 or 2, classical swine fever virus, pestivirus giraffe, pestivirus reindeer, GB virus C, hepatitis G virus, hepatitis GB virus, bacteriophage Mi 1, bacteriophage Qbeta, bacteriophage SP, enterobacteria phage MX1, enterobacteria NL95, bacteriophage AP205, enterobacteria phage fr, enterobacteria phage GA, enterobacteria phage KU1, enterobacteria phage M1 2, enterobacteria phage MS2, pseudomonas phage PP7, pea enation mosaic virus-1, barley yellow dwarf virus, barley yellow dwarf virus-GAV, barley yellow dwarf virus-MAW, barley yellow dwarf virus-PAS, barley yellow dwarf virus-PAV, bean leafroll virus, soybean dwarf virus, beet chlorosis virus, beet mild yellowing virus, beet western yellows virus, cereal yellow dwarf virus-RPS, cereal yellow dwarf virus-RPV, cucurbit aphid-borne yellows virus, potato leafroll virus, turnip yellows virus, sugarcane yellow leaf virus, equine rhinitis A virus, foot-and-mouth disease virus, encephalomyocarditis virus, theilovirus, bovine enterovirus, human enterovirus A, B, C, D or E, poliovirus, porcine enterovirus A or B, unclassified enterovirus, equine rhinitis B virus, hepatitis A virus, aichi virus, human parechovirus 1, 2 or 3, ljungan virus, equine rhinovirus 3, human rhino virus A and B, porcine teschovirus 1, 2-7, 8, 9, 10 or 11, avian encephalomyehtis virus, kakugo virus, simian picornavirus 1, aura virus, barmah forest virus, chikungunya virus, eastern equine encephalitis virus, igbo ora virus, mayaro virus, ockelbo virus, onyong-nyong virus, Ross river virus, sagiyama virus, salmon pancrease disease virus, semliki forest virus, sindbis virus, sindbus-like virus, sleeping disease virus, Venezuelan equine encephalitis virus, Western equine encephalomyehtis virus, rubella virus, grapevine fleck virus, maize rayado fino virus, oat blue dwarf virus, chayote mosaic tymovirus, eggplant mosaic virus, erysimum latent virus, kennedya yellow mosaic virus, ononis yellow mosaic virus, physalis mottle virus, turnip yellow mosaic virus and pomsettia mosaic virus.

Alternatively, or in addition, the nucleic acid fragments are derived from one or two or more well-characterized genomes. A well-characterized genome may be a compact genome of a eukaryote e.g., a protist, dinoflagellate, alga, plant, fungus, mould, invertebrate, vertebrate, etc., or a prokaryote e.g., a bacterium, eubacterium, cyanobacterium, etc., or a virus. By “well-characterized” is meant that the genome is substantially-sequenced e.g., at least about 60% of each contributing genome has been sequenced and/or that the genome has a C-value (pg) of less than about 120. Methods for determining the amount of a genome that has been sequenced are known in the art. Furthermore, information regarding those sequences that have been sequenced is readily obtained from publicly available sources, such as, for example, the databases of NCBI or TIGR, thereby facilitating determination of the diversity of the genome. The skilled artisan will be aware that the term “C-value” refers to a haploid or gametic nuclear DNA content of an organism in picograms (Swift, 1950), determined e.g., by reference to a C-value Database such as, for example, the Plant DNA C-values Database (Bennett and Leitch, 2003) or the Animal Genome Size Database (Gregory, 2001).

Preferably at least about 70% of each contributing genome has been sequenced, and more preferably at least about 75% of each contributing genome has been sequenced. Even more preferably at least about 80% of each contributing genome has been sequenced.

Alternatively, or in addition to their characterization by a proportion of sequenced genome, preferred organisms from which the nucleic acids are derived have a C-value less than 100 or less than 60 or less than 40 or less than 30 or less than 20 or less than 18 or less than 16 or less than 14 or less than 12 or less than 10 or less than 9 or less than 8 or less than 7 or less than 6 or less than 5 or less than 4 or less than 3 or less than 2 or less than 1 or less than 0.9 or less than 0.8 or less than 0.7 or less than 0.6 or less than 0.5 or less than 0.4 or less than 0.3 or less than 0.2 or less than 0.1.

Preferred organisms having well-characterized genomes include, for example, an organism selected from the group consisting of Actinobacillus pleuropneumoniae serovar, Aeropyrum pernix, Agrobacterium lumeficians, Anopheles gambiae, Aquifex aeolicus, Arabidopsis thaliana, Archeglobus fulgidis, Bacillus anthracis, Bacillus cereus, Baccilus halodurans, Bacillus subtilis, Bacteroides thetaiotaomicron, Bdellovibrio bacteriovorus, Bifidobacterium longum, Bordetella bronchiseptica, Bordetella parapertussis, Borrelia burgdorferi, Bradyrhizobium japonicum, Brucella melitensis, Brucella suis, Bruchnera aphidicola, Brugia malayi, Caenorhabditis elegans, Campylobacter jejuni, Candidatus blochmannia floridanus, Caulobacter crescentus, Chlamydia muridarum, Chlamydia trachomatis, Chlamydophilia caviae, Chlamydia pneumoniae, Chlorobium tepidum, Chromobacterium violaceum, Clostridium acetobutylicum, Clostridium perfringens, Clostridium tetani, Corynebacterium diphtheriae, Corynebacterium efficient, Corynebacterium glutamicum, Coxiella burnetii, Danio rerio, Dechloromonas aromatica, Deinococcus radiodurans, Drosophila melanogaster, Eimeria tenella, Eimeria acervulina, Entamoeba histolytica, Enterococcus faecalis, Escherichia coli, Fusobacterium nucleatum, Geobacter sulfurreducens, Gloeobacter violaceus, Haemophilis ducreyi, Haemophilus injluenzae, Halobacterium, Helicobacter hepaticus, Helicobacter pylori, Lactobacillus johnsonii, Lactobacillus plantarum, Lactococcus lactis, Leptospira interrogans serovar lai, Listeria innocua, Listeria monocytogenes, Mesorhizobium loti, Methanobacterium thermoautotrophicum, Met hanocaldocossus jannaschii, Methanococcoides burtonii, Methanopyrus kandleri, Methanosarcina acetivorans, Methanosarcina mazei Goel, Methanothermobacter thermautotrophicus, Mycobacterium avium, Mycobacterium Bovis, Mycobacterium leprae, Mycobacterium tuberculosis, Mycoplasma gallisepticum strain R, Mycoplasnia genitalium, Mycoplasma penetrans, Mycoplasma pneumoniae, Mycoplasma pulmonis, Nanoarchaeum eqziitans, Neisseria meningitidis, Nitrosomonas europaea, Nostoc, Oceanobacillus iheyensis, Onion yellows phytoplasma, Oryzias latipes, Oryza sativa, Pasteurella multocida, Photorhabdus luminescens, Pirellula, Plasmodium falciparum, Plasmodium vivax, Plasmodium yoelii, Porphyromonas gingivalis, Prochlorococcus marinus, Prochlorococcus marinus, Prochlorococcus, Pseudomonas aeruginosa, Pseudomonas putida, Pseudomonas syringae, Pyrobaculum aerophilum, Pyrococcus abyssi, Pyrococcus furiosus, Pyrococcus horikoshii, Ralstonia solanacearum, Rhodopseudomonas palustris, Rickettsia conorii, Rickettsia prowazekii, Rickettsia rickettsii, Saccharomyces cerevisiae, Salmonella enterica, Salmonella typhimurium, Sarcocystis cruzi, Schistosoma mansoni, Schizosaccharomyces pombe, Shewanella oneidensis, Shigella flexneri, Sinorhizobium meliloti, Staphylococcus aureus, Staphylococcus epidermidis, Streptococcus agalactiae, Streptococcus agalactiae, Streptococcus mutans, Streptococcus pneumoniae, Streptococcus pyogenes, Streptomyces avermitilis, Streptomyces coelicolor, Sulfolobus solfataricus, Sulfolobus tokodaii, Synechocystis sp., Takifugu rubripes, Tetraodon fluviatilis, Theileria parva, Thermoanaerobacter tengcongensis, Thermoplasma acidophilum, Thermoplasma volcanium, Thermosynechococcus elongatus, Thermotoga maritima, Toxoplasma gondii, Treponema denticola, Treponema pallidum, Tropheryma whipplei, Tryponosoma brucei, Trypanosoma cruzi, Ureaplasma urealyticum, Vibrio cholerae, Vibro parahaemolyticus, Vibro vulnificus, Wigglesworthia brevipalpis, Wolbachia endosymbiont of Drosophilia melanogaster, WOlinella succinogenes, Xanthomonas axonopodis pv. Citri, Xanthomonas campestris pv. Campestris, Xylella fastidiosa, and Yersinia pestis.

Further examples of organisms having well-characterized genomes include:

a) bacterial species selected from Pseudomonas aeruginosa, Clostridium difficile, Acinetobacter baumannii, Aeromonas hydrophila, Bacillus cereus, Bacillus subtilis, Bacteroides thetaiotaomicron, Bordetella pertussis, Borrelia burgdorferi, Campylobacter jejuni subsp. Jejuni, Caulobacter vibrioides (crescentus), Chlorobium tepidum, Clostridium acetobutylicum, Clostridium difficile, Clostridium perfringens, Corynebacterium diphtheria, Deinococcus radiodurans, Desulfovibrio vulgaris, Geobacter sulfurreducens, Haemophilus influenza, Helicobacter pylori, Legionella pneumophila subsp. Pneumophila, Listeria innocua, Listeria monocytogenes, Mycobacterium avium subsp. paratuberculosis, Mycobacterium tuberculosis, Neisseria gonorrhoeae, Neisseria menigitidis, Porphyromonas gingivalis, Rhodobacter sphaeroides, Rhodopseudomonas palustris, Salmonella enterica subsp. enterica serovar Thyphimurium, Streptomyces avermitilis, Staphylococcus aureus, Streptococcus pyogenes and Thermotoga maritime; and
b) archael species selected from Haloarcula marismortui, Haloferax volcanii, Sulfolobus solfataricus, Halobacterium salinarum, Archeaoglobus fulgidis, Pyrococcus horikoshii, Methanococcus jannaschii, Aeropyrum pernix and Thermoplasma volcanicum; and
c) viruses selected from Human herpes virus 5 (CMV) (strain AD-169), Vaccinia virus, Human herpes virus 1 (HSV-1) (strain KOS), Human herpes virus 3 (Varicella-zoster virus) (strain Ellen), Human adenovirus C serotype 1 (HAdV-1) (strain adenoid 71), Human adenovirus B, subspecies B2, serotype 14 (HAdV-14), Coronavirus (strain 229E), Parainfluenza virus 4b, Measles virus (Ichinose-B95a), Parainfluenza virus 2, Parainfluenza virus 1 strain C35), Parainfluenza virus 3, Mumps (strain Enders), Human respiratory syncytial virus B (strain B1), Rhinovirus B17 (common cold), Human papillomavirus type 16, Human papillomavirus type 18, Human papillomavirus type 6b, Hepatitis B virus (clone AM6), Influenza A virus (H1N1), Human adenovirus C serotype 2 (HAdV-2), Dengue type 1 virus, Human herpesvirus 4(Ebstein-Barr virus), Human herpes virus 8 (Karposis sarcoma virus), Zaire ebola virus, Lake Victoria marburgvirus, Newcastle disease virus, Human respiratory syncytial virus B, Vesicular stomatitis Indiana virus, Influenza C virus, Adeno-associated virus 2, Foot-and-mouth virus, Hepatitis A virus, Human parechovirus 1 (echovirus 22), Simian Virus 40, Rotavirus A, Reovirus type 1, Avian leukosis virus RSA (RSV-SRA)/Rous sarcoma virus, Human immunodeficiency virus 1 and Sindbis virus.

In a further example, combinations of nucleic acid fragments from one or more eukaryote genomes and/or one or more prokaryote genomes and/or one or more viruses described according to any example hereof may be used.

Once produced, the nucleic acid fragments may be normalized to reduce any bias toward more highly-expressed genes amongst the contributing genomes. Methods of normalizing nucleic acids are known in the art, and are described, for example in, Ausubel et al. (In: Current Protocols in Molecular Biology. Wiley Interscience, ISBN 047 150338, 1987) or Sambrook et al. (In: Molecular Cloning: Molecular Cloning: A Laboratory Manual, Cold Spring Harbor Laboratories, New York, Third Edition 2001) and Soares et al. Curr. Opinion Biotechnol. 8, 542-546, (1997), and references cited therein. One of the methods described by Soares uses reassociation-based kinetics to reduce the bias of the library toward highly expressed sequences. Alternatively, cDNA is normalized through hybridization to genomic DNA that has been bound to magnetic beads, as described in Kopczynski et al, Proc. Natl. Acad. Sci. USA, 95, 9973-9978, (1998). This provides an approximately equal representation of cDNA sequences in the eluate from the magnetic beads. Normalized expression libraries produced using cDNA from one or two or more prokaryotes or compact eukaryotes are clearly contemplated by the present invention. Alternatively, fragments from each contributing genome are combined into a pool in amounts by weight in proportion to their relative genome size or C-value.

The nucleic acid fragments may be enriched for a subset of nucleic acid fragments to produce one or more enriched samples. As used herein, the term “enriched” is used in its broadest context to refer to any process that reduces the complexity of nucleic acids in a sample, generally by increasing the relative concentration of particular nucleic acid species in the sample. In one example, the nucleic acid fragments may be enriched for lower-copy regions by removing repetitive and/or hypo-methylated regions (Rabinowicz et al. Nature Genet. 23, 305-308, 1999; Peterson et al. Genome Res. 12, 795-807, 2002; Springer et al. Plant Physiol. 136, 3023-3033, 2004; Shagina et al. Biotechniques. 45, 455-459, 2010).

The nucleic acid fragments may also be modified by a process comprising mutagenesis or substitution or deletion or insertion of one of more nucleotides or codons such that the encoded candidate peptide moiety varies by one or more amino acids compared to the peptide encoded by the original nucleic acid fragment. The original nucleic acid fragment may have the same nucleotide sequence as in nature i.e., in the gene from which it was derived, or it may comprise a different sequence i.e., it may itself be an intermediate variant. Preferred mutations result in a different amino acid in the encoded peptide such as to satisfy codon preferences of host cells. Various methods may be employed to introduce one or more mutations into the open reading frame of nucleic acid e.g., mutagenic PCR, expressing nucleic acid in bacterial cells that induce random mutations, site directed mutagenesis, or exposure of host cells mutagenic agents such as radiation, bromo-deoxy-uridine (BrdU), ethylnitrosurea (ENU), ethylmethanesulfonate (EMS) hydroxylamine, or trimethyl phosphate. In mutagenic PCR, the nucleic acid fragments are preferably amplified in the presence of manganese and concentrations of dNTPs sufficient to result in their misincorporation. See e.g., Dieffenbach (ed) and Dveksler (ed) (In: PCR Primer: A Laboratory Manual, Cold Spring Harbour Laboratories, N Y, 1995), Leung et al., Technique 1, 11-15 (1989), Shafkhani et al. BioTechniques 23, 304-306, (1997) each of which is incorporated herein by way of reference. Commercially available means for performing kits mutagenic PCR are publicly-available e.g., Diversify PCR Random Mutagenesis Kit (Clontech) or the GeneMorph Random Mutagenesis Kit (Stratagene).

It will be apparent from the preceding description that preferred nucleic acid fragments for use in producing candidate peptides will comprise open reading frames having lengths consisting of about 45 to about 600 contiguous nucleotides or an average length consisting of about 300 contiguous nucleotides. It is to be understood that some variation from this range is permitted, the only requirement being that, on average, nucleic acid fragments generated encode a candidate peptide moiety comprising about at least about 15 to about 100 amino acids in length, and more preferably at least about 20 to about 100 amino acids in length and still more preferably at least about 30 to about 100 amino acids in length.

Methods of separating nucleic acid fragments according to their size or molecular weight are known in the art and include, for example, the fragmentation methods supra and a method of separation selected from the group comprising, agarose gel electrophoresis, pulse field gel electrophoresis, polyacrylamide gel electrophoresis, density gradient centrifugation and size exclusion chromatogram.

### Biotin Ligase Substrate Domain

Biotin is an essential cofactor of cell metabolism serving as a protein-bound coenzyme in ATP-dependent carboxylation, in transcarboxylation, and certain decarboxylation reactions. In particular, the carboxyl group of biotin is covalently attached to the epsilon-amino group of a specific lysine residue of an acceptor protein, i.e. a biotin ligase substrate domain Used as fusion tags at the C-terminus or the N-terminus, biotin ligase substrate domains allow the in vivo or in vitro site-directed biotinylation of fusion proteins.

The biotin ligase substrate domain may comprise a well-characterised biotin ligase substrate domain such as, for example, the biotin binding domain of the biotin carboxyl carrier protein of acetyl-CoA carboxylase from E. coli (Swiss-Prot No. P0ABD8; Chapman-Smith and Cronan, J. Nutr. 129, 477S-484S, 1999), the biotin binding domain of the oxaloacetate decarboxylase subunit from Klebsiella pneumoniae (Swiss-Prot No. P13187; Schwarz et al. J. Biol. Chem. 263, 9640-9645, 1988), the biotin binding domain of the 1.3 S subunit of transcarboxylase of Propionibacterium shermanii (Swiss-Prot No. P02904; Samols et al., J. Biol. Chem 263, 6461-6464, 1988), the biotin binding domain of the acetyl-CoA carboxylase biotin carboxyl carrier protein subunit from Pyrococcus horikoshii OT3 (Swiss-Prot No. 057883; Bagautdinov et al. Acta Crystallogr Sect F Struct Biol Cryst Commun. 63, 334-337, 2007), the biotin binding domain of the biotin carboxyl carrier protein from Aquifex aeolicus (067375; Clarke et al. Eur J Biochem. 270, 1277-87, 2003), the biotin binding domain of the biotin carboxyl carrier protein of acetyl-CoA carboxylase from Bacillus subtilis (P49786; Bower et al. J Bacteriol. 177, 7003-7006, 1995), the biotin binding domain of the acetyl-coenzyme A carboxylase carboxyl transferase subunit alpha from Paracoccus denitrificans (A1B4I6), the biotin binding domain of the human pyruvate carboxylase (P11498; Campeau and Gravel, J. Biol. Chem. 276, 12310-12316, 2001, the biotin binding domain of the human propionyl-CoA carboxylase (P05165; Campeau and Gravel, J. Biol. Chem. 276, 12310-12316, 2001), the biotin binding domain of the pyruvic carboxylase from Methanocaldococcus jannaschii (Q58628), the biotin binding domain of the biotin carboxyl carrier protein of acetyl-CoA carboxylase from Lycopersicon esculentum (Hoffman et al., Nucleic Acid Res. 15, 3928, 1987) or the biotin binding domain of ARC1 from Saccharomyces cerevisiae (P46672; Kim J Biol Chem. 279, 42445-42452, 2004).

In another example, the biotin ligase substrate domain may comprise a minimal peptide recognition sequence that is capable of being enzymatically biotinylated such as, for example, the 13 amino acid sequence that is capable of being enzymatically biotinylated by the biotin ligase from E. coli (SEQ ID NO: 3), the 15 amino acid sequence that is capable of being enzymatically biotinylated by the biotin ligase from E. coli (SEQ ID NO: 4), the 15 amino acid sequence that is capable of being enzymatically biotinylated by the biotin ligase from B. subtilis (SEQ ID NO: 6), the 15 amino acid sequence that is capable of being enzymatically biotinylated by the biotin ligase from M. jannaschii (SEQ ID NO: 8), the 15 amino acid sequence that is capable of being enzymatically biotinylated by the biotin ligase from S. cerevisiae (SEQ ID NO: 10), or the 15 amino acid that is sequence capable of being enzymatically biotinylated by the biotin ligase from S. cerevisiae (SEQ ID NO: 12).

Methods of identifying a minimal peptide recognition sequence are known in the art and are described for example in Kim et al. J. Biol. Chem. 279, 42445-42452 (2004) and Schwarz et al. J. Biol. Chem. 263, 9640-9645, (1988).

In yet another example, commercially available biotin binding domains recognisable capable of being enzymatically biotinylated by the biotin ligase from E. coli may be used such as, for example, the Bioease Tag (Invitrogen), the AviTag (Avidity) or the PinPoint vectors (Promega).

Nucleic acid encoding the biotin ligase substrate domain may be preferably isolated or synthesized. In this respect, the nucleotide sequence of a nucleic acid encoding the biotin ligase substrate domain may be identified using a method known in the art and/or described herein, e.g., reverse translation. Such a nucleic acid is then produced by synthetic means or recombinant means. For example, the nucleic acid is isolated using a known method, such as, for example, amplification (e.g., using PCR or splice overlap extension). Methods for such isolation will be apparent to the ordinary skilled artisan and/or described in Ausubel et al. (In: Current Protocols in Molecular Biology. Wiley Interscience, ISBN 047 150338, 1987), Sambrook et al. (In: Molecular Cloning: Molecular Cloning: A Laboratory Manual, Cold Spring Harbor Laboratories, New York, Third Edition 2001).

Other methods for the production of nucleic acid encoding the biotin ligase substrate domain will be apparent to the skilled artisan and are encompassed by the present invention. For example, the nucleic acid may be produced by synthetic means. Methods for synthesizing a nucleic acid are described, in Gait (Ed) (In: Oligonucleotide Synthesis: A Practical Approach, IRL Press, Oxford, 1984). Methods for oligonucleotide synthesis include, for example, phosphotriester and phosphodiester methods (e.g. Narang et al. Meth. Enzymol 68, 90, 1979) and synthesis on a support (e.g. Beaucage et al. Tetrahedron Letters 22, 1859-1862, 1981) as well as phosphoramidate technique, Caruthers, M. H., et al., “Methods in Enzymology,” Vol. 154, pp. 287-314 (1988), and others described in “Synthesis and Applications of DNA and RNA,” S. A. Narang, editor, Academic Press, New York, 1987, and the references contained therein.

### Fusion Proteins

The candidate peptide moiety and biotin ligase substrate domain may be linked by a covalent bond. A covalent bond, as defined herein, may be, for example, a peptide bond, which may be obtained by expressing the candidate peptide moiety and biotin ligase substrate domain as a fusion protein. The relative positions of candidate peptide and the biotin ligase substrate domain may be modified. In one example, the biotin ligase substrate domain is positioned upstream the N-terminus of the candidate peptide moiety. In another example, the biotin ligase substrate domain is adjacent the N-terminus of the candidate peptide moiety. In yet another example, the biotin ligase substrate domain is adjacent the C-terminus of the candidate peptide moiety. In yet another example, the biotin ligase substrate domain is positioned downstream of the C-terminus of the candidate peptide moiety.

Methods for construction of fusion proteins are known to the skilled artisan. See e.g., Sambrook et al. (In: Molecular Cloning: Molecular Cloning: A Laboratory Manual, Cold Spring Harbor Laboratories, New York, Third Edition 2001).

In one example, the candidate peptide moiety and at least one biotin ligase substrate domain are linked contiguously i.e., without intervening linker molecule, spacer molecule, detectable label, or other amino acids. In such a configuration, the candidate peptide moiety and biotin ligase substrate domain are generally adjacent.

In another example, the candidate peptide moiety and at least one biotin ligase substrate domain are linked non-contiguously i.e., separated by an additional molecule. In such a configuration, the candidate peptide moiety and biotin ligase substrate domain(s) are generally not adjacent but upstream or downstream relative to each other.

The candidate peptide moiety and biotin ligase substrate domain may be both present in a single copy in the fusion protein, and it is particularly preferred for the candidate peptide moiety to be present as a single copy.

In some examples, a plurality of copies of the candidate peptide moiety and/or biotin ligase substrate domain are present in the fusion protein. Preferably, multiple copies of a biotin ligase substrate domain may be represented in the fusion protein. Preferably, when multiple copies of a biotin ligase substrate domain are present, these are the same biotin ligase substrate domain. Preferably two or three or four or five or six or seven or eight or nine or ten or more copies of a biotin ligase substrate domain are present and fused to a single copy of a candidate peptide moiety. For example, a plurality of biotin ligase substrate domains may be linked contiguously or non-contiguously to each other and these may be linked contiguously or non-contiguously to the candidate peptide moiety. The plurality of biotin ligase substrate domains may be positioned at or after the C-terminus of the candidate peptide moiety or at or before the N-terminus of the candidate peptide moiety. Alternatively, the candidate peptide moiety may be positioned between a plurality of biotin ligase substrate domains such that one or more biotin ligase substrate domains is positioned at or before the N-terminus of the candidate peptide moiety and one or more biotin ligase substrate domains is positioned at or after the C-terminus of the candidate peptide moiety.

Preferred molecules for achieving non-contiguous linkages between a candidate peptide moiety and a biotin ligase substrate domain and for achieving non-contiguous linkages between biotin ligase substrate domains are selected from a linker molecule, a spacer molecule, and a detectable label, and/or other amino acids.

In one example, an amino acid linker such as a polyglycine or polyasparagine or polyarginine or polylysine or polyglutamine or polyornithine or polyalanine or polyserine or a mixmer comprising glycine and/or asparagine and/or arginine and/or lysine and/or glutamine and/or ornithine and/or alanine and/or serine is employed. Preferred amino acid linkers comprise two or three or four or five or six contiguous amino acids to separate a candidate peptide from a biotin ligase substrate domain or separate a plurality of biotin ligase substrate domains from each other. Preferred linkers do not form the sequence of a recognition site for a host cell protease enzyme and/or provide a more flexible linkage Polyglycine and/or polyserine and/or polyalanine linkers and mixmers thereof are particularly preferred.

In another example, a carbon spacer is employed e.g., an aliphatic molecule comprising two or three or four or five or six or seven or eight or nine or ten carbon atoms in tandem, and optionally a heteroaliphatic molecule comprising two or three or four or five or six or seven or eight or nine or ten carbon atoms and one or more additional heteroatoms e.g., sulfur, oxygen, or NH group. Aromatic diamine spacers comprising p-phenylenediamine and/or m-phenylenediamine may also be employed. Preferred spacers comprise bonds having rotational freedom to prevent steric interference between the candidate peptide and biotin ligase substrate domain.

In yet another example, a detectable label comprising a peptide tag may be employed e.g., a poly-histidine tag such as a hexahistidine tag, or dodecahistidine tag, FLAG tag, Myc tag, hemagglutinin (HA) tag, a glutathione-S-transferase (GST) tag, V5 epitope tag, or fluorescent protein. Fluorescent proteins are known in the art and include, for example, Green Fluorescent Protein (GFP) and colour variants thereof like YFP (Yellow Fluorescent Protein) and DsRed.

For example, one or more linkers and/or spacers and/or detectable labels may be positioned upstream of an N-terminus of a candidate peptide moiety or adjacent an N-terminus of a candidate peptide moiety or adjacent a C-terminus of a candidate peptide moiety or downstream of a C-terminus of a candidate peptide moiety or upstream an N-terminus of a biotin ligase substrate domain or adjacent an N-terminus of a biotin ligase substrate domain or adjacent a C-terminus of a biotin ligase substrate domain or downstream of a C-terminus of a biotin ligase substrate domain. Depending on the number and relative orientation of the candidate peptide and biotin ligase substrate domain(s) in the fusion peptide, one or more linkers and/or spacers and/or detectable labels may be positioned upstream of an N-terminus of a candidate peptide moiety and downstream of a C-terminus of a biotin ligase substrate domain or downstream of a C-terminus of a candidate peptide moiety and upstream an N-terminus of a biotin ligase substrate domain.

In yet another example, the fusion protein comprises one or more additional moieties that interact with a protein or polysaccharide on the surface of the host cells. See e.g., Ziello et al. Mol. Med. 16, 222-229 (2010); Sahay et al. J. Control. Release. 145, 182-195 (2010). Positioning of the moiety may be at an N-terminus or C-terminus of the fusion protein. Alternatively, or in addition, a moiety may be positioned internal to the fusion protein at any position suitable for introducing a linker or spacer or detectable label as described herein above. In one example, the interaction between such a moiety and the surface bound protein or polysaccharide induces or promotes or enhances binding of the fusion protein to the host cell. In another example, the interaction between such a moiety and the surface bound protein or polysaccharide induces or promotes or enhances cellular uptake of the fusion protein. In yet another example, the interaction between such a moiety and the surface bound protein or polysaccharide induces or promotes or enhances (i) binding of the fusion protein to the host cell and (ii) cellular uptake of the fusion protein.

### Production of Non-Biotinylated Members

As exemplified herein, a pool of non-biotinylated members is produced using phage display technology wherein fusion proteins are displayed on the surface of a bacteriophage, as described, for example, in U.S. Pat. No. 5,821,047 and U.S. Pat. No. 6,190,908. The basic principle described relates to the fusion of a first nucleic acid comprising a sequence encoding a peptide or protein to a second nucleic acid comprising a sequence encoding a phage coat protein, such as, for example a pIII coat protein, a pVI coat protein, a pVII coat protein, a pVIII coat protein, a pIX coat protein, or a 10B capsid protein. These sequences are then inserted into an appropriate vector, e.g., a vector capable of replicating in bacterial cells. Suitable cells, such as, for example E. coli, are then transformed with the recombinant vector. These cells are may also be infected with a helper phage particle encoding an unmodified form of the coat protein to which a nucleic acid fragment is operably linked. Transformed, infected host cells are cultured under conditions suitable for forming recombinant phagemid particles comprising more than one copy of the fusion protein on the surface of the particle. This system has been shown to be effective in the generation of virus particles such as, for example, a virus particle selected from the group comprising λ phage, T4 phage, M13 phage, T7 phage and baculovirus.

An alternative method for producing a pool of non-biotinylated members comprises in vitro translation of mRNA. Suitable extracts such as, for example, rabbit reticulocyte lysates, wheat germ extract, canine pancreatic microsomal membranes, E. coli S30 extract, SF9 or SF21 insect cell lysates, Leishmania tarentolae extract as well as coupled transcription/translation systems may be used for cell-free protein expression. Corresponding assay systems are commercially available from various suppliers.

In an alternative example, a pool of non-biotinylated members is produced using ribosome display technology. Such methods require that the nucleic acid encoding the fusion protein be placed in operable connection with an appropriate promoter sequence and ribosome binding sequence, e.g. from a gene construct. Preferred promoter sequences are the bacteriophage T3 and T7 promoters. Preferably, the nucleic acid encoding the fusion protein is placed in operable connection with a spacer sequence and a modified terminator sequence with the terminator sequence removed. As used herein the term “spacer sequence” shall be understood to mean a series of nucleic acids that encode a peptide that is fused to the peptide. The spacer sequence is incorporated into the gene construct, as the peptide encoded by the spacer sequence remains within the ribosomal tunnel following translation, while allowing the peptide to freely fold and interact with another protein or a nucleic acid. A preferred spacer sequence is, for example, a nucleic acid that encodes amino acids 211-299 of gene III of filamentous phage M13. The display library is transcribed and translated in vitro using methods well known in the art and are described for example, in Ausubel et al. (In: Current Protocols in Molecular Biology. Wiley Interscience, ISBN 047 150338, 1987) and Sambrook et al. (In: Molecular Cloning: Molecular Cloning: A Laboratory Manual, Cold Spring Harbor Laboratories, New York, Third Edition 2001).

Examples of systems for in vitro transcription and translation include, for example, the TNT in vitro transcription and translation systems from Promega. Cooling the expression reactions on ice generally terminates translation. The ribosome complexes are stabilized against dissociation from the peptide and/or its encoding mRNA by the addition of reagents such as, for example, magnesium acetate or chloroamphenicol.

Alternatively, a pool of non-biotinylated members is produced using ribosome inactivation display technology, e.g., as described in Tabuchi, Biochem Biophys Res Commun. 305, 1-5, 2003 or a covalent display technology.

In yet another example, production of a pool of non-biotinylated members comprises a process comprising bacterial display, wherein fusion proteins are displayed on the surface of a bacterial cell. The cells displaying the expressed fusion proteins are then used for biopanning as described, for example, in U.S. Pat. No. 5,516,637. Alternatively, the pool of non-biotinylated members may be produced using yeast display technology, e.g., as described in U.S. Pat. No. 6,423,538 or mammalian display technology, e.g., as described in Strenglin et al. EMBO J. 7, 1053-1059, 1988.

The cells used for the production of the pool of non-biotinylated members may vary e.g., depending on the biotin ligase substrate domain to be expressed in the fusion protein. In one example, the biotin ligase substrate domain is derived from a different organism to the cells used to produce the non-biotinylated members. For example, should the non-biotinylated members be produced in a mammalian cell, the biotin ligase substrate domain is preferably derived from an organism from a different kingdom such as, for example, Prokaryotae Monera (e.g., bacterium), Protista (e.g., a protozoan), Fungi or Plantae. In another example, should the non-biotinylated members be produced in a bacterial cell, the biotin ligase substrate domain is preferably derived from an organism from a kingdom such as, for example, Fungi, Plantae or Animalia. For example, Cronan et al. FEMS Microbio. Lett. 130, 221-229, 1995 describe production of E. coli CY918 cells expressing a recombinant biotin ligase.

In another example, non-biotinylated members are produced in cells expressing a biotin ligase having a reduced level of expression as compared to a wild-type biotin ligase e.g., at less than 50% or less than 60% or less than 70% or less than 80% or less than 90% or less than 95% of the expression level of a wild-type biotin ligase. In yet another example, non-biotinylated members are produced in cells expressing a biotin ligase having a reduced activity as compared to a wild-type biotin ligase e.g., less than 50% or less than 60% or less than 70% or less than 80% or less than 90% or less than 95% activity as compared a wild-type biotin ligase. In yet another example, non-biotinylated members are produced in cells that lack endogenous biotin ligase activity e.g., cells expressing a non-functional endogenous biotin ligase or cells that do not express a level of biotin ligase activity sufficient to biotinylate the biotin ligase substrate domain(s) of the fusion peptide. Cells that lack endogenous biotin ligase activity may express a recombinant biotin ligase. Biotin ligase activity is generally determined by monitoring the time-dependent incorporation of radiolabelled biotin into a biotin ligase substrate domain as described e.g., by Purushothaman et al. PLoS ONE 3, e2320 (2008).

Methods for altering gene expression and/or activity will be apparent to the skilled artisan and include, for example, deletion or disruption of genome sequence encoding biotin ligase, mutagenesis e.g., transposon mutagenesis or radiation mutagenesis or chemical mutagenesis, gene inactivation or gene silencing.

In one preferred example, gene silencing is employed to reduce biotin ligase expression in a cell. Gene silencing is induced using “knock-out” technology, for example, as described in Hogan et al (In: Manipulating the Mouse Embryo. A Laboratory Manual, 2^{nd} Edition or Porteus et al, Mol. Cell. Biol, 23: 3558-3565, 2003. In this example, a cell or animal in which a biotin ligase gene is knocked-out is produced using a replacement vector comprising two regions of homology to a biotin ligase target gene located on either side of a heterologous nucleic acid encoding one or more positive selectable markers, such as, for example, a fluorescent protein e.g., enhanced green fluorescent protein, or β-galactosidase, or antibiotic resistance protein e.g., for neomycin or zeocin resistance, or a fusion protein e.g., β-galactosidase-neomycin resistance protein, β-geo, amongst others. The vector is introduced into a cell expressing biotin ligase under conditions sufficient for homologous recombination between the regions of homology in the vector and the target biotin ligase gene. Homologous recombination proceeds generally by at least two recombination events or a double cross-over event leading to replacement of biotin ligase gene sequence encoding functional enzyme with replacement vector sequence encoding sequence that is non-functional for biotin ligase activity, or less-functional. More specifically, each region of homology in the vector induces at least one recombination event that leads to the heterologous nucleic acid in the vector replacing the nucleic acid located between the regions of homology in the target gene.

Alternative methods for knocking out a gene of interest are apparent to the skilled person, for example, using recombination e.g., recombination of nucleic acid located between two LoxP sites using the enzyme Cre.

Alternatively, gene silencing is induced using, for example, using RNA interference e.g., Hannon and Conklin, Methods Mol Biol. 257, 255-266 (2004), or antisense technology e.g., Sahu et al. Curr. Pharm. Biotechnol. 8, 291-304 (2007), or ribozymes e.g., Barrel and Szostak, Science 261, 1411-1418 (1993), or nucleic acid capable of forming a triple helix e.g., Helene, Anticancer Drug Res. 6, 569-584 (1991), or PNA oligonucleotides e.g., Hyrup et al. Bioorganic & Med. Chem. 4, 5-23 (1996) or O'Keefe et al. Proc. Natl Acad. Sci. USA 93, 14670-14675 (1996), or site-directed mutagenesis e.g., Yan et al., Gene Therapy 16, 581-588 (2009), or zinc finger nucleases e.g., Durai et al., Nucleic Acids Res. 33, 5978-5990 (2005).

In yet another example, non-biotinylated members are produced in cells that express a biotin ligase that has a low affinity for the biotin ligase substrate domain, e.g., an affinity of less than 25% the affinity that the enzyme has for its canonical biotin ligase substrate domain. Preferred biotin ligases for use in this example have less than 20% or less than 15% or less than 10% or less than 5% or less than 4% or less than 3% or less than 2% or less that 1% the affinity that the enzyme has for its canonical biotin ligase substrate domain By “canonical biotin ligase substrate domain” is meant a biotin ligase substrate domain comprising an amino acid sequence on which the biotin ligase is known to act in nature e.g., by virtue of being from the same organism. Exemplary biotin ligases having a low affinity for a biotin ligase substrate domain derived from E. coli include Saccharomyces cerevisiae biotin ligase (Swiss-Prot No. P48445), Bacillus subtilis biotin ligase (Swiss-Prot No. POC175), or Methanococcus jannaschii biotin ligase (Swiss-Prot No. Q59014). In another example, E. coli biotin ligase (Swiss-Prot No. P06709) has a low affinity for the biotin ligase substrate domain derived from yeast.

In yet another example, non-biotinylated members are produced in cells expressing a second fusion polypeptide comprise a plurality of biotin ligase substrate domains to thereby provide preferential biotinylation of the polypeptide relative to the biotin ligase substrate domain of the fusion protein. For example, the polypeptide may comprise 2 or 3 or 4 or 5 or 6 or 7 or 8 or 9 or 10 biotin ligase substrate domains. In accordance with this example, it is preferred for the second fusion polypeptide to comprise a sufficient number of biotin ligase substrate domains to compete with the non-biotinylated member for cellular biotin ligases. Alternatively, or in addition, the second fusion polypeptide will generally comprise one or more canonical biotin ligase substrate domains to compete with non-canonical biotin ligase substrate domains of the non-biotinylated member for cellular biotin ligase having a higher affinity for the canonical biotin ligase substrate domains relative to the non-canonical biotin ligase substrate domains. For example, the non-biotinylated member may be produced in E. coli cells expressing a second fusion polypeptide comprising one or more biotin ligase substrate domains derived from E. coli, wherein the non-biotinylated member comprises one or more biotin ligase substrate domains derived from yeast.

### Biotinylation of the Non-Biotinylated Members

### Host Cells

Preferred host cells for biotinylating the non-biotinylated members are prokaryotic cells.

Suitable prokaryotic host cells include, for example, strains of E. coli (e.g., BL21, DH5α, XL-1-Blue, JM105, JM110, and Rosetta), Bacillus subtilis, Salmonella sp., and Agrobacterium tumefaciens. More preferably, host cells are eukaryotic cells. Suitable mammalian cells include cell lines, such as, for example, human GM12878, K562, H1 human embryonic, Hela, HUVEC, HEPG2, HEK-293, H9, MCF7, and Jurkat cells, mouse NIH-3T3, C127, and L cells, simian COS1 and COS7 cells, quail QC1-3 cells, and Chinese hamster ovary (CHO) cells. In one example, the host cells are primary mammalian cells, that is, cells directly obtained from an organism (at any developmental stage including inter alia blastocytes, embryos, larval stages, and adults). In some examples, the host cell of the present invention constitutes a part of a multi-cellular organism. In other words, the invention encompasses the use of transgenic organisms comprising at least one host cell as defined herein. Preferred multicellular organisms for this purpose will include organisms having a short life cycle to facilitate rapid high throughput screening, such as, for example, a plant (e.g., Arabidopsis thaliana or Nicotinia tabacum) or an animal selected from the group consisting of Caenorhabditis elegans, Danio rerio, Drosophila melanogaster, Takifugu rubripes, Mus sp. and Rattus sp.

Appropriate culture media and conditions for culturing the cell populations and cell lines are known in the art. With respect to the conditions necessary and sufficient for enzymatic biotinylation of the biotin ligase substrate domain by the biotin ligase expressed by the host cell may be determined empirically. In some examples, culture media may be supplemented with biotin. For example, culture media may be supplemented with biotin to a final concentration in the culture media of 1 μM or 2 μM or 3 μM or 4 μM or 5 μM or 6 μM or 7 μM or 8 μM or 9 μM or 10 μM or 20 μM or 30 μM or 40 μM or 50 μM or 60 μM or 70 μM or 80 μM or 90 μM or 100 μM or 200 μM. The skilled artisan will also be aware that some reagents commonly present in biological buffers reduce biotin ligase activity, such as, for example, 100 mM NaCl or 5% glycerol or 50 mM ammonium sulfate.

### Biotin Ligase

Any biotin ligase known in the art may be used for the methods of the present invention provided that the biotin ligase is capable of enzymatically biotinylating the biotin ligase substrate domain of the fusion protein. It will be understood by the skilled artisan that the biotin ligase is an enzyme that catalyzes the covalent attachment of biotin to a fusion protein comprising a biotin ligase substrate domain via an amide linkage between the biotin carboxyl group and the amino group of a lysine of the fusion protein.

In one example, the biotin ligase is expressed endogenously by the host cell.

Alternatively, the biotin ligase expressed by the host cells is a recombinant biotin ligase. In some examples, the recombinant biotin ligase is a prokaryotic biotin ligase. Alternatively, the biotin ligase is a eukaryotic biotin ligase. Suitable biotin ligases include, for example, the biotin ligase from Bacillus subtilis (Swiss-Prot No. P0C175), the biotin ligase from Candida albicans (Swiss-Prot No. Q5ACJ7), the biotin ligase from E. coli (Swiss-Prot No. P06709), the biotin ligase from Haemophilus influenza (Swiss-Prot No. P46363), the biotin ligase from Homo sapiens (Swiss-Prot No. P50747), the biotin ligase from Methanococcus jannaschii (Swiss-Prot No. Q59014), the biotin ligase from Mus musculus (Swiss-Prot No. Q920N2), the biotin ligase from Neisseria meningitidis serogroup A (Swiss Prot Q9JWI7), the biotin ligase from Neisseria meningitidis serogroup B (Swiss-ProtQ9JXF1), the biotin ligase from Paracoccus denitrificans (Swiss-Prot No. P29906), the biotin ligase from Saccharomyces cerevisiae (Swiss-Prot No. P48445), the biotin ligase from Salmonella typhimurium (Swiss-Prot No. P37416) or the biotin ligase from Schizosaccharomyces pombe (Swiss-Prot No. 014353). As used herein the term “Swiss-Prot” shall be taken to mean the protein sequence database of the Swiss Institute of Bioinformatics at Basel University 4056. Basel, Switzerland.

The biotin ligase expressed by the host cells may be varied e.g., depending on the biotin ligase substrate domain to be expressed in the fusion protein. In one example, the biotin ligase expressed by the host cells is derived from a different organism to the host cells. For example, should the host cells be mammalian cells, the biotin ligase substrate domain may be derived from an organism from a different kingdom such as, for example, Prokaryotae Monera (e.g., bacterium), Protista (e.g., a protozoan), Fungi or Plantae.

Methods for the identification of biotin ligases are known in the art. For example, biotin ligases may be identified using sequence comparison algorithms provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn” that is used to align a known nucleotide sequence with other polynucleotide sequences from a variety of databases and “blastp” used to align a known amino acid sequence with one or more sequences from one or more databases. Also available is a tool called “BLAST 2 Sequences” that is used for direct pairwise comparison of two nucleotide sequences.

The nucleic acid encoding the biotin ligase may be isolated using polymerase chain reaction (PCR). Methods of PCR are known in the art and described, for example, in Dieffenbach (ed) and Dveksler (ed) (In: PCR Primer: A Laboratory Manual, Cold Spring Harbour Laboratories, N Y, 1995). Generally, for PCR two non-complementary nucleic acid primer molecules comprising at least about 20 nucleotides in length and more preferably at least 25 nucleotides in length are hybridized to different strands of a nucleic acid template molecule, and specific nucleic acid molecule copies of the template are amplified enzymatically. Following amplification, the amplified nucleic acid is isolated using methods known in the art and described, for example, in Ausubel et al. (In: Current Protocols in Molecular Biology. Wiley Interscience, ISBN 047 150338, 1987) or Sambrook et al. (In: Molecular Cloning: Molecular Cloning: A Laboratory Manual, Cold Spring Harbor Laboratories, New York, Third Edition 2001).

Alternatively, the nucleic acid encoding the biotin ligase may be synthesized using a chemical method known to the skilled artisan. For example, synthetic peptides are prepared using known techniques of solid phase, liquid phase, or peptide condensation, or any combination thereof, and can include natural and/or unnatural amino acids.

It is also understood in the art that the coding sequence of the biotin ligase may be modified for use in host cell (e.g. bacterial cells, insect cells, yeast cells, mammalian cells or plant cells) in accordance with known codon usage preferences. Codon usage preferences is a technique to maximize the protein expression in living organism by increasing the translational efficiency of gene of interest by transforming DNA sequence of nucleotides of one species into DNA sequence of nucleotides of another species (Puigbo et al. Nucleic Acids Res. 35, W126-W131, 2007).

In one example, the biotin ligase is fused to a polypeptide localisation signal capable of directing the biotin ligase to a particular subcellular location of the host cell. Sub-cellular polypeptide localisation sequences are known in the art, and are described, for example, on the Signal Sequence Database website which provides a direct access to the signal sequence domain of Mammals, Drosophila, Bacteria and Viruses. Methods for predicting sub-cellular polypeptide localisation sequences using a computer program or algorithm are also known in the art and are accessed through online software packages such as, for example, SIGNAL-BLAST (Frank and Sippl, Bioinformatics 24, 2171-2176, 2008).

Following amplification/synthesis, the biotin ligase may be expressed by recombinant means. For example, the nucleic acid encoding the biotin ligase may be placed in operable connection with a promoter or other regulatory sequence capable of regulating expression in cellular system or organism.

Typical promoters suitable for expression in bacterial cells include, for example, the lacz promoter, the Ipp promoter, temperature-sensitive λ_{L} or λ_{R} promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are well-known in the art and are described, for example, in Ausubel et al. (In: Current Protocols in Molecular Biology. Wiley Interscience, ISBN 047 150338, 1987), U.S. Pat. No. 5,763,239 (Diversa Corporation) and Sambrook et al. (In: Molecular Cloning: Molecular Cloning: A Laboratory Manual, Cold Spring Harbor Laboratories, New York, Third Edition 2001).

Numerous expression vectors for expression of recombinant polypeptides in bacterial cells have been described, and include, for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen) the pBAD/TOPO or pBAD/thio-TOPO series of vectors containing an arabinose-inducible promoter (Invitrogen), the latter of which is designed to also produce fusion proteins with a Trx loop for conformational constraint of the expressed protein; the pFLEX series of expression vectors (Pfizer); the pQE series of expression vectors (QIAGEN), or the pL series of expression vectors (Invitrogen), amongst others.

Typical promoters suitable for expression in yeast cells such as, for example, a yeast cell selected from the group comprising Pichia pastoris, S. cerevisiae and S. pombe, include, but are not limited to, the ADH1 promoter, the GAL1 promoter, the GAL4 promoter, the CUP1 promoter, the PH05 promoter, the nmt promoter, the RPR1 promoter, or the TEF1 promoter.

Expression vectors for expression in yeast cells are preferred and include, for example, the pACT vector (Clontech), the pDBleu-X vector, the pPIC vector suite (Invitrogen), the pGAPZ vector suite (Invitrogen), the pHYB vector (Invitrogen), the pYD 1 vector (Invitrogen), and the pNMT 1, pNMT41, pNMT81 TOPO vectors (Invitrogen), the pPC86-Y vector (Invitrogen), the pRH series of vectors (Invitrogen), pYESTrp series of vectors (Invitrogen).

Preferred vectors for expression in mammalian cells include, for example, the pcDNA vector suite (Invitrogen), the pTARGET series of vectors (Promega), and the pSV vector suite (Promega).

Commercially available vectors for expression of the biotin ligase in bacterial cells are also available and include, for example, E. coli strains AVB 99 and AVB 101 (Avidity).

Suitable methods for transforming and transfecting host cells can be found in Sambrook et al. (In: Molecular Cloning: Molecular Cloning: A Laboratory Manual, Cold Spring Harbor Laboratories, New York, Third Edition 2001) and other laboratory textbooks.

In one example, nucleic acid is introduced into prokaryotic cells using for example, electroporation or calcium-chloride mediated transformation. In another example, nucleic acid is introduced into mammalian cells using, for example, microinjection, calcium phosphate or calcium chloride co-precipitation, DEAE-dextran mediated transfection, transfection mediated by liposomes such as by using Lipofectamine(Invitrogen) and/or cellfectin (Invitrogen), PEG mediated DNA uptake, electroporation, transduction by Adenoviuses, Herpesviruses, Togaviruses or Retroviruses and microparticle bombardment such as by using DNA-coated tungsten or gold particles. In yet another example, nucleic acid is introduced into plant cells using conventional techniques such as, for example, Agrobacterium mediated transformation, electroporation of protoplasts, PEG mediated transformation of protoplasts, particle mediated bombardment of plant tissues, and microinjection of plant cells or protoplasts. Alternatively, nucleic acid is introduced into yeast cells using conventional techniques such as, for example, electroporation, and PEG mediated transformation.

### Determining or Identifying Biotinylated Members

The presence of a biotinylated fusion protein may be determined by detecting the presence of biotin covalently attached to the biotin ligase substrate domain of a fusion protein. Biotin-binding molecules such as, for example, avidin, streptavidin, neutravidin, or captavidin may be used to detect the presence of detected biotinylated proteins. See e.g. Laitinen et al. Trends Biotechnol. 25, 269-277 (2007), Morag et al. Anal. Biochem. 243, 257-263 (1996), Morag et al. Biochem. J. 316, 193-199 (1996), Vermette et al. J. Colloid Interface Sci. 259, 13-26 (2003). In other examples, biotin-binding molecules such as, for example, anti-biotin antibodies may be used to detect biotinylated proteins.

Biotinylated fusion proteins may be visualised using fluorochrome-labelled biotin-binding molecules. Suitable fluorochromes may include for example, TAMRA dyes (e.g. Hsu et al. Clin. Chem. 47, 1373-1377, 2001), BODIPY dyes (e.g. Hecht et al. ChemistryOpen 2, 25-38, 2013), CHROMEO dyes (e.g. Active Motif), DyLight Fluor dyes (e.g. Sarkar et al. J. Photochem. Photobiol. B. 98, 35-39, 2010), sulforhodamine dyes such as for example, Texas Red, Lissamine rhodamine B-sulfonyl chloride, fluorescein and derivatives thereof including for example, fluorescein isothiocyanate (FITC), dichlorotriazinyl aminofluorescein (DTAF), carboxyfluorescein succinimidyl ester (CFSE) (e.g., Liu J. Fluoresc. 19, 915-920, 2009), cyanine dyes such as for example Cy2, Cy3, Cy3.5 Cy5, Cy5.5 (e.g. Kricka Ann. Clin Biochem. 39 114-129, 2002) or Alexa Fluor Dyes (e.g. Panchuck-Voloshina et al. J. Histochem. Cytochem. 47, 1179-1188, 1999).

Alternatively, biotinylated fusion proteins may be visualised using biotin-binding molecules labelled with an enzyme. In some examples, the enzyme may be a peroxidase such as horseradish peroxidase (HRP) or chloramphenicol acetyl transferase (CAT) or β-glucuronidase (GUS) or beta-galactosidase or xanthium oxidase or a phosphatase such as alkaline phosphatase, or a luciferase such as, for example, the firefly luciferase of Photinus pyralis or the Renilla luciferase of Renilla reniformis, Gaussia luciferase, Oplophorus luciferase, luciferin-utilizing luciferases, coelenterazine-utilizing luciferases, and any suitable variants or mutants thereof.

Other methods for detecting the presence of biotin are known in the art and are described, for example, by Haugland and Bhalgat, Methods Mol. Biol. 4, 1-12 (2008), Mason et al. Methods Mol. Biol. 303, 35-50 (2005), Hofstetter Anal. Biochem. 284, 354-366 (2000), Praul et al., Biochem Biophys Res Commun 247, 312-314 (1988), Santos and Chaves, Braz. J. Med. Biol. Res. 30, 837-842 (1997), Kin and Suh, Biochem. Physiol. B. Biochem. Mol. Biol. 115, 57-61 (1996), Hoeltke Biotechniques 18, 900-907 (1994) and Dunn Methods Mol. Biol. 32, 227-232(1994).

In some examples, prior to detecting the presence of biotin covalently attached to the biotin ligase substrate domain of a fusion protein, the host cells may be incubated with an agent to inhibit the activity of the biotin ligase. Inhibiting the activity of the biotin ligase may prevent promiscuous biotinylation from occurring in a host cell lysate. Agents that inhibit the activity of a biotin ligase will be apparent to the ordinary skilled artisan, such as, for example, pyrophosphate, biotinyl-5′AMP, biotinol-adenylate and biotin analogues.

Methods for isolating fusion proteins are well known in the art and include inter alia ion exchange chromatography, affinity chromatography, gel filtration chromatography (size exclusion chromatography), high-pressure liquid chromatography (HPLC), reversed phase HPLC, disc gel electrophoresis, and immune-precipitation. See e.g. Sambrook et al. (In: Molecular Cloning: Molecular Cloning: A Laboratory Manual, Cold Spring Harbor Laboratories, New York, Third Edition 2001) and other laboratory textbooks. These methods may be applied to isolating biotinylated fusion proteins of the invention.

### Functional Assays

In a preferred example, the method of identifying a cell penetrating peptide may comprise performaining one or more additional functional assays to confirm the functionality of a candidate peptide moiety that is identified by virtue od being biotinylated in the host cells. Exemplary functional assays comprise linking the cell penetrating peptide to a cargo molecule and assaying for delivery of the cargo to a test cell or a subcellular location within a test cell.

In one example, a cargo is covalently-linked to a candidate peptide moiety. Methods for covalently linking a cargo and a candidate peptide moiety include performing native chemical ligation, click chemistry, thio-amine coupling, carbodiimide conjugation, enzymatic conjugation, sulfosuccinimidylsuberyl linkage, biochemical protein ligation or soluble handling conjugation. Other means for conjugating a cargo to a candidate peptide moiety include methods described generally by Nagahara et al., Nat Med. 4, 1449-1453 (1998); Gait, Cell Mol Life Sci. 60, 844-853 (2003); Moulton and Moulton, Drug Discovery Today. 9, 870-875 (2004); Zatsepin et al., Curr Pharmaceutical Design. 11, 3639-3654 (2005).

Alternatively, a cargo may be non-covalently-linked to a candidate peptide moiety e.g., by virtue of a biotin-streptavidin interaction or electrostatic interaction or metal-affinity interaction e.g., Morris et al., Nucleic Acids Res. 35, e49-e59 (2007).

In one example, the cargo comprises a fluorochrome. Suitable fluorochromes include, for example, TAMRA dyes (e.g. Hsu et al. Clin. Chem. 47, 1373-1377, 2001), BODIPY dyes (e.g. Hecht et al. ChemistryOpen 2, 25-38, 2013), CHROMEO dyes (e.g. Active Motif), DyLight Fluor dyes (e.g. Sarkar et al. J. Photochem. Photobiol. B. 98, 35-39, 2010), sulforhodamine dyes such as for example, Texas Red, Lissamine rhodamine B-sulfonyl chloride, fluorescein and derivatives thereof including for example, fluorescein isothiocyanate (FITC), dichlorotriazinyl aminofluorescein (DTAF), carboxyfluorescein succinimidyl ester (CFSE) (e.g. Liu J. Fluoresc. 19, 915-920, 2009), cyanine dyes such as for example Cy2, Cy3, Cy3.5 Cy5, Cy5.5 (e.g. Kricka Ann. Clin Biochem. 39 114-129, 2002) or Alexa Fluor Dyes (e.g. Panchuck-Voloshina et al. J. Histochem. Cytochem. 47, 1179-1188, 1999).

In another example, the cargo comprises a toxin. Suitable toxins may include, for example, domains from plant, bacterial or fungal protein toxins. As used herein, “plant toxins”, “bacterial toxins” and “fungal toxins” respectively refer any toxin produced by a plant, bacteria or fungus. Such toxins include, for example, toxins classified according to their mechanism of action and/or structural organization, such as, for example, ADP-ribosylating toxins; N-glycosidase containing ribosome inactivating toxins; and binary bacterial toxins that comprise separate cell binding and catalytic domains, including, for example, anthrax toxin, pertussis toxins, cholera toxin, E. coli heat-labile enterotoxin, Shiga toxin, pertussis toxin, Clostridium perfringens iota toxin, Clostridium spiroforme toxin, Clostridium difficile toxin, Clostridium botulinum C2 toxin, and Bacillus cereus vegetative insecticidal protein. Preferably, the toxin may cause cell death or impaired cell survival when internalised in a test cell. In some examples, the toxin conjugate may induce cell death in more than 50% or more 60% or more than 70% or more than 80% or more than 90% or more than 95% or more than 97% or more than 98% or more than 99% of cells in which it is internalized.

Methods to determine cell viability or cytotoxicity are known in the art such as, for example, plate viability assays, colony regression assays, plating assays, and fluorometric/colorimetric growth indicator assays based on detection of metabolic activity. In one example, cell viability is determined based on the ability of the membrane of viable test cells to exclude dyes, such as, for example, tryptan blue or propidium iodide. Living test cells exclude such dyes and do not become stained. In contrast, dead or dying test cells that have lost membrane integrity allow these dyes to enter the cytoplasm and stain various compounds or organelles within the test cell. As will be apparent to the skilled artisan, a number of cell viability assays and cytotoxicity assays are also commercially available.

In another example, the cargo comprises an oligonucleotide such as, for example, an antisense oligonucleotide or an antisense phosphorothioate oligodeoxynucleotides (Kretschmer-Kazemi and Sczakiel Nucleic Acids Res. 31, 4417-4424, 2003) or a phosphorodiamidate morpholino oligonucleotide e.g., Popplewell et al., Methods Mol. Bio. 867, 143-167 (2012), or a short interfering RNA e.g., Juliano et al., J. Drug. Target. 21, 27-43 (2013) or a microRNA e.g., Deleavey and Damha, Chem. Bio 19, 937-954 (2012) or a peptide-nucleic acid (PNA) e.g., Nielsen Curr. Opin. Biotechnol. 10, 71-75 (1999) or a phosphorothioate antisense oligonucleotide e.g., Kole et al. Nat. Rev. Drug Discov. 11, 125-140 or a locked nucleic acid e.g., Koshkin et al. Tetrahedron 54, 3607-3630 (1998).

In yet another example, the cargo comprises a magnetic nanoparticle. Methods for conjugating candidate peptide moieties to magnetic nanoparticle are known in the art and are described, for example, by Lewin et al. Nat Biotechnol. 18, 410-414 (2000).

In a further example, the cargo comprises a quantum dot. Methods for coupling quantum dots and candidate peptide moieties are known in the art and are described, for example, by Liu et al., J. Nanosci. Nanotechnol. 10, 7897-7905 (2010).

In another example, the cargo comprises a particle comprising e.g., a cross-linked polystyrene, a cross-linked N-(2-hydroxypropyl) methacrylamide, a cross-linked dextran, a liposome, or a micelle. In some examples, the particle may serve as a carrier or container for a functional molecule. The functional molecule may be any molecule capable of exerting a function inside cell, e.g., a chemotherapeutic molecule such as doxorubicin (e.g. Rousselle et al., J Pharmacol Exp Ther. 296, 124-131 (2001).

In other examples, the cargo comprises a virus particle e.g., Nigatu et al., J Pharm Sci. 102, 1981-1993 (2013) or a protein e.g., Snyder and Dowdy, Expert Opin. Drug Deliv. 2, 43-51 (2005) or Elliott and O'Hare, Cell 88, 223-233 (1997) or a plasmid e.g., Rittner et al., Mol Ther. 5, 104-114 (2002) or a liposome e.g., Joliot and Prochiantz Nat. Cell Biol. 6, 189-196 (2004).

The present invention is described further in the following non-limiting examples.

### Example 1

### Production of a Candidate Peptide Moiety

This example demonstrates the production of a candidate peptide moiety such as a peptide library e.g., a bacteriophage display library or other peptide display scaffold, using nucleic acid encoding candidate peptides.

A highly diverse mixture of nucleic acids encoding candidate peptides was produced from coding and non-coding regions of bacterial genomes and eukaryotes having compact genomes, essentially as described in U.S. Pat. No. 7,270,969, and subject to the variations in the choice of source genomes as described herein below, and in the vectors employed for expression of peptides encoded by the nucleic acids as described in the following examples. The contents of U.S. Pat. No. 7,270,969 are incorporated herein by reference in their entirety.

Briefly, nucleic acid was isolated from the following bacterial and archaea species:

| | |
|---|---|
| 1 | Acinetobacter baumannii [ATCC_17978; uid58731] |
| 2 | Aeromonas hydrophila [ATCC_7966; uid58617] |
| 3 | Aeropyrum pernix K1 [uid57757] |
| 4 | Archaeglobus fulgidis [DSM_4304; uid57717] |
| 5 | Bacillus cereus [ATCC_10987; uid57673] |
| 6 | Bordetella pertussis strain Tohama I [uid57617] |
| 7 | Borrelia burgdorferi B31 [uid57581] |
| 8 | Campylobacter jejuni subsp. jejuni [NCTC_11168; |
| | ATCC_700819; uid57587] |
| 9 | Clostridium difficile 630 [uid57679] |
| 10 | Clostridium perfringens [ATCC_13124; uid57901] |
| 11 | Corynebacterium diphtheriae [NCTC_13129; uid57691] |
| 12 | Haemophilus influenzae Rd_KW20 [uid57771] |
| 13 | Haloarcula marismortui [ATCC_43049; uid57719] |
| 14 | Halobacterium salinarum R1 [uid61571] |
| 15 | Haloferax volcanii DS2 [uid46845] |
| 16 | Helicobacter pylori 26695 [uid57787] |
| 17 | Legionella pneumophila subsp. pneumophila |
| | Philadelphia_1 [uid57609] |
| 18 | Listeria monocytogenes EGD_e [uid61583] |
| 19 | Methanococcus jannaschii [DSM_2661; uid57713] |
| 20 | Mycobacterium avium subsp. paratuberculosis K_10 [uid57699] |
| 21 | Mycobacterium tuberculosis H37Ra [uid58853] |
| 22 | Neisseria gonorrhoeae FA_1090 [uid57611] |
| 23 | Neisseria meningitidis FAM18 [uid57825] |
| 24 | Porphyromonas gingivalis W83 [uid57641] |
| 26 | Pseudomonas aeruginosa PAO1 [uid57945] |
| 27 | Pyrococcus horikoshii OT3 [uid57753] |
| 28 | Salmonella enterica subsp. enterica serovar |
| | Typhimurium LT2 [uid57799] |
| 29 | Staphylococcus aureus Mu50 [uid57835] |
| 30 | Streptococcus pyogenes M1_GAS [uid57845] |
| 31 | Sulfolobus solfataricus P2 [uid57721] |

Nucleic acid fragments were generated from each of these genomes using multiple consecutive rounds of PCR using tagged random oligonucleotides and mixture of nucleic acid fragments produced from diverse genome sources were digested with the restriction endonuclease MfeI, purified e.g., using a QIAquick PCR purification column (QIAGEN) as per manufacturer's instructions, and retained for ligation into a compatible EcoRI site of a gene construct for subsequent display on a scaffold.

Alternatively, or in addition, the same procedures are employed to produce a scaffold such as a bacteriophage library, using the following bacteria and archaea:

| | |
|---|---|
| 1 | Acinetobacter baumannii [ATCC_17978; uid58731] |
| 2 | Aeromonas hydrophila [ATCC_7966; uid58617] |
| 3 | Aeropyrum pernix K1 [uid57757] |
| 4 | Archaeglobus fulgidis DSM 4304 [uid57717] |
| 5 | Bacillus cereus [ATCC_10987; uid57673] |
| 6 | Bacillus subtilis 168 [uid57675] |
| 7 | Bacteroides thetaiotaomicron VPI_5482 [uid62913] |
| 8 | Bordetella pertussis Tohama_I [uid57617] |
| 9 | Borrelia burgdorferi B31 [uid57581] |
| 10 | Campylobacter jejuni subsp. jejuni [NCTC_11168; |
| | ATCC_700819; uid57587] |
| 11 | Caulobacter vibrioides [C. crescentus CB15; uid57891] |
| 12 | Chlorobium tepidum TLS [uid57897] |
| 13 | Clostridium acetobutylicum [ATCC_824; uid57677] |
| 14 | Clostridium difficile 630 [uid57679] |
| 15 | Clostridium perfringens [ATCC_13124; uid57901] |
| 16 | Corynebacterium diphtheriae [NCTC_13129; uid57691] |
| 17 | Cryptosporidium parvum Iowa, chromosomes 1-8 |
| 18 | Deinococcus radiodurans R1 [uid57665] |
| 19 | Desulfovibrio vulgaris Hildenborough [uid57645] |
| 20 | Escherichia coli K_12_substr_{——}MG1655 [uid57779] |
| 21 | Geobacter sulfureducens PCA [uid57743] |
| 22 | Haemophilus influenzae Rd_KW20 [uid57771] |
| 23 | Haloarcula marismortui [ATCC_43049; uid57719] |
| 24 | Halocobacterium NRC I [uid57769] |
| 25 | Halobacterium salinarum R1 [uid 61571] |
| 26 | Haloferax volcanii DS2 [uid46845] |
| 27 | Helicobacter pylori 26695 [uid57787] |
| 28 | Legionella pneumophila subsp. pneumophila |
| | Philadelphia_I [uid57609] |
| 29 | Listeria monocytogenes EGD_e [uid61583] |
| 30 | Listeria innocua Clip11262 [uid61567] |
| 31 | Methanococcus jannaschii DSM_2661 [uid57713] |
| 32 | Mycobacterium avium subsp. paratuberculosis K10 |
| | [uid57699] |
| 33 | Mycobacterium tuberculosis H37Ra [uid58853] |
| 34 | Neisseria gonorrhoeae FA1090 [uid57611] |
| 35 | Neisseria meningitidis FAM18 [uid57825] |
| 36 | Porphyromonas gingivalis W83 [uid57641] |
| 37 | Pseudomonas aeruginosa PAO1 [uid57945] |
| 38 | Pyrococcus horikoshii OT3 [uid57753] |
| 39 | Rhodobacter sphaeroides 2_4_1 [uid57653] |
| 40 | Rhodopseudomonas palustris CGA009 [uid62901] |
| 41 | Salmonella enterica subsp. enterica serovar |
| | Typhimurium LT2 [uid57799] |
| 42 | Shigella flexneri 2a_2457T [uid57991] |
| 43 | Staphylococcus aureus Mu50 [uid57835] |
| 44 | Streptococcus pyogenes M1_GAS [uid57845] |
| 45 | Streptomyces avermitilis MA_4680 [uid57739] |
| 46 | Sulfolobus solfataricus P2 [uid57721] |
| 47 | Thermoplasma volcanicum GSS1 [uid57751] |
| 48 | Thermotoga maritima MSB8 [uid57723] |

Alternatively, or in addition to the foregoing genome sources, a library of candidate peptides is produced by expressing amplified nucleic acid fragments derived from at least about 20 of the following genomes on a bacteriophage scaffold in according with the teaching provided in U.S. Pat. No. 7,270,969:

a) fragments derived from bacterial species selected from Pseudomonas aeruginosa, Clostridium difficile, Acinetobacter baumannii, Aeromonas hydrophila, Bacillus cereus, Bacillus subtilis, Bacteroides thetaiotaomicron, Bordetella pertussis, Borrelia burgdorferi, Campylobacter jejuni subsp. Jejuni, Caulobacter vibrioides (crescentus), Chlorobium tepidum, Clostridium acetobutylicum, Clostridium difficile, Clostridium perfringens, Corynebacterium diphtheria, Deinococcus radiodurans, Desulfovibrio vulgaris, Geobacter sulfurreducens, Haemophilus influenza, Helicobacter pylori, Legionella pneumophila subsp. Pneumophila, Listeria innocua, Listeria monocytogenes, Mycobacterium avium subsp. paratuberculosis, Mycobacterium tuberculosis, Neisseria gonorrhoeae, Neisseria menigitidis, Porphyromonas gingivalis, Rhodobacter sphaeroides, Rhodopseudomonas palustris, Salmonella enterica subsp. enterica serovar Thyphimurium, Streptomyces avermitilis, Staphylococcus aureus, Streptococcus pyogenes and Thermotoga maritime; and
b) fragments derived from archael species selected from Haloarcula marismortui, Haloferax volcanii, Sulfolobus solfataricus, Halobacterium salinarum, Archeaoglobus fulgidis, Pyrococcus horikoshii, Methanococcus jannaschii, Aeropyrum pernix and Thermoplasma volcanicum; and
c) fragments derived from viruses selected from Human herpes virus 5 (CMV) (strain AD-169), Vaccinia virus, Human herpes virus 1 (HSV-1) (strain KOS), Human herpes virus 3 (Varicella-zoster virus) (strain Ellen), Human adenovirus C serotype 1 (HAdV-1) (strain adenoid 71), Human adenovirus B, subspecies B2, serotype 14 (HAdV-14), Coronavirus (strain 229E), Parainfluenza virus 4b, Measles virus (Ichinose-B95a), Parainfluenza virus 2, Parainfluenza virus 1 strain C35), Parainfluenza virus 3, Mumps (strain Enders), Human respiratory syncytial virus B (strain B1), Rhinovirus B17 (common cold), Human papillomavirus type 16, Human papillomavirus type 18, Human papillomavirus type 6b, Hepatitis B virus (clone AM6), Influenza A virus (H1N1), Human adenovirus C serotype 2 (HAdV-2), Dengue type 1 virus, Human herpesvirus 4(Ebstein-Barr virus), Human herpes virus 8 (Karposis sarcoma virus), Zaire ebola virus, Lake Victoria marburgvirus, Newcastle disease virus, Human respiratory syncytial virus B, Vesicular stomatitis Indiana virus, Influenza C virus, Adeno-associated virus 2, Foot-and-mouth virus, Hepatitis A virus, Human parechovirus 1 (echovirus 22), Simian Virus 40, Rotavirus A, Reovirus type 1, Avian leukosis virus RSA (RSV-SRA)/Rous sarcoma virus, Human immunodeficiency virus 1 and Sindbis virus.

### Example 2

### Production of a Non-Biotinylated Member Using Expression Vector pNp3

This example demonstrates the production of a non-biotinylated member employing expression vector pNp3 or derivative thereof to produce a filamentous bacteriophage displaying the non-biotinylated member.

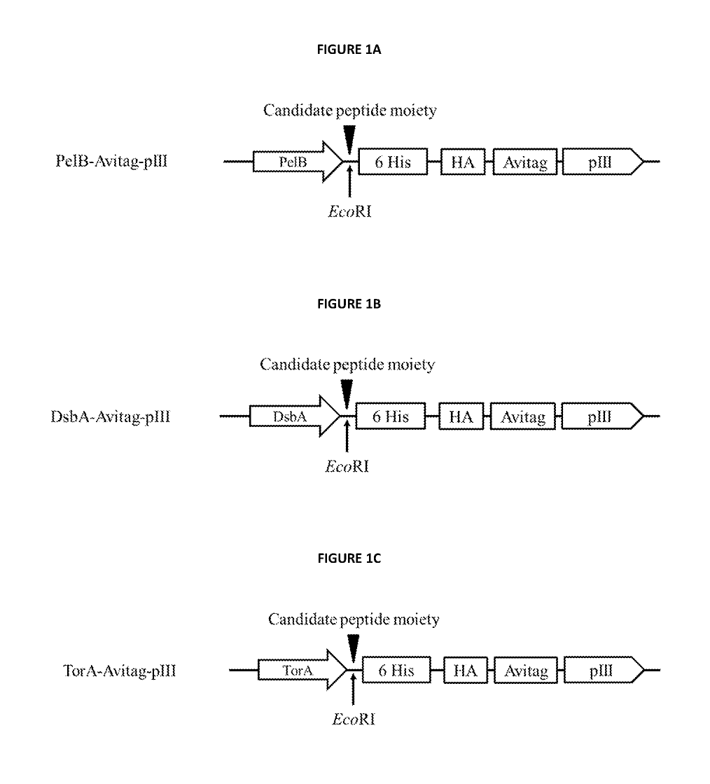

Vector construct designated, pNp3 is an M13 vector comprising nucleic acid encoding a fusion protein comprising a hexahistidine (6 His) tag, hemagglutinin (HA) tag, a biotin ligase substrate domain and M13 pIII coat protein. The vector pNp3 was modified to express fusion proteins comprising candidate peptide moieties fused in-frame to the 15-amino acid biotin ligase substrate domain having the amino acid sequence set forth in SEQ ID NO: 4, as shown in FIGS. 1a, 1b and 1c. Fusion proteins produced using pNp3 are subsequent displayed on a scaffold comprising the filamentous bacteriophage M13.

FIG. 1a shows the encoded pIII fusion protein of the pNp3 derivative vector PelB-Avitag-pIII, which comprises the following components in-frame:

1. Erwinia carotovora CE Pectate lyase B (PelB) leader peptide or signal peptide (SEQ ID NO: 31) for targeting the expressed fusion protein to the bacterial periplasm and cell surface for the purpose of phage display.
2. Hexahistidine tag (6 His; SEQ ID NO: 33) for detection and/or purification of the fusion protein.
3. Hemagglutinin tag (HA; SEQ ID NO: 39) for detection and/or purification of the fusion protein.
4. A biotin ligase substrate domain comprising an Avitag sequence set forth in SEQ ID NO: 4.

In one example, nucleic acid encoding a candidate peptide moiety produced as described in Example 1 is introduced to the EcoRI site of the vector construct (SEQ ID NO: 50) positioned between PelB leader peptide and hexahistidine tag-encoding sequence.

In another example, the expression construct designated pNp3 was modified further to produce vector DsbA-Avitag-pIII, comprising nucleic acid encoding a signal peptide of the DsbA protein (SEQ ID NO: 20) e.g., Steiner et al., Nat. Biotechnol. 24, 823-831 (2006). Then, nucleic acid encoding a candidate peptide moiety produced as described in Example 1 is introduced to the EcoRI site of the vector construct (SEQ ID NO: 44), as shown in FIG. 1b.

In another example, the expression construct designated pNp3 was modified further to produce vector TorA-Avitag-pIII, comprising nucleic acid encoding a signal peptide of the TorA protein (SEQ ID NO: 29) e.g., Buchanan et al., FEBS. 582, 3979-3984 (2003). Then, nucleic acid encoding a candidate peptide moiety produced as described in Example 1 is introduced to the EcoRI site of the vector construct (SEQ ID NO: 47), as shown in FIG. 1c.

In another example, the expression construct designated pNp3 is modified so as to generate a unique EcoRI site positioned between nucleic acid encoding the hexahistidine tag-encoding sequence and nucleic acid encoding the hemagglutinin tag. Nucleic acid encoding a candidate peptide moiety as described in Example 1 is introduced to the EcoRI site of the modified vector.

In another example, the expression construct designated pNp3 is modified so as to generate a unique EcoRI site positioned between nucleic acid encoding the nucleic acid encoding the hemagglutinin tag and nucleic acid encoding the Avitag. Nucleic acid encoding a candidate peptide moiety as described in Example 1 is introduced EcoRI site of the modified vector.

In another example, the expression construct designated pNp3 is modified so as to generate a unique EcoRI site positioned between nucleic acid encoding the Avitag domain and nucleic acid encoding the pIII coat protein. Nucleic acid encoding a candidate peptide moiety as described in Example 1 is introduced to the EcoRI site of the modified vector.

Standard site-directed mutagenesis in performed to introduce the unique EcoRI site into any region of the pNp3 expression vector.

In an alternative example, the expression construct designated pNp3 or derivative thereof as described in any example hereof is modified further to replace nucleic acid encoding the hexahistidine tag (6 His) with nucleic acid encoding a dodecahistidine tag (10 His; SEQ ID NO: 35) for detection and/or purification of the fusion protein. Alternatively, these vectors are modified by introducing nucleic acid encoding up to four (4) additional histidine residues to produce corresponding vectors encoding ten (10) histidine residues in tandem. Standard procedures are employed on such modifications.

In another examples, the positions of candidate peptide and the Avitag domain in the vector are modified with respect to each other and other domains positioned upstream of the coat protein. For example, the Avitag domain is positioned adjacent the C-terminus of the candidate peptide moiety and N-terminal of the 6 His or 10 His domain or the HA domain. The relative positions of the tag domains in these vectors is variable and not essential to their performance Standard procedures are employed on such modifications.

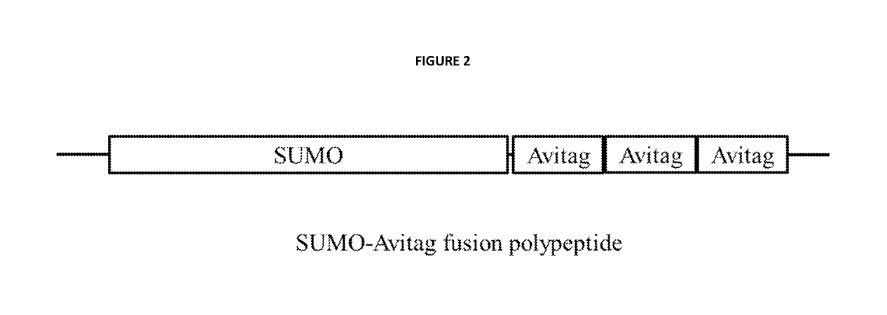

In yet another example, a non-biotinylated member is produced by expressing the pNp3 expression vector or derivative vector thereof as described according to any embodiment hereof in E. coli cells. In such an example, the bacterial cells are transformed so as to express a SUMO-(Avitag)₃ fusion decoy polypeptide (FIG. 2) comprising three tandem copies of a biotin ligase substrate domain comprising an Avitag domain (SEQ ID NO: 4) fused to a Small Ubiquitin-like Modifier (SUMO) protein e.g., Hay et al., Mol. Cell 18, 1-12 (2005). In this example, the expressed tandem copies of the biotin ligase substrate domain are biotinylated in preference of the biotin ligase substrate domain of the pNp3 vector derivative e.g., by virtue of the endogenous biotin ligase enzyme being exposed to a molar excess of substrate via expression of the bacterial cells having a higher affinity for the tandem copies of the Avitag domain, as opposed to than for a single copy of the Avitag domain present on the pNp3 vector derivative which is stochastic terms is less able to compete for biotinylation activity.

Western blot analysis was performed for the detection of in vitro biotinylated proteins. Briefly, samples were diluted in Laemmli buffer and boiled for 5 minutes. Denatured samples were resolved on a 4-12% Bis-tris gel and blotted onto PVDF membrane (Life Technologies, Invitrogen) by using standard procedures. Membranes were blocked in 5% skim milk/PBS at 4° C. overnight. Membranes were rinsed in 1×PBS with 0.05% Tween-20 (PBS-T) and incubated at room temperature for 1 hour with anti-biotin streptavidin conjugated to horseradish peroxidase (SA-HRP) (dilution 1:1,000). Membranes were washed in PBS-T and developed by using a Western C kit (Bio-Rad).

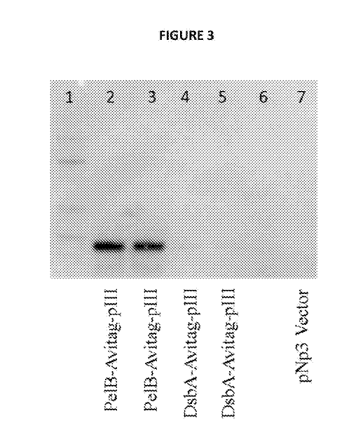

As shown in FIG. 3, fusion proteins comprising the DsbA signal peptide are not biotinylated in E. coli cells that do not express the SUMO-(Avitag)₃ fusion decoy polypeptide shown in FIG. 2 hereof, whereas vectors expressing fusion proteins comprising the PelB signal peptide are biotinylated in such cells. See e.g., FIG. 3, lanes 2-5 and 7. This supports the conclusion that non-biotinylated members are displayed on M13 expressing a fusion protein that comprises the DsbA signal peptide.

To produce a non-biotinylated member from the PelB-Avitag-pIII vector, M13 assembled using the vector is produced using E. coli cells expressing the SUMO-(Avitag)₃ fusion decoy polypeptide shown in FIG. 2 hereof.

To produce a non-biotinylated member from the TorA-Avitag-pIII vector, M13 assembled using the vector is produced using E. coli cells expressing the SUMO-(Avitag)₃ fusion decoy polypeptide shown in FIG. 2 hereof.

### Example 3

### Production of a Non-Biotinylated Member Using Expression Vector pNp8

This example demonstrates the production of a non-biotinylated member employing expression vector pNp8 or derivative thereof to produce a filamentous bacteriophage displaying the non-biotinylated member.

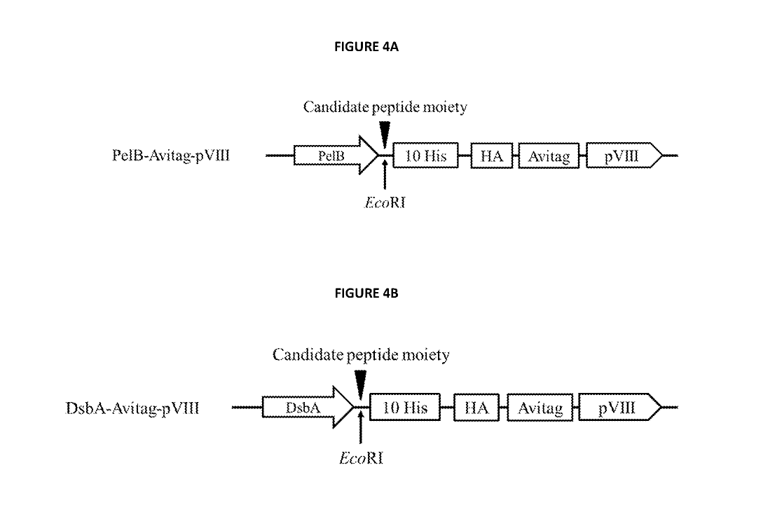

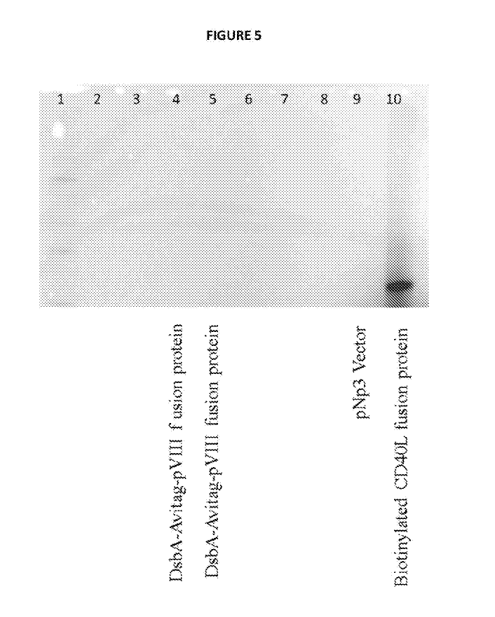

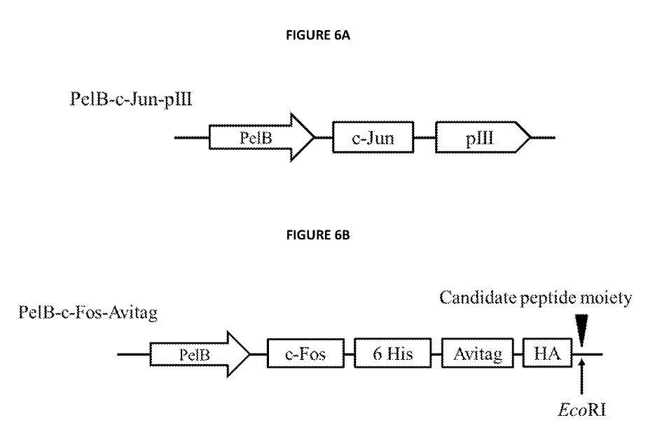

Vector construct designated, pNp8 is an M13 vector comprising nucleic acid encoding a fusion protein comprising a hexahistidine (10 His) tag, hemagglutinin (HA) tag, a biotin ligase substrate domain and M13 pVIII coat protein. The vector pNp8 was modified to express fusion proteins comprising candidate peptide moieties fused in-frame to the 15-amino acid biotin ligase substrate domain having the amino acid sequence set forth in SEQ ID NO: 4, as shown in FIGS. 4a, and 4b. Fusion proteins produced using pNp8 are subsequent displayed on a scaffold comprising the filamentous bacteriophage M13.

FIG. 4a shows the encoded pVIII fusion protein of the pNp8 derivative vector PelB-Avitag-pVIII, which comprises the following components in-frame:

1. Erwinia carotovora CE Pectate lyase B (PelB) leader peptide or signal peptide (SEQ ID NO: 31) for targeting the expressed fusion protein to the bacterial periplasm and cell surface for the purpose of phage display.
2. Dodecahistidine tag (10 His; SEQ ID NO: 35) for detection and/or purification of the fusion protein.
3. Hemagglutinin tag (HA; SEQ ID NO: 39) for detection and/or purification of the fusion protein.
4. A biotin ligase substrate domain comprising an Avitag sequence set forth in SEQ ID NO: 4.

In one example, nucleic acid encoding a candidate peptide moiety produced as described in Example 1 is introduced to the EcoRI site of the vector construct (SEQ ID NO: 56) positioned between PelB leader peptide and hexahistidine tag-encoding sequence.

In another example, the expression construct designated pNp8 was modified further to produce vector DsbA-Avitag-pVIII, comprising nucleic acid encoding a signal peptide of the DsbA protein (SEQ ID NO: 20) e.g., Steiner et al., Nat. Biotechnol. 24, 823-831 (2006). Then, nucleic acid encoding a candidate peptide moiety produced as described in Example 1 is introduced to the EcoRI site of the vector construct (SEQ ID NO: 44), as shown in FIG. 4b.

In another example, the expression construct designated pNp8 is modified so as to generate a unique EcoRI site positioned between nucleic acid encoding the dodecahistidine tag-encoding sequence and nucleic acid encoding the hemagglutinin tag. Nucleic acid encoding a candidate peptide moiety as described in Example 1 is introduced to the EcoRI site of the modified vector.

In another example, the expression construct designated pNp8 is modified so as to generate a unique EcoRI site positioned between nucleic acid encoding the nucleic acid encoding the hemagglutinin tag and nucleic acid encoding the Avitag domain. Nucleic acid encoding a candidate peptide moiety as described in Example 1 is introduced EcoRI site of the modified vector.

In another example, the expression construct designated pNp8 is modified so as to generate a unique EcoRI site positioned between nucleic acid encoding the Avitag domain and nucleic acid encoding the pVIII coat protein. Nucleic acid encoding a candidate peptide moiety as described in Example 1 is introduced to the EcoRI site of the modified vector.

Standard site-directed mutagenesis in performed to introduce the unique EcoRI site into any region of the pNp8 expression vector.

In an alternative example, the expression construct designated pNp8 or derivative thereof as described in any example hereof is modified further to replace nucleic acid encoding the dodecahexahistidine tag (10 His) domain with nucleic acid encoding a hexahistidine tag (6 His; SEQ ID NO: 33) for detection and/or purification of the fusion protein. Alternatively, these vectors are modified by removing nucleic acid encoding up to four (4) additional histidine residues to produce corresponding vectors encoding six (6) histidine residues in tandem. Standard procedures are employed on such modifications.

In another examples, the positions of candidate peptide and the Avitag domain in the vector are modified with respect to each other and other domains positioned upstream of the coat protein. For example, the Avitag domain is positioned adjacent the C-terminus of the candidate peptide moiety and N-terminal of the 6 His or 10 His domain or the HA domain. The relative positions of the various tag domains in these vectors is variable and not essential to their performance. Standard procedures are employed on such modifications.

In one example, a non-biotinylated member is produced by expressing a pNp8 derivative vector as described according to any embodiment hereof in E. coli cells. In such an example, the bacterial cells are transformed so as to express a SUMO-(Avitag)₃ fusion decoy polypeptide (FIG. 2) comprising three tandem copies of a biotin ligase substrate domain comprising an Avitag domain (SEQ ID NO: 4) fused to a Small Ubiquitin-like Modifier (SUMO) protein e.g., Hay et al., Mol. Cell 18, 1-12 (2005). In this example, the expressed tandem copies of the biotin ligase substrate domain are biotinylated in preference of the biotin ligase substrate domain of the pNp8 vector derivative e.g., by virtue of the endogenous biotin ligase enzyme being exposed to bacterial cells having a molar excess of substrate via expression of higher affinity for the tandem copies of the Avitag domain, as opposed to than for a single copy of the Avitag domain present on the pNp8 vector derivative which is stochastic terms is less able to compete for biotinylation activity.

Western blot analysis was performed for the detection of in vitro biotinylated proteins. Briefly, samples were diluted in Laemmli buffer and boiled for 5 minutes. Denatured samples were resolved on a 4-12% Bis-tris gel and blotted onto PVDF membrane (Life Technologies, Invitrogen) by using standard procedures. Membranes were blocked in 5% skim milk/PBS at 4° C. overnight. Membranes were rinsed in 1×PBS with 0.05% Tween-20 (PBS-T) and incubated at room temperature for 1 hour with anti-biotin streptavidin conjugated to horseradish peroxidase (SA-HRP) (dilution 1:1,000). Membranes were washed in PBS-T and developed by using a Western C kit (Bio-Rad).

As shown in FIG. 5, fusion proteins comprising the DsbA signal peptide are not biotinylated in E. coli cells that do not express the SUMO-(Avitag)₃ fusion decoy polypeptide shown in FIG. 2 hereof. See e.g., FIG. 3, lanes 4 and 5. This supports the conclusion that non-biotinylated members are displayed on M13 expressing a fusion protein that comprises the DsbA signal peptide.

To produce a non-biotinylated member from the PelB-Avitag-pVIII vector, M13 assembled using the vector is produced using E. coli cells expressing the SUMO-(Avitag)₃ fusion decoy polypeptide shown in FIG. 2 hereof.

### Example 4

### Production of a Non-Biotinylated Member Using Expression Vector pJuFo-pIII or Expression Vector pJuFo-pVIII

This example demonstrates the production of a non-biotinylated member employing expression vector pJuFo-pIII, pJuFo-pVIII or derivative thereof to produce a filamentous bacteriophage displaying the non-biotinylated member.

Vector constructs designated pJuFo-pIII encodes a first fusion protein comprising a PelB leader peptide, a C-terminal leucine zipper domain of c-Jun and a M13 capsid protein, pIII (FIG. 6a) (SEQ ID NO: 60) and a second fusion protein comprising the PelB leader peptide, a C-terminal leucine zipper domain of c-Fos, a hexahistidine (6 His) tag, a biotin ligase substrate domain (Avitag domain) and a hemagglutinin (HA) tag (FIG. 6b) (SEQ ID NO:61).

M13 phage comprising pJuFo-pIII display the PelB-cJun-pIII fusion protein, and express the PelB-cFos-Avitag fusion protein in trans in E. coli. Dimerization of the leucine zipper domain of c-Jun and c-Fos produce a heterodimetric fusion protein comprising the Avitag domain. The vector pJuFo-pIII comprises an EcoRI site positioned 3′ of the nucleic acid encoding the PelB-cFos-Avitag domain fusion e.g. 3′ of nucleic acid encoding the HA tag of the PelB-cFos-Avitag fusion protein, to provide for insertion of nucleic acid encoding the candidate peptide moiety produced as described in Example 1. This insertion results in the candidate peptide being expressing as an in-frame fusion with the PelB-cFos-Avitag fusion protein. The nucleotide sequence of pJuFo-pIII is set for in SEQ ID NO: 59.

In one example, nucleic acid encoding a candidate peptide moiety as described in Example 1 is introduced to the EcoRI site of the vector construct (SEQ ID NO: 59) as shown in FIG. 6b.

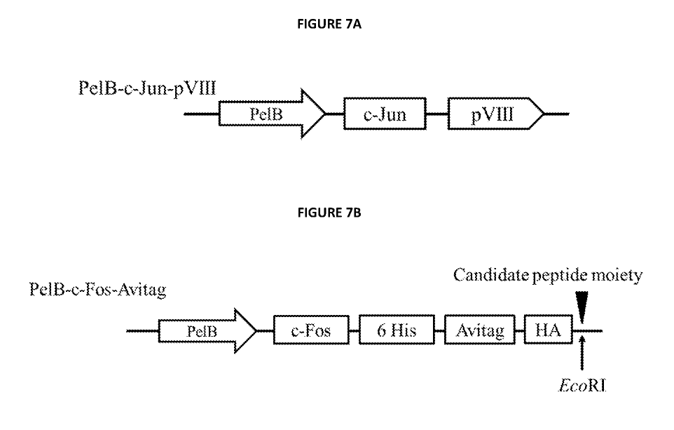

Vector constructs designated pJuFo-pVIII encodes a first fusion protein comprising a PelB leader peptide, a C-terminal leucine zipper domain of c-Jun and a M13 capsid protein, pVIII (FIG. 7a) (SEQ ID NO: 63) and a second fusion protein comprising the PelB leader peptide, a C-terminal leucine zipper domain of c-Fos, a hexahistidine (6 His) tag, a biotin ligase substrate domain (Avitag domain) and a hemagglutinin (HA) tag (FIG. 7b) (SEQ ID NO:64).

M13 phage comprising pJuFo-pVIII display the PelB-cJun-pVIII fusion protein, and express the PelB-cFos-Avitag fusion protein in trans in E. coli. Dimerization of the leucine zipper domain of c-Jun and c-Fos produce a heterodimeric fusion protein comprising the Avitag domain. The vector pJuFo-pVIII comprises an EcoRI site positioned 3′ of the nucleic acid encoding the PelB-cFos-Avitag domain fusion e.g. 3′ of nucleic acid encoding the HA tag of the PelB-cFos-Avitag fusion protein, to provide for insertion of nucleic acid encoding the candidate peptide moiety produced as described in Example 1. This insertion results in the candidate peptide being expressing as an in-frame fusion with the PelB-cFos-Avitag fusion protein. The nucleotide sequence of pJuFo-pVIII is set for in SEQ ID NO: 62.

In one example, nucleic acid encoding a candidate peptide moiety as described in Example 1 is introduced to the EcoRI site of the vector construct (SEQ ID NO: 62) as shown in FIG. 7b.

In another example, the expression vector designated pJuFo-pIII or pJuFo-pVIII is modified further to replace the nucleic acid encoding the hexahistidine tag (6 His) domain with nucleic acid encoding a dodecahistidine tag (10 His; SEQ ID NO: 35) for detection and/or purification of the fusion protein. Alternatively, these vectors are modified by introducing nucleic acid encoding up to four (4) additional histidine residues to produce corresponding vectors encoding ten (10) histidine residues in tandem. Standard procedures are employed on such modifications.

In another example, the expression construct designated pJuFo-pIII, pJuFo-pVIII are modified further to replace the nucleic acid encoding the PelB signal peptide with nucleic acid encoding a signal peptide of the DsbA protein (SEQ ID NO: 20) e.g., Steiner et al., Nat. Biotechnol. 24, 823-831 (2006). Standard procedures are employed on such modifications.

In yet another example, the positions of candidate peptide and the Avitag domain in the vector is modified with respect to each other and other domains. For example, the Avitag domain is positioned adjacent the C-terminus of the candidate peptide moiety and N-terminal of the 6 His or 10 His domain or the HA domain. The relative positions of the various tag domains in these vectors is variable and not essential to their performance. Standard procedures are employed on such modifications.

In one example, a non-biotinylated member is produced by expressing pJuFo-pIII, pJuFo-pVIII or derivative thereof as described according to any embodiment hereof in E. coli cells. In such an example, the bacterial cells are transformed so as to express a SUMO-(Avitag)₃ fusion decoy polypeptide (FIG. 2) comprising three tandem copies of a biotin ligase substrate domain comprising an Avitag domain (SEQ ID NO: 4) fused to a Small Ubiquitin-like Modifier (SUMO) protein e.g., Hay et al., Mol. Cell 18, 1-12 (2005). In this example, the expressed tandem copies of the biotin ligase substrate domain are biotinylated in preference of the biotin ligase substrate domain of the PelB-cFos-Avitag fusion protein e.g., by virtue of the endogenous biotin ligase enzyme of the bacterial cells having a higher affinity for the tandem copies of the Avitag domain than for a single copy of the Avitag domain present on pJuFo-pIII, pJuFo-pVIII or derivative thereof.

To produce a non-biotinylated member from a derivative pJuFo-pIII, pJuFo-pVIII expression vector comprising the signal peptide of the DsbA protein, M13 is assembled in E. coli cells that do not express the SUMO-(Avitag)₃ fusion decoy polypeptide shown in FIG. 2 hereof.

### Example 5

### Production of a Non-Biotinylated Member Using Expression Vector pJuFo-pIII or Expression Vector pJuFo-pVIII

This example demonstrates the production of a non-biotinylated member employing expression vector pJuFo-pIII, pJuFo-pVIII or derivative thereof to produce a filamentous bacteriophage displaying the non-biotinylated member.

Vector constructs designated pJuFo-pIII encodes a first fusion protein comprising a PelB leader peptide, a C-terminal leucine zipper domain of c-Jun and a M13 capsid protein, pIII (FIG. 6a) (SEQ ID NO: 60) and a second fusion protein comprising the PelB leader peptide, a C-terminal leucine zipper domain of c-Fos, a hexahistidine (6 His) tag, a biotin ligase substrate domain (Avitag domain) and a hemagglutinin (HA) tag (FIG. 6b) (SEQ ID NO:61).

M13 phage comprising pJuFo-pIII display the PelB-cJun-pIII fusion protein, and express the PelB-cFos-Avitag fusion protein in trans in E. coli. Dimerization of the leucine zipper domain of c-Jun and c-Fos produce a heterodimetric fusion protein comprising the Avitag domain. The vector pJuFo-pIII comprises an EcoRI site positioned 3′ of the nucleic acid encoding the PelB-cFos-Avitag domain fusion e.g. 3′ of nucleic acid encoding the HA tag of the PelB-cFos-Avitag fusion protein, to provide for insertion of nucleic acid encoding the candidate peptide moiety produced as described in Example 1. This insertion results in the candidate peptide being expressing as an in-frame fusion with the PelB-cFos-Avitag fusion protein. The nucleotide sequence of pJuFo-pIII is set for in SEQ ID NO: 59.

In one example, nucleic acid encoding a candidate peptide moiety as described in Example 1 is introduced to the EcoRI site of the vector construct (SEQ ID NO: 59) as shown in FIG. 6b.

Vector constructs designated pJuFo-pVIII encodes a first fusion protein comprising a PelB leader peptide, a C-terminal leucine zipper domain of c-Jun and a M13 capsid protein, pVIII (FIG. 7a) (SEQ ID NO: 63) and a second fusion protein comprising the PelB leader peptide, a C-terminal leucine zipper domain of c-Fos, a hexahistidine (6 His) tag, a biotin ligase substrate domain (Avitag domain) and a hemagglutinin (HA) tag (FIG. 7b) (SEQ ID NO:64).

M13 phage comprising pJuFo-pVIII display the PelB-cJun-pVIII fusion protein, and express the PelB-cFos-Avitag fusion protein in trans in E. coli. Dimerization of the leucine zipper domain of c-Jun and c-Fos produce a heterodimetric fusion protein comprising the Avitag domain. The vector pJuFo-pVIII comprises an EcoRI site positioned 3′ of the nucleic acid encoding the PelB-cFos-Avitag domain fusion e.g. 3′ of nucleic acid encoding the HA tag of the PelB-cFos-Avitag fusion protein, to provide for insertion of nucleic acid encoding the candidate peptide moiety produced as described in Example 1. This insertion results in the candidate peptide being expressing as an in-frame fusion with the PelB-cFos-Avitag fusion protein. The nucleotide sequence of pJuFo-pVIII is set for in SEQ ID NO: 62.

In one example, nucleic acid encoding a candidate peptide moiety as described in Example 1 is introduced to the EcoRI site of the vector construct (SEQ ID NO: 62) as shown in FIG. 7b.

In another example, the expression vector designated pJuFo-pIII or pJuFo-pVIII is modified further to replace the nucleic acid encoding the hexahistidine tag (6 His) domain with nucleic acid encoding a dodecahistidine tag (10 His; SEQ ID NO: 35) for detection and/or purification of the fusion protein. Alternatively, these vectors are modified by introducing nucleic acid encoding up to four (4) additional histidine residues to produce corresponding vectors encoding ten (10) histidine residues in tandem. Standard procedures are employed on such modifications.

In another example, the expression construct designated pJuFo-pIII, pJuFo-pVIII are modified further to replace the nucleic acid encoding the PelB signal peptide with nucleic acid encoding a signal peptide of the DsbA protein (SEQ ID NO: 20) e.g., Steiner et al., Nat. Biotechnol. 24, 823-831 (2006). Standard procedures are employed on such modifications.

In yet another example, the positions of candidate peptide and the Avitag domain in the vector is modified with respect to each other and other domains. For example, the Avitag domain is positioned adjacent the C-terminus of the candidate peptide moiety and N-terminal of the 6 His or 10 His domain or the HA domain. The relative positions of the various tag domains in these vectors is variable and not essential to their performance. Standard procedures are employed on such modifications.

In one example, a non-biotinylated member is produced by expressing pJuFo-pIII, pJuFo-pVIII or derivative thereof as described according to any embodiment hereof in E. coli cells. In such an example, the bacterial cells are transformed so as to express a SUMO-(Avitag)₃ fusion decoy polypeptide (FIG. 2) comprising three tandem copies of a biotin ligase substrate domain comprising an Avitag domain (SEQ ID NO: 4) fused to a Small Ubiquitin-like Modifier (SUMO) protein e.g., Hay et al., Mol. Cell 18, 1-12 (2005). In this example, the expressed tandem copies of the biotin ligase substrate domain are biotinylated in preference of the biotin ligase substrate domain of the PelB-cFos-Avitag fusion protein e.g., by virtue of the endogenous biotin ligase enzyme of the bacterial cells having a higher affinity for the tandem copies of the Avitag domain than for a single copy of the Avitag domain present on pJuFo-pIII, pJuFo-pVIII or derivative thereof.

To produce a non-biotinylated member from a derivative pJuFo-pIII, pJuFo-pVIII expression vector comprising the signal peptide of the DsbA protein, M13 is assembled in E. coli cells that do not express the SUMO-(Avitag)₃ fusion decoy polypeptide shown in FIG. 2 hereof.

### Example 5

### Production of a Non-Biotinylated Member Using Expression Vector T7Select

This example demonstrates the production of a non-biotinylated member employing expression vector T7Select-Avitag-N, T7Select*-Avitag-N or derivative thereof to produce a T-bacteriophage displaying the non-biotinylated member.

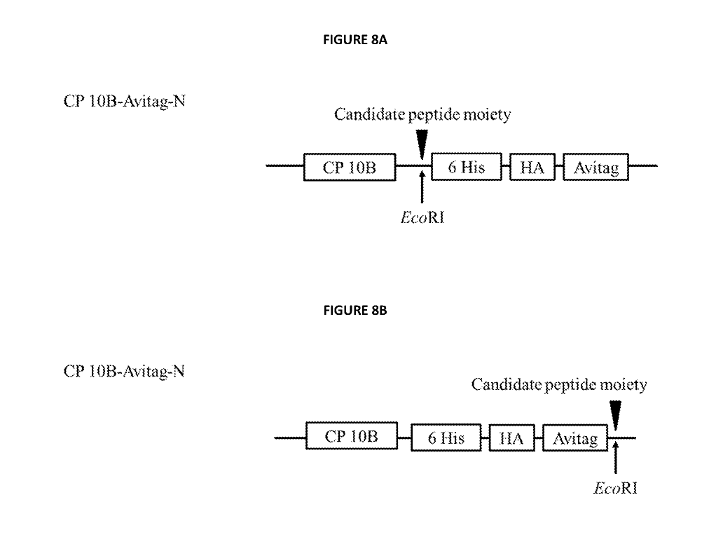

Vector construct designated T7Select-Avitag-N was generated for mid-copy number display of fusion proteins using T7Select 10-3b (Novagen) (SEQ ID NO: 81) as a template. The T7Select-Avitag-N vector encodes a fusion protein comprising a hexahistidine (6 His) tag (SEQ ID NO: 33), a hemagglutinin (HA) tag (SEQ ID NO: 39), a biotin ligase substrate domain (Avitag domain) (SEQ ID NO: 4) and a 10B capsid protein (CP 10B) (FIG. 8a). The vector T7Select-Avitag-N comprises an EcoRI site positioned 5′ of the nucleic acid encoding the Avitag domain to provide for insertion of nucleic acid encoding the candidate peptide moiety produced as described in Example 1. The nucleotide sequence of T7Select-Avitag-N is set for in SEQ ID NO: 65.

In another example, the expression construct designated T7Select-Avitag-N was modified so as to generate a unique EcoRI site positioned downstream of the Avitag domain (T7Select-Avitag-C) (FIG. 8b). The nucleotide sequence of T7Select-Avitag-N is set for in SEQ ID NO: 65. Nucleic acid encoding a candidate peptide moiety as described in Example 1 is introduced to the EcoRI site of the modified vector construct.

Vector construct designated T7Select*-Avitag-N was generated for low-copy display of fusion proteins using T7Select 1-1b (Novagen) (SEQ ID NO: 82) as a template. The T7Select*-Avitag-N vector encodes a fusion protein comprising a hexahistidine tag (6 His; SEQ ID NO: 33), a hemagglutinin tag (HA; SEQ ID NO: 39), a biotin ligase substrate domain (Avitag domain; SEQ ID NO: 4) and a 10B capsid protein (CP 10B) (FIG. 8a). The vector T7Select-Avitag-N comprises an EcoRI site positioned 5′ of the nucleic acid encoding the Avitag domain to provide for insertion of nucleic acid encoding the candidate peptide moiety produced as described in Example 1. The nucleotide sequence of T7Select*-Avitag-N is set for in SEQ ID NO: 67.

In another example, the expression construct designated T7Select*-Avitag-N is modified so as to generate a unique EcoRI site positioned downstream of the Avitag domain (T7Select-Avitag-C). Nucleic acid encoding a candidate peptide moiety as described in Example 1 is introduced to the EcoRI site of the modified vector construct.

In another example, constructs designated T7Select-Avitag-N or T7Select*-Avitag-N, or derivative thereof as described in any example hereof is modified so as to generate a unique EcoRI site positioned between nucleic acid encoding the hexahistidine tag and nucleic acid encoding the hemagglutinin tag. Nucleic acid encoding a candidate peptide moiety as described in Example 1 is introduced to the EcoRI site of the modified vector.

In another example, constructs designated T7Select-Avitag-N or T7Select*-Avitag-N, or derivative thereof as described in any example hereof is modified so as to generate a unique EcoRI site positioned between nucleic acid encoding the hemagglutinin tag and the Avitag domain. Nucleic acid encoding a candidate peptide moiety as described in Example 1 is introduced to the EcoRI site of the modified vector.

Standard site-directed mutagenesis in performed to introduce the unique EcoRI site into T7Select-Avitag-N or T7Select*-Avitag-N or derivative thereof.

In another example, constructs designated T7Select-Avitag-N or T7Select*-Avitag-N, or derivative thereof as described in any example hereof is modified to replace nucleic acid encoding the hexahistidine tag with nucleic acid encoding a dodecahistidine tag (10 His; SEQ ID NO: 35) for detection and/or purification of the fusion protein. Alternatively, these vectors are modified by introducing nucleic acid encoding up to four (4) additional histidine residues to produce corresponding vectors encoding ten (10) histidine residues in tandem. Standard procedures are employed on such modifications.

In another example, the position of candidate peptide and the Avitag domain in the vector are modified with respect to each other and other domains positioned downstream of the coat protein. For example, the Avitag domain is positioned adjacent the C-terminus of the candidate peptide moiety and N-terminal of the 6 His or 10 His domain or the HA domain. The relative positions of the various tag domains in these vectors is variable and not essential to their performance. Standard procedures are employed on such modifications.

A non-biotinylated member is produced by expressing the T7Select-Avitag-N or T7Select*-Avitag-N, or derivative thereof as described in any example hereof in E. coli cells. In such an example, the bacterial cells are transformed so as to express a SUMO-(Avitag)₃ fusion decoy polypeptide (FIG. 2) comprising three tandem copies of an Avitag domain fused to a Small Ubiquitin-like Modifier (SUMO) protein e.g., Hay et al., Mol. Cell 18, 1-12 (2005). In this example, the expressed tandem copies of the biotin ligase substrate domain are biotinylated in preference of the biotin ligase substrate domain of the T7Select derivative e.g., by virtue of the endogenous biotin ligase enzyme of the bacterial cells being exposed to having a molar excess of substrate via expression from a multicopy vector multiple products of higher affinity for the tandem copies of the Avitag domain, as opposed to than for a single copy of the Avitag domain present on the pNp3 vector derivative, which in stochastic terms is less able to compete for biotinylation activity.

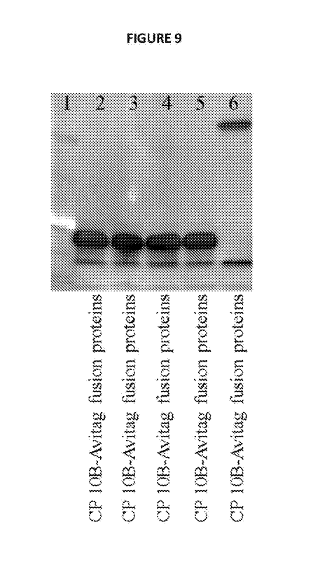

As shown in FIG. 9, CP 10B Avitag fusion proteins expressed from the T7Select vectors described herein are not biotinylated in E. coli cells in the presence of a SUMO-(Avitag)₃ fusion decoy polypeptide set forth in FIG. 2. See e.g., FIG. 9, lanes 2-5. In contrast, the T7Select vector is biotinylated in E. coli cells not expressing the SUMO-(Avitag)₃ fusion decoy polypeptide. This supports the conclusion that non-biotinylated members are displayed on T7 phage.

This example demonstrates the production of a non-biotinylated member employing expression vector T7Select to produce a filamentous bacteriophage displaying the non-biotinylated member.

### Example 6

### Production of a Non-Biotinylated Member Using Cells Expressing an Endogenous Biotin Ligase that has a Low Affinity for the Biotin Ligase Substrate Domain

This example demonstrates the production of a non-biotinylated member employing E. coli cells expressing an endogenous biotin ligase that has a low affinity for the biotin ligase substrate domain to produce a bacteriophage displaying the non-biotinylated member.

The expression constructs designated pNp3, pNp8, pJuFo-pIII, pJuFo-pVIII, T7Select-Avitag-N and T7Select*-Avitag-N or any derivative thereof as described according to any preceding example hereof are modified by replacing the Avitag domain thereof with nucleic acid encoding further to encode a 15-amino acid yeast biotin ligase substrate domain set forth in SEQ ID NO: 12 (Chen et al. J. Am. Chem. Soc. 129, 6619-6620, 2007).

A non-biotinylated member is generated by producing the bacteriophage in E. coli cells such as those cells expressing endogenous E. coli biotin ligase and/or expressing a mammalian biotin ligase.

### Example 7

### Production of a Non-Biotinylated Member Using Cells that Lack Endogenous Biotin Ligase Activity

This example demonstrates the production of a non-biotinylated member employing E. coli cells lacking endogenous biotin ligase activity and expressing a recombinant biotin ligase to produce a bacteriophage displaying the non-biotinylated member.

A non-biotinylated member is generated by expressing pNp3, pNp8, pJuFo-pIII, pJuFo-pVIII, T7Select-Avitag-N and T7Select*-Avitag-N or any derivative thereof as described according to any preceding example hereof are produced in E. coli CY918 cells (Cronan et al. FEMS Microbio. Lett. 130 221-229, 1995) that are transformed with a biotin protein ligase of Saccharomyces cerevisiae set forth in SEQ ID NO: 9.

In this example, the Avitag of the fusion proteins is not biotinylated by virtue of the bacterial cells lacking endogenous biotin ligase activity and the expressed biotin ligase of S. cerevisiae having insufficient activity for the Avitag domain present on the vector.

### Example 8

### Production of a Non-Biotinylated Member Using Cell-Free Protein Synthesis

This example demonstrates the production of a non-biotinylated member employing a eukaryotic cell-free protein expression system.

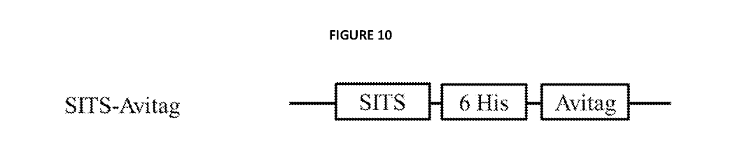

Vector construct designated SITS-Avitag was generated for use in a combined transcription-translation system using pLTE-6H-N(PEF Brisbane). The SITS-Avitag vector encodes a fusion protein comprising a species independent translation domain (SITS), a hexahistidine tag (6 His; SEQ ID NO: 33), and a biotin ligase substrate domain (Avitag domain) (SEQ ID NO: 4) (FIG. 10). The nucleotide sequence of SITS-Avitag is set for in SEQ ID NO: 76.

In one example, the expression construct designated SITS-Avitag is modified further to replace nucleic acid encoding the hexahistidine tag (6 His) domain with nucleic acid encoding a dodecahistidine tag (10 His; SEQ ID NO: 35) for detection and/or purification of the fusion protein. Alternatively, these vectors are modified by introducing nucleic acid encoding up to four (4) additional histidine residues to produce corresponding vectors encoding ten (10) histidine residues in tandem. Standard procedures are employed on such modifications.

In one example, nucleic acid encoding a candidate peptide moiety as described in Example 1 is introduced to the expression construct designated SITS-Avitag or derivative thereof between nucleic acid encoding the hexahistidine tag and nucleic acid encoding the Avitag domain using overlap extension PCR.

In another example, nucleic acid encoding a candidate peptide moiety as described in Example 1 is introduced to the expression construct designated SITS-Avitag or derivative thereof downstream of the Avitag domain using overlap extension PCR.

A non-biotinylated member is produced by expressing the SITS-Avitag, or derivative thereof as described in any example hereof in a Leishmania tarentolae extract (LTE) in vitro translation system according to manufacturer's instructions (PEF Brisbane).

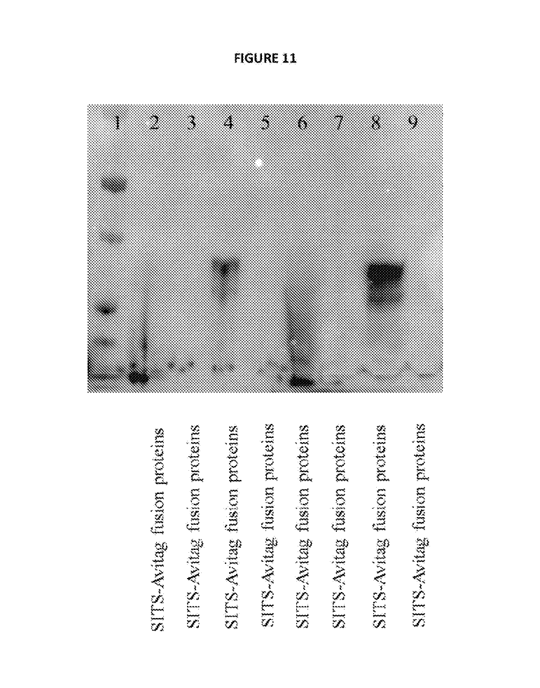

As shown in FIG. 11, fusion proteins comprising the species independent translation domain are not biotinylated a Leishmania tarentolae extract (LTE) in vitro translation system. See e.g., FIG. 11, lanes 3, 5, 7 and 9. This supports the conclusion that non-biotinylated members are produced in a eukaryotic cell-free protein expression system.

### Example 9

### Production of Host Cells Expressing a Recombinant Biotin Ligase

This example demonstrates the production of eukaryotic host cell expressing a recombinant biotin ligase.

Vector construct designated pBirA was generated for expression a recombinant E. coli biotin ligase (BirA; SEQ ID NO: 2) using pACYC-184 (New England BioLabs) (SEQ ID NO: 80) as a template. The nucleotide sequence of pBirA is set for in SEQ ID NO: 71.

Vector construct designated pBirA* was generated for expression of a mammalian codon optimised biotin ligase (BirA*; SEQ ID NO: 79) as previously described by Mechold et al. J. Biotech. 116, 245-249 (2005). The nucleotide sequence of pBirA* is set for in SEQ ID NO: 77.

Vectors pBirA and pBirA* were transfected into HEK 293 cells using electroporation. Cells stably expressing either BirA or BirA* were selected using standard molecular biology protocols.

In another example, vectors pBirA and pBirA* are transfected into CHO-K1, NIH-3T3, HeLa and COS-7 cells. Cells stably expressing either BirA or BirA* are selected using standard molecular biology protocols.

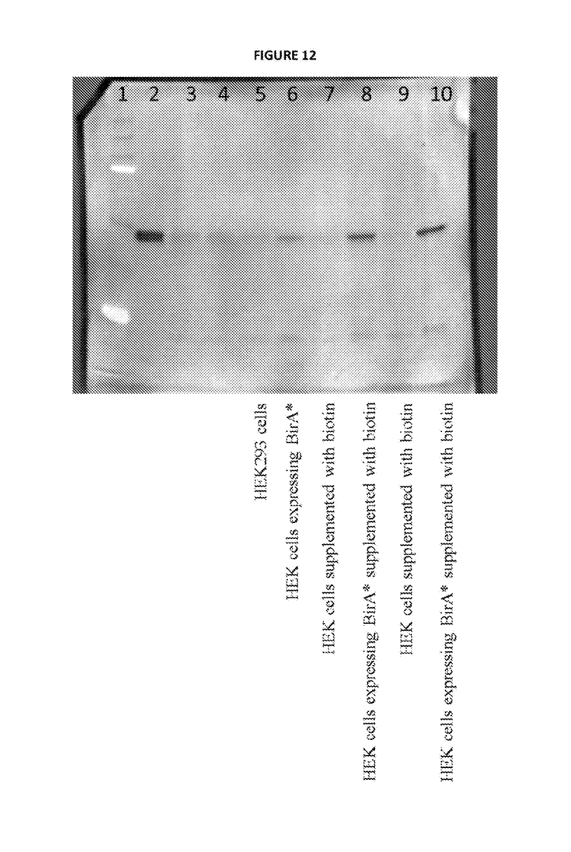

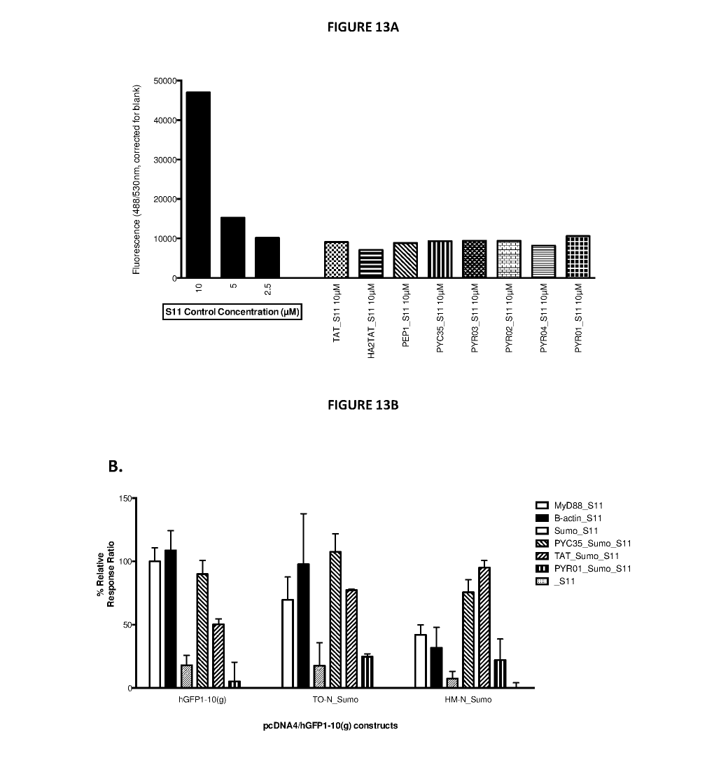

As shown in FIG. 12, transfected HEK 293 cells expressing BirA* biotinylate the non-biotinylated members with or without exogenous biotin, being added to intact HEK 293 cells in culture or to M-PER cell lysates, albeit at a lower level in the absence of exogenous biotin.

### Example 10

### Enhancing Host Cell Expression of Recombinant Biotin Ligase

This example demonstrates preferred leader sequences and expression conditions for producing recombinant biotin ligase in host cells at sufficient levels for detectable biotinylation of a biotin ligase substrate.

A codon-optimised E. coli BirA gene was cloned into the high-copy, rhamnose-inducible plasmid pD864 (DNA2.0, Inc., USA), behind the strong RBS of that plasmid, to thereby produce plasmid pD864_BirA in which expression of BirA is under operable control of a rhamnose-inducible promoter (pRham). The recombinant expression construct was transformed into E. coli BL21 cells, and cells were cultured at 25° C. for 16 hours in Luria Broth (LB) containing carbenicillin (LB/Carb50) and 0.15% (w/v) glucose to prevent induction of BirA expression, or alternatively under the same conditions albeit in LB/Carb50 media comprising 0.05% (w/v) glucose and 0.1% (w/v) rhamnose to provide for early induction of BirA expression, or in LB/Carb50 media comprising 0.15% (w/v) glucose and 0.1% (w/v) rhamnose to provide for late induction of BirA expression. Under these conditions, BirA expression was detectable using SDS/PAGE of whole cell lysates or soluble fractions thereof when rhamnose was added to media. Cells cultured at 25° C. for 16 hours in LB/Carb50 media comprising 0.15% (w/v) glucose and 0.1% (w/v) rhamnose expressed BirA at a high level in the soluble fraction of cell lysates without detectable promoter leakage.

To demonstrate that the expressed BirA protein was functional, an in vitro biotinylation test (IVB) was performed, wherein 2 μl or 6 μl of cell lysate was incubated, for 90 min each at 30° C. in 50 mM bicine buffer pH 8.3, 10 mM MgOAc/ATP, 50 μM D-biotin, and 40 μM biotin ligase substrate consisting of an avi-tagged peptide designated V5-avi (GLINDIFEAQKIEWHEGSSGKPIPNPLLGLDST), in a final reaction volume of 60 Reactions were mixed continuously in a mixer set at 600 rpm. Following incubation, 30 μl of each reaction was withdrawn for DELFIA according to standard procedures, wherein biotinylated peptide s detected by binding of Europium-labeled streptavidin (1:500) and time-resolved fluorescence of bound peptide is determined using a plate reader (excitation at 340 nm wavelength; emission at 615 nm wavelength). Data demonstrate that lysates from autoinduced pD864_BirA cultures biotinylate test peptide Avi-V5 at a level equivalent to commercially-sold, purified BirA enzyme (Genecopeia).

To demonstrate that the expressed BirA also biotinylates a phage-displayed avi-tag, the pNp3 derivative vector pNp3 DsbA 6His CG3avi (Example 2) was mixed with the cytoplasmic BirA lysate produced in E. coli at dilutions of 1/30, 1/60, 1/120, 1/240, 1/480 and 1/960, and reactions were incubated as described in the preceding paragraph. Data indicated that BirA lysate possessed detectable biotinylation activity towards the phage-displayed biotin ligase substrate, even when diluted to 1/960 (v/v).

In summary, by expressing BirA as a rhamnose-inducible enzyme from the high-copy plasmid pD864, about 50-100 times higher levels of soluble BirA enzyme were obtainable compared to the level obtained by expression from pBirAcm (data not shown). Lysates of pD864_BirA were shown to be capable of biotinylating avi-tagged peptides and phage to the same degree as commercially-sold, purified BirA enzyme.

To determine the effect of leader peptide on BirA expression level in the periplasm, BirA was expressed as a fusion protein with one of eleven different leader peptides, from the low-copy plasmid pD881 (DNA2.0 Inc., USA). The plasmid vector pD881 comprises a kanamycin-resistance selectable marker gene, a strong RBS and the low copy p15a origin of replication. A codon-optimised E. coli BirA gene was cloned into plasmid pD881, behind the strong RBS of that plasmid, to thereby produce plasmid pD881_BirA in which expression of BirA is under operable control of a rhamnose-inducible promoter (pRham). Each leader sequence was inserted separately between the promoter and BirA-encoding sequences to produce a family of pD881_peri_BirA vectors. The 11 leader sequences tested were as follows:

a. SEC pathway leader sequences (posttranslational translocation-unfolded proteins)
pelB: Erwinia carotovora pectate lyase leader (22 amino acid residues in length)
gIII: M13-derived gIII leader (18 amino acid residues in length)
ompA: E. coli outer membrane protein 3a leader (21 amino acid residues in length)
phoA: E. coli alkaline phosphatase PhoA leader (21 amino acid residues in length)
malE: maltose binding protein leader (26 amino acid residues in length)
ompC: E. coli outer membrane protein C leader (21 amino acid residues in length)
ompT: E. coli outer membrane protease leader (20 amino acid residues in length)
B. SRP pathway leader sequences (cotranslational translocation—proteins fold in periplasm)
dsbA: protein disulphide isomerase I leader (19 amino acid residues in length)
torT: regulatory protein of torCAD leader (18 amino acid residues in length)
C. TAT pathway leader sequences (posttranslational translocation—folded proteins) torA: TMAO reductase leader (43 amino acid residues in length)
sufI: (Ftsp) E. coli component of cell division apparatus leader (31 amino acid residues in length).

Cells were cultured and expression induced using rhamnose and glucose in the media as described herein above. SDS/PAGE of cell lysates indicated that BirA was expressed except when the SRP pathway leader sequences TorT or DsbA were employed.

To demonstrate that the expressed BirA protein was functional in each case, an in vitro biotinylation test (IVB) was performed as described herein above, employing soluble fractions from autoinduced pD881_peri_BirA cultures. Data indicated measurable BirA activities in pD881_peri_BirA lysates of cells wherein BirA was expressed as a fusion protein with a SEC pathway leader viz. pelB, gIII, ompA, phoA, or malE, or a TAT pathway leader torA or sun. In contrast, there was not measurable activity, or only low activity from cell lysates wherein BirA was expressed as a fusion protein with SEC pathway leaders ompC or ompT, or the SRP pathway leader dsbA or torT. Western blot immune-detection of BirA protein indicated that the SEC pathway leaders are processed correctly, whereas the SRP pathway leaders and TAT pathway leaders are only partially-processed and are thus not transported as efficiently into the periplasm of bacterial cells.

### Example 11

### Determining or Identifying Peptides that Translocate a Membrane of a Host Cell

This example demonstrates determination/identification of peptides capable of translocating a membrane of a cell, by contacting host cells expressing a biotin ligase with a plurality of non-biotinylated members, then incubating the host cells such that a biotin ligase substrate domain of fusion proteins expressed by the members that have translocated a membrane of the host cell are enzymatically biotinylated by the expressed biotin ligase, and determining or identifying those biotinylated members by detecting the fusion protein in a biotinylated form, and isolating/recovering the biotinylated fusion proteins using paramagnetic streptavidin beads.

Non-biotinylated members are produced as described in the preceding examples and then contacted with HEK-293, CHO-K1, NIH-3T3, HeLa or COS-7 cells expressing a biotin ligase enzyme.

In one example, biotinylation of the members is followed by recovering HEK 293 cells, lysing an aliquot of the recovered cells, and subjecting the cell lysates to Western blot analysis as described in Example 2. Samples comprising biotinylated members are diluted in Laemmli buffer and boiled for 5 minutes. Denatured biotinylated members are resolved on a 4-12% Bis-tris gel and blotted onto PVDF membrane (Life Technologies, Invitrogen) by using standard procedures. Membranes are blocked overnight in 5% skim milk/PBS at 4° C. overnight. Membranes are rinsed in 1×PBS with 0.05% Tween-20 (PBS-T) and incubated at room temperature for 1 hour with anti-biotin streptavidin conjugated to horseradish peroxidase (SA-HRP) (dilution 1:1,000). Membranes are washed in PBS-T and developed by using a Western C kit (Bio-Rad).

In another example, biotinylation of the members is followed by recovering HEK 293 cells, lysing an aliquot of the recovered cells, and subjecting the cell lysates to a pull-down assay. Briefly, paramagnetic streptavidin beads [Dynabeads M-280 SA or MyOne] are blocked by washing in 1 mL of 1% BSA/PBS/0.05% Tween-20 (PBT) at 4° C. for 1 hour and resuspended in 1 mL of PBT. 2.5 mg/mL of beads are added to each preparation of biotinylated phage-displayed peptides (2×1010 CFU). Binding is performed at 4° C. for 1 hour on a rocking platform, followed by three washes in binding 1 mL of PBS.

### Example 12

### Recovery of Peptides Capable of Translocating Cell Membranes

This example demonstrates determination/identification of peptides capable of translocating a membrane of a cell, by contacting host cells expressing a biotin ligase with a plurality of non-biotinylated members, then incubating the host cells such that a biotin ligase substrate domain of fusion proteins expressed by the members that have translocated a membrane of the host cell are enzymatically biotinylated by the expressed biotin ligase, and determining or identifying those biotinylated members by detecting the fusion protein in a biotinylated form, and isolating/recovering the biotinylated fusion proteins.

A highly diverse mixture of nucleic acids was produced as described in Example 1 and cloned into the vector DsbA-Avitag-pIII and DsbA-Avitag-pVIII as described in Example 2 and Example 3, respectively, to produce pluralities of non-biotinylated members i.e., bacteriophage libraries, comprising bacteriophage scaffolds displaying fusion proteins, wherein the fusion proteins each comprise a candidate peptide moiety and a biotin ligase substrate domain.

To biotinylate the members, HEK 293 cells expressing E. coli biotin ligase (BirA) were grown for 24 hours, washed once with phosphate-buffered saline (PBS) to remove debris before contacting with the phage display libraries (approximately 5×10¹² phage) for sufficient time for at least the displayed fusion proteins to enter the HEK 293 cells. To stop the reactions, the cells were washed twice with DMEM, incubated with subtilisin in HBSS at 37° C. for 30 min to 1 hour, and then PMSF in HBSS was added to the cultures, which were incubated for 15 min at room temperature. The treated cells with extrinsically-bound phage removed were collected by centrifugation, washed twice with DMEM and lysed in M-PER buffer supplemented with 10 mM pyrophosphate solution (PPi) at room temperature to inhibit or reduce biotin ligase activity.

To determine or identify those peptides that are biotinylated and have translocated the cell membrane, the biotinylated fusion proteins in the cell lysates were detected. In summary, pull-downs were performed essentially as described in Example 10 hereof on the cell lysates to recover internalized biotinylated members. Between 4-5 iterative rounds of biopanning were performed for each screen.

The fusion peptides were characterized by recovering the bacteriophage displaying the fusion peptides, recovering the members by nucleic acid amplification, and determining the nucleotide sequences of the members encoding the fusion peptides. The deduced amino acid sequences of the candidate peptide moieties of the fusion peptides were then analyzed by:
i. pairwise alignment using the CD Hit clustering program;
ii characterization of the peptides for amphipathicity, hydrophobicity, charge, size, and amino acid composition e.g., presence of arginine and lysine residues;
iii. characterization of predicted secondary structures; and
iv. database query to determine novelty of the peptides.

Bioinformatics employed PSIPRED algorithm, e.g., McGuffin et al., Bioinformatics 16 404-405 (2000). Database queries were performed using a database of known CPPs available at the database CellPPD: Designing of Cell Penetrating Peptides, which provides in silico prediction of cell penetration efficiency based on a dataset of 708 experimentally-validated CPPs. In particular, CellPPD permits prediction of peptides having CPP-like properties in each pool of isolated or identified peptides based on their sequences, including the identification of CPP-like motifs in peptides. See e.g., Gautam et al., J. Translational Med. 11, 74 (2013).

The CD Hit clustering program was run employing various clustering thresholds, including a sequence identity of greater than 50% to identify CPP-like motifs, and a clustering threshold of greater than 90% to prevent mismatch errors, and at a sequence identity of greater than 100%, to eliminate redundant sequences. The clustering analyses performed revealed that the vast majority of peptides identified by employing the present screening method are unique or represented once e.g. “singletons”. This indicates the power of the method for identifying CPP-like peptides represented at low frequency, or that are rare, in the population of members. High levels of sequence diversity were also observed within the recovered peptides, suggesting that the plurality of members will provide a source of new and rare classes of CPP-like peptides identifiable by employing the inventive method. Multiple copies of certain sequences were also present in the recovered fusion peptides, indicating reproducibility of the method.

Bioinformatics analyses of the bacteriophage library i.e., plurality of members prior to selection, and of the recovered peptides encoded by the biotinylated members prior to their validation by functional assay(s), are provided in Table 2. The data provided show CPP-like properties of peptide pools at each stage.

Data presented in Table 2 and Table 3 indicate that performance of the method of the invention resulted in recovery of a higher average length and molecular weight of peptide than contained in the phage library, and a distinct shift towards recovery of charged peptides having reduced hydrophobicity and forming alpha-helices. In contrast, representation of β-sheet conformations in the recovered peptides may be reduced relative to the proportion of β-sheet conformations encoded by the input non-biotinylated members. This may be a reflection of a generally higher representation of alpha-helical structures and lower representation of β-sheet conformations in the recovered peptide pool, indicative of a higher proportion of peptides having CPP-like properties relative to other protein functionalities. Specific enrichment for positively-charged residues as opposed to negatively-charged residues, and for alpha helices, is entirely consistent with properties of peptides that are capable of translocating lipid bilayers such as those of cell membranes.

Sequence analysis of the recovered peptides also indicated that 49 peptides having CPP-like properties were recovered using the method of the invention described herein, from a pool of approximately 5×10¹² bacteriophage screened, whereas about 29 peptides of a random pool of the input phage library had CPP-like properties. This demonstrates enrichment for peptides having CPP-like properties by performing the inventive method.

TABLE 2
Characterization of peptides encoded by input phage
display and recovered biotinylated members

| | | Input Phage | Recovered |
|---|---|---|---|
| | | Library (non- | members |
| Peptide property | | biotinylated pool) | (biotinylated) |
| Number of peptides [n] | | 173 | 176 |
| Ave. Length [amino acid residues] | | 23 | 44 |
| Ave. Molecular Weight of | | 2598.8 | 4967.3 |
| encoded peptide [Da] | | | |
| Ave. Isoelectric point (pI) | | 8.7 | 10.3 |
| Ave. Charge | | 1.9 | 4.2 |
| Ave. Hydrophobicity (pH 6.8) | | 475.9 | 330.6 |
| Ave. Amphipathicity | | 0.2 | 0.3 |
| Amino acid | Acidic | 6.8 | 9.5 |
| composition | Aliphatic | 28.8 | 21.0 |
| (Ave. No of | Aromatic | 11.1 | 9.6 |
| residues | Basic | 16.5 | 21.0 |
| adjusted for | Charged | 23.3 | 30.5 |
| length, %) | Non-polar | 54.0 | 43.1 |
| | Polar | 46.0 | 56.9 |
| | Small | 52.0 | 53.4 |
| | Tiny | 31.1 | 29.7 |
| Raw amino acid | A | 1.7 [3.6] | 3.5 [6.1] |
| counts for | C | 0.6 [1.3] | 0.6 [1.1] |
| different amino | D | 0.8 [1.7] | 1.8 [3.2] |
| acids of the 20 | E | 0.8 [1.7] | 2.3 [4.1] |
| common amino | F | 0.9 [1.9] | 0.9 [1.5] |
| acids [Ave. No | G | 1.4 [3.1] | 2.6 [4.6] |
| amino acid | H | 0.8 [1.6] | 1.8 [3.1] |
| counts for | I | 1.1 [2.3] | 1.1 [1.8] |
| different amino | K | 0.7 [1.5] | 1.9 [3.4] |
| acids of the 20 | L | 2.4 [5.1] | 2.4 [4.2] |
| common amino | M | 0.3 [0.6] | 0.4 [0.7] |
| acids adjusted | N | 0.8 [1.7] | 2.8 [4.9] |
| for length, %] | P | 1.7 [3.7] | 3.5 [6.2] |
| | Q | 1.0 [2.1] | 2.5 [4.4] |
| | R | 2.3 [5.1] | 5.5 [9.6] |
| | S | 2.2 [4.7] | 3.4 [6.0] |
| | T | 1.3 [2.9] | 2.8 [5.0] |
| | V | 1.5 [3.3] | 2.3 [4.0] |
| | W | 0.3 [0.7] | 0.5 [0.9] |
| | Y | 0.6 [1.3] | 1.1 [1.8] |

Results of secondary structure prediction analyses, undertaken using the PSIPRED algorithm, are provided in Table 3.

TABLE 3
Summary of secondary structure prediction analysis

| | Input Phage | Recovered |
|---|---|---|
| Predicted Secondary | Library (non- | members |
| Structure | biotinylated pool) | (biotinylated) |
| Coil | 0.774 | 0.738 |
| Sheet | 0.133 | 0.095 |
| Helix | 0.094 | 0.167 |

The inventors have also compared the results obtained employing the method of the invention, relative to the results obtained employing a method that does not rely upon selective biotinylation of non-biotinylated members to recover those members that have entered the cells, and exemplary data are provided in Table 4. Such comparative methods are described in WO 2012/159164. Data indicate that the inventive method provides improved qualitative and quantitative recovery of peptides having CPP-like properties.

### Example 13

### Recovery and Characterisation of Peptides Capable of Translocating a Membrane of a Cell

This example demonstrates determination/identification of peptides capable of translocating a membrane of a cell, by contacting host cells expressing a biotin ligase with a plurality of non-biotinylated members, then incubating the host cells such that a biotin ligase substrate domain of fusion proteins expressed by the members that have translocated a membrane of the host cell are enzymatically biotinylated by the expressed biotin ligase, and determining or identifying those biotinylated members by detecting the fusion protein in a biotinylated form, and isolating/recovering the biotinylated fusion proteins.

A highly diverse mixture of nucleic acids was produced as described in Example 1 and cloned into the vector T7Select-Avitag-N as described in Example 5 to produce pluralities of non-biotinylated members i.e., bacteriophage libraries, comprising bacteriophage scaffolds displaying fusion proteins, wherein the fusion proteins each comprise a candidate peptide moiety and a biotin ligase substrate domain.

To biotinylate the members, HEK 293 cells expressing E. coli biotin ligase (BirA) were grown for 24 hours, washed once with phosphate-buffered saline (PBS) to remove debris before contacting with the phage display libraries (approximately 5×10¹² phage) for sufficient time for at least the displayed fusion proteins to enter the HEK 293 cells. To stop the reactions, the cells

TABLE 4
Comparison of methods that rely upon selective biotinylation of non-biotinylated
members to a method that does not employ selective biotinylation

| | | Non- | Recovered | | Comparator | | Comparator |
|---|---|---|---|---|---|---|---|
| | | biotinylated | members | Comparator | process | Comparator | process |
| Property | | pool | (biotinylated) | library #1 | result #1 | library #2 | result #2 |
| Number of peptides [n] | | 173 | 176 | 218 | 230 | 219 | 113 |
| Ave. Length [amino acid residues] | | 23 | 44 | 34 | 38 | 33 | 26 |
| Ave. Molecular Weight of encoded | | 2598.8 | 4967.3 | 3817.3 | 4376.2 | 3722.3 | 2988.1 |
| peptide [Da] | | | | | | | |
| Ave. Isoelectric point (pI) | | 8.7 | 10.3 | 8.4 | 7.6 | 8.3 | 8.5 |
| Ave. Charge | | 1.9 | 4.2 | 1.7 | −1.3 | 1.1 | 1.9 |
| Ave. Hydrophobicity (pH 6.8) | | 475.9 | 330.6 | 702.3 | 650.6 | 636.2 | 590.8 |
| Ave. Amphipathicity | | 0.2 | 0.3 | 0.3 | 0.2 | 0.3 | 0.2 |
| Amino acid | Acidic | 6.8 | 9.5 | 8.9 | 14.9 | 10.6 | 7.9 |
| composition | Aliphatic | 28.8 | 21.0 | 28.8 | 24.2 | 29.0 | 28.2 |
| (Ave. No of | Aromatic | 11.1 | 9.6 | 10.5 | 14.0 | 10.1 | 12.6 |
| residues | Basic | 16.5 | 21.0 | 15.3 | 12.8 | 15.7 | 16.8 |
| adjusted for | Charged | 23.3 | 30.5 | 24.2 | 27.7 | 26.3 | 24.7 |
| length, %) | Non-polar | 54.0 | 43.1 | 54.9 | 50.7 | 53.6 | 55.7 |
| | Polar | 46.0 | 56.9 | 45.1 | 49.3 | 46.4 | 44.3 |
| | Small | 52.0 | 53.4 | 52.6 | 49.9 | 52.1 | 47.6 |
| | Tiny | 31.1 | 29.7 | 32.6 | 27.8 | 30.9 | 28.3 |
| Raw amino | A | 1.7 [3.6] | 3.5 [6.1] | 2.8 [6.3] | 2.7 [5.5] | 2.7 [5.8] | 1.8 [5.8] |
| acid counts | C | 0.6 [1.3] | 0.6 [1.1] | 0.9 [2.0] | 0.6 [1.2] | 0.9 [1.9] | 0.7 [1.9] |
| for different | D | 0.8 [1.7] | 1.8 [3.2] | 1.5 [3.4] | 2.8 [5.7] | 1.7 [3.6] | 0.8 [3.6] |
| amino acids | E | 0.8 [1.7] | 2.3 [4.1] | 1.5 [3.4] | 2.9 [5.8] | 1.8 [4] | 1.2 [4.0] |
| of the 20 | F | 0.9 [1.9] | 0.9 [1.5] | 1.1 [2.5] | 2.2 [4.4] | 1.0 [2.1] | 1.1 [2.1] |
| common | A | 1.7 [3.6] | 3.5 [6.1] | 2.8 [6.3] | 2.7 [5.5] | 2.7 [5.8] | 1.8 [5.8] |
| amino acids | C | 0.6 [1.3] | 0.6 [1.1] | 0.9 [2.0] | 0.6 [1.2] | 0.9 [1.9] | 0.7 [1.9] |
| [Ave. No | D | 0.8 [1.7] | 1.8 [3.2] | 1.5 [3.4] | 2.8 [5.7] | 1.7 [3.6] | 0.8 [3.6] |
| amino acid | E | 0.8 [1.7] | 2.3 [4.1] | 1.5 [3.4] | 2.9 [5.8] | 1.8 [4] | 1.2 [4.0] |
| counts for | F | 0.9 [1.9] | 0.9 [1.5] | 1.1 [2.5] | 2.2 [4.4] | 1.0 [2.1] | 1.1 [2.1] |
| different | G | 1.4 [3.1] | 2.6 [4.6] | 2.7 [6.0] | 2.2 [4.5] | 2.4 [5.2] | 1.7 [5.2] |
| amino | H | 0.8 [1.6] | 1.8 [3.1] | 0.9 [2.1] | 1.0 [2.0] | 1.1 [2.5] | 0.9 [2.5] |
| acids of the 20 | I | 1.1 [2.3] | 1.1 [1.8] | 1.5 [3.3] | 1.2 [2.4] | 1.5 [3.2] | 1.4 [3.2] |
| common | K | 0.7 [1.5] | 1.9 [3.4] | 1.3 [2.8] | 1.4 [2.9] | 1.0 [2.2] | 1.0 [2.2] |
| amino acids | L | 2.4 [5.1] | 2.4 [4.2] | 3.3 [7.3] | 3.8 [7.6] | 3.1 [6.7] | 2.6 [6.7] |
| adjusted for | M | 0.3 [0.6] | 0.4 [0.7] | 0.6 [1.4] | 0.4 [0.8] | 0.6 [1.3] | 0.6 [1.3] |
| length, %] | N | 0.8 [1.7] | 2.8 [4.9] | 1.0 [2.2] | 1.5 [3.0] | 0.9 [2.0] | 0.9 [2.0] |
| | P | 1.7 [3.7] | 3.5 [6.2] | 2.1 [4.6] | 2.6 [5.2] | 2.1 [4.5] | 1.8 [4.5] |
| | Q | 1.0 [2.1] | 2.5 [4.4] | 1.4 [3.1] | 1.7 [3.5] | 1.5 [3.2] | 1.0 [3.2] |
| | R | 2.3 [5.1] | 5.5 [9.6] | 3.1 [6.8] | 2.5 [5.1] | 3.1 [6.6] | 2.5 [6.6] |
| | S | 2.2 [4.7] | 3.4 [6.0] | 3.0 [6.6] | 3.3 [6.6] | 2.5 [5.4] | 1.9 [5.4] |
| | T | 1.3 [2.9] | 2.8 [5.0] | 1.8 [3.9] | 1.8 [3.7] | 1.8 [3.9] | 1.4 [3.9] |
| | V | 1.5 [3.3] | 2.3 [4.0] | 2.3 [5.0] | 1.6 [3.2] | 2.4 [5.1] | 1.5 [5.1] |
| | W | 0.3 [0.7] | 0.5 [0.9] | 0.7 [1.6] | 1.2 [2.4] | 0.5 [1.1] | 0.6 [1.1] |
| | Y | 0.6 [1.3] | 1.1 [1.8] | 0.8 [1.8] | 1.0 [2.1] | 0.8 [1.7] | 0.7 [1.7] |
| Secondary | Coil | 0.774 | 0.738 | 0.729 | 0.755 | 0.732 | 0.748 |
| Structure | Sheet | 0.133 | 0.095 | 0.134 | 0.118 | 0.122 | 0.116 |
| | Helix | 0.094 | 0.167 | 0.137 | 0.129 | 0.146 | 0.137 |
| Peptides | Number of peptides | 29 [16.763] | 49 [27.841] | 46 [21.101] | 38 [16.522] | 53 [24.201] | 26 [23.009] |
| having CPP- | having CPP-like | | | | | | |
| like properties | properties | | | | | | |
| | [proportion, %] | | | | | | |

were washed with DMEM, and incubated with 2 mL of 0.25% trypsin/EDTA at 37° C. for 1-5 min Cells were collected by centrifugation, washed twice with DMEM and lysed in M-PER buffer supplemented with 1 mM pyrophosphate solution (PPi) at room temperature to inhibit or reduce biotin ligase activity.

To determine or identify those peptides that are biotinylated and have translocated the cell membrane, the biotinylated fusion proteins in the cell lysates were detected. In summary, pull-downs were performed essentially as described in Example 10 hereof on the cell lysates to recover internalized biotinylated members. Between 4-5 iterative rounds of biopanning were performed for each screen.

The fusion peptides were characterized by recovering the bacteriophage displaying the fusion peptides, recovering the members by nucleic acid amplification, and determining the nucleotide sequences of the members encoding the fusion peptides. The deduced amino acid sequences of the candidate peptide moieties of the fusion peptides were then analyzed by:
(i) pairwise alignment using the CD Hit clustering program;
(ii) characterization of the peptides for amphipathicity, hydrophobicity, charge, size, and amino acid composition e.g., presence of arginine and lysine residues;
(iii) characterization of predicted secondary structures; and
(iv) database query to determine novelty of the peptides.

Bioinformatics employed PSIPRED algorithm, e.g., McGuffin et al., Bioinformatics 16 404-405 (2000). Database queries were performed using a database of known CPPs available at the database CellPPD: Designing of Cell Penetrating Peptides, which provides in silico prediction of cell penetration efficiency based on a dataset of 708 experimentally-validated CPPs. In particular, CellPPD permits prediction of peptides having CPP-like properties in each pool of isolated or identified peptides based on their sequences, including the identification of CPP-like motifs in peptides. See e.g., Gautam et al., J. Translational Med. 11, 74 (2013).

The CD Hit clustering program was run employing various clustering thresholds, including a sequence identity of greater than 50% to identify CPP-like motifs, and a clustering threshold of greater than 90% to prevent mismatch errors, and at a sequence identity of greater than 100%, to eliminate redundant sequences. The clustering analyses performed revealed that the vast majority of peptides identified by employing the present screening method are unique or represented once e.g. “singletons”. This indicates the power of the method for identifying CPP-like peptides represented at low frequency, or that are rare, in the population of members. High levels of sequence diversity were also observed within the recovered peptides, suggesting that the plurality of members will

TABLE 5
Characterization of peptides encoded by input phage
display and recovered biotinylated members

| | | Input Phage | Recovered |
|---|---|---|---|
| | | Library (non- | members |
| Peptide property | | biotinylated pool) | (biotinylated) |
| Number of peptides (n) | | 173 | 261 |
| Ave. Length (amino acid residues) | | 22 | 38 |
| Ave. Molecular Weight of | | 2507.9 | 4317.7 |
| encoded peptide (Da) | | | |
| Ave. Isoelectric point (pI) | | 8.5 | 10.6 |
| Ave. Charge | | 1.7 | 6.6 |
| Ave. Hydrophobicity (pH 6.8) | | 419.2 | 123.3 |
| Ave. Amphipathicity | | 0.2 | 0.3 |
| Amino acid | Acidic | 7.7 | 7.0 |
| composition | Aliphatic | 27.2 | 18.2 |
| (Ave. No of | Aromatic | 10.5 | 7.2 |
| residues | Basic | 16.8 | 26.4 |
| adjusted for | Charged | 24.5 | 33.4 |
| length, %) | Non-polar | 52.6 | 41.7 |
| | Polar | 47.4 | 58.3 |
| | Small | 51.6 | 53.1 |
| | Tiny | 31.6 | 31.8 |
| Raw amino acid | A | 1.6 [3.4] | 2.9 [5.0] |
| counts for | C | 0.6 [1.2] | 0.9 [1.6] |
| different amino | D | 0.9 [1.9] | 1.5 [2.6] |
| acids of the 20 | E | 0.8 [1.7] | 1.2 [2.0] |
| common amino | F | 0.8 [1.6] | 0.5 [0.8] |
| acids [Ave. No | G | 1.4 [3.0] | 2.5 [4.3] |
| amino acid | H | 0.7 [1.4] | 1.5 [2.6] |
| counts for | I | 0.8 [1.7] | 0.7 [1.1] |
| different amino | K | 0.7 [1.5] | 1.2 [2.1] |
| acids of the 20 | L | 2.4 [5.0] | 2.0 [3.4] |
| common amino | M | 0.4 [0.8] | 0.3 [0.5] |
| acids adjusted | N | 0.7 [1.4] | 1.2 [2.1] |
| for length, %] | P | 1.7 [3.5] | 4.0 [6.8] |
| | Q | 1.0 [2.1] | 2.5 [4.2] |
| | R | 2.4 [5.0] | 7.2 [12.4] |
| | S | 2.2 [4.6] | 3.9 [6.7] |
| | T | 1.2 [2.6] | 1.8 [3.2] |
| | V | 1.2 [2.6] | 1.4 [2.3] |
| | W | 0.4 [0.8] | 0.4 [0.6] |
| | Y | 0.5 [1.1] | 0.3 [0.6] |

provide a source of new and rare classes of CPP-like peptides identifiable by employing the inventive method. Multiple copies of certain sequences were also present in the recovered fusion peptides, indicating reproducibility of the method. Bioinformatic analyses of the bacteriophage library i.e., plurality of members prior to selection, and of the recovered peptides encoded by the biotinylated members prior to their validation by functional assay(s), are provided in Table 5. The data provided show CPP-like properties of peptide pools at each stage. Results of secondary structure prediction analyses, undertaken using the PSIPRED algorithm, are provided in Table 6.

TABLE 6
Summary of secondary structure prediction analysis

| | Input Phage | Recovered |
|---|---|---|
| Predicted Secondary | Library (non- | members |
| Structure | biotinylated pool) | (biotinylated) |
| Coil | 0.784 | 0.843 |
| Sheet | 0.106 | 0.052 |
| Helix | 0.111 | 0.105 |

Data presented in Table 5 and Table 6 indicate that performance of the method of the invention resulted in recovery of a higher average length and molecular weight of peptide than contained in the phage library, and a distinct shift towards recovery of charged peptides having reduced hydrophobicity and forming alpha-helices. In contrast, representation of β-sheet conformations in the recovered peptides may be reduced relative to the proportion of β-sheet conformations encoded by the input non-biotinylated members. This may be a reflection of a generally higher representation of alpha-helical structures and lower representation of β-sheet conformations in the recovered peptide pool, indicative of a higher proportion of peptides having CPP-like properties relative to other protein functionalities. Specific enrichment for positively-charged residues as opposed to negatively-charged residues, and for alpha helices, is entirely consistent with properties of peptides that are capable of translocating lipid bilayers such as those of cell membranes.

Sequence analysis of the recovered peptides also indicated that 66 peptides having CPP-like properties were recovered using the method of the invention described herein, from a pool of approximately 5×10¹² bacteriophage screened, whereas only 26 peptides encoded by a random pool of the input phage library had CPP-like properties. This demonstrates enrichment for peptides having CPP-like properties by performing the inventive method.

The inventors have also compared the results obtained employing the method of the invention, relative to the results obtained employing a method that does not rely upon selective biotinylation of non-biotinylated members to recover those members that have entered the cells, and exemplary data are provided in Table 7. Such comparative methods are described in WO 2012/159164.

TABLE 7
Comparison of methods that rely upon selective biotinylation of non-biotinylated
members to a method that does not employ selective biotinylation

| | | Non- | Recovered | | Comparator |
|---|---|---|---|---|---|
| | | biotinylated | members | Comparator | process |
| | | pool | (biotinylated) | library #1 | result #1 |
| Number of peptides [n] | | 173 | 261 | 289 | 450 |
| Length [amino acid residues] | | 22 | 38 | 19 | 16 |
| Molecular Weight [Da] | | 2507.9 | 4317.7 | 2076.4 | 1746.1 |
| Isoelectric point (pI) | | 8.5 | 10.6 | 8.1 | 8.3 |
| Charge | | 1.7 | 6.6 | 1.3 | 1.3 |
| Hydrophobicity (pH 6.8) | | 419.2 | 123.3 | 299.4 | 223.9 |
| Amphipathicity | | 0.2 | 0.3 | 0.2 | 0.2 |
| Amino acid | Acidic | 7.7 | 7.0 | 8.3 | 7.7 |
| composition | Aliphatic | 27.2 | 18.2 | 25.2 | 24.0 |
| (Ave. No of | Aromatic | 10.5 | 7.2 | 10.8 | 10.1 |
| residues | Basic | 16.8 | 26.4 | 173 | 17.4 |
| adjusted for | Charged | 24.5 | 33.4 | 25.6 | 25.1 |
| length, %) | Non-polar | 52.6 | 41.7 | 52.6 | 51.9 |
| | Polar | 47.4 | 58.3 | 47.4 | 48.1 |
| | Small | 51.6 | 53.1 | 54.3 | 54.5 |
| | Tiny | 31.6 | 31.8 | 33.8 | 34.2 |
| Raw amino | A | 1.6 [3.4] | 2.9 [5.0] | 1.5 [3.1] | 1.2 [2.6] |
| acid counts | C | 0.6 [1.2] | 0.9 [1.6] | 0.7 [1.5] | 0.6 [1.1] |
| for different | D | 0.9 [1.9] | 1.5 [2.6] | 0.7 [1.6] | 0.6 [1.2] |
| amino acids | E | 0.8 [1.7] | 1.2 [2.0] | 0.8 [1.7] | 0.6 [1.3] |
| of the 20 | F | 0.8 [1.6] | 0.5 [0.8] | 0.7 [1.4] | 0.4 [0.9] |
| common | A | 1.6 [3.4] | 2.9 [5.0] | 1.5 [3.1] | 1.2 [2.6] |
| amino acids | C | 0.6 [1.2] | 0.9 [1.6] | 0.7 [1.5] | 0.6 [1.1] |
| [Ave. No | D | 0.9 [1.9] | 1.5 [2.6] | 0.7 [1.6] | 0.6 [1.2] |
| amino acid | E | 0.8 [1.7] | 1.2 [2.0] | 0.8 [1.7] | 0.6 [1.3] |
| counts for | F | 0.8 [1.6] | 0.5 [0.8] | 0.7 [1.4] | 0.4 [0.9] |
| different | G | 1.4 [3.0] | 2.5 [4.3] | 1.5 [3.2] | 1.2 [2.6] |
| amino | H | 0.7 [1.4] | 1.5 [2.6] | 0.7 [1.5] | 0.5 [1.0] |
| acids of the 20 | I | 0.8 [1.7] | 0.7 [1.1] | 0.7 [1.5] | 0.6 [1.3] |
| common | K | 0.7 [1.5] | 1.2 [2.1] | 0.8 [1.8] | 0.7 [1.4] |
| amino acids | L | 2.4 [5.0] | 2.0 [3.4] | 1.5 [3.1] | 1.1 [2.4] |
| adjusted for | M | 0.4 [0.8] | 0.3 [0.5] | 0.3 [0.6] | 0.2 [0.5] |
| length, %] | N | 0.7 [1.4] | 1.2 [2.1] | 0.7 [1.5] | 0.6 [1.2] |
| | P | 1.7 [3.5] | 4.0 [6.8] | 1.3 [2.8] | 1.3 [2.6] |
| | Q | 1.0 [2.1] | 2.5 [4.2] | 0.7 [1.5] | 0.7 [1.5] |
| | R | 2.4 [5.0] | 7.2 [12.4] | 1.7 [3.5] | 1.5 [3.2] |
| | S | 2.2 [4.6] | 3.9 [6.7] | 1.5 [3.3] | 1.4 [2.8] |
| | T | 1.2 [2.6] | 1.8 [3.2] | 1.1 [2.3] | 0.9 [1.9] |
| | V | 1.2 [2.6] | 1.4 [2.3] | 1.0 [2.1] | 0.8 [1.6] |
| | W | 0.4 [0.8] | 0.4 [0.6] | 0.3 [0.5] | 0.3 [0.6] |
| | Y | 0.5 [1.1] | 0.3 [0.6] | 0.4 [0.8] | 0.3 [0.7] |
| Secondary | Coil | 0.784 | 0.843 | 0.847 | 0.873 |
| Structure | Sheet | 0.106 | 0.052 | 0.086 | 0.065 |
| | Helix | 0.111 | 0.105 | 0.068 | 0.062 |
| Peptides | Number of peptides | 26 [15.029] | 66 [25.287] | 41 [14.187] | 50 [11.111] |
| having CPP- | having CPP-like | | | | |
| like properties | properties | | | | |
| | [proportion, %] | | | | |

Data provided on Table 7 indicate that the inventive method provides improved qualitative and quantitative recovery of peptides having CPP-like properties.

### Example 14

### Alternate Protocol for Recovery and Characterisation of Peptides Capable of Translocating a Membrane of a Cell

This example demonstrates determination/identification of peptides capable of translocating a membrane of a cell, by contacting bacterial host cells expressing a biotin ligase with a plurality of non-biotinylated members, then incubating the host cells such that a biotin ligase substrate domain of fusion proteins expressed by the members that have translocated a membrane of the host cell are enzymatically biotinylated by the expressed biotin ligase, and determining or identifying those biotinylated members by detecting the fusion protein in a biotinylated form, and isolating/recovering the biotinylated fusion proteins.

A highly diverse mixture of nucleic acids was produced as described in Example 1 and cloned into the vector T7Select-Avitag-N as described in Example 5 to produce pluralities of non-biotinylated members i.e., bacteriophage libraries, comprising bacteriophage scaffolds displaying fusion proteins, wherein the fusion proteins each comprise a candidate peptide moiety and a biotin ligase substrate domain.

To biotinylate the members, E. coli comprising the vector pD864_BirA or pD881_BirA vectors described in Example 10 are induced to over-express codon-optimized BirA in the periplasm in accordance with that example. Cells expressing BirA are collected by centrifugation. A Library of PelB-Avitag-pVIII derivative phage (FIG. 4a) expressing candidate peptides (Example 3) are precipitated using PEG, resuspended in 400 ul PBS, and passed through a Streptavidin-SpinTrap column (GE healthcare) to remove any traces of endogenously biotinylated phage. The eluent is collected by centrifugation, adjusted to a concentration of about 1×10¹³ cfu/ml in PBS, and the collected cell pellet is resuspended in the bacteriophage. Biotinylation reactions are performed on mixtures of as described in the preceding examples. The cells are then collected by centrifugation, washed in PBS/pyrophosphate, lysed by suspension in BugBuster protein extraction reagent (Merck/Millipore) and incubation with shaking for 20 min. The soluble fraction of the cellular lysate, comprising biotinylated bacteriophage, is collected by centrifugation and retained. The biotinylated bacteriophage are bound to magnetic Streptavidin-Dynabeads (MyOne, Invitrogen) according to manufacturer's instructions. Bead-captured phage clones are amplified for subsequent rounds of biopanning by infecting bacterial cell cultures directly. Phage are purified by repeating the procedure on serial dilutions of aliquots of positive clones. to enrich for individual phage clones displaying peptides that enable the phage to enter the periplasm or cytoplasm of bacterial cells.

Screening may be monitored by assaying aliquots (20 μl) of the Dynabead eluents obtained in each round of biopanning. The phage are separated SDS-PAGE, and proteins transferred to nylon membrane by western blotting, and the membrane blocked using 3% (w/v) BSA in TBS-Tween, and biotinylated fusion peptides detected using Streptavidin-HRP conjugate (1:1000 in TBST) and ECL detection.

Isolated and purified bacteriophage are characterised by primary sequence, analyzed for enriched sequences, and subjected to validation assays.

### Example 15

### Structural Analysis of Peptides Capable of Translocating a Membrane of a Cell

This example demonstrates primary and secondary structure analysis of 38 representative peptides shown to be capable of translocating a membrane of a cell in accordance with the preceding examples. The peptides were isolated by contacting host cells expressing a biotin ligase with a plurality of non-biotinylated members, then incubating the host cells such that a biotin ligase substrate domain of fusion proteins expressed by the members that have translocated a membrane of the host cell are enzymatically biotinylated by the expressed biotin ligase, and determining or identifying those biotinylated members by detecting the fusion protein in a biotinylated form, and isolating/recovering the biotinylated fusion proteins. The primary sequences and CD spectra of the isolated peptides were determined. Data are summarized in Table 8.

To determine the conformation of the peptides presented in Table 8 (SEQ ID Nos: 83-119), CD spectrophotometry was performed under various conditions including different pH conditions and in the presence of membrane-mimetic SDS micelles. The secondary structure characteristics of synthesised and purified FITC-labelled peptides (Mimotopes, Australia), e.g., the peptides designated T08_HBM_0103_0031, T08_HBM_0104_0084, T09_HBM0103_0167, C10_ABH_0203_0169, C20_ABH_0404_1869 and C20_ABH_0304_1746 set forth in Table 8, and a further peptide designated PYCJX-0901, were determined by collecting CD spectra at pH4.5 and 7.2 in 10 mM NaF, and at pH4.5 and 7.2 in 25 mM SDS/10 mM NaF. Control peptides were TAT, transportan and penetratin. Briefly, peptide stock solutions were solubilised in Baxter water to a concentration of 1 mM. For CD spectra, peptides were diluted to 0.3 mg/ml, final volume 300 ul, in either 10 mM NaF pH4.5 or pH7.2 so as to evaluate the effect of pH on peptide structure. The effect of a micellar medium on peptide conformation was determined by adding 30 ul 275 mM SDS/10 mM NaF pH4.5 or pH7.2 to the original peptide/buffer solutions. Spectra were recorded between 190 and 260 nm, with 4 scans recorded per peptide. All spectra were averaged and baseline corrected by subtraction of averaged blank CD spectra of the appropriate buffer and buffer/SDS mixes. Data processing was done in Xcel and graphs plotted with Prism. Data are summarized in Table 9.

TABLE 8
Structural characterization of identified CPPs

| | | | | Hydro- | | | |
|---|---|---|---|---|---|---|---|
| | SEQ | | | phobic | | | |
| Peptide | ID | Length | Net | residues | Cys | ORF Blastp | |
| ID | NO | (aa) | charge | (%) | [n] | homology | Psi prediction |
| T08_HBM_ | 83 | 33 | 5 | 12.1 | 1 | fibronectin-binding | CCCHHHHHHHHHHHCCCCCCCCCHHHHHHHHHC |
| 0103_0031 | | | | | | A domain-containing | |
| | | | | | | protein fragment | |
| | | | | | | [Halcarcula | |
| | | | | | | amylolytica JCM | |
| | | | | | | 13557] | |
| T08_HBM_ | 84 | 33 | 11 | 21.2 | 0 | | CHHHHHHHHHHCCCCCCCHHHCCCHHHHHHHHC |
| 0104_0084 | | | | | | | |
| T09_HBM_ | 85 | 32 | 6 | 31.3 | 0 | | CCCCCCCCCCCCCCCCCCCCEEEEEECCCCCC |
| 0103_0167 | | | | | | | |
| C10_ABH_ | 86 | 43 | 14 | 18.6 | 0 | | CCHHHHHHHCCCHHHHHHHHHHHHCCCCCCCCEEEE |
| 0203_0169 | | | | | | | ECCCCCC |
| C20_ABH_ | 87 | 31 | 6 | 29 | 1 | | CCCCCCCCEEEEECCCCEEEEECCCCCCCCC |
| 0403_1788 | | | | | | | |
| C20_ABH_ | 88 | 59 | 10 | 25.4 | 0 | hypothetical protein | CCCCCCCCCCCCHHHHHCCCCCCCCCHHHHHHHHHC |
| 0103_1267 | | | | | | BCE_1797 fragment | CCCEEECCCCCCCEEEEEEEECC |
| | | | | | | (Bacillus) | |
| C20_ABH_ | 89 | 47 | 10 | 21.3 | 0 | S34 Sindbis virus | CCCCCCCCCHHHHHHHHHCCCCCCCCCCCCCCCCCC |
| 0404_1869 | | | | | | protein C fragment | |
| C20_ABH_ | 90 | 38 | 17 | 2.6 | 0 | Transposase fragment | CCCCCCCCHHHHHHCCCCCCCCCCHHHHHHCCCCCC |
| 0304_1746 | | | | | | [Bordetella | CC |
| | | | | | | pertussis] | |
| C10_HBM_ | 91 | 44 | 12 | 18.2 | 0 | polyprotein fragment | CHHHHHHHHHCCCCCCCCCCCCCCCCCCCCCCCCCC |
| 0104_0481 | | | | | | [Sindbis virus] | CCCCCCCC |
| C10_ABH_ | 92 | 24 | 6 | 25.0 | 0 | | CCEEEEEEEEEEEECCCCCCCCCC |
| 0202_0113 | | | | | | | |
| C10_ABP_ | 93 | 20 | 4 | 35.0 | 0 | | CCCEEEECCCCCCCEEEEEC |
| 0103_0330 | | | | | | | |
| C11_HBM_ | 94 | 27 | 2 | 33.3 | 0 | | CCCCCCCCCCCCCCCCCCEEECCCCCC |
| 0102_0297 | | | | | | | |
| C12_ABH_ | 95 | 35 | 3 | 29 | 0 | | CCCCCCCCCCCCCCEEEHHCCCCCCCCCCCCCCCC |
| 0302_0966 | | | | | | | |
| C12_ABH_ | 96 | 38 | 6 | 10.5 | 0 | | CCCCCCCCCCCCCCCCCCCCCCCCHHHHCCCCCCCC |
| 0101_0561 | | | | | | | CC |
| C12_HEB_ | 97 | 32 | 2 | 37.5 | 0 | putative ATP-binding | CHHCHHHHHHHHHHHHHHHHHCCCCCEEEECC |
| 0103_0130 | | | | | | protein fragment | |
| C11_ABH_ | 98 | 23 | 1 | 17.4 | 0 | | CCCCCCCCCCCCHHHHHHHHHCC |
| 0202_0784 | | | | | | | |
| C13_ABH_ | 99 | 8 | 0 | 12.5 | 0 | | CCCCCCCCC |
| 0101_0642 | | | | | | | |
| C12_HEB_ | 100 | 37 | 4 | 13.5 | 0 | | CCCCCCCCCCCCCCCCCCCCCCCCCCCCCHHHHHHC |
| 0202_0228 | | | | | | | C |
| C10_ABP_ | 101 | 31 | 2 | 22.6 | 1 | | CCCCCCCCCCCCCCCEEECCCCCCCCCCCCC |
| 0104_0034 | | | | | | | |
| C10_A43_ | 102 | 9 | 0 | 33.3 | 0 | | CCEECCCCC |
| 0202_0296 | | | | | | | |
| C10_ABH_ | 103 | 15 | 3 | 26.7 | 0 | | CCCCCCCHHHHHHCC |
| 0101_0546 | | | | | | | |
| C10_ABH_ | 104 | 57 | 8 | 17.5 | 3 | | CCCCCCCCCCCCCCCCCCCCCCCCCCCEEEECCCCC |
| 0102_0034 | | | | | | | CCCCCCCCCCCCCCCCCCCC |
| C11_HBM_ | 105 | 12 | 3 | 25 | 0 | | CCCEEEECCCCC |
| 0103_0350 | | | | | | | |
| M52_ABH_ | 106 | 60 | 4 | 35 | 0 | VF1 protein | CCCCCCCCCCCCCCCCCCEEECCCCCCCCEEEEEEEE |
| 0103_1436 | | | | | | fragment [Foot-and- | CCCCCCCCCCCCCCCCEEEEECC |
| | | | | | | mouth disease virus - | |
| | | | | | | type O] | |
| C12_HBM_ | 107 | 42 | 6 | 21.4 | 0 | | CCCCCCCCCHHHHHHHHHHHHHHHHHCCCCCCCCCCC |
| 0204_0525 | | | | | | | CCCCC |
| C11_HBM_ | 108 | 12 | 3 | 25.0 | 0 | | CCCEEEECCCCC |
| 0103_0350 | | | | | | | |
| C12_A43_ | 109 | 87 | 3 | 27.6 | 1 | | CCCCCHHHHHHHHHHHHHHCCCCCCCHHHHCCCCCCC |
| 0101_0234 | | | | | | | CEECCCCCCCCCCCCCCCCCCCCCCCCCCCCCCCCCC |
| | | | | | | | CCCCCHHHHHCC |
| C12_ABP_ | 110 | 52 | −1 | 44.2 | 0 | GntR family protein | CCCCCCEECCCCCEEECCCHHHHHHHHHHHHHHHHHH |
| 0102_0162 | | | | | | fragment- | HHHHHHHHHCCCCCC |
| | | | | | | [Porphyromonas | |
| | | | | | | gingivalis W83] | |
| C12_ABP_ | 111 | 34 | 2 | 20.6 | 0 | | CCCCCCCCEEEECCCCCCCCCCEECCCCCCCCCC |
| 0102_0148 | | | | | | | |
| C12_HEK_ | 112 | 45 | 5 | 26.7 | 0 | | CCCCCCCHHHHHHHHHHHHCCEECCCCCCCCCCCCCC |
| 0104_0234 | | | | | | | CCCCCCCC |
| C11_HBM_ | 113 | 45 | −5 | 35.6 | 1 | transposase | CCCCCCCCCCCCHHHHHHHCCCCHHHHHHHHHCCCHH |
| 0203_0575 | | | | | | fragment (ISH51) | HHHHHCCC |
| | | | | | | [Haloferax volcanii | |
| | | | | | | DS2] | |
| M52_ABH_ | 114 | 29 | 2 | 31.0 | 0 | envelope protein | CCCCCCCCHHHHHCCCCEEEEEEHHHCCC |
| 0103_1419 | | | | | | fragment [Dengue | |
| | | | | | | virus 1] | |
| M52_ABH_ | 115 | 48 | 4 | 39.6 | 0 | hemagglutinin | CCCCCCHHHHHHHHCCCCCEEEEEEEEECCCCCCEEE |
| 0102_1365 | | | | | | fragment [Measles | EECCCCCCCCC |
| | | | | | | virus] | |
| M52_ABH_ | 116 | 45 | 3 | 22.2 | 2 | | CCCCCCCCCEECCCEEEEEEEEEECCEEEEECCHHHH |
| 0104_1468 | | | | | | | HHHHCCCC |
| M52_ABH_ | 117 | 47 | 3 | 29.8 | 0 | VP3 fragment | CCCEEEECCCCCCCEEEEEECCCCCCCCCCCCCCCCC |
| 0104_1494 | | | | | | [Adeno-associated | CCCCCCCCCC |
| | | | | | | virus - 2] | |
| M52_ABH_ | 118 | 58 | 5 | 37.9 | 0 | Chain A, Sindbis | CCCCCCEEEEECCCCEEEEECCCCCEEEEEEEECCEE |
| 0103_1441 | | | | | | Virus Capsid | EEECEEEEECCHHHHHCCCCC |
| | | | | | | protein fragment | |
| M52_ABH_ | 119 | 37 | 6 | 24.3 | 1 | nonstructural | CCCCCCCHHHHHHHHCCCEEEEECCCCHHHHHHHHCC |
| 0102_1382 | | | | | | protein 3 | |
| | | | | | | fragment | |
| | | | | | | [Dengue virus 1] | |

TABLE 9
Summary of CD spectral analysis

| | NaF Buffer | NaF Buffer | SDS Micelles | SDS Micelles |
|---|---|---|---|---|
| Peptide | pH 4.5 | pH 7.2 | pH 4.5 | pH 7.2 |
| T08_HBM_0103_0031 | Random coil/Beta- | Helical | Helical | Helical |
| | turn with some | | | |
| | helicity | | | |
| T08_HBM_0104_0084 | Random coil and | Partially helical | Helical | Helical |
| | Beta-turn | | | |
| T09_HBM_0103_0167 | Random coil | Coil with Beta-turn | Random coil | Coil with Beta-turn |
| PYCJX-0901 | Random coil/Beta- | Beta-turn and some | Strong helix | Strong helix |
| | turn | helix | | |
| C20_ABH_0304_1746 | Predominantly | Strong Beta-turn | Predominantly | Strong Beta-turn |
| | Beta-turn | | Beta-turn | |
| C20_ABH_0404_1869 | Random coil | Beta-turn | Increased Helicity | Increased Helicity |
| TAT | Random coil/ | Strong poly-Pro | Random coil/ | Strong poly-Pro |
| | unstructured | helix | unstructured | helix |
| Penetratin | Unstructured | Random coil and | Increased Helicity | Increased Helicity |
| | | Beta-turn | | |
| Transportan | Weakly helical | Predominantly | Strong helix | Strong helix |
| | | helical | | |

Data presented in Table 8 and Table 9 hereof demonstrate that the screening method of the present invention isolates CPPs having novel structural properties compared to known CPPs, especially those that are reference CPPs used in the art such as HIV-1 TAT, transportan and penetratin. In particular, peptides isolated using the biotin ligase endosomal trap methodology described herein display unique and different conformational characteristics at different pH and in the presence of SDS micelles, and do not generally conform to the canonical helical secondary structure paradigm for CPPs.

### Example 16

### Development of a Split GFP Complementation Assay

This example demonstrates reduction to practice of a split GFP complementation assay for validating CPP functionality by: (i) detecting CPP-cargo-GFP 11 fusion polypeptide uptake into cells by determining fluorescence of the reconstituted GFP; and/or (ii) determining the ability of the CPP to modulate escape of a linked cargo protein from the endosome of the cell.

A split GFP assay, wherein a functional green fluorescent protein (GFP) or enhanced green fluorescent protein (EGFP) or AcGFP or TurboGFP is reconstituted in a manner that is dependent on CPP-mediated uptake into the cell, from a first moiety comprising a GFP 11 tag (SEQ ID NO: 81) fused to a test CPP and, optionally a scaffold protein, and a second moiety comprising a GFP 1-10 detector (SEQ ID NO: 86). In general, the GFP 1-10 is expressed in the cytoplasm of the cells and the GFP 11-test CPP peptide is contacted with the cells for reconstitution to occur in a CPP-dependent manner. Reconstituted GFP is detected by fluorescence-activated cell sorting (FACS) or fluorescence microscopy or live confocal microscopy or a combination thereof.

In the experiments reported herein for development of the split GFP complementation assay, CHO-K1 cells or HCC-827 cells were transfected with GFP1-10-encoding constructs and GFP11 fusion protein-encoding constructs, or transfected cells expressing GFP 1-10 which are then contacted with GFP 11 fusion protein. The inventors realized that, in practical applications for CPP screening, the protocols would be modified to employ transfected cells expressing GFP 1-10 which are then contacted with a GFP 11 fusion protein.

In the experiments reported herein for development of the split GFP complementation assay, reconstitution of GFP activity was evaluated by fluorescence microscopy. For fluorescence microscopy in test assays, cells were seeded into chamber slides having a charged surface at 5-7.5×10⁴ cells/well in 250 uL of media lacking antibiotic, and left to settle and adhere overnight. Following adherence, recombinant GFP11 fusion protein was added by removing 60 μL media from the wells and adding an approximately equivalent volume of 40 μM working stock solution of protein. Following a further overnight incubation period, media were removed from the cells gently such as using a pipette, and the cells were fixed or permeabilized using Image-iT Fix-Perm kit (Molecular Probes, Life Tech) according to the manufacturer's instructions. Slides were washed and blocked using BSA in DPBS, and fluorescence was visualized by incubating the cells in the presence of ActinRed 555 Ready Probes Reagent, then washed, stained using DAPI/PBS, and washed, flicked dry, and visualised by fluorescence microscopy.

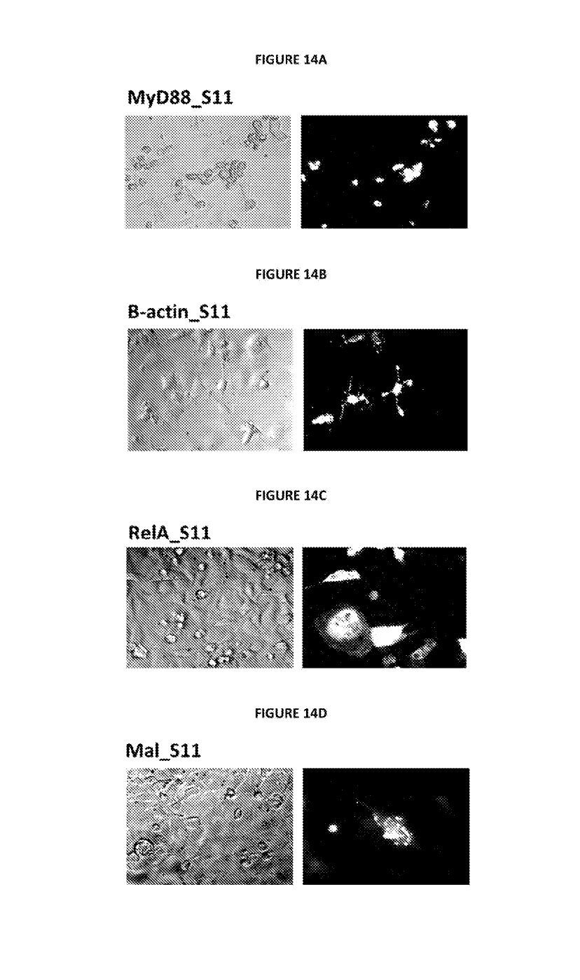

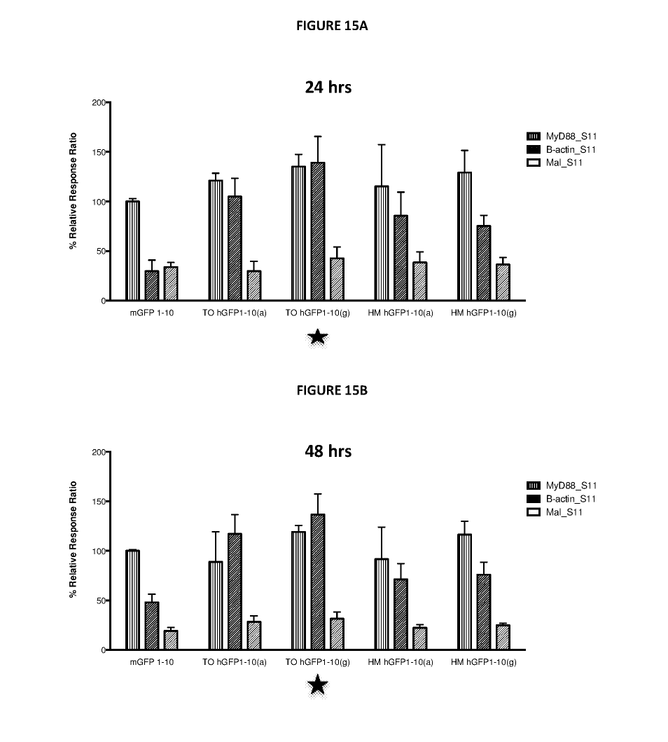

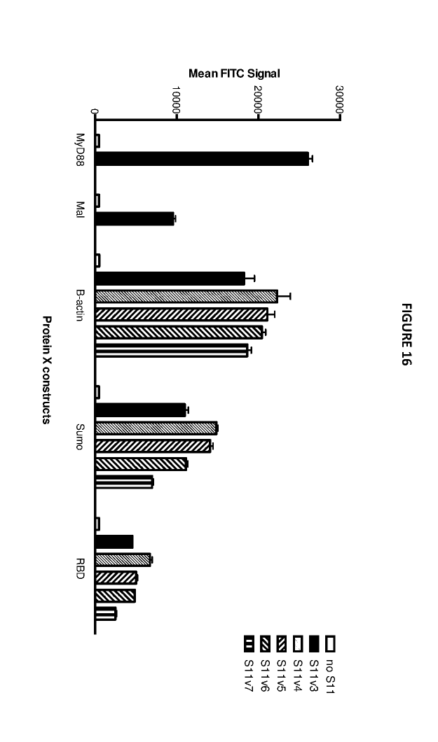

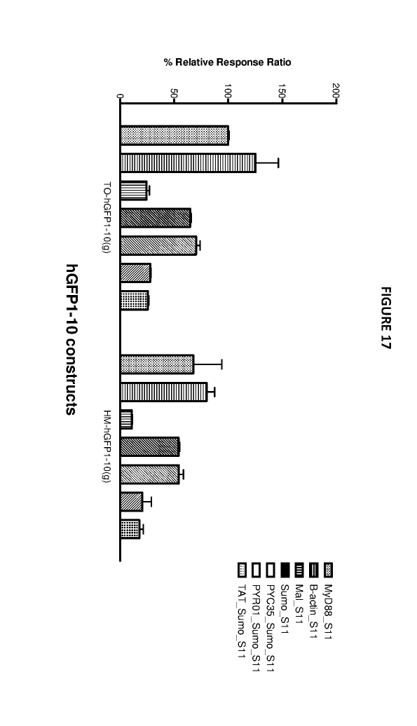

In one set of experiments, the inventors tested the effect of a scaffold moiety on reconstitution of GFP activity in a functional assay of the invention employing constructs that separately encode GFP 1-10 and GFP 11 fragments. Data presented in FIG. 13 indicate that transient transfection of HEK293 cells with constructs expressing mGFP1-10 and GFP 11 does not produce detectable levels of GFP fluorescence, however the addition of a scaffold-encoding nucleic acid to the GFP11-encoding construct improves reconstitution of functional GFP. Data presented in FIG. 14 hereof demonstrate that:
1. co-transfection of cells with constructs for GFP 1-10 and MyD88-GFP 11 produces dense pockets of reconstituted intracellular GFP mainly in rounded cells;
2. co-transfection of cells with constructs for GFP 1-10 and β-actin-GFP 11 produces diffuse localization of split GFP labelling throughout the cytoplasm, concentrated at dendritic features;
3. co-transfection of cells with constructs for GFP 1-10 and RelA-GFP 11 produces diffuse localization of split GFP throughout cytoplasm and sometimes excluded from nucleus; and
4. co-transfection of cells with constructs for GFP 1-10 and Mal-GFP 11 produces split GFP expression that is diffuse throughout cytoplasm and concentrated in multiple small foci.

Cellular viability was shown to be higher for cells expressing Mal-GFP 11 fusions or β-actin-GFP 11 fusions, whereas expression of MyD88-GFP 11 fusions or RelA-GFP 11 fusions reduced cellular viability. Accordingly, the inventors considered that a preferred split GFP complementation assay protocol for validating CPP activity would employ cells transfected to express GFP 1-10 which are then contacted with recombinant CPP-Mal-GFP 11 fusion protein or recombinant CPP-β-actin-GFP 11 fusion protein or recombinant Mal-CPP-GFP 11 fusion protein or recombinant β-actin-CPP-GFP 11 fusion protein or recombinant Mal-GFP11-CPP fusion protein or recombinant β-actin-GFP11-CPP fusion protein.

Data provided in FIG. 15 demonstrate that human codon optimization of GFP, by substituting a mutant nucleotide A of the commercially-available GFP 1-10 for G at the appropriate position to produce a human-optimized and corrected amino acid sequence (herein “hGFP1-10(g)”), improves the reconstituted GFP signal in human cells from reconstituted GFP 11 and GFP 1-10 fragments. The data also indicate that higher levels of GFP reconstitution occur when the codon-optimized GFP 1-10 is expressed from a pcDNA4/TO vector in human cells (“hGFP1-10(g)/TO”). Accordingly, the inventors considered that a preferred split GFP complementation assay protocol for validating CPP activity would employ cells transfected to express hGFP1-10(g) by virtue of being transfected with vector hGFP1-10(g)/TO, and contacting those cells with recombinant CPP-Mal-GFP 11 fusion protein or recombinant CPP-β-actin-GFP 11 fusion protein or recombinant Mal-CPP-GFP 11 fusion protein or recombinant β-actin-CPP-GFP 11 fusion protein or recombinant Mal-GFP11-CPP fusion protein or recombinant β-actin-GFP11-CPP fusion protein. More preferably, the cells are contacted with recombinant CPP-Mal-GFP 11 fusion protein or recombinant Mal-CPP-GFP 11 fusion protein or recombinant Mal-GFP11-CPP fusion protein to achieve elevated reconstitution of functional GFP with enhanced cell viability.

The inventors have also examined the effect of placing a linker between the Mal or β-actin scaffold and the GFP 11 moiety of the fusion protein. The inventors tested the effect of nucleic acids encoding a 16-mer amino acid sequence consisting of GSSGGSSGGSSGGSSG (S11v4), an 18-mer amino acid sequence consisting of GGTGGSGGAGGTGGSGGA (S11v5), a 14-mer amino acid sequence consisting of GTTGGTTGGGTGGS (S11v6), or a 10-mer amino acid sequence consisting of APAPAPAPAP (S11v7), each in the context of a construct encoding a MyD88-GFP 11 fusion, Mal-GFP 11 fusion, a β-actin-GFP 11 fusion, a Sumo-GFP 11 fusion, or a receptor binding domain (RBD)-GFP 11 fusion. Average fluorescence for each construct is shown in FIG. 16. Data provided in FIG. 16 indicate that, for the MyD88-GFP11 fusion protein-encoding constructs or Mal-GFP11 fusion protein-encoding constructs, it is preferable not to employ a linker to obtain optimum reconstitution of GFP, whereas for recombinant β-actin-GFP11 fusion protein-encoding constructs or Sumo-GFP11 fusion protein-encoding constructs or RBD-GFP11 fusion protein-encoding constructs, a linker having a length of up to 18 residues in length may be tolerated with little or no adverse affect on reconstitution of GFP. Accordingly, the inventors considered that a preferred split GFP complementation assay protocol for validating CPP activity would employ cells transfected to express hGFP1-10(g) by virtue of being transfected with vector hGFP1-10(g)/TO, and contacting those cells with either a linker-less recombinant CPP-Mal-GFP 11 or Mal-CPP-GFP 11 or Mal-GFP11-CPP fusion proteins, or alternatively, with recombinant CPP-β-actin-GFP 11 or β-actin-CPP-GFP 11 or β-actin-GFP11-CPP fusion proteins with or without linkers of up to about 18 residues in length.

The inventors have also considered the effect of cargo protein on reconstitution of split GFP activity in isolated HEK-293 cells expressing GFP 11+GFP 1-10 fragments. HEK-293 cells were transfected with GFP 1-10 vectors pcDNA4/TO vector [TO hGFP1-10(a)] or pcDNA4/HM vector [HM hGFP1-10(a)], and recombinant GFP 11-encoding constructs were added to the cells, and fluorescence activity was determined as a normalized value relative to fluorescence obtained for transfections employing MyD88-GFP11 and mGFP1-10 constructs. Data presented in FIG. 17 indicate that a cargo peptide can modulate reconstitution of split GFP activity in isolated HEK-293 cells expressing GFP 11+GFP 1-10 fragments, independent of cell-penetrating activity of the peptide. These data suggest that there is an advantage of performing in vitro complementation to test the effect of specific cargo fusion peptides on reconstitution of split GFP activity in vitro.

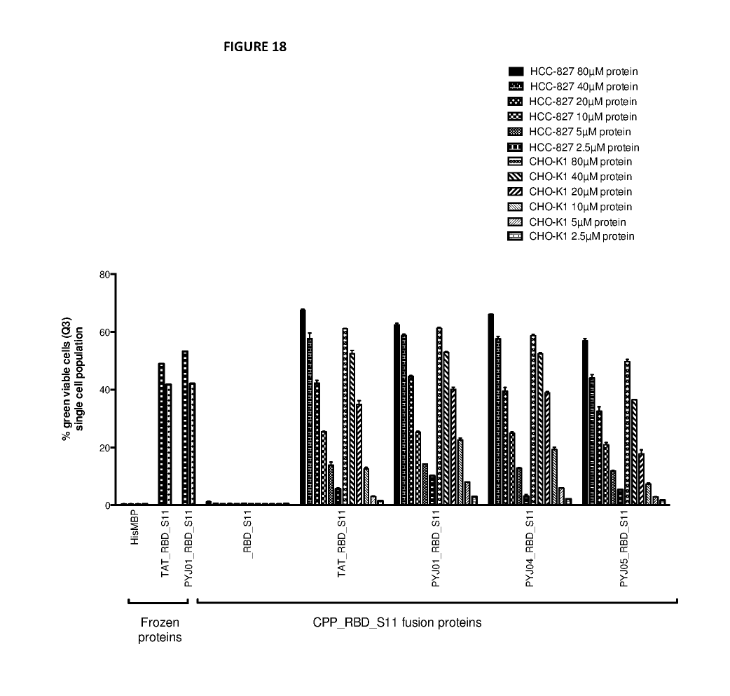

The inventors have also shown that reconstitution of split GFP activity in cells expressing GFP 11+GFP 1-10 fragments detects uptake of CPP-cargo-GFP 11 fusion polypeptides into different cell lines. The inventors have determined the percentages of GFP-positive cells in total live cell populations, normalized for transfection efficiency as determined in independent transfections of each cell line with pcDNA3-eGFP. Fluorescence was determined on HCC-827 (high receptor expression) and CHO-K1 (negative receptor expression) cells that had been transiently-transfected with hGFP1-10(g)/TO and then contacted with 2.5-80 μM recombinant fusion protein comprising a CPP and a receptor binding domain (RBD) cargo protein and GFP 11. Split GFP complementation was detected by measuring GFP fluorescence using flow cytometry, gating on the live cell population. Data presented in FIG. 18 indicate that the fluorescence signal was dose-responsive for each construct tested, and obtainable for fresh and frozen protein samples.

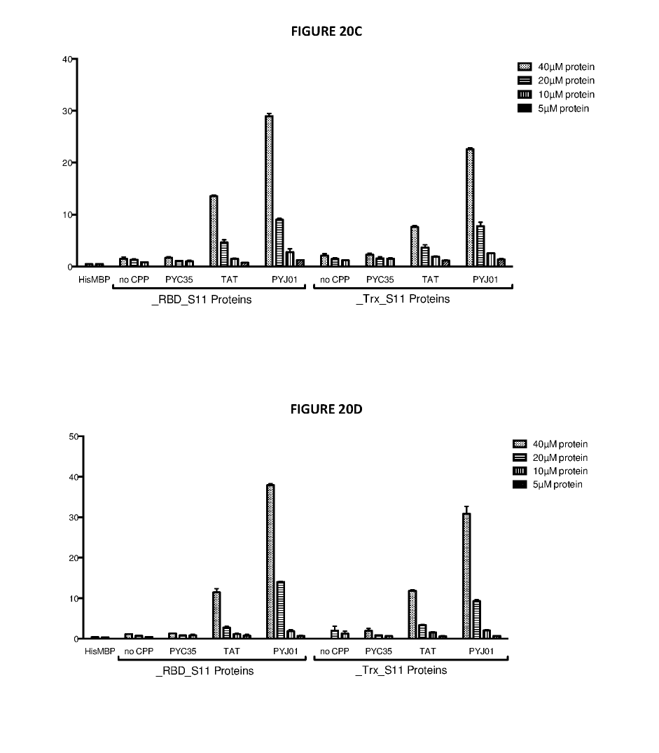

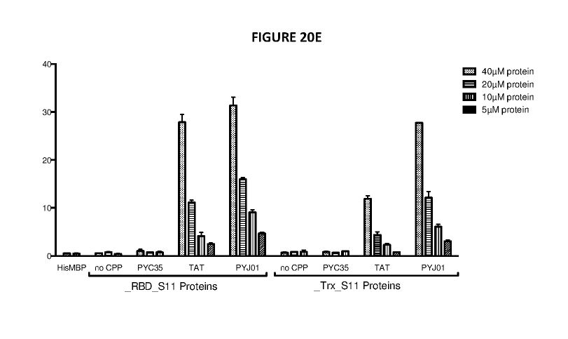

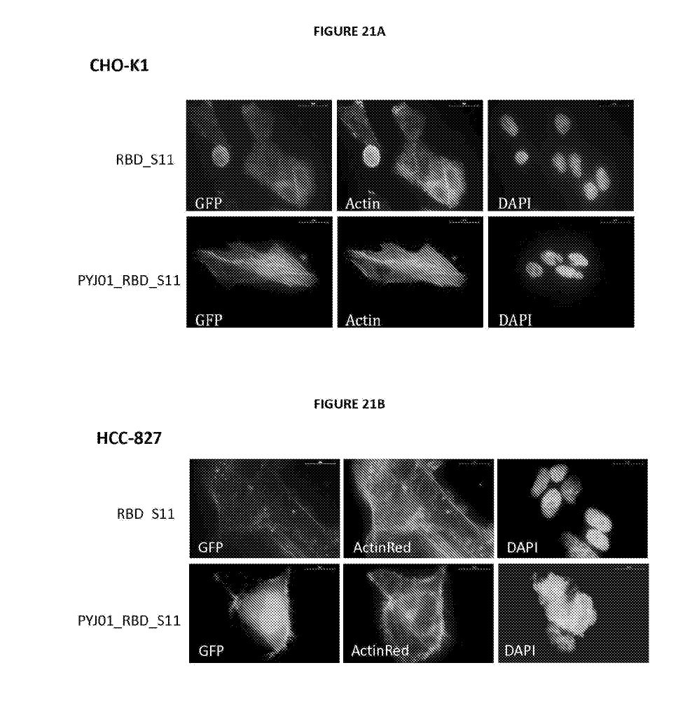

The inventors have also shown that the split GFP complementation assay of the invention is effective for validating or testing CPP-mediated uptake of GFP 11 and reconstitution of functional GFP activity in different cell lines, including CHO-K1 cells (adherent, rodent, negative for receptor expression); HCC-827 cells (adherent, human, strongly positive for receptor expression); HEK293 cells (adherent, human, moderate/low positive for receptor expression); HEK293/GFP1-10 cells (adherent, human, moderate/low positive for receptor expression, monoclonal stable transformed with hGFP1-10(g)/TO); and K562 cells (non-adherent, human, moderate/low positive for receptor expression). Each cells line was transiently transfected with hGFP1-10(g)/TO vector, to which was added a known CPP (TAT or PYJ01) linked to the RBD-GFP 11 cargo fusion polypeptide (RBD_S11) or thioredoxin-GFP 11 cargo fusion polypeptide. Negative controls were HisMBP or the cargo fusion polypeptides lacking a CPP or comprising the second cargo protein PYC35 in lieu of a CPP. Fluorescence was determined on 5-40 μM cellular protein, and the percentages of GFP-positive cells in each total live cell population were determined, normalized for transfection efficiency as determined in independent transfections of each cell line with pcDNA3-eGFP. Data presented in FIG. 20 indicate baseline fluorescence for assays that lacked CPP, with only validated CPPs TAT and PYJ01 providing reconstitution of GFP activity in the functional assay, in a dose-dependent manner and for each different cell lines tested.

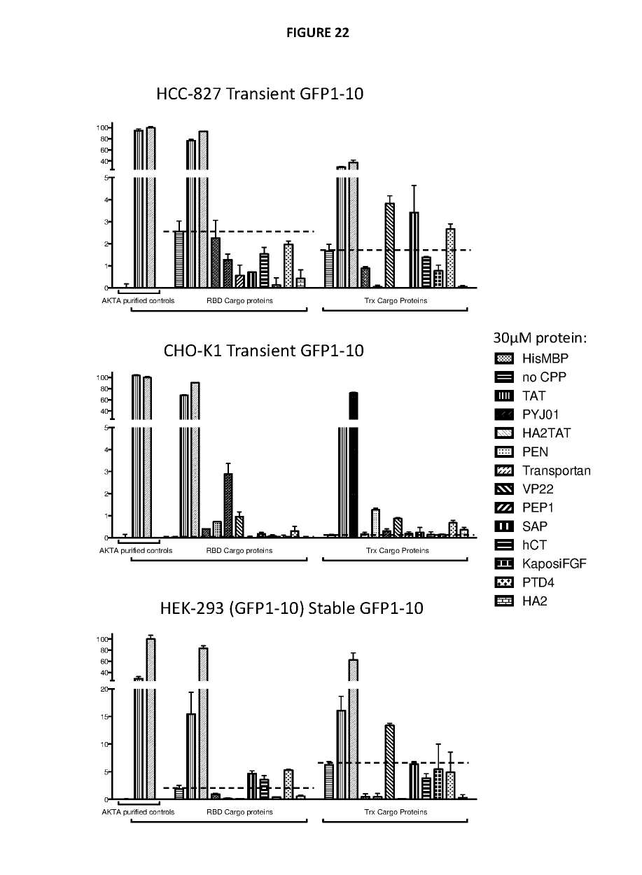

Data presented in FIG. 21 also confirm uptake of highly-purified, recombinant PYJ01-RBD-GFP11 fusion protein into CHO-K1 cells or HCC-827 cells that have been transiently transfected with hGFP1-10(g)/TO. Negative controls employed a RBD-GFP11 fusion polypeptide lacking the PYJ01 CPP. Similarly, data provided in FIG. 22 validate the split GFP complementation assay of the invention, by verifying the activities of several different known CPPs including TAT, PYJ01, VP22, SAP, and PTD4.

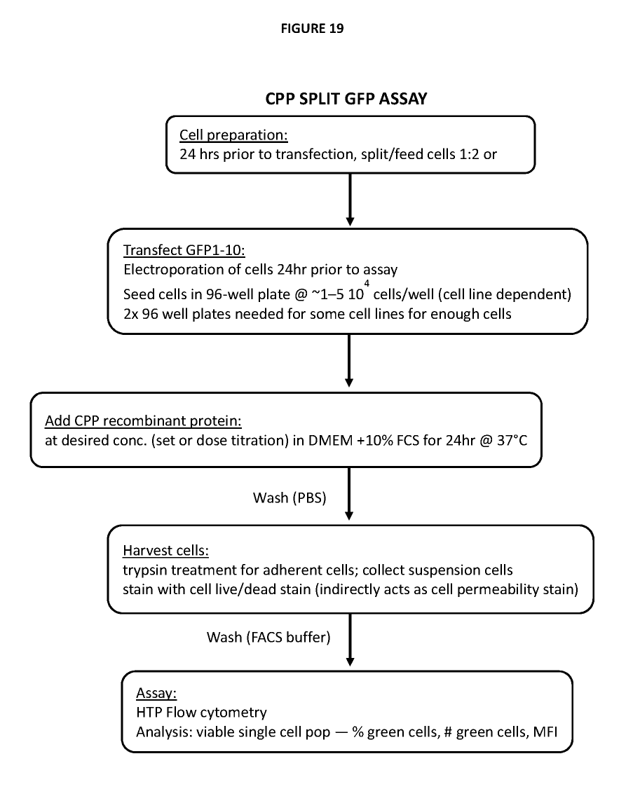

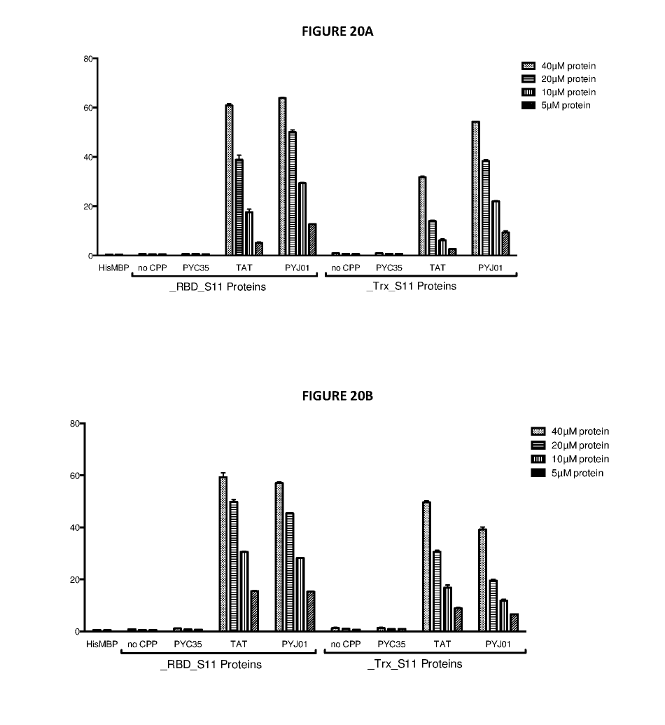

The data provided in this example thus demonstrate utility of the split GFP complementation assay for determining CPP activity. Proceeding on the basis of this finding, the inventors developed the work flow presented in FIG. 19 hereof. In accordance with this work flow, the split GFP complementation assay comprises expressing a test CPP as a fusion with GFP11 and, optionally, a scaffold such as Mal or β-actin, in human cells or non-human cells. The cells may be HCC-827 (high receptor expression) or CHO-K1 (negative receptor expression) cells that are transfected with human codon-optimized hGFP1-10(g)/TO construct. Split GFP complementation is then detected by measuring GFP fluorescence such as by flow cytometry, gating on the live cell population. The signal may be expressed as percent GFP-positive cells in the total live cell population, and normalized for the level of transfection efficiency as determined for an independent transfection of each cell line with a different construct such as pcDNA3-eGFP. An exemplary workflow of this preferred testing is provided by way of FIG. 19 hereof.

### Example 17

### Validation of CPP Activity of Peptides Using a Split GFP Complementation Assay

This example demonstrates validation of CPP functionality using a split GFP complementation assay developed as described herein above, and demonstrates that the CPPs identified by the inventive method described herein are structurally-distinct to the structures of known or so-called “canonical” CPPs, including transportan, VP22, human calcitonin (9-32), Ypep, PEP1, SAP, Kaposi FGF, and PTD4.

The split-GFP complementation assay as described herein was performed according to the following protocol. Briefly, HCC-827, CHO-K1, K562, H292 and Jurkat cells were cultured in RPMI (Gibco) plus Glutamax (Gibco) media supplemented with 10% FCS (Novagen) and 100 U/mL Pen/Strep (Gibco). H292 cells also received 10 mM HEPES (Gibco) in their media, and HCC-827 cells also received 10 mM HEPES (Gibco), 1 mM Sodium Pyruvate (Gibco) and NEAA (Gibco). HEK-293, A549, C3H10T1/2, NIH3T3, SW620 and HEK-293 cells expressing GFP1-10 were cultured in DMEM (Gibco)+Glutamax (Gibco) media supplemented with 10% FCS (Bovogen) and Pen/Strep (Gibco), with the stable cell lines also receiving 200 μM Zeocin (Invitrogen) as a selective agent.

Cells were prepared for electroporation by splitting cultures 1:2 (v/v) or 1:3 (v/v) one day beforehand (CHO-K1 cells), or by splitting cultures 1:8 (v/v) 4 days beforehand (HCC-827 cells) and replacing the media one day beforehand, or by splitting cultures 1:2 (v/v) one day prior to seeding (HEK-293 cells stably transformed to express GFP1-10). On the day of transfection, cells were harvested, pelleted by centrifugation, washed with PBS and pelleted by centrifugation again before resuspending in Buffer R (Invitrogen) at a concentration of 2×10⁷ cells/ml.

Cells were variously combined with equal volumes of column purified pcDNA4/TO_hGFP1-10 g DNA (200 μg/mL) in Buffer R (Invitrogen), resulting in a mixture consisting of 1×10⁷ cells/mL and 100 μg/mL DNA. Using 100 μL Neon Transfection system (Invitrogen) transfection tips, 100 μL of the cell/DNA mixture was mixed, withdrawn and transfected using one of three sets of transfection conditions: 1450V, 20 ms, 1 pulse (HCC-827 and HEK-293); 1230V, 30 ms, 2 pulses (A549); or 1620V, 10 ms, 3 pulses (all other cell lines). Transfected cells were then diluted in antibiotic-free versions of their culture media and seeded 75 μL per well in flat-bottomed (U-bottomed for suspension cells) 96 well plates at densities ranging from 7,500 to 30,000 cells per well. GFP1-10 stable HEK-293 cells were seeded at 5,000 cells/well. Plates were seeded in duplicate for all cell lines except CHO-K1.

Plates were incubated for 16-24 hours at 37° C., 5% CO₂, and then GFP11 fusion protein (25 μL per well, diluted in filter sterile pH 7.4 PBS) was added with gentle oscillation by hand. Plates were returned to the incubator for a further 20-24 hours at 37° C., 5% CO₂. To prepare plates for flow cytometry, they were washed with PBS, incubated in the presence of trypsin, quenched, resuspended and transferred to FACS plates, prior to a further wash with cold PBS. Cells were stained with Violet Live/Dead stain (diluted 1:1000 (v/v) in PBS comprising 1% FCS), 50 μL per well, and incubated at 4° C. for 30 minutes, and protected from light. Plates were then washed twice with cold PBS comprising 1% FCS before resuspending each well in 100 μL cold PBS comprising 1% FCS.

Flow cytometry was performed on a BD Fortessa flow cytometer with laser settings of FSC: 360V, SSC: 250V, Pacific Blue: 250V, FITC: 230V (for Jurkat cells, these settings were varied due to these cells being smaller). The maximum number of events to collect was set at 100,000 or 24 seconds of injection per well, whichever was reached first. Analysis of data was performed using FlowJo 10. For most cell lines, the single cell population was gated by plotting FSC-H vs FSC-W, excluding debris and doublets from the population. The single cell population was then plotted FITC-A vs Pacific Blue-A, with quadrant gates arranged such that the healthy GFP complemented cell population would appear in the bottom left hand corner, and this population would be as close to 0.5% (but not exceeding) of the single cell population in GFP1-10 transfected cells with HisMBP protein added.

Of 23 peptides tested from an initial screen of 38 peptides (SEQ ID Nos: 83-119) that were positive for uptake into cells as determined by their biotinylation in the endosome trap assay, nine peptides were also clearly-positive for CPP activity as determined by the Split-GFP complementation assay, and fourteen peptides were weakly-positive for CPP activity as determined by the Split-GFP complementation assay. This represent a high level of validation for the discriminatory ability of the primary screening by endosome trapping.

To determine whether or not the split GFP complementation assay of the invention has a discriminatory bias for structural features that are present in known or so-called “canonical” CPPs, the inventors compared the structural properties of CPPs that are positive for split GFP complementation activity to those peptides that are negative for split GFP complementation activity.

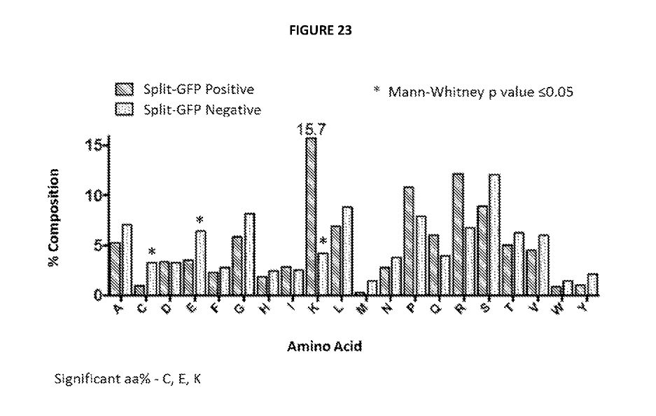

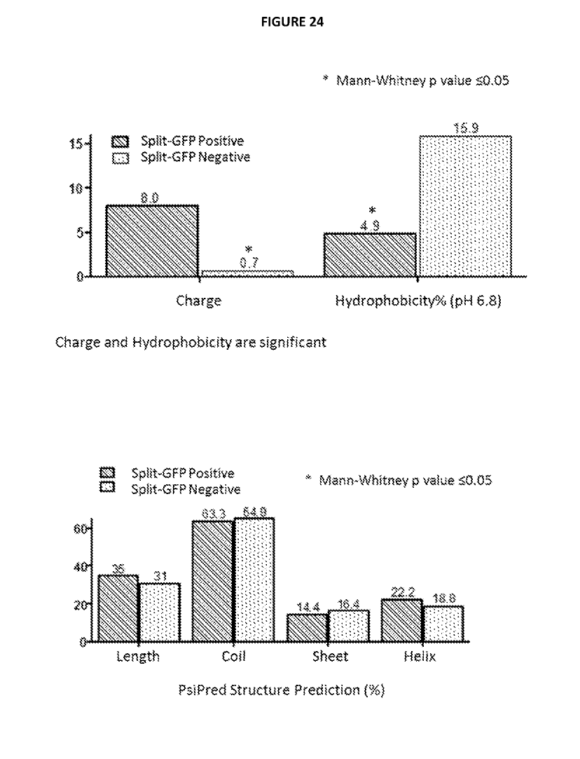

In one set of experiments, the inventors compared the amino acid compositions, net charges, hydrophobicities, lengths and predicted secondary structures of peptides that have been demonstrated herein as having an ability to transport GFP11 into the cytoplasm of cells as determined by reconstitution of functional GFP in the split GFP complementation assay of the present invention (“Split-GFP Positive”), to the amino acid compositions, net charges, hydrophobicities, lengths and predicted secondary structures of peptides that have been demonstrated herein not to have this functionality (“Split-GFP negative”). The data presented in FIG. 23 indicate that, in general the assay does not discriminate in terms of amino acid composition, however may select against peptides that have a higher composition of cysteine (C), glutamate (E) or lysine (K). Data presented in FIG. 24 indicate that there are significant differences in terms of net charge, hydrophobicity at pH 6.8, and that the split GFP complementation assay does not discriminate in terms of predicted structures for peptides, or peptide length. The inventors do not rule out the possibility that peptides that are Split-GFP negative are inherently less likely to exhibit CPP activity.

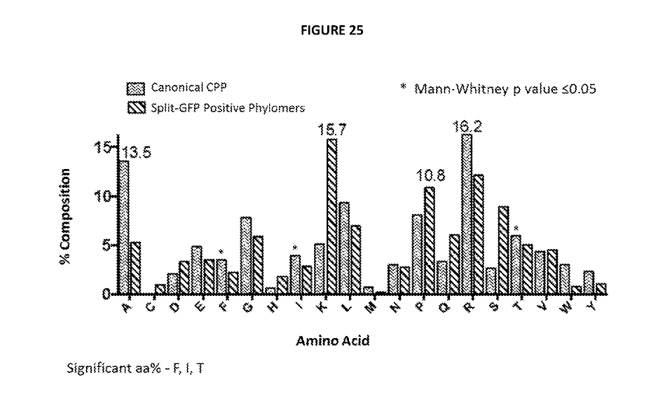

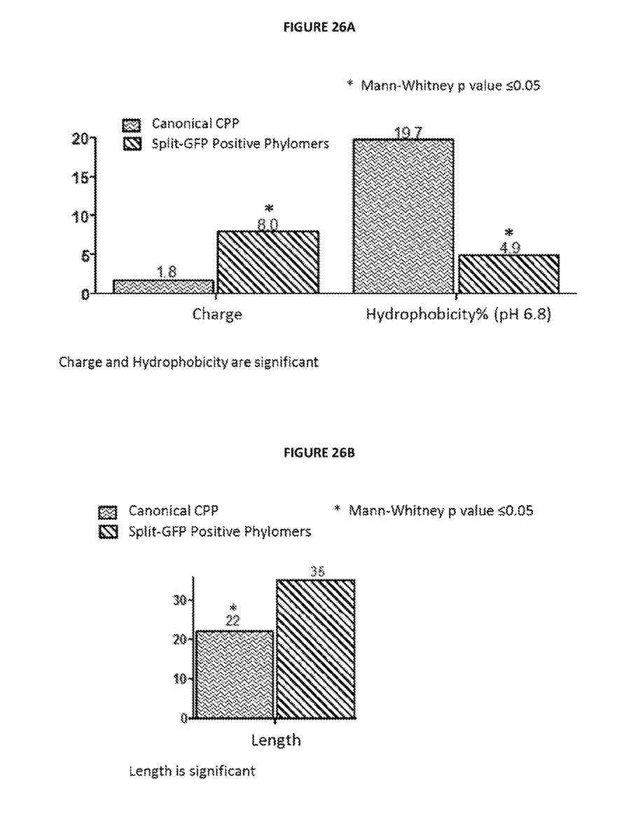

In a further set of experiments, the inventors sought to compare the amino acid compositions, net charges, hydrophobicities, lengths and predicted secondary structures of isolated CPPs of the present invention (SEQ ID Nos: 83-119) to the amino acid compositions, net charges, hydrophobicities, lengths and predicted secondary structures of known CPPs (“canonical CPP”). Data presented in FIG. 25 indicate that canonical CPPs have high levels of alanine (A) and arginine (R), whereas the CPPs of the present invention that are positive in both the endosomal biotinylation trap and split GFP complementation assay of the invention have high levels of lysine (K), arginine (R), and proline (P). Differences in levels of phenylalanine (F), isoleucine (I) and threonine (T) between the CPPs of the present invention and canonical CPPs are also highly-significant. Data presented in FIG. 26 also indicate significant differences in each of net charge, hydrophobicity and peptide length between canonical CPPs and CPPs of the present invention (SEQ ID Nos: 83-119), suggesting that the peptides of the present invention may represent a new structural class of non-canonical CPPs.

### Example 18

### Development of a Protein Inhibition Assay for Validating CPP Functionality

This example demonstrates reduction to practice of a protein inhibition assay for validating CPP functionality by: (i) detecting apoptosis and reduced viability of cells expressing a fusion polypeptide comprising a Bouganin polypeptide and a CPP, and optionally a scaffold protein moiety, wherein transport of the bouganin to the cell is mediated by the CPP.

The inventors produced a range of different nucleic acid constructs to perform this assay, which encode the fusion proteins set forth in SEQ ID Nos: 120-132 hereof as follows:
1. A His-bouganin fusion protein construct (SEQ ID NO: 120), comprising a sequence encoding bouganin, and further comprising: (i) a sequence encoding a hexahistidine in-frame with and N-terminal to the sequence encoding bouganin; and (ii) a sequence encoding the sequence GSGATAGSAATGGATGGSTS in-frame with and C-terminal to the sequence encoding bouganin to facilitate and optional addition of a CPP sequence at a C-terminal portion thereof;
2. A His-Bouganin-LPETGG fusion protein construct (SEQ ID NO: 121), being similar to SEQ ID NO: 120 albeit wherein the sequence encoding GSGATAGSAATGGATGGSTS is replaced with a sequence encoding GGSGGTLPETGG in-frame with and C-terminal to the sequence encoding bouganin to facilitate sortase-mediated labelling of the fusion protein;
3. A His-Bouganin-RBD-LPETGG fusion protein construct (SEQ ID NO: 122), being similar to SEQ ID NO: 120 albeit wherein the sequence encoding GSGATAGSAATGGATGGSTS is replaced with a sequence encoding GGSGGTRBDGSSGGAGGAGGSLPETGG in-frame with and C-terminal to the sequence encoding bouganin to facilitate RBD receptor binding and sortase-mediated labelling of the fusion protein;
4. A His-Bouganin-RBD (Generation 1) fusion protein construct (SEQ ID NO: 123), being similar to SEQ ID NO: 120 albeit wherein the sequence encoding GSGATAGSAATGGATGGSTS is replaced with a sequence encoding GGSGGTGGSRBDGTSGGTGGS in-frame with and C-terminal to the sequence encoding bouganin to facilitate RBD receptor binding and optional addition of a CPP sequence at a C-terminal portion thereof;
5. A His-Bouganin-RBD (Generation 2) fusion protein construct (SEQ ID NO: 124), being similar to SEQ ID NO: 120 albeit wherein the sequence encoding GSGATAGSAATGGATGGSTS is replaced with a sequence encoding GSGTGSATSGSLAGSGATAGTGSGGSRBDGTGTASGGAGTGSGTS in-frame with and C-terminal to the sequence encoding bouganin to facilitate RBD receptor binding and optional addition of a CPP sequence at a C-terminal portion thereof;
6. A His-RBD-Bouganin fusion protein (Generation 1) construct (SEQ ID NO: 125), being similar to SEQ ID NO: 120 albeit wherein a sequence encoding GSRBDGTGSGTGSATSGSLAGSGATAGTGSG is inserted downstream of the sequence encoding hexahistidine and upstream of sequence encoding bouganin to produce an in-frame Hexahistidine-RBD-bouganin protein to facilitate RBD receptor binding and optional addition of a CPP sequence at a C-terminal portion thereof;
7. A His-RBD-Bouganin fusion protein (Generation 2) construct (SEQ ID NO: 126), being similar to SEQ ID NO: 125 albeit lacking the sequence encoding TGSATSGSLAGSGATAGTGSG immediately upstream of sequence encoding bouganin, and such that there remains capacity for an optional addition of a CPP sequence at a C-terminal portion thereof;
8. A bouganin-His fusion protein construct (SEQ ID NO: 127) comprising sequence encoding the linker GGTSASGGAGTGSG upstream and in-frame with sequence encoding bouganin to facilitate optional insertion of sequence encoding a CPP after residue 2 of the fusion protein, and a sequence encoding hexahistidine downstream and in-frame with sequence encoding bouganin;
9. A RBD-Bouganin-His (Generation 1) fusion protein construct (SEQ ID NO: 128), being similar to SEQ ID NO: 127 albeit wherein the sequence encoding ASGGAGTGSG is replaced with sequence encoding GGGRBDGSSGGSSGGT to facilitate sortase conjugation and RBD receptor binding;
10. A RBD-Bouganin-His (Generation 2) fusion protein construct (SEQ ID NO: 129), being similar to SEQ ID NO: 127 albeit wherein the sequence encoding GGTSASGGAGTGSG is replaced with sequence encoding GGTGGSRBDGGSGGTGGS to facilitate RBD receptor binding without disrupting the capacity to introduce sequence encoding a CPP after residue 2 of the fusion protein;
11. A RBD-Bouganin-His (Generation 3) fusion protein construct (SEQ ID NO: 130), being similar to SEQ ID NO: 127 albeit wherein the sequence encoding the N-terminal sequence MGGTSASGGAGTGSG is replaced with sequence encoding the N-terminal sequence RBDGTGSGTGSATSGSLAGSGATAGTGSG to facilitate RBD receptor binding;
12. A RBD-Bouganin-His (Generation 4) fusion protein construct (SEQ ID NO: 131), being similar to SEQ ID NO: 130 albeit further comprising a sequence encoding MGGTSASGGAGTGSGGS upstream of the RBD receptor binding domain to facilitate introduction of sequence encoding a CPP after residue 2 of the fusion protein; and
13. A Bouganin-RBD-His fusion protein construct (SEQ ID NO: 132), being similar to SEQ ID NO: 127 albeit wherein the sequence encoding the N-terminal sequence MGGTSASGGAGTGSG is replaced with sequence encoding the N-terminal sequence MGGTSGSGATAGSAATGGATGGS to facilitate introduction of sequence encoding a CPP after residue 2 of the fusion protein, and wherein a sequence encoding a linker and RBD-receptor binding domain is positioned upstream of the C-terminal linker sequence GGS and hexahistidine-encoding sequence.

To test the ability of CPPs to translocate a bouganin protein into cells and reduce cell viability and/or induce apoptosis, CPPs including those listed in Table 9 hereof were clones into vector encoding the protein construct set forth in SEQ ID NO: 123 such that the CPPs were expressed in-frame with the encoded His-Bouganin-RBD fusion protein. Nucleic acid encoding the peptide designated T08_HBM_0104_0084 in Table 9 was also introduced independently into vectors encoding the fusion protein constructs set forth in each of SEQ ID Nos: 15-127 and 131 such that the CPPs were expressed in-frame with and at a C-terminal portion of the encoded His-RBD-Bouganin fusion protein (SEQ ID Nos: 125-126), or alternatively, such that the CPPs were expressed in-frame with and at an N-terminal portion of Bouganin-His fusion protein (SEQ ID NO: 127) or RBD-Bouganin-His (SEQ ID NO: 131) fusion protein i.e., after residue 2 of the fusion proteins.

For expression of Bouganin fusion protein constructs, bacterial cell cultures were established in Luria Broth (LB) comprising 50 μg/ml kanamycin. Briefly, 1 ml of culture medium was added to the wells of a 96 deep-well plate and bacterial glycerol stock inoculum added, and cultures were incubated overnight at 30° C. with shaking at 250 r.p.m. Overnight cultures were then used to inoculate 1.8 L of the same medium, and 100 ml aliquots of the expression cultures were transferred to 250 ml flasks. Following culture of the cells, they were collected by centrifugation at 4000 r.p.m. for 15 mins, the media decanted, and 25 ml of chilled PBS was added to each cell pellet. The pellets were resuspended and transferred to 50 ml Falcon tubes. Cells were harvested by centrifugation as before, and the supernatants decanted and cell pellets frozen. Cells were then lysed by suspension in 2 ml of BugBuster MasterMix comprising protease inhibitors, and the lysates transferred to 24 well plates, centrifuged at 17,000×g for 15 mins (4° C.), and the supernatants retained. For purification of expressed hexahistidine-containing fusion proteins from the lysates, 0.5 ml Ni Sepharose resin columns in a 24-well plate were washed with 5 ml water, and equilibrated with 5 ml 20 mM sodium phosphate comprising 300 mM NaCl and 20 mM imidazole. The lysates were added to the Ni Sepharose resin columns, mixed thoroughly, and unbound material was allowed to flow through under gravity flow. The unbound samples were washed with 2 aliquots of 10 ml each of the same buffer i.e., 20 mM sodium phosphate comprising 300 mM NaCl and 20 mM imidazole. Bound hexahistidine-containing fusion proteins were eluted using 0.5 ml of 20 mM sodium phosphate comprising 300 mM NaCl and 500 mM imidazole. The eluted proteins were desalted 600 μl PhyTIps. The expressed fusion proteins (2 μl of each desalted sample) were analyzed by SDS-PAGE (12% TGX gels, BioRad) using Tris-glycine running buffer at 25 mA per gel for 50 min. For quantitation of protein, samples were passed through a 0.22 micron PVDF filter (Millipore), and quantitated using BCA protein assay.

Data (not shown) indicate that expression of Bouganin in cells inhibits protein expression in a dose-dependent manner. Whereas CPPs alone do not adversely affect protein expression, linkage of a CPP at the N-terminus or C-terminus of bouganin results in a significant reduction in protein synthesis over a 72 hour period, and the effect can be attributed to the activity of a CPP in mediating entry of bouganin to the cells.

1. A method of determining or identifying a peptide capable of translocating a membrane of a cell, the method comprising the steps:
(i) contacting host cells expressing a biotin ligase with a plurality of non-biotinylated members, wherein the members comprise scaffolds displaying fusion proteins, each of the fusion proteins comprising a candidate peptide moiety and a biotin ligase substrate domain, and wherein said contacting is for a time and under conditions sufficient for at least the displayed fusion proteins of members to enter the host cells;
(ii) incubating the host cells for a time and under conditions such that the biotin ligase substrate domain of the at least fusion proteins that have translocated a membrane of the host cell are enzymatically biotinylated by the expressed biotin ligase; and
(iii) determining or identifying a candidate peptide moiety that has translocated a membrane of the host cell by performing a process comprising:
(a) detecting the presence of a biotinylated fusion protein in a host cell or cell lysate or extract thereof, wherein the presence of a biotinylated fusion protein indicates that the candidate peptide moiety has translocated the cell membrane; and/or
(b) isolating at least a biotinylated fusion protein from a host cell or cell lysate or extract thereof; and/or
(c) recovering at least a biotinylated fusion protein from a host cell or cell lysate or extract thereof.

2. The method according to claim 1, wherein members further comprise an covalent link between the scaffold and the fusion protein, wherein the covalent link is cleavable by exposure to an environment within a cell or an intracellular compartment thereof.

3. The method according to claim 2, wherein the intracellular environment comprises a reducing environment of the cytoplasm of a cell.

4. The method according to claim 3, wherein the covalent link is a disulphide bond.

5. The method according to any one of claims 1 to 4, wherein members do not enter endosomes of the host cells.

6. The method according to any one of claims 1 to 4, wherein contacting at step (i) is for a time and under conditions sufficient for at least the displayed fusion proteins of members to enter the endosome of host cells, and wherein incubating at step (ii) is for a time and under conditions such that the biotin ligase substrate domain of the at least fusion proteins that have translocated the endosome of the host cells are enzymatically biotinylated by the expressed biotin ligase and wherein determining or identifying at step (iii) comprises determining or identifying a candidate peptide moiety at step (iii) that has translocated the endosome of the host.

7. The method according to claim 6, wherein members translocate the endosome of the hosts intact.

8. The method according to claim 6, wherein members further comprise an amino acid sequence between the scaffold and the fusion protein, wherein the sequence comprises an enzyme substrate site, and wherein said members are reacted with an enzyme that acts on said enzyme substrate site to cleave the scaffold from the fusion protein, and wherein the cleaved fusion protein enters the endosome of the host cells.

9. The method according to claim 8, wherein the cleaved fusion protein translocates the endosome of the host cells.

10. The method according to any one of claims 5 to 7, wherein the method comprises detecting and/or isolating and/or recovering a biotinylated member.

11. The method according to any one of claims 1 to 9, wherein the method comprises detecting and/or isolating and/or recovering a biotinylated fusion protein.

12. The method according to any one of claims 1 to 11, wherein the non-biotinylated members are non-biotinylated by virtue of being produced in cells having no endogenous biotin ligase activity.

13. The method according to claim 12 further comprising producing the non-biotinylated members in cells having no endogenous biotin ligase activity.

14. The method according to any one of claims 1 to 11, wherein the non-biotinylated members are non-biotinylated by virtue of being produced in cells having a biotin ligase that has a low affinity for the biotin ligase substrate domain.

15. The method according to claim 14 further comprising producing the non-biotinylated members in cells having a biotin ligase that has a low affinity for the biotin ligase substrate domain.

16. The method according to any one of claims 1 to 15, further comprising incubating the host cells after step (ii) and prior to step (iii) with an agent to inhibit the activity of the biotin ligase.

17. The method according to claim 16, wherein the agent comprises a pyrophosphate salt or adenosine 5′ monophosphate (AMP) salt.

18. The method according to claim 17, wherein the pyrophosphate salt is a colloidal metal pyrophosphate salt, disodium pyrophosphate salt, tetrasodium pyrophosphate salt, potassium pyrophosphate salt, calcium pyrophosphate salt or inositol pyrophosphate salt.

19. The method according to claim 17, wherein the AMP salt is a disodium salt, calcium salt or magnesium salt.

20. The method according to claim 16, wherein the agent comprises a chaotropic salt.

21. The method according to claim 16, wherein the agent comprises a biotin analogue capable of competing with the biotin ligase substrate domain for binding of the expressed biotin ligase.

22. The method according to claim 16, wherein the agent comprises ethylenediaminetetraacetic acid (EDTA).

23. The method according to claim 16, wherein the agent comprises acetonitrile.

24. The method according to any one of claims 1 to 23 further comprising treating the host cells at step (i) to remove members that are associated with the membrane of the host cells without disrupting the cell membranes.

25. The method according to claim 24, wherein treating the host cells comprises incubating the host cells with a protease for a time and under conditions sufficient to remove and/or inactivate extrinsic members to the host cells without disrupting the cell membrane.

26. The method according to claim 25, wherein the protease is trypsin or chymotrypsin or thermolysis or heparinase or subtilisin or proteinase K.

27. The method according to any one of claims 24 to 26, wherein treating the cell comprises washing the host cells for a time and under conditions sufficient to remove members that are associated with the membrane of the host cells.

28. The method according to any one of claims 1 to 28 further comprising fractionating the plurality of non-biotinylated members prior to step (i) to thereby obtain one or more pools of members each having a net positive or net negative or net neutral charge and then performing step (i) using the one or more pools of members.

29. The method according to claim 28, wherein fractionating the plurality of non-biotinylated members comprises performing ion exchange chromatography and recovering the one or more pools of members.

30. The method according to claim 29, wherein the ion exchange chromatography comprises use of an anion exchanger.

31. The method according to claim 29, wherein the ion exchange chromatography comprises use of a cation exchanger.

32. The method according to any one of claims 28 to 31, wherein the ion exchange chromatography is a batch process.

33. The method according to any one of claims 28 to 31, wherein the ion exchange chromatography is a moving bed process.

34. The method according to any one of claims 28 to 33, wherein a pool of members has an isoelectric point (pI) of 2 or 3 or 4 or 5 or 6 or 7 or 8 or 9 or 10 or 11 or 12, or a pI in the range of 2-10 or 2-9 or 2-8 or 2-7 or 2-6 or 2-5 or 2-4 or 2-3 or 3-10 or 4-10 or 5- 10 or 6-10 or 7-10 or 8-10 or 9-10 or 3-9 or 4-9 or 5-9 or 6-9 or 7-9 or 8-9 or 3-8 or 3-7 or 3-6 or 3-5 or 3-4 or 4-8 or 5-8 or 6-8 or 7-8 or 4-7 or 4-6 or 4-5 or 5-7 or 6-7 or 5-6.

35. The method according to any one of claims 1 to 34, wherein the biotin ligase expressed at step (i) is an endogenous biotin ligase of the host cells.

36. The method according to any one of claims 1 to 34, wherein the host cells express an endogenous biotin ligase that has a low affinity for the biotin ligase substrate domain and wherein the biotin ligase expressed at step (i) is a recombinant biotin ligase that has a high affinity for the biotin ligase substrate domain.

37. The method according to any one of claims 1 to 35, wherein the host cells lack endogenous biotin ligase activity, and wherein the biotin ligase expressed at step (i) is a recombinant biotin ligase.

38. The method according to claim 36 or 37, wherein the recombinant biotin ligase is encoded by a gene construct comprising a promoter operably connected to nucleic acid encoding the biotin ligase, and wherein the promoter confers constitutive expression of the biotin ligase on the host cells.

39. The method according to claim 36 or 37, wherein the recombinant biotin ligase is encoded by a gene construct comprising a promoter operably connected to nucleic acid encoding the biotin ligase, and wherein the promoter confers inducible expression of the biotin ligase on the host cells, and wherein said method further comprising growing the host cells at (i) under conditions sufficient to induce expression of the biotin ligase in the host cells.

40. The method according to any one of claims 36 to 39, wherein the method further comprises producing host cells that are stably or transiently transformed with a gene construct encoding the biotin ligase.

41. The method according to any one of claims 1 to 41, wherein the biotin ligase expressed at step (i) is encoded by the amino acid sequence set forth in SEQ ID NO: 2 or a variant thereof having an amino acid sequence that is at least 70% identical to SEQ ID NO: 2 and wherein said variant has biotin ligase activity.

42. The method according to claim 41, wherein the biotin ligase substrate domain comprises an amino acid sequence defined by: LX₁X₂IX₃X₄X₅X₆KX₇X₈X₉X₁₀ (SEQ ID NO: 3), where X₁ is any amino acid; X₂ is any amino acid other than L, V, I, W, F, Y; X₃ is F or L; X₄ is E or D; X₅ is A, G, S, or T; X₆ is Q or M; X₇ is I, M, or V; X₈ is E, L, V, Y, or I; X₉ is W, Y, V, F, L, or I; and X₁₀ is preferably R, H, or any amino acid other than D or E.

43. The method according to claim 42, wherein X₁ is N; X₂ is D; X₃ is F; X₄ is E; X₅ is A; X₆ is Q; X₇ is I; X₈ is E; X₉ is W; X₁₀ is H.

44. The method according to claim 42 or 43, wherein the biotin ligase substrate domain comprises the sequence GLNDIFEAQKIEWHE (SEQ ID NO: 4).

45. The method according to any one of claims 1 to 41, wherein the biotin ligase expressed at step (i) is encoded by the amino acid sequence set forth in SEQ ID NO: 5 or a variant thereof having an amino acid sequence that is at least 70% identical to the sequence of SEQ ID NO: 5 and wherein said variant has biotin ligase activity.

46. The method according to claim 45, wherein the biotin ligase substrate domain comprises the amino acid sequence TVVCIVEAMKLFIEI (SEQ ID NO: 6).

47. The method according to any one of claims 1 to 40, wherein the biotin ligase expressed at step (i) is encoded by the amino acid sequence set forth in SEQ ID NO: 7 or a variant thereof having an amino acid sequence that is at least 70% identical to the sequence of SEQ ID NO: 7 and wherein said variant has biotin ligase activity.

48. The method according to claim 47, wherein the biotin ligase substrate domain comprises the amino acid sequence DVIVVLEAMKMEHPI (SEQ ID NO: 8).

49. The method according to any one of claims 1 to 40, wherein the biotin ligase expressed at step (i) is encoded by the amino acid sequence set forth in SEQ ID NO: 9 or a variant thereof having an amino acid sequence that is at least 70% identical to the sequence of SEQ ID NO: 9 and wherein said variant has biotin ligase activity.

50. The method according to claim 49, wherein the biotin ligase substrate domain comprises the amino acid sequence QPVAVLSAMKMEMII (SEQ ID NO: 10).

51. The method according to any one of claim 41, 45, 47 or 49, wherein the biotin ligase substrate domain comprises the amino acid sequence DTLCIVEAMKMMNQI (SEQ ID NO: 13).

52. The method according to any one of claims 1 to 40, wherein the biotin ligase expressed at step (i) is encoded by the amino acid sequence set forth in SEQ ID NO: 14 or a variant thereof having an amino acid sequence that is at least 70% identical to the sequence of SEQ ID NO: 14 and wherein said variant has biotin ligase activity.

53. The method according to any one of claims 1 to 40, wherein the biotin ligase expressed at step (i) is encoded by the amino acid sequence set forth in SEQ ID NO: 15 or a variant thereof having an amino acid sequence that is at least 70% identical to the sequence of SEQ ID NO: 15 and wherein said variant has biotin ligase activity.

54. The method according to any one of claims 1 to 40, wherein the biotin ligase expressed at step (i) is encoded by the amino acid sequence set forth in SEQ ID NO: 16 or a variant thereof having an amino acid sequence that is at least 70% identical to the sequence of SEQ ID NO: 16 and wherein said variant has biotin ligase activity.

55. The method according to any one of claims 1 to 40, wherein the biotin ligase expressed at step (i) is encoded by the amino acid sequence set forth in SEQ ID NO: 17 or a variant thereof having an amino acid sequence that is at least 70% identical to the sequence of SEQ ID NO: 17 and wherein said variant has biotin ligase activity.

56. The method according to any one of claims 1 to 40, wherein the biotin ligase expressed at step (i) is encoded by the amino acid sequence set forth in SEQ ID NO: 18 or a variant thereof having an amino acid sequence that is at least 70% identical to the sequence of SEQ ID NO: 18 and wherein said variant has biotin ligase activity.

57. The method according to any one of claims 37 to 57, wherein the biotin ligase is fused to a polypeptide localisation signal capable of directing the biotin ligase to a particular subcellular location of the host cells.

58. The method according to claim 57, wherein the polypeptide localisation signal is a nuclear localisation signal.

59. The method according to claim 57, wherein the polypeptide localisation signal is a golgi localisation sequence.

60. The method according to claim 57, wherein the polypeptide localisation signal is a mitochondria localisation sequence.

61. The method according to claims 1 to 57, wherein the host cells are bacterial cells.

62. The method according to claims 1 to 60, wherein the host cells are eukaryotic cells.

63. The method according to claim 60, wherein the eukaryotic cells are plant cells.

64. The method according to claim 60, wherein the eukaryotic cells are mammalian cells.

65. The method according to claim 60, wherein the eukaryotic cells are primate cells.

66. The method according to claim 64, wherein the mammalian cells are murine cells.

67. The method according to claim 64, wherein mammalian cells are human cells.

68. The method according to claim 67, wherein the human cells are HEK293 cells.

69. The method according to any one of claims 1 to 68, wherein the scaffold is a bacteriophage.

70. The method according to claim 69, wherein the bacteriophage is produced in bacterial cells that do not express a biotin ligase.

71. The method according to claim 69, wherein the bacteriophage is produced in bacterial cells expressing a biotin ligase that biotinylates the biotin ligase substrate domain inefficiently and wherein said method further comprises isolating the non-biotinylated members from biotinylated members prior to step (i) to thereby provide the non-biotinylated members.

72. The method according to claim 69, wherein the bacteriophage is produced in bacterial cells expressing a biotin ligase, wherein said cells further comprise a polypeptide comprising a biotin ligase substrate domain, and wherein the cellular biotin ligase biotinylates the polypeptide in preference to the members to thereby provide the non-biotinylated members.

73. The method according to claim 72, wherein the polypeptide comprises a plurality of biotin ligase substrate domains to thereby provide preferential biotinylation of the polypeptide relative to the biotin ligase substrate domain of the fusion protein.

74. The method according to claim 73, wherein the polypeptide comprises three biotin ligase substrate domains.

75. The method according to claim 74 or 75, wherein the fusion protein has one biotin ligase substrate domain.

76. The method according to any one of claims 72 to 75, wherein the polypeptide further comprises a scaffold moiety.

77. The method according to claim 76, wherein the scaffold moiety is a small ubiquitin-related modifier peptide.

78. The method according to any one of claims 69 to 77, wherein the bacteriophage is a filamentous phage.

79. The method according to claim 78, wherein the filamentous phage comprises nucleic acid encoding the fusion protein operably linked to a nucleic acid sequence encoding a signal peptide that promotes translocation of the fusion protein across an inner membrane of a cell.

80. The method according to claim 79, wherein the encoded fusion protein is linked to a coat protein of the filamentous phage.

81. The method according to claim 80, wherein the coat protein is pIII or pVII or pVIII or pIX.

82. The method according to any one of claims 79 to 81, wherein the filamentous phage is M13.

83. The method according to any one of claims 79 to 82, wherein the signal peptide directs the fusion protein to the signal recognition particle (SRP) pathway.

84. The method according to claim 83, wherein the signal peptide is a DsbA signal peptide, a TorT signal peptide, or a TolB signal peptide or a Sfm signal peptide.

85. The method according to claim 84, wherein the signal peptide is a DsbA signal peptide and wherein the DsbA signal peptide comprises the amino acid sequence set forth in SEQ ID NO: 20.

86. The method according to claim 84, wherein the signal peptide is a TorT signal peptide and wherein the TorT signal peptide comprises the amino acid sequence set forth in SEQ ID NO: 21.

87. The method according to claim 84, wherein the signal peptide is a TolB signal peptide and wherein the TolB signal peptide comprises the amino acid sequence set forth in SEQ ID NO: 22.

88. The method according to claim 84, wherein the signal peptide is a Sfm signal peptide and wherein the Sfm signal peptide comprises the amino acid sequence set forth in SEQ ID NO: 23.

89. The method according to any one of claims 79 to 82, the signal peptide directs the fusion protein to a general secretory (SEC) pathway.

90. The method according to claim 89, wherein the signal peptide is a Lam signal peptide, a MalE signal peptide, a MglB signal peptide, a OmpA signal peptide, or a Pel signal peptide.

91. The method according to claim 90, wherein the signal peptide is a Lam signal peptide and wherein the Lam signal peptide comprises the amino acid sequence set forth in SEQ ID NO: 24.

92. The method according to claim 90, wherein the signal peptide is a MalE signal peptide and wherein the MalE signal peptide comprises the amino acid sequence set forth in SEQ ID NO: 25.

93. The method according to claim 90, wherein the signal peptide is a MglB signal peptide and wherein the MglB signal peptide comprises the amino acid sequence set forth in SEQ ID NO: 26.

94. The method according to claim 90, wherein the signal peptide is an OmpA signal peptide and wherein the OmpA signal peptide comprises the amino acid sequence set forth in SEQ ID NO: 27.

95. The method according to claim 90, wherein the signal peptide is a PelB signal peptide and wherein the PelB signal peptide comprises the amino acid sequence set forth in SEQ ID NO: 31.

96. The method according to any one of claims 79 to 82, wherein the signal peptide directs the fusion protein to the twin-arginine translocation (TAT) pathway.

97. The method according to claims 69 to 78, wherein the bacteriophage is T phage.

98. The method according to claim 97, wherein the T phage is T3.

99. The method according to claim 97, wherein the T phage is T4.

100. The method according to claim 97, wherein the T phage is T7.

101. The method according to any one of claims 1 to 69, wherein the non-biotinylated members are produced for in vitro display method of the fusion proteins on the scaffolds.

102. The method according to claim 101, wherein the scaffold is a ribosome.

103. The method according to claim 101, wherein the scaffold is a RepA protein.

104. The method according to claim 101, wherein the scaffold is a DNA puromycin linker.

105. The method according to any one of claims 1 to 104, wherein the fusion protein further comprises a moiety that interacts with a surface bound protein of the host cells, wherein the interaction between the moiety and the surface bound protein induces binding of at least the fusion protein to the host cell and/or induces cellular uptake of at least the fusion protein.

106. The method according to any one of claims 1 to 104, wherein the fusion protein further comprises a moiety that interacts with a polysaccharide displayed on a surface of the host cells, wherein the interaction between the moiety and the polysaccharide induces binding of at least the fusion protein to the host cell and/or induces cellular uptake of at least the fusion protein.

107. The method according to any one of claims 1 to 104, wherein the fusion protein further comprises a moiety that directs targeting of the member to a specific cell type.

108. The method according to any one of claims 1 to 104, wherein the fusion protein further comprises a moiety capable of inducing a phenotype upon entry into the host cell.

109. The method according to claim 108, wherein the phenotype is a lethal phenotype.

110. The method according to claim 108, wherein the moiety is shepherdin.

111. The method according any one of claims 1 to 110, wherein determining or identifying a candidate peptide moiety at step (iii) comprises contacting the host cell or cell lysate or extract thereof with a biotin-binding molecule attached to a solid support for a time and under conditions sufficient for binding of the biotinylated fusion protein to the biotin binding molecule and recovering the biotinylated fusion protein.

112. The method according to claim 111, wherein the biotin-binding molecule comprises avidin or neutravidin or streptavidin or a variant thereof.

113. The method according to claim 111 or 112, wherein the solid support is in the form of a bead, column, membrane, microwell or centrifuge tube.

114. The method according to claim 113, wherein the solid support is a bead and wherein the bead is a glass bead, or microbead, magnetic bead, or paramagnetic bead.

115. A method of identifying a cell penetrating peptide capable of transporting a cargo moiety to a subcellular location, the method comprising the steps:
(a) performing the method of according to any one of claims 1 to 114 to determine or identify a candidate peptide moiety that has translocated the cell membrane;
(b) recovering at least a biotinylated fusion protein comprising a peptide capable of translocating a cell membrane;
(c) obtaining a nucleic acid sequence encoding at least the peptide of the recovered biotinylated fusion protein;
(d) producing the peptide; and
(e) performing a functional assay to determine the ability of the peptide to translocate a cargo moiety to a subcellular location of a cell.

116. The method according to claim 116, wherein the functional assay comprises:
(f) contacting test cells with a toxin conjugate, wherein the toxin conjugate comprises
the peptide linked to a cargo comprising a toxin or catalytic subunit thereof, and wherein said contacting is for a time and under conditions sufficient for toxin conjugates to enter the test cells;
(g) incubating the test cells for a time and under conditions sufficient for toxin conjugates to reduce viability of the test cells;
(h) detecting reduced viability of the test cells, wherein reduced viability of the test cells indicates that the peptide has translocated the toxin or catalytic subunit to a subcellular location of the cell.

117. The method according to claim 116, wherein the toxin conjugate is lethal to the test cells.

118. The method according to claim 117, wherein detecting expression of a toxin conjugate comprises performing fluorescence-activated cell sorting.

119. The method according to any one of claims 116 to 118, wherein the toxin comprises a Diphtheria toxin fragment A.

120. The method according to any one of claims 116 to 118, wherein the toxin comprises a Cholera toxin subunit A1.

121. The method according to any one of claims 116 to 118, wherein the toxin is a Pseudomonas exotoxin.

122. The method according to any one of claims 116 to 118, wherein the toxin comprises a ribosome inactivating protein.

123. The method according to claim 122, wherein the ribosome inactivating protein is a type I ribosome inactivating protein.

124. The method according to claim 123, wherein type I ribosome inactivating protein is bargaining.

125. The method according to claim 123, wherein type I ribosome inactivating protein is gelonin.

126. The method according to claim 123, wherein type I ribosome inactivating protein is saporin.

127. The method according to claim 122, wherein the ribosome inactivating protein is a type II ribosome inactivating protein.

128. The method according to claim 127, wherein the type II ribosome inactivating protein is a fragment A1 of the Shiga toxin.

129. The method according to claim 127, wherein the type II ribosome inactivating protein is ricin.

130. The method according to claim 127, wherein the type II ribosome inactivating protein is abrin.

131. The method according to claim 127, wherein the type II ribosome inactivating protein is nigrin.

132. The method according to claim 122, wherein the ribosome inactivating protein is a type III ribosome inactivating protein.

133. The method according to any one of claims 116 to 127, further comprising producing the toxin conjugate.

134. The method according to claim 115, wherein the functional assay comprises
(f) expressing a first moiety in a test cell, the first moiety comprising a first fragment of a detectable molecule;
(g) contacting the test cell with a second moiety comprising the peptide linked to a cargo moiety comprising a second fragment of the detectable molecule for a time and under conditions sufficient for binding of the second moiety to the test cell and uptake of the second moiety by the test cell;
(h) incubating the test cells for a time and under conditions sufficient for the first moiety and second moiety to constitute the detectable molecule or produce an activity of the detectable moiety; and
(i) detecting the detectable molecule in the test cell, wherein said detection indicates that the peptide has translocated the second fragment to a subcellular location of the test cell.

135. The method according to claim 134, wherein the constituted detectable molecule is a fluorescent molecule.

136. The method according to claim 135, wherein the fluorescent protein is a green fluorescent protein.

137. The method according to claim 136, wherein a fragment of the detectable molecule comprises an amino acid sequence comprising a GFP 11 tag and a fragment of the detectable molecule comprises an amino acid sequence comprising a GFP 1-10 detector.

138. The method according to claim 137, wherein the GFP 11 tag comprises an amino acid sequence set forth in SEQ ID NO: 81.

139. The method according to claim 136 or 137, wherein the GFP 11 tag is linked to a nucleic acid encoding a scaffold molecule.

140. The method according to claim 139, wherein the scaffold molecule comprises a small ubiquitin-related modifier peptide or a tubulin peptide or a β-actin peptide or a centyrin or Mal or Sumo or MyD88.

141. The method according to claims 137 to 140, wherein the GFP 1-10 detector comprises an amino acid sequence set forth in SEQ ID NO: 86.

142. The method according to claim 115, wherein the functional assay comprises:
(f) contacting test cells comprising fibroblasts with a fusion protein comprising the peptide and a transcription factor that is functional in a subcellular localisation of the cell and mediates differentiation of the fibroblasts to a different cell type;
(g) incubating the test cells for a time and under conditions sufficient for their differentiation to occur; and
(h) detecting the differentiated cells, wherein the differentiated cells indicate that the peptide has translocated the transcription factor to a subcellular location of the test cells.

143. The method according to claim 142, wherein the transcription factor is OCT-4 and wherein the differentiation cells are lymphocytes.

144. The method according to claim 142, wherein the transcription factor is MYOD1 and wherein the differentiation cells are myoblasts.

145. The method according to any one of claims 142 to 144, wherein the fibroblasts are primary fibroblasts of human origin.

146. The method according to any one of claims 142 to 145, wherein the differentiated cells are detected by microscopy or fluorescence-activated cell sorting (FACS).
